(19) United States
(12) Patent Application Publication
(10) Pub. No.: US 2026/0258027 A1
(43) Pub. Date: Sep. 3, 2026

(54) 5- SUBSTITUTED FUSED BICYCLIC HETEROARYL DERIVATIVES CONTAINING ACRYLAMIDE, COMPOSITIONS THEREOF, AND USES THEREOF

(71) Applicant: BeOne Medicines I GmbH, Basel (CH)

(72) Inventors: Qiuwen WANG, Beijing (CN); Yunhang GUO, Beijing (CN); Zhiwei WANG, Beijing (CN)

(73) Assignee: BeOne Medicines I GmbH, Basel (CH)

(21) Appl. No.: 19/550,952

(22) Filed: Feb. 26, 2026

(30) Foreign Application Priority Data
Feb. 27, 2025 (WO) PCT/CN2025/079488
Jun. 10, 2025 (WO) PCT/CN2025/100142
Oct. 16, 2025 (WO) PCT/CN2025/128102
Jan. 15, 2026 (WO) PCT/CN2026/072849

(51) Int. Cl. C07D 413/14; A61K 31/423; A61K 31/501; A61K 31/5377; A61K 31/5386; C07D 405/14; C07D 417/14

(52) U.S. Cl. CPC C07D 413/14; A61K 31/423; A61K 31/501; A61K 31/5377; A61K 31/5386; C07D 405/14; C07D 417/14

(57) ABSTRACT

Compounds of Formula (I) or Formula (II) for enhancing the binding of KEAP1 to CUL3 and their uses.

### CROSS-REFERENCE TO RELATED APPLICATIONS

This application claims priority from International Patent Application Nos. PCT/CN2025/079488, filed Feb. 27, 2025, PCT/CN2025/100142, filed Jun. 10, 2025, PCT/CN2025/128102, filed Oct. 16, 2025, and PCT/CN2026/072849, filed Jan. 15, 2026. The contents of these applications are incorporated herein by reference in their entirety.

### FIELD

Provided herein are compounds that can modulate the binding of Kelch-like ECH-associated protein 1 (KEAP1) to Cullin 3 (CUL3)-based E3 ubiquitin ligase as well as their pharmaceutical compositions and methods of use.

### BACKGROUND

NF-E2-related factor 2 (NRF2) is a transcription factor that can regulate genes involved in oxidative stress response, detoxification, metabolism, and inflammation. Abnormal NRF2 activation can enhance the survival and proliferation of cancer cells. Uncontrolled NRF2 activity grants cancer cells (such as lung cancers) significant resistance to anticancer drugs and reactive oxygen species (ROS), while also steering them towards metabolic reprogramming.

The level of NRF2 can be controlled by the ubiquitination degradation pathway through the complexes formed by KEAP1 and Cullin 3 (CUL3)-based E3 ubiquitin ligase. KEAP1-CUL3 complexes function as NRF2-specific E3 ubiquitin ligases, guiding the polyubiquitination and subsequent degradation of NRF2 through the 26S proteasome.

Research has shown that KEAP1 can act as an adaptor that bridges NRF2 to the CUL3-based E3 ubiquitin ligase. Therefore, the KEAP1-NRF2 system has been investigated as a potential therapeutic target in cancer treatment. Compounds that mediate the binding activity of KEAP1 to CUL3, for example, KEAP1 activators enhancing the binding of KEAP1 to CUL3, can therefore have therapeutic effects in cancer patients with high NRF2 activity.

### SUMMARY

Provided are compounds that can enhance the binding of KEAP1 to CUL3, which is a compound of Formula (I) or Formula (II):

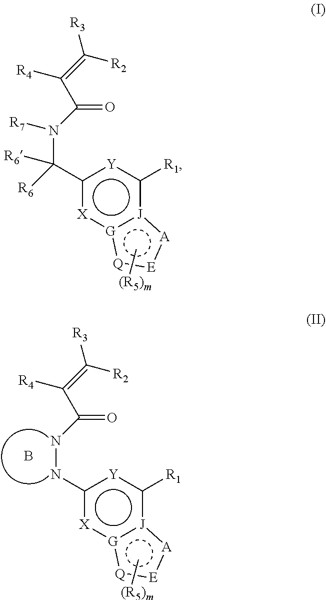

or a pharmaceutically acceptable salt thereof, or a hydrate thereof, or a solvate thereof, wherein ring B, R₁, R₂, R₃, R₄, R₅, R₆, R₆′, R₇, A, E, Q, G, J, X, Y, and m are as defined herein.

Also provided herein is a pharmaceutical composition comprising a compound as provided herein or a pharmaceutically acceptable salt thereof, or a hydrate thereof, or a solvate thereof, and a pharmaceutically acceptable excipient.

Also provided is a method of inhibiting or degrading NRF2 by mediating the activation of KEAP1, comprising administering to a subject in need thereof a therapeutically effective amount of a compound as provided herein or a pharmaceutically acceptable salt thereof, or a hydrate thereof, or a solvate thereof.

Also provided herein is a method of treating a disease that is treatable by inhibition or degradation of NRF2, comprising administering to a subject in need thereof a therapeutically effective amount of a compound as provided herein or a pharmaceutically acceptable salt thereof, or a hydrate thereof, or a solvate thereof.

Also provided is a compound as described herein for use as a medicament.

### DETAILED DESCRIPTION

### I. Definitions

Unless specifically defined elsewhere in this document, all other technical and scientific terms used herein have the meaning commonly understood by one of ordinary skill in the art.

It is meant to be understood that proper valences are maintained for all moieties and combinations thereof, that monovalent moieties having more than one atom are drawn from left to right and are attached through their left ends.

It is also meant to be understood that a specific embodiment of a variable moiety may be the same or different as another specific embodiment having the same identifier.

The indefinite articles “a” and “an” and the definite article “the” include plural as well as single referents, unless the context clearly indicates otherwise.

“Alkyl” means a saturated straight or branched hydrocarbon chain having from 1 to 12 carbon atoms, typically from 1 to 8 carbon atoms, for example from 1 to 6 carbon atoms, and further for example from 1 to 4 carbon atoms. Representative alkyl groups include, but are not limited to, -methyl, -ethyl, -n-propyl, -n-butyl, -n-pentyl, -n-hexyl, -isopropyl, -sec-butyl, -isobutyl, tert-butyl, -isopentyl, - neopentyl, tert-pentyl, -2methylpentyl, -3methylpentyl, 4methylpentyl, 2,3dimethylbutyl and the like.

“Alkoxy” or “alkoxyl” means —O-alkyl, wherein alkyl is defined herein.

“Bridged ring” means a ring system formed by connecting two non-adjacent atoms (usually carbon atoms) of a ring (mono- or fused multi cyclic) with an atom or chain of atoms. These two non-adjacent atoms are called “bridgeheads”. A bridged ring system may include only carbon atoms as ring atoms or may include carbon atoms and one to four heteroatoms (i.e., N, O, or S) as ring atoms. Examples of bridged ring systems include bicyclo[1.1.1]pentyl, bicyclo[2.1.1]hexyl, bicyclo[2.2.1]heptyl, bicyclo[2.2.2]octyl, adamantanyl, norbomanyl, bicyclo [3.2.1] octyl, bicyclo [2.2.2] octyl, bicyclo[3.3.1] nonyl, bicyclo [3.2.3] nonyl, 2-oxa-bicyclo [2.2.2] octyl, 1-aza-bicyclo [2.2.2] octyl, 3-aza-bicyclo [3.2.1] octyl, and 2, 6-dioxa-tricyclo [3.3.1.03,7] nonyl.

“Cycloalkyl” means a saturated monocyclic or multiple fused ring system having from 3 to 14 ring carbon atoms and no heteroatoms. Typically, the cycloalkyl group is a monocyclic or bicyclic group having 3 to 10 ring carbon atoms, for example, a monocyclic group having 3 to 8 ring carbon atoms, further for example a monocyclic group having 3 to 6 ring carbon atoms, and even further for example a monocyclic group having 5 to 6 ring carbon atoms. Cycloalkyl further can be a bridged or spiro ring system. Depending on the structure, a cycloalkyl group can be monovalent, divalent (i.e., cycloalkylene), or multivalent. Representative cycloalkyl groups include, but are not limited to cyclopropyl, cyclobutyl, cyclopentyl, cyclohexyl, cycloheptyl, cyclooctyl, bicyclo[1.1.1]pentyl, bicyclo[2.1.1]hexyl, bicyclo[2.2.1]heptyl, bicyclo[2.2.2]octyl, spiro[3.5]nonyl, and spiro[3.3]heptyl.

“Halogen” or “halo” means fluorine, chlorine, bromine or iodine.

“Heteroaryl” means an aromatic monocyclic or aromatic multiple fused ring system in which at least one, typically one to four, for example one to three, further for example one to two, or even further for example one of the ring atoms are independently selected from the group consisting of O, S and N. Typically, the heteroaryl group is a monocyclic or bicyclic ring system having 5 to 14 ring atoms, for example a monocyclic or bicyclic ring system having 5 to 10 ring atoms, and further for example a monocyclic ring having 5 to 6 ring atoms. Heteroaryl groups can be bonded to other groups at any ring atom (i.e., at any carbon atom or heteroatom of the heteroaryl ring system), provided that proper valences are maintained. Depending on the structure, a heteroaryl group can be monovalent, divalent (i.e., heteroarylene), or multivalent. Representative heteroaryl groups include, but are not limited to, pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indolyl-2-onyl or isoindolin-1-onyl), azaindolyl (pyrrolopyridyl or 1H-pyrrolo[2,3-b]pyridyl), indazolyl, benzimidazolyl (e.g., 1Hbenzo[d]imidazolyl), imidazopyridyl (e.g., azabenzimidazolyl or 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, purinyl, quinolinyl, isoquinolinyl, quinoxalinyl, and quinazolinyl groups.

“Heterocyclyl” means a non-aromatic monocyclic or multiple fused ring system in which at least one, for example one to four, further for example one to three, or even further for example one to two of the ring carbon atoms are independently replaced with a heteroatom selected from the group consisting of O, S and N, wherein the nitrogen and sulfur heteroatoms may optionally be oxidized and/or the nitrogen heteroatom may be optionally be quarternized. The heterocyclyl group can be saturated or unsaturated. Typically, the heterocyclyl group is a monocyclic or bicyclic ring system having 3 to 14 ring atoms, for example a monocyclic or bicyclic ring system having 3 to 10 ring atoms, further for example a monocyclic ring having 3 to 6 ring atoms and even further for example a monocyclic ring having 5 to 6 ring atoms. For clarity, if a fused ring system contains a ring that is heterocyclyl, or if a fused ring system contains a ring that is heteroaryl and a ring that is both non-aromatic and only has carbon as ring atoms, this fused ring system is considered as heterocyclyl. Heterocyclyl further can be a bridged or spiro ring system. Depending on the structure, a heterocyclyl group can be monovalent, divalent (i.e., heterocyclylene), or multivalent. The heterocyclyl group can be bonded to other groups at any ring atom (i.e., at any carbon atom or heteroatom of the heterocyclic ring system), provided that proper valences are maintained. Representative heterocyclyl groups include, but are not limited to, aziridinyl, azetidinyl, azepanyl, oxetanyl (or oxetyl), pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, dioxolyl, pyrrolinyl, imidazolinyl, pyrazolinyl, thiazolinyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dioxyl, dithianyl, pyranyl, dihydropyridyl, dihydrodithiinyl, dihydrodithionyl, 1,4-dioxaspiro[4.5]decanyl, 2-oxo-1-oxa-3,8-diazaspiro[4.5]decyl, 1-oxo-2,8-diazaspiro[4.5]decyl, 3-oxo-2,8-diazaspiro[4.5]decyl, 3-oxo-1-oxa-4,9-diazaspiro[5.5]undecyl, 2-oxo-1-oxa-3,9-diazaspiro[5.5]undecyl, homopiperazinyl, quinuclidyl, indolinyl, isoindolinyl, quinolizinyl, dihydrobenzothiazinyl, dihydrobenzofuranyl, dihydroindolyl, dihydrobenzodioxinyl, tetrahydroquinolinyl groups, 2-oxa-bicyclo [2.2.2] octyl, and 1-aza-bicyclo [2.2.2] octyl.

“Hydrate” means a solvate wherein the solvent molecule is H₂O.

“Oxo” means ═O wherein the double bond is attached to a carbon, sulfur, or nitrogen.

“Pharmaceutically acceptable” means suitable for use in contact with the tissues of human beings without excessive toxicity, irritation, allergic response, or other problem or complication, and commensurate with a reasonable benefit/risk ratio.

“Pharmaceutically acceptable excipient” means a pharmaceutically acceptable inert ingredient or vehicle, such as a liquid or solid filler, diluent, excipient, solvent or encapsulating material. Each excipient must be “acceptable” in the sense of being compatible with the other ingredients of the formulation and not deleterious to the recipients thereof.

“Pharmaceutically acceptable salt(s)” means a salt prepared from a pharmaceutically acceptable non-toxic acid or base including an inorganic acid and base and an organic acid and base.

“Solvate” means a physical association of a compound of Formula (I) or Formula (II) or a pharmaceutically acceptable salt, hydrate, or solvate thereof with one or more pharmaceutically acceptable solvent molecules. This physical association involves varying degrees of ionic and non-covalent bonding, including hydrogen bonding. In certain instances, one or more solvent molecules are incorporated in the crystal lattice of the crystalline solid.

“Spiro ring” means a ring system that contains a bicyclic ring sharing a single ring atom (usually a quaternary carbon atom) between two ring members. Typically the spiro ring is a bicyclic ring system. The individual rings within a spiro ring system may be identical or different, may contain carbon atoms only as ring atoms, or may contain one to four heteroatoms as ring atom(s). Examples of spiro ring systems include spiropentane, spirohexane, spiro[5.4]decane, spiro[4.3]octane, spiro[5.2]octane, and spiropyran.

“Stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound.

“Subject” means a human.

“Substituted” or “substitute” means that any one or more hydrogens on the designated atom or group is replaced with a selection (i.e., substituent) from the indicated groups, provided that the designated atom's normal valence is not exceeded. It will be understood by those skilled in the art, with respect to any group containing one or more substituents, that such groups are not intended to introduce any substitution or substitution patterns that are sterically impractical, synthetically non-feasible and/or inherently unstable. For example, the substituents on a heterocyclyl are not expected to be a moiety having halogen, CN, oxygen (O), nitrogen (N) or sulfur (S) bonded to a ring heteroatom (O, N, or S) of the heterocyclyl or single bonded to a ring carbon next to a ring heteroatom of the heterocycyl. Further for example, a chain carbon atom is not expected to be single bonded to two heteroatoms (O, N, or S) unless the carbon is also double bonded to a heteroatom.

“Tautomer” means an isomeric form of a compound that is in equilibrium with another isomeric form. The concentration of the isomeric form will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

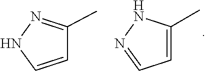

“Therapeutically effective amount” means an amount capable of treating a disorder, disease or condition, or symptoms thereof, disclosed herein.

“Treating” means alleviating, in whole or in part, of a disorder, disease or condition, or one or more of the symptoms associated with a disorder, disease, or condition, or slowing or halting of further progression or worsening of those symptoms, or alleviating the cause(s) of the disorder, disease, or condition itself.

### II. Compounds

In one aspect, provided are compounds of Formula (I). In another aspect, provided are compounds of Formula (II). Compounds of both formulas have the potential to degrade or inhibit NRF2 by enhancing the binding of KEAP1 to CUL3, hence can be used to treat a disease that can be treated through inhibition or degradation of NRF2. It is believed that the compounds provided herein can enhance the binding of KEAP1 to CUL3 by forming a covalent bond with a cysteine of the KEAP1.

These compounds can be administered to a subject in need thereof by way of a pharmaceutical composition which further comprises a pharmaceutically acceptable excipient. Suitable pharmaceutical excipients can be found in the literature and their formulations can be prepared by any of the methods known in the art.

The following embodiments of the above aspects are not intended to be an explicit or implicit admission that these aspects or embodiments are independent or distinct nor should it be interpreted as such. Rather, it is intended to convey information so that the full breadth of the present disclosure can be understood. Furthermore, the following embodiments are not meant to be limiting on the full breadth of the disclosure as recited herein. More specifically, the embodiments are numbered for convenience and clarity of reference, intending to provide every combination of technically compatible embodiments, even if they are not expressly disclosed in combination or linked to each other.

1. A compound of Formula (I),

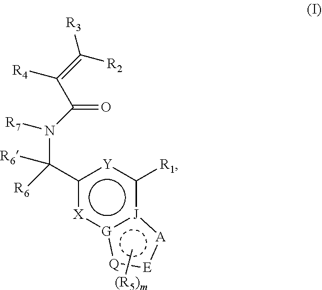

or a pharmaceutically acceptable salt thereof, or a hydrate thereof, or a solvate thereof,
wherein
each of A, E, and Q is independently O, S, N, NH, CH₂, or CH (for clarification, if R₅ substitutes at the corresponding position, NH is N, CH₂ is CH or C, and CH is C. In other words, the options for A, E, and Q as listed here do not take substitutions into consideration),
each of G and J is independently C or N,
X is N or CR₁″,
Y is N or CR₁′, provided that the ring formed by A, E, Q, G, and J is heterocyclyl or heteroaryl, and the ring formed by J, G, X, Y and the two carbons to which Y is aromatic,
R₁, R₁′, and R₁″ are independently H, halogen, CN, C₁₋₆ alkoxyl, C₃₋₈ cycloalkyl, —O—C₃₋₈ cycloalkyl, —OH, —O-4 to 7 membered heterocyclyl, or C₁₋₆ alkyl, wherein each of C₁₋₆ alkoxyl, C₃₋₈ cycloalkyl, —O—C₃₋₈ cycloalkyl, —O-4 to 7 membered heterocyclyl, and C₁₋₆ alkyl is optionally substituted with one or more groups independently selected from halogen, C₁₋₆ alkoxyl, CN, NH₂, NHC₁₋₆ alkyl, N(C₁₋₆ alkyl)₂, OH, —O—C₃₋₈ cycloalkyl, and 4 to 7 membered heterocyclyl that is optionally substituted with one or two groups independently selected from C₁₋₆ alkyl and halogen,
R₂ and R₃ are H,
R₄ is H, F, Cl, CN, NO₂, or C₁₋₆ alkyl substituted with one or more halogen,
R₅ substitutes at A, E, and/or Q, each R₅ is independently (1) oxo or C₁₋₆ alkyl optionally substituted with one or more groups independently selected from halogen, C₁₋₆ alkoxyl, OH, NH₂, NHC₁₋₆ alkyl, N(C₁₋₆ alkyl)₂, and CN, (2) CONR₅ₐR_{5b}, (3) 5-10 membered heteroaryl optionally substituted with one or more R_{5c} independently, (4) C₃₋₈ cycloalkyl optionally substituted with one or more R_{5c}′ independently, or (5) 4-10 membered heterocyclyl optionally substituted with one or more R_{5c}″ independently,
R_{5c}, R_{5c}′, and R_{5c}″ are independently C₁₋₆ alkyl, C₁₋₆ alkoxyl, oxo, CN, OH, halogen, CONR_{5d}R₅ₑ, NHCONR_{5d}R₅ₑ, SO₂NR_{5d}R₅ₑ, SO(NR_{5f})C₁₋₆alkyl, NR_{5d}R₅ₑ, C₃₋₈ cycloalkyl, 5-10 membered heteroaryl, or 4-10 membered heterocyclyl, wherein each of C₁₋₆ alkoxyl, C₁₋₆ alkyl, C₃₋₈ cycloalkyl, 5-10 membered heteroaryl, and 4-10 membered heterocyclyl is optionally substituted with one or more groups independently selected from halogen, C₁₋₆ alkyl, C₁₋₆ alkoxyl, CN, NH₂, NHC₁₋₆ alkyl, N(C₁₋₆ alkyl)₂, oxo, and OH,
R_{5f} is H, C₁₋₆ alkyl, or C₃₋₈ cycloalkyl,
m is 0, 1, or 2,
R₆ is H, C₁₋₆ alkyl, 5-10 membered heteroaryl, —CONR₆ₐR_{6b}, or CN, wherein (1) the C₁₋₆ alkyl is optionally substituted with one or more groups independently selected from halogen, C₁₋₆ alkoxyl, SO₂C₁₋₆ alkyl, CN, NH₂, NHC₁₋₆alkyl, N(C₁₋₆alkyl)₂, and OH, and (2) the 5-10 membered heteroaryl is optionally substituted with one or more groups independently selected from halogen, C₁₋₆ alkoxyl, CN, and C₁₋₆ alkyl,
R₆′ is H,
R₇ is H or C₁₋₆ alkyl optionally independently substituted with one or more groups independently selected from halogen, C₁₋₆ alkoxyl, CN, NH₂, NHC₁₋₆ alkyl, N(C₁₋₆ alkyl)₂, and OH, or R₆′ is H, R₆ and R₇, together with the atoms to which they are attached, form a 4-10 membered nitrogen-containing heterocyclyl comprising additional 0-2 heteroatoms selected from O and N as ring atoms, wherein the 4-10 membered nitrogen-containing heterocyclyl is optionally substituted with one or more groups independently selected from halogen, oxo, CN, and -L-W, wherein L is a bond, CO, or —NHCO, W is a C₁₋₆ alkyl, C₃₋₈ cycloalkyl, 4-10 membered heterocyclyl, or 5-10 membered heteroaryl, and wherein the C₁₋₆ alkyl optionally has one CH2 replaced with O, NH, or NC₁₋₆ alkyl, each of the C₃₋₈ cycloalkyl, 4-10 membered heterocyclyl, and 5-10 membered heteroaryl is optionally substituted with one or more groups independently selected from halogen, C₁₋₆ alkyl, CN, and C₁₋₆ alkoxy,
R₅ₐ, R_{5b}, R₅ₐ, R₅ₑ, R₆ₐ, and R_{6b} are independently H, SO₂C₁₋₆ alkyl, OH, C₁₋₆ alkoxy, or C₁₋₆ alkyl optionally substituted with one or more groups independently selected from halogen, C₁₋₆ alkoxyl, CN, NH₂, NHC₁₋₆alkyl, N(C₁₋₆alkyl)₂, and OH,
or R₅ₐ and R_{5b}, R₅ₐ and R₅ₑ, and R₆ₐ and R_{6b}, together with the nitrogen to which they are attached independently form a 4-6 membered heterocyclyl optionally substituted with one or more groups independently selected from halogen, CN, OH, C₁₋₆ alkoxyl, or C₁₋₆ alkyl, wherein each of C₁₋₆ alkoxyl and C₁₋₆ alkyl is optionally independently substituted with one or more groups independently selected from halogen, C₁₋₆alkoxyl, CN, NH₂, NHC₁₋₆alkyl, N(C₁₋₆alkyl)₂, and OH.

2. The compound of Embodiment 1, or a pharmaceutically acceptable salt thereof, or a hydrate thereof, or a solvate thereof, wherein R₆′ is H, R₆ and R₇, together with the atoms to which they are attached, form a 4-10 membered nitrogen-containing heterocyclyl comprising additional 0-2 heteroatoms selected from O and N as ring atoms, wherein the 4-10 membered nitrogen-containing heterocyclyl is mono- or bi-cyclic and is optionally substituted with one or more groups independently selected from CN, halogen, and -L-W, wherein L is a bond, CO, or —NHCO, W is a C₁₋₆ alkyl, C₃₋₈ cycloalkyl, 4-10 membered heterocyclyl, or 5-10 membered heteroaryl, and wherein the C₁₋₆ alkyl optionally has one CH₂ replaced with O, NH, or NC₁₋₆ alkyl, each of the C₃₋₈ cycloalkyl, 4-10 membered heterocyclyl, and 5-10 membered heteroaryl is optionally substituted with one or more groups independently selected from halogen, C₁₋₆ alkyl, CN, and C₁₋₆ alkoxy.

3. The compound of Embodiment 1, or a pharmaceutically acceptable salt thereof, or a hydrate thereof, or a solvate thereof, wherein R₆′ is H, R₆ and R₇, together with the atoms to which they are attached, form a morpholine, piperidine, 4,5-dihydro-1-yl-pyrazole, piperazine, pyrrolidine, 1,4-oxazepane, 5,6,7,8-tetrahydroimidazo[1,2-a]pyrazine, 2-oxa-6-azaspiro[3.5]nonane, 2-oxa-7-azaspiro[3.5]nonane, 6-azaspiro[3.5]nonane, 7-azaspiro[3.5]nonane, 1-oxa-8-azaspiro[4.5]decane, 2-oxa-8-azaspiro[4.5]decane, 1-oxa-7-azaspiro[4.5]decane, 2-oxa-7-azaspiro[4.5]decane, 4-oxa-7-azaspiro[2.5]octane, 7-oxa-4-azaspiro[2.5]octane, 2,5-dioxa-8-azaspiro[3.5]nonane, or 4,5,6,7-tetrahydro-3H-imidazo[4,5-c]pyridine, each of which is optionally substituted with one or more groups independently selected from -L-W, wherein L is a bond, CO, or —NHCO, W is a C₁₋₆ alkyl, C₃₋₈ cycloalkyl, 4-10 membered heterocyclyl, or 5-10 membered heteroaryl, and wherein the C₁₋₆ alkyl optionally has one CH2 replaced with O, NH, or NC₁₋₆alkyl, each of the C₃₋₈ cycloalkyl, 4-10 membered heterocyclyl, and 5-10 membered heteroaryl is optionally substituted with one or more groups independently selected from halogen, C₁₋₆alkyl, CN, and C₁₋₆alkoxy.

4. The compound of Embodiment 1, or a pharmaceutically acceptable salt thereof, or a hydrate thereof, or a solvate thereof, wherein R₆′ is H, R₆ and R₇, together with the atoms to which they are attached, form a morpholine, piperidine, 4,5-dihydro-1-yl-pyrazole, piperazine, pyrrolidine, 1,4-oxazepane, 5,6,7,8-tetrahydroimidazo[1,2-a]pyrazine, 2-oxa-6-azaspiro[3.5]nonane, 2-oxa-7-azaspiro[3.5]nonane, 6-azaspiro[3.5]nonane, 7-azaspiro[3.5]nonane, 1-oxa-8-azaspiro[4.5]decane, 2-oxa-8-azaspiro[4.5]decane, 1-oxa-7-azaspiro[4.5]decane, 2-oxa-7-azaspiro[4.5]decane, 4-oxa-7-azaspiro[2.5]octane, 7-oxa-4-azaspiro[2.5]octane, 2,5-dioxa-8-azaspiro[3.5]nonane, or 4,5,6,7-tetrahydro- 3H-imidazo[4,5-c]pyridine, each of which is optionally substituted with one or more groups independently selected from C₁₋₆alkyl with one CH₂ being optionally replaced with O, —CO—C₁₋₆ alkyl, —CO—C₃₋₈ cycloalkyl, —CO-4-10 membered heterocyclyl, —CO-5-10 membered heteroaryl, —NHCOC₁₋₆alkyl, 4-10 membered heterocyclyl, and 5-10 membered heteroaryl, each of the C₃₋₈ cycloalkyl, 4-10 membered heterocyclyl, and 5-10 membered heteroaryl, as itself or part of a substituent, is optionally substituted with a C₁₋₆ alkyl or C₁₋₆ alkoxy.

5. The compound of Embodiment 1, or a pharmaceutically acceptable salt thereof, or a hydrate thereof, or a solvate thereof, wherein R₆′ is H, R₆ and R₇, together with the atoms to which they are attached, form a morpholine, piperidine, 4,5-dihydro-1-yl-pyrazole, piperazine, pyrrolidine, 1,4-oxazepane, 5,6,7,8-tetrahydroimidazo[1,2-a]pyrazine, 2-oxa-6-azaspiro[3.5]nonane, 2-oxa-7-azaspiro[3.5]nonane, 6-azaspiro[3.5]nonane, 7-azaspiro[3.5]nonane, 1-oxa-8-azaspiro[4.5]decane, 2-oxa-8-azaspiro[4.5]decane, 1-oxa-7-azaspiro[4.5]decane, 2-oxa-7-azaspiro[4.5]decane, 4-oxa-7-azaspiro[2.5]octane, 7-oxa-4-azaspiro[2.5]octane, 2,5-dioxa-8-azaspiro[3.5]nonane, 5-oxa-8-azaspiro[3.5]nonane, or 4,5,6,7-tetrahydro-3H-imidazo[4,5-c]pyridine, each of which is optionally substituted with one or more groups independently selected from —CH₃, —OCH₃, —CH₂OCH₃, —COCH₃, —CO-cyclopropyl, —CO-oxetyl, —CO-pyrazolyl, —NHCOCH₃, oxetyl, or pyrazolyl, further wherein each of the cyclopropyl, oxetyl, and pyrazolyl, as itself or part of a substituent, is optionally substituted with a C₁₋₆ alkyl or C₁₋₆ alkoxy.

6. The compound of Embodiment 1, or a pharmaceutically acceptable salt thereof, or a hydrate thereof, or a solvate thereof, wherein R₆′ is H, R₆ and R₇, together with the atoms to which they are attached, form a morpholine, piperidine, or piperazine, each of which is optionally substituted with one or two groups independently selected from —CH₃, —OCH₃, —CH₂OCH₃, —COCH₃, —CO-cyclopropyl, —CO-oxetyl, —CO-pyrazolyl, —NHCOCH₃, oxetyl, or pyrazolyl, further wherein each of the cyclopropyl, oxetyl, and pyrazolyl, as itself or part of a substituent, is optionally substituted with a C₁₋₆ alkyl or C₁₋₆ alkoxy.

7. The compound of Embodiment 1, or a pharmaceutically acceptable salt thereof, or a hydrate thereof, or a solvate thereof, wherein R₆′ is H, R₆ and R₇, together with the atoms to which they are attached, form a morpholine optionally substituted with one or two C₁₋₆ alkyl or form a 4-oxa-7-azaspiro[2.5]octane.

8. The compound of Embodiment 1, or a pharmaceutically acceptable salt thereof, or a hydrate thereof, or a solvate thereof, wherein R₆′ is H, R₆ and R₇, together with the atoms to which they are attached, form a

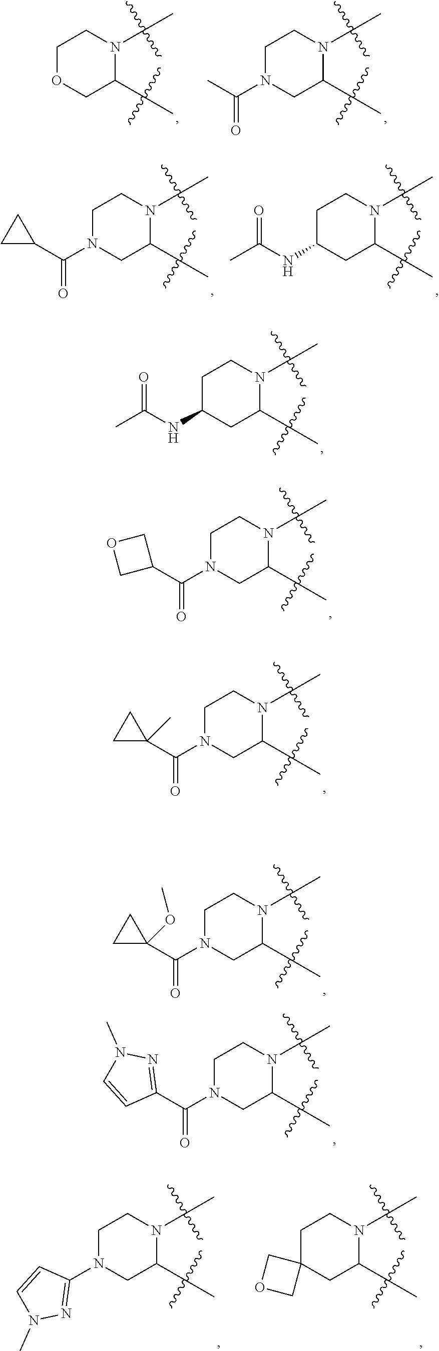

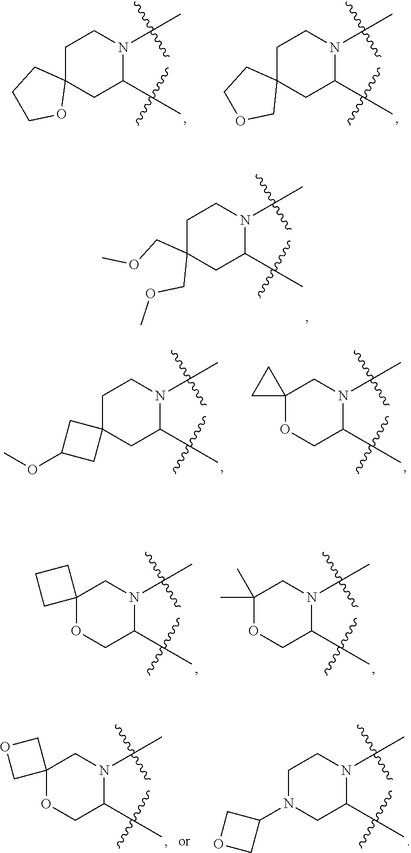

9. The compound of Embodiment 1, or a pharmaceutically acceptable salt thereof, or a hydrate thereof, or a solvate thereof, wherein R₆′ is H, R₇ is H, and R₆ is a 5-10 membered heteroaryl optionally substituted with one or more groups independently selected from halogen, C₁₋₆ alkoxyl, CN, and C₁₋₆ alkyl.

10. The compound of Embodiment 1, or a pharmaceutically acceptable salt thereof, or a hydrate thereof, or a solvate thereof, wherein R₆′ is H, R₇ is H, and R₆ is pyrazolyl optionally substituted with one or more groups independently selected from halogen, C₁₋₆ alkoxyl, CN, and C₁₋₆ alkyl.

11. The compound of any of Embodiments 1-10, or a pharmaceutically acceptable salt thereof, or a hydrate thereof, or a solvate thereof, wherein R₁ is a halogen.

12. The compound of any of Embodiments 1-10, or a pharmaceutically acceptable salt thereof, or a hydrate thereof, or a solvate thereof, wherein R₁ is F or Cl.

13. The compound of any of Embodiments 1-10, or a pharmaceutically acceptable salt thereof, or a hydrate thereof, or a solvate thereof, wherein R₁ is C₁₋₆ alkyl or C₁₋₆ alkoxyl or C₃₋₈ cycloalkyl.

14. The compound of any of Embodiments 1-10, or a pharmaceutically acceptable salt thereof, or a hydrate thereof, or a solvate thereof, wherein R₁ is methyl or methoxyl or cyclopropyl.

15. The compound of any of Embodiments 1-14, or a pharmaceutically acceptable salt thereof, or a hydrate thereof, or a solvate thereof, wherein Y is CR₁′ and R′ is H.

16. The compound of any of Embodiments 1-14, or a pharmaceutically acceptable salt thereof, or a hydrate thereof, or a solvate thereof, wherein Y is N or CR₁′ and R₁′ is a halogen.

17. The compound of any of Embodiments 1-14, or a pharmaceutically acceptable salt thereof, or a hydrate thereof, or a solvate thereof, wherein Y is CR₁′ and R₁′ is F or Cl.

18. The compound of any of Embodiments 1-14, or a pharmaceutically acceptable salt thereof, or a hydrate thereof, or a solvate thereof, wherein Y is CR₁′ and R₁′ is C₁₋₆ alkyl.

19. The compound of any of Embodiments 1-14, or a pharmaceutically acceptable salt thereof, or a hydrate thereof, or a solvate thereof, wherein Y is CR₁′ and R₁′ is methyl.

20. The compound of any of Embodiments 1-19, or a pharmaceutically acceptable salt thereof, or a hydrate thereof, or a solvate thereof, wherein X is CH.

21. The compound of any of Embodiments 1-19, or a pharmaceutically acceptable salt thereof, or a hydrate thereof, or a solvate thereof, wherein X is (1)N, C—CN, C—C₁₋₆alkoxyl, C—C₁₋₆alkyl, C—F, or C—Cl, or (2) N, C—CN, C—OCH₃, C—OCD₃, C—CH₃, C—F, or C—Cl.

22. The compound of any of Embodiments 1-19, or a pharmaceutically acceptable salt thereof, or a hydrate thereof, or a solvate thereof, wherein X is CR₁″, and R₁″ is H, OH, CN, —C₁₋₆ alkyl, halogen, C₁₋₆ alkoxyl, C₃₋₈ cycloalkyl, —O—C₃₋₈ cycloalkyl, or —O-4 to 7 membered heterocyclyl, wherein each of the —C₁₋₆ alkyl, C₁₋₆ alkoxyl, C₃₋₈ cycloalkyl, —O—C₃₋₈ cycloalkyl, and —O-4 to 7 membered heterocyclyl is optionally substituted with —N(C₁₋₆ alkyl)₂, C₁₋₆ alkoxyl, —O—C₃₋₈ cycloalkyl, or 4 to 7 membered heterocyclyl that is optionally substituted with one or two groups independently selected from C₁₋₆alkyl and halogen.

23. The compound of any of Embodiments 1-19, or a pharmaceutically acceptable salt thereof, or a hydrate thereof, or a solvate thereof, wherein X is CR₁″, and R₁″ is H, OH, CN, methyl, ethyl, F, Cl, methoxy, ethoxy, —OCH(CH₃)₂, —OCH₂CH₂CH₃, —OCD₃, —O-cyclobutyl, cyclopropyl, —OCH₂CH₂N(CH₃)₂, —OCH₂CH₂OCH₃,

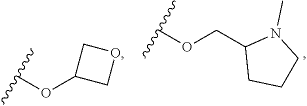

or —OCH₂CH₂O-cyclopropyl.

24. The compound of any of Embodiments 1-19, or a pharmaceutically acceptable salt thereof, or a hydrate thereof, or a solvate thereof, wherein X is N.

25. The compound of any of Embodiments 1-24, or a pharmaceutically acceptable salt thereof, or a hydrate thereof or a solvate thereof, wherein either A or Q is O or S so that

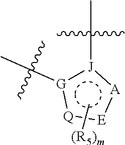

is

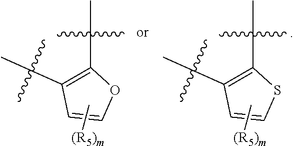

In other words,

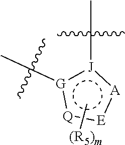

is

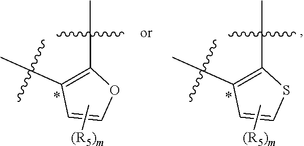

wherein * indicates the position of G or J.

26. The compound of any of Embodiments 1-24, or a pharmaceutically acceptable salt thereof, or a hydrate thereof, or a solvate thereof, wherein

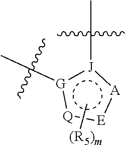

is

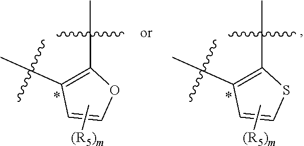

* indicates the position of G.

27. The compound of any of Embodiments 1-24, or a pharmaceutically acceptable salt thereof, or a hydrate thereof, or a solvate thereof, wherein A or Q is O or S so that

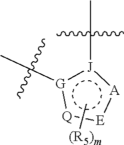

is

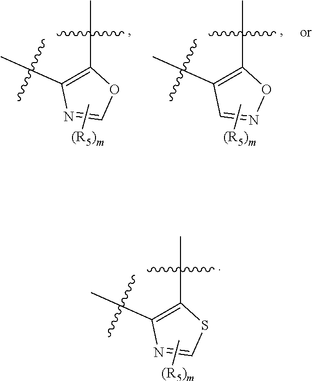

In other words,

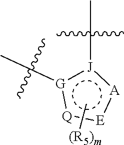

is

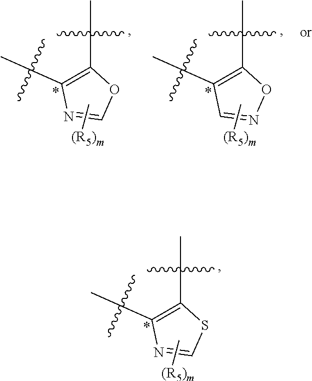

wherein * indicates the position of G or J.

28. The compound of any of Embodiments 1-24, or a pharmaceutically acceptable salt thereof, or a hydrate thereof, or a solvate thereof, wherein G is N so that

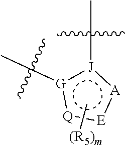

is

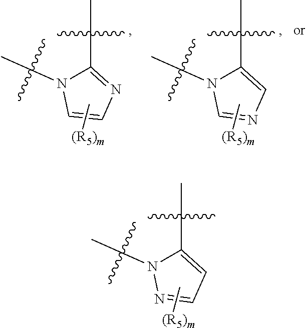

29. The compound of any of Embodiments 1-24, or a pharmaceutically acceptable salt thereof, or a hydrate thereof, or a solvate thereof, wherein J is N so that

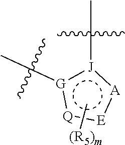

is

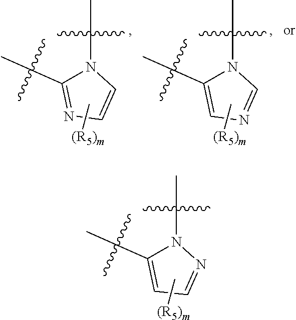

30. The compound of any of Embodiments 1-24, or a pharmaceutically acceptable salt thereof, or a hydrate thereof, or a solvate thereof, wherein either Q or A is N so that

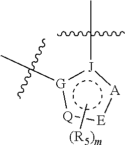

is

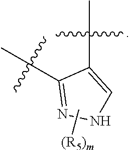

31. The compound of any of Embodiments 1-24, or a pharmaceutically acceptable salt thereof, or a hydrate thereof, or a solvate thereof, wherein

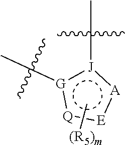

is

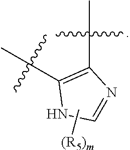

32. The compound of any of Embodiments 1-24, or a pharmaceutically acceptable salt thereof, or a hydrate thereof, or a solvate thereof, wherein

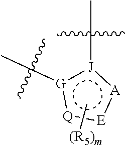

is

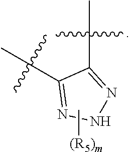

33. The compound of any of Embodiments 1-24, or a pharmaceutically acceptable salt thereof, or a hydrate thereof, or a solvate thereof, wherein

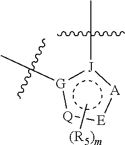

is

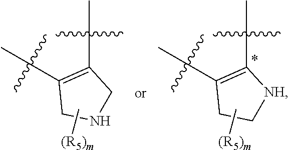

* represents G or J.

34. The compound of any of Embodiments 1-33, or a pharmaceutically acceptable salt thereof, or a hydrate thereof, or a solvate thereof, wherein m is 1, R₅ is a 5-10 membered heteroaryl optionally substituted with one or more R_{5c} independently or is a 4-10 membered heterocyclyl optionally substituted with one or more R_{5c}″ independently, with R_{5c} and R_{5c}″ being the same as in Embodiment 1.

35. The compound of any of Embodiments 1-33, or a pharmaceutically acceptable salt thereof, or a hydrate thereof, or a solvate thereof, wherein m is 1, R₅ is pyridinyl, pyrazinyl, pyridazinyl, pyrimidinyl, 1,3,5-triazinyl, indazolyl, pyrazolyl, triazolyl, thiophenyl, pyrrolyl, imidazolyl, furanyl, isothiazolyl, thiazolyl, pyrrolidinyl, piperidinyl, tetrahydrofuranyl, or 1,2,3,6-tetrahydropyridinyl, wherein each of the pyridinyl, pyrazinyl, pyridazinyl, pyrimidinyl, 1,3,5-triazinyl, indazolyl, pyrazolyl, triazolyl, thiophenyl, pyrrolyl, imidazolyl, furanyl, isothiazolyl, and thiazolyl is optionally substituted with one or more R_{5c} independently, wherein each of the pyrrolidinyl, piperidinyl, tetrahydrofuranyl, and 1,2,3,6 -tetrahydropyridinyl is optionally substituted with one or more R_{5c}″ independently, with R_{5c} and R_{5c}″ being the same as in Embodiment 1.

36. The compound of any of Embodiments 1-33, or a pharmaceutically acceptable salt thereof, or a hydrate thereof, or a solvate thereof, wherein m is 1, R₅ is (1) pyridinyl, pyrazinyl, pyridazinyl, pyrimidinyl, 1,3,5-triazinyl, indazolyl, pyrazolyl, triazolyl, thiophenyl, pyrrolyl, imidazolyl, furanyl, isothiazolyl, or thiazolyl, each of which is optionally substituted with one or more groups independently selected from CONHC₁₋₆ alkoxyl, CONHOH, CONR_{5d}′R₅ₑ′, NR_{5d}′R₅ₑ′, C₁₋₆ alkyl optionally substituted with CN or C₁₋₆ alkoxyl, C₁₋₆ alkoxyl, CHF₂, CF₃, halogen, OH, CN, SO₂NH₂, SO(NH)C₁₋₆ alkyl, SO(NCH₃)C₁₋₆ alkyl, CONHSO₂C₁₋₆ alkyl, NHCONR_{5d}′R₅ₑ′, and 4-6 membered heterocyclyl optionally substituted with C₁₋₆ alkyl, with R_{5d}′ and R₅ₑ′ independently being H or C₁₋₆ alkyl optionally substituted with CN, or R_{5d}′ and R₅ₑ′ together with the nitrogen to which they are attached form a pyrrolidine, piperidine or morpholine, or (2) pyrrolidinyl, piperidinyl, tetrahydrofuranyl, or 1,2,3,6-tetrahydropyridinyl, each of which is optionally substituted with one or more groups independently selected from C₁₋₆ alkyl and CONR_{5d}′R₅ₑ′, with R_{5d}′ and R₅ₑ′ independently being H or C₁₋₆ alkyl optionally substituted with CN, or R_{5d}′ and R₅ₑ′ together with the nitrogen to which they are attached form a pyrrolidine, piperidine or morpholine.

37. The compound of any of Embodiments 1-33, or a pharmaceutically acceptable salt thereof, or a hydrate thereof, or a solvate thereof, wherein m is 1, R₅ is pyridinyl, pyrazolyl, imidazolyl, or triazolyl, each of which is optionally substituted with one or more groups independently selected from C₁₋₆ alkyl optionally substituted with CN or C₁₋₆ alkoxyl, C₁₋₆ alkoxyl, CHF₂, CF₃, halogen, OH, CN, CONR_{5d}′R₅ₑ′, NR_{5d}′R₅ₑ′, and 4-6 membered heterocyclyl optionally substituted with C₁₋₆ alkyl, with R_{5d}′ and R₅ₑ′ independently being H or C₁₋₆ alkyl.

38. The compound of any of Embodiments 1-33, or a pharmaceutically acceptable salt thereof, or a hydrate thereof, or a solvate thereof, wherein m is 1, and R₅ is pyridinyl, pyrazolyl, imidazolyl, or triazolyl, each of which is optionally substituted with one or more groups independently selected from NH₂, methyl, F, Cl, CF₃, methoxyl, —CONH₂, CHF₂, CN, OH, oxetyl optionally substituted with C₁₋₆ alkyl, pyrrolidinyl optionally substituted with C₁₋₆ alkyl, and tetrahydrofuranyl optionally substituted with C₁₋₆ alkyl.

39. The compound of any of Embodiments 1-38, or a pharmaceutically acceptable salt thereof, or a hydrate thereof, or a solvate thereof, wherein m is 1 and R₅ substitutes at E.

40. The compound of any of Embodiments 1-33, or a pharmaceutically acceptable salt thereof, or a hydrate thereof, or a solvate thereof, wherein m is 1, and R₅ is

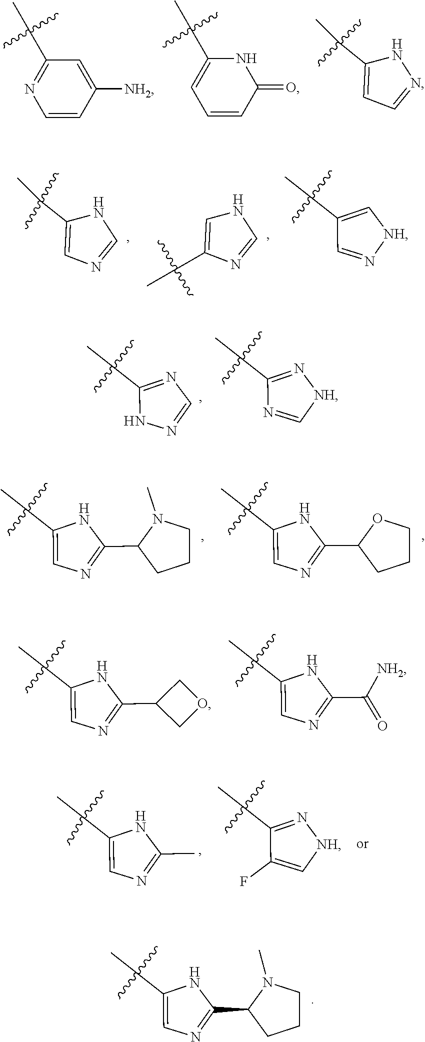

41. The compound of Embodiment 40, or a pharmaceutically acceptable salt thereof, or a hydrate thereof, or a solvate thereof, wherein R₅ substitutes at E.

42. The compound of Embodiment 33, or a pharmaceutically acceptable salt thereof, or a hydrate thereof, or a solvate thereof, wherein m is 2, one R₅ is F, Cl, Br, C₁₋₆alkyl, or oxo (such as when one of A, E, and Q is NH and the ring formed by J, G, Q, E, and A is not aromatic, oxo substituted at the carbon next to the NH), and the other R₅ is pyridinyl, pyrazolyl, imidazolyl, or triazolyl, each of the pyridinyl, pyrazolyl, imidazolyl, and triazolyl is optionally substituted with one or more groups independently selected from NH₂, methyl, F, Cl, CF₃, methoxyl, CHF₂, CN, OH, oxetyl optionally substituted with C₁₋₆alkyl, pyrrolidinyl optionally substituted with C₁₋₆alkyl, and tetrahydrofuranyl optionally substituted with C₁₋₆alkyl.

43. The compound of any of Embodiments 1-42, or a pharmaceutically acceptable salt thereof, or a hydrate thereof, or a solvate thereof, wherein R₄ is H or D.

44. The compound of any of Embodiments 1-42, or a pharmaceutically acceptable salt thereof, or a hydrate thereof, or a solvate thereof, wherein R₄ is F.

45. The compound of any of Embodiments 1-42, or a pharmaceutically acceptable salt thereof, or a hydrate thereof, or a solvate thereof, wherein R₄ is CN or NO₂.

46. The compound of any of Embodiments 1-45, or a pharmaceutically acceptable salt thereof, or a hydrate thereof, or a solvate thereof, wherein the compound has one or more hydrogens replaced by deuterium.

47. The compound of any of Embodiments 1-45, or a pharmaceutically acceptable salt thereof, or a hydrate thereof, or a solvate thereof, wherein at least one of R₂ and R₃ is deuterium.

48. The compound of Embodiment 1, or a pharmaceutically acceptable salt thereof, or a hydrate thereof, or a solvate thereof, wherein the compound is selected from the compounds in Table 1A below, or a pharmaceutically acceptable salt thereof, or a hydrate thereof, or a solvate thereof.

TABLE 1A

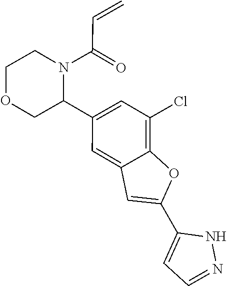 [SMILES: C=CC(=O)N1CCOCC1c1cc(Cl)c2oc(-c3ccn[nH]3)cc2c1]

B1

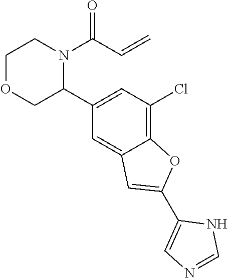 [SMILES: C=CC(=O)N1CCOCC1c1cc(Cl)c2oc(-c3cnc[nH]3)cc2c1]

B2

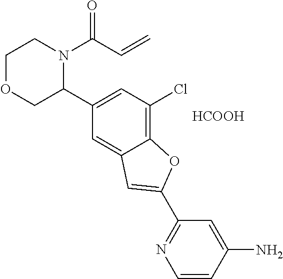 [SMILES: C=CC(=O)N1CCOCC1c1cc(Cl)c2oc(-c3cc(N)ccn3)cc2c1 ; "HCOOH"]

B3

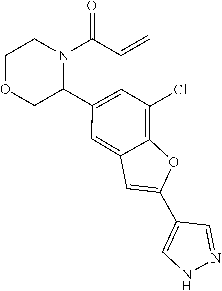 [SMILES: C=CC(=O)N1CCOCC1c1cc(Cl)c2oc(-c3cn[nH]c3)cc2c1]

B4

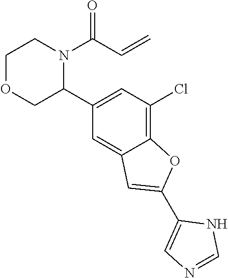 [SMILES: C=CC(=O)N1CCOCC1c1cc(Cl)c2oc(-c3cnc[nH]3)cc2c1]

B5
(absolute
configuration
assumed)

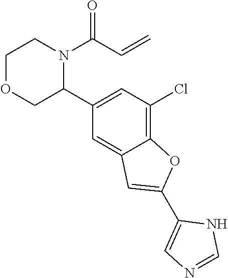 [SMILES: C=CC(=O)N1CCOCC1c1cc(Cl)c2oc(-c3cnc[nH]3)cc2c1]

B6
(absolute
configuration
assumed)

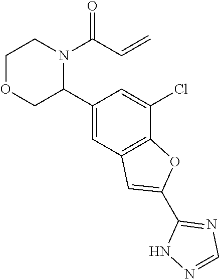 [SMILES: C=CC(=O)N1CCOCC1c1cc(Cl)c2oc(-c3ncn[nH]3)cc2c1]

B7

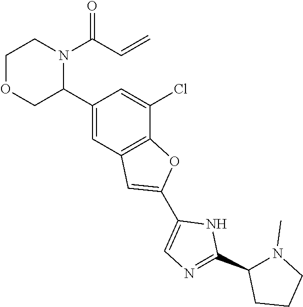 [SMILES: C=CC(=O)N1CCOCC1c1cc(Cl)c2oc(-c3cnc([C@@H]4CCCN4C)[nH]3)cc2c1]

B8

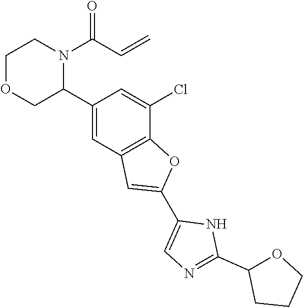 [SMILES: C=CC(=O)N1CCOCC1c1cc(Cl)c2oc(-c3cnc(C4CCCO4)[nH]3)cc2c1]

B9

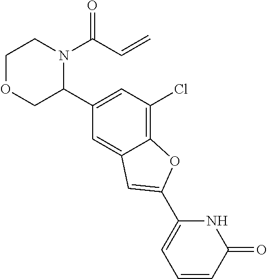 [SMILES: C=CC(=O)N1CCOCC1c1cc(Cl)c2oc(-c3cccc(=O)[nH]3)cc2c1]

B10

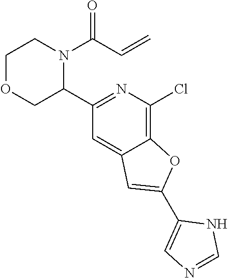 [SMILES: C=CC(=O)N1CCOCC1c1cc2cc(-c3cnc[nH]3)oc2c(Cl)n1]

B11

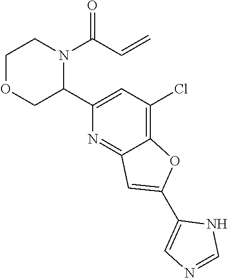 [SMILES: C=CC(=O)N1CCOCC1c1cc(Cl)c2oc(-c3cnc[nH]3)cc2n1]

B12

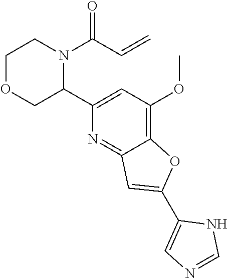 [SMILES: C=CC(=O)N1CCOCC1c1cc(OC)c2oc(-c3cnc[nH]3)cc2n1]

B13

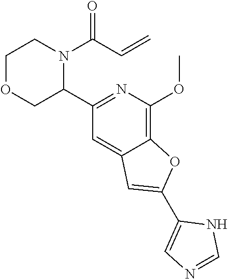 [SMILES: C=CC(=O)N1CCOCC1c1cc2cc(-c3cnc[nH]3)oc2c(OC)n1]

B14

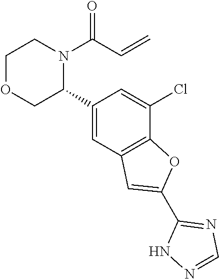 [SMILES: C=CC(=O)N1CCOC[C@H]1c1cc(Cl)c2oc(-c3ncn[nH]3)cc2c1]

B7A
(absolute
configuration
assumed)

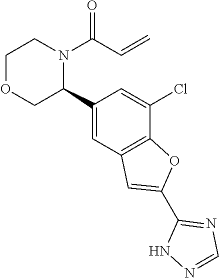 [SMILES: C=CC(=O)N1CCOC[C@@H]1c1cc(Cl)c2oc(-c3ncn[nH]3)cc2c1]

B7B (absolute
configuration
assumed)

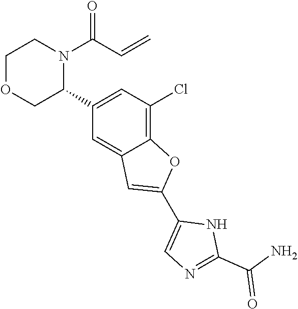 [SMILES: C=CC(=O)N1CCOC[C@H]1c1cc(Cl)c2oc(-c3cnc(C(N)=O)[nH]3)cc2c1]

B15A
(absolute
configuration
assumed)

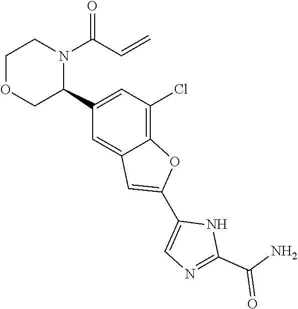 [SMILES: C=CC(=O)N1CCOC[C@@H]1c1cc(Cl)c2oc(-c3cnc(C(N)=O)[nH]3)cc2c1]

B15B
(absolute
configuration
assumed)

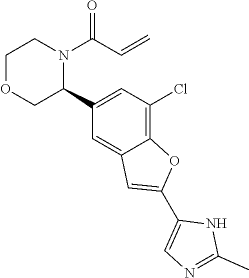 [SMILES: C=CC(=O)N1CCOC[C@@H]1c1cc(Cl)c2oc(-c3cnc(C)[nH]3)cc2c1]

B16A
(absolute
configuration
assumed)

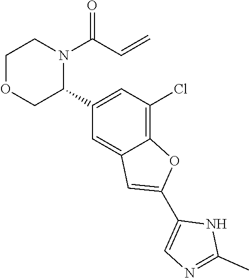 [SMILES: C=CC(=O)N1CCOC[C@H]1c1cc(Cl)c2oc(-c3cnc(C)[nH]3)cc2c1]

B16B
(absolute
configuration
assumed)

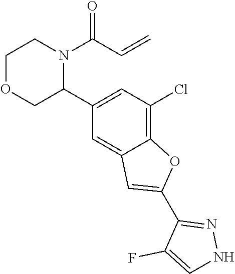 [SMILES: C=CC(=O)N1CCOCC1c1cc(Cl)c2oc(-c3n[nH]cc3F)cc2c1]

B17

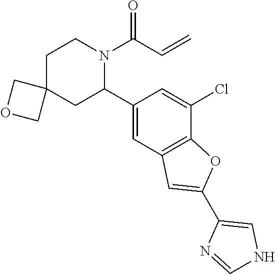 [SMILES: C=CC(=O)N1CCC2(COC2)CC1c1cc(Cl)c2oc(-c3c[nH]cn3)cc2c1]

B18

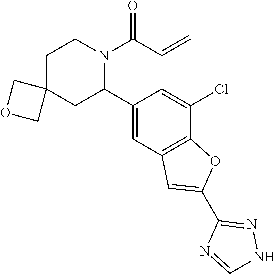 [SMILES: C=CC(=O)N1CCC2(COC2)CC1c1cc(Cl)c2oc(-c3nc[nH]n3)cc2c1]

B19

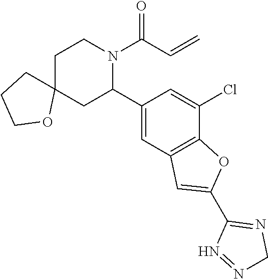 [SMILES: C=CC(=O)N1CCC2(CCCO2)CC1c1cc(Cl)c2oc(C3=NCN=N3)cc2c1]

B20

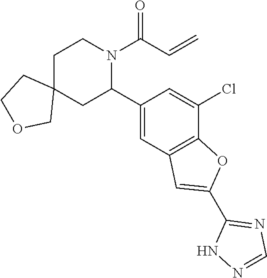 [SMILES: C=CC(=O)N1CCC2(CCOC2)CC1c1cc(Cl)c2oc(-c3ncn[nH]3)cc2c1]

B21

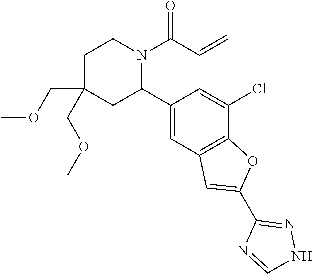 [SMILES: C=CC(=O)N1CCC(COC)(COC)CC1c1cc(Cl)c2oc(-c3nc[nH]n3)cc2c1]

B22

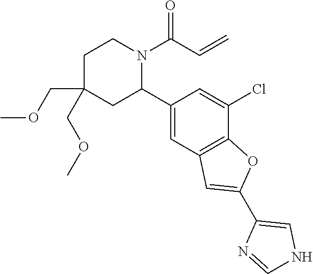 [SMILES: C=CC(=O)N1CCC(COC)(COC)CC1c1cc(Cl)c2oc(-c3c[nH]cn3)cc2c1]

B23

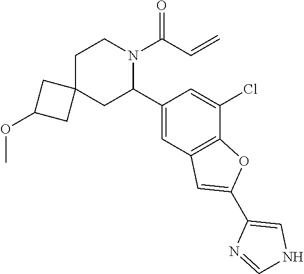 [SMILES: C=CC(=O)N1CCC2(CC(OC)C2)CC1c1cc(Cl)c2oc(-c3c[nH]cn3)cc2c1]

B24

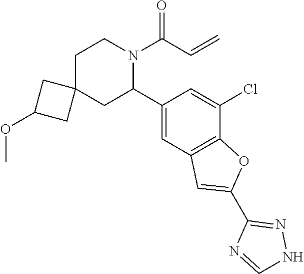 [SMILES: C=CC(=O)N1CCC2(CC(OC)C2)CC1c1cc(Cl)c2oc(-c3nc[nH]n3)cc2c1]

B25

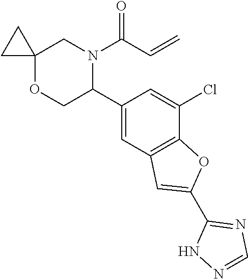 [SMILES: C=CC(=O)N1CC2(CC2)OCC1c1cc(Cl)c2oc(-c3ncn[nH]3)cc2c1]

B26

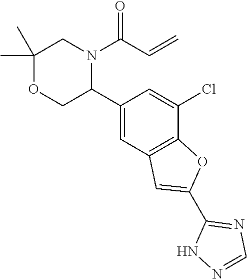 [SMILES: C=CC(=O)N1CC(C)(C)OCC1c1cc(Cl)c2oc(-c3ncn[nH]3)cc2c1]

B27

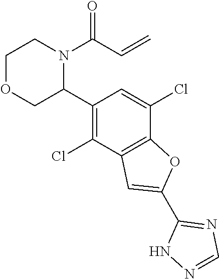 [SMILES: C=CC(=O)N1CCOCC1c1cc(Cl)c2oc(-c3ncn[nH]3)cc2c1Cl]

B28

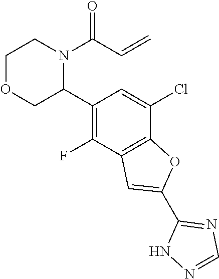 [SMILES: C=CC(=O)N1CCOCC1c1cc(Cl)c2oc(-c3ncn[nH]3)cc2c1F]

B29

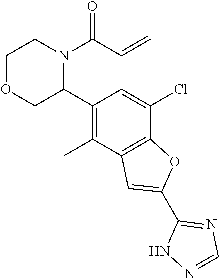 [SMILES: C=CC(=O)N1CCOCC1c1cc(Cl)c2oc(-c3ncn[nH]3)cc2c1C]

B30

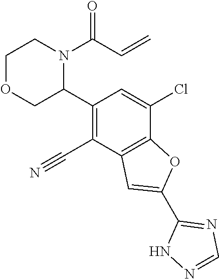 [SMILES: C=CC(=O)N1CCOCC1c1cc(Cl)c2oc(-c3ncn[nH]3)cc2c1C#N]

B31

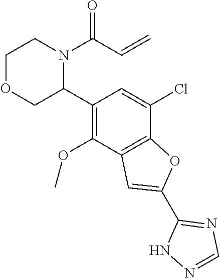 [SMILES: C=CC(=O)N1CCOCC1c1cc(Cl)c2oc(-c3ncn[nH]3)cc2c1OC]

B32

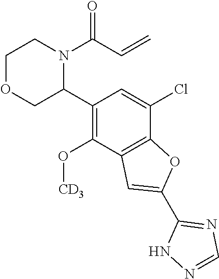 [SMILES: [2H]C([2H])([2H])Oc1c(C2COCCN2C(=O)C=C)cc(Cl)c2oc(-c3ncn[nH]3)cc12]

B33

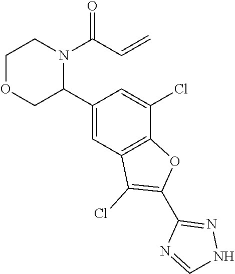 [SMILES: C=CC(=O)N1CCOCC1c1cc(Cl)c2oc(-c3nc[nH]n3)c(Cl)c2c1]

B34

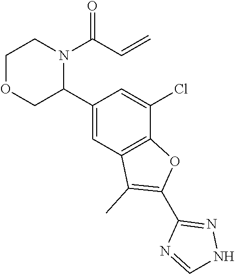 [SMILES: C=CC(=O)N1CCOCC1c1cc(Cl)c2oc(-c3nc[nH]n3)c(C)c2c1]

B35

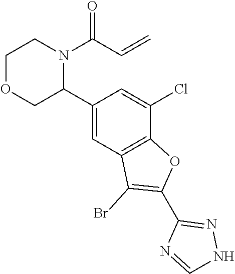 [SMILES: C=CC(=O)N1CCOCC1c1cc(Cl)c2oc(-c3nc[nH]n3)c(Br)c2c1]

B36

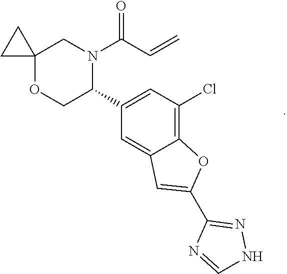 [SMILES: C=CC(=O)N1CC2(CC2)OC[C@H]1c1cc(Cl)c2oc(-c3nc[nH]n3)cc2c1]

B26A
(absolute
configuration
assumed)

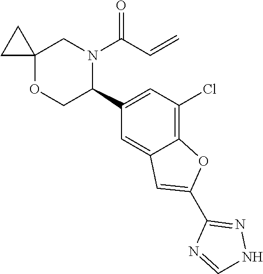 [SMILES: C=CC(=O)N1CC2(CC2)OC[C@@H]1c1cc(Cl)c2oc(-c3nc[nH]n3)cc2c1]

B26B
(absolute
configuration
assumed)

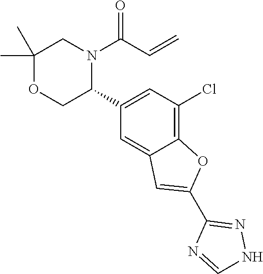 [SMILES: C=CC(=O)N1CC(C)(C)OC[C@H]1c1cc(Cl)c2oc(-c3nc[nH]n3)cc2c1]

B27A
(absolute
configuration
assumed)

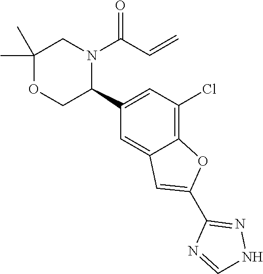 [SMILES: C=CC(=O)N1CC(C)(C)OC[C@@H]1c1cc(Cl)c2oc(-c3nc[nH]n3)cc2c1]

B27B
(absolute
configuration
assumed)

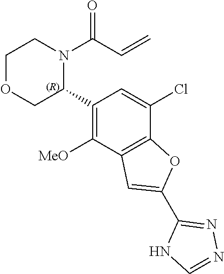

B32A
(absolute
configuration
assumed)

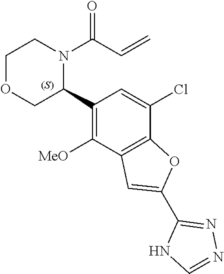

B32B
(absolute
configuration
assumed)

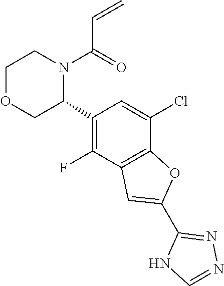 [SMILES: C=CC(=O)N1CCOC[C@H]1c1cc(Cl)c2oc(-c3nnc[nH]3)cc2c1F]

B29A
(absolute
configuration
assumed)

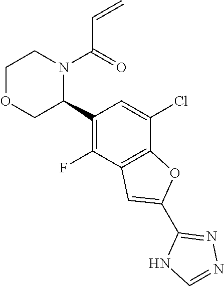 [SMILES: C=CC(=O)N1CCOC[C@@H]1c1cc(Cl)c2oc(-c3nnc[nH]3)cc2c1F]

B29B
(absolute
configuration
assumed)

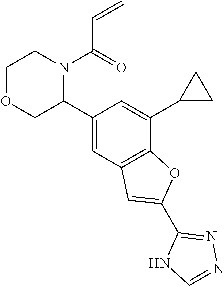 [SMILES: C=CC(=O)N1CCOCC1c1cc(C2CC2)c2oc(-c3nnc[nH]3)cc2c1]

B33

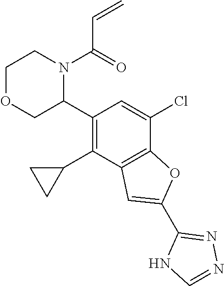 [SMILES: C=CC(=O)N1CCOCC1c1cc(Cl)c2oc(-c3nnc[nH]3)cc2c1C1CC1]

B34

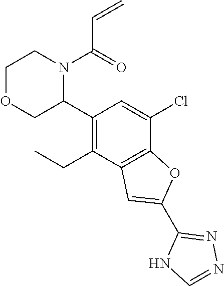 [SMILES: C=CC(=O)N1CCOCC1c1cc(Cl)c2oc(-c3nnc[nH]3)cc2c1CC]

B35

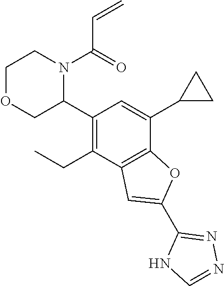 [SMILES: C=CC(=O)N1CCOCC1c1cc(C2CC2)c2oc(-c3nnc[nH]3)cc2c1CC]

B36

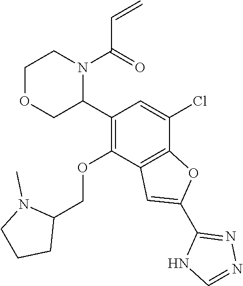 [SMILES: C=CC(=O)N1CCOCC1c1cc(Cl)c2oc(-c3nnc[nH]3)cc2c1OCC1CCCN1C]

B37

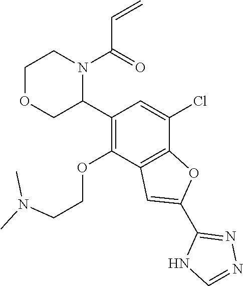 [SMILES: C=CC(=O)N1CCOCC1c1cc(Cl)c2oc(-c3nnc[nH]3)cc2c1OCCN(C)C]

B38

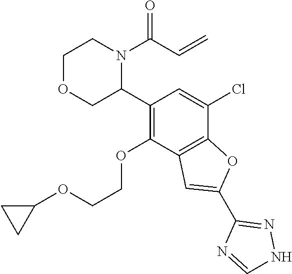 [SMILES: C=CC(=O)N1CCOCC1c1cc(Cl)c2oc(-c3nc[nH]n3)cc2c1OCCOC1CC1]

B39

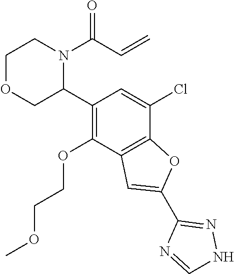 [SMILES: C=CC(=O)N1CCOCC1c1cc(Cl)c2oc(-c3nc[nH]n3)cc2c1OCCOC]

B40

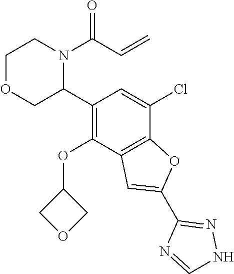 [SMILES: C=CC(=O)N1CCOCC1c1cc(Cl)c2oc(-c3nc[nH]n3)cc2c1OC1COC1]

B41

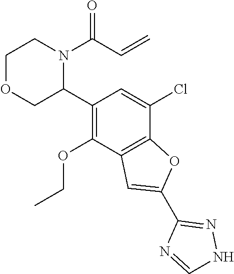 [SMILES: C=CC(=O)N1CCOCC1c1cc(Cl)c2oc(-c3nc[nH]n3)cc2c1OCC]

B42

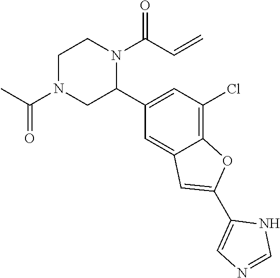 [SMILES: C=CC(=O)N1CCN(C(C)=O)CC1c1cc(Cl)c2oc(-c3cnc[nH]3)cc2c1]

C1

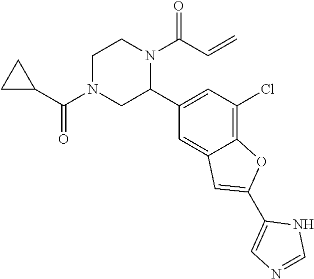 [SMILES: C=CC(=O)N1CCN(C(=O)C2CC2)CC1c1cc(Cl)c2oc(-c3cnc[nH]3)cc2c1]

C2

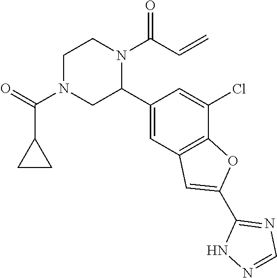 [SMILES: C=CC(=O)N1CCN(C(=O)C2CC2)CC1c1cc(Cl)c2oc(-c3ncn[nH]3)cc2c1]

C3

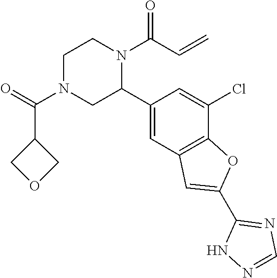 [SMILES: C=CC(=O)N1CCN(C(=O)C2COC2)CC1c1cc(Cl)c2oc(-c3ncn[nH]3)cc2c1]

C4

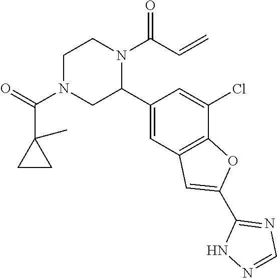 [SMILES: C=CC(=O)N1CCN(C(=O)C2(C)CC2)CC1c1cc(Cl)c2oc(-c3ncn[nH]3)cc2c1]

C5

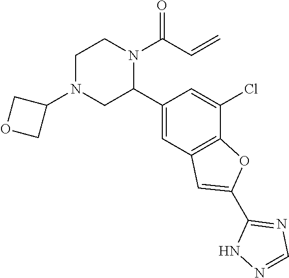 [SMILES: C=CC(=O)N1CCN(C2COC2)CC1c1cc(Cl)c2oc(-c3ncn[nH]3)cc2c1]

C6

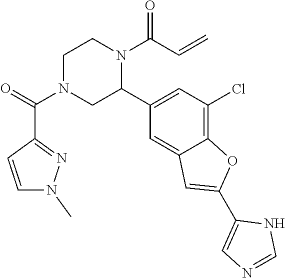 [SMILES: C=CC(=O)N1CCN(C(=O)c2ccn(C)n2)CC1c1cc(Cl)c2oc(-c3cnc[nH]3)cc2c1]

C7

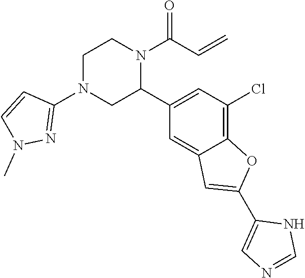 [SMILES: C=CC(=O)N1CCN(c2ccn(C)n2)CC1c1cc(Cl)c2oc(-c3cnc[nH]3)cc2c1]

C8

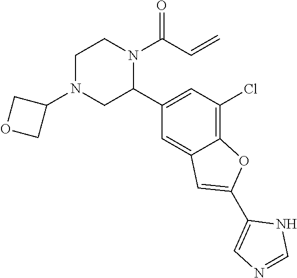 [SMILES: C=CC(=O)N1CCN(C2COC2)CC1c1cc(Cl)c2oc(-c3cnc[nH]3)cc2c1]

C9

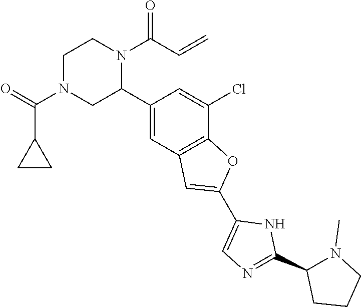 [SMILES: C=CC(=O)N1CCN(C(=O)C2CC2)CC1c1cc(Cl)c2oc(-c3cnc([C@@H]4CCCN4C)[nH]3)cc2c1]

C10

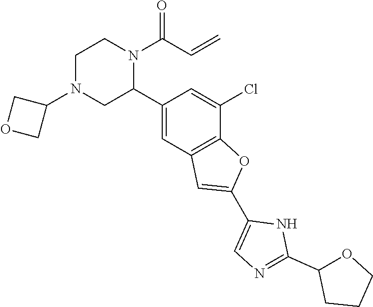 [SMILES: C=CC(=O)N1CCN(C2COC2)CC1c1cc(Cl)c2oc(-c3cnc(C4CCCO4)[nH]3)cc2c1]

C11

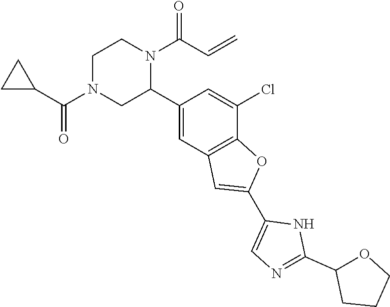 [SMILES: C=CC(=O)N1CCN(C(=O)C2CC2)CC1c1cc(Cl)c2oc(-c3cnc(C4CCCO4)[nH]3)cc2c1]

C12

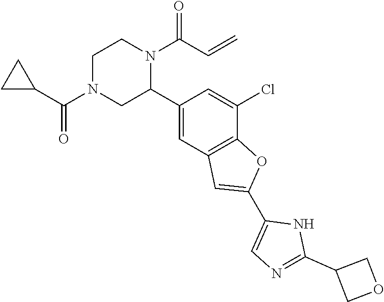 [SMILES: C=CC(=O)N1CCN(C(=O)C2CC2)CC1c1cc(Cl)c2oc(-c3cnc(C4COC4)[nH]3)cc2c1]

C13

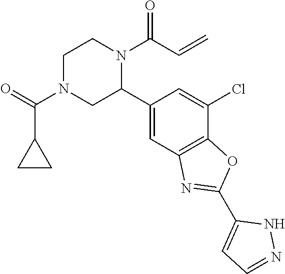 [SMILES: C=CC(=O)N1CCN(C(=O)C2CC2)CC1c1cc(Cl)c2oc(-c3ccn[nH]3)nc2c1]

D1

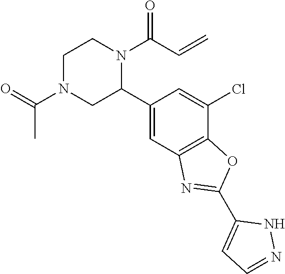 [SMILES: C=CC(=O)N1CCN(C(C)=O)CC1c1cc(Cl)c2oc(-c3ccn[nH]3)nc2c1]

D2

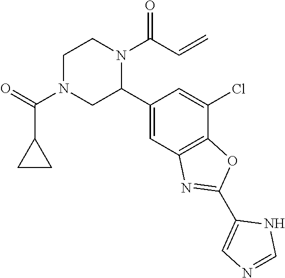 [SMILES: C=CC(=O)N1CCN(C(=O)C2CC2)CC1c1cc(Cl)c2oc(-c3cnc[nH]3)nc2c1]

D3

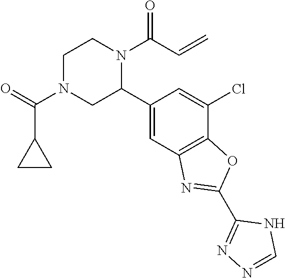 [SMILES: C=CC(=O)N1CCN(C(=O)C2CC2)CC1c1cc(Cl)c2oc(-c3nnc[nH]3)nc2c1]

D4

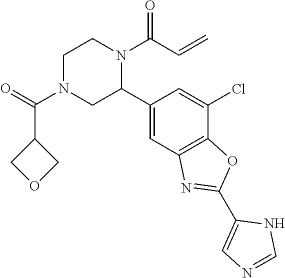 [SMILES: C=CC(=O)N1CCN(C(=O)C2COC2)CC1c1cc(Cl)c2oc(-c3cnc[nH]3)nc2c1]

D5

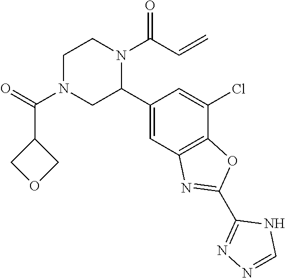 [SMILES: C=CC(=O)N1CCN(C(=O)C2COC2)CC1c1cc(Cl)c2oc(-c3nnc[nH]3)nc2c1]

D6

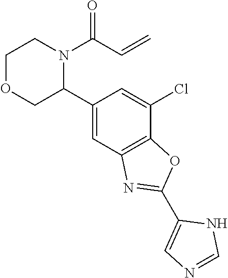 [SMILES: C=CC(=O)N1CCOCC1c1cc(Cl)c2oc(-c3cnc[nH]3)nc2c1]

D7

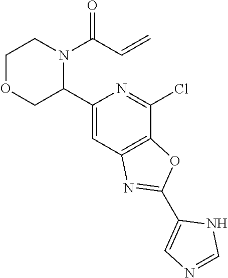 [SMILES: C=CC(=O)N1CCOCC1c1cc2nc(-c3cnc[nH]3)oc2c(Cl)n1]

D8

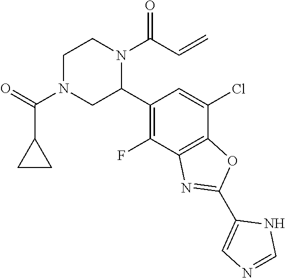 [SMILES: C=CC(=O)N1CCN(C(=O)C2CC2)CC1c1cc(Cl)c2oc(-c3cnc[nH]3)nc2c1F]

D9

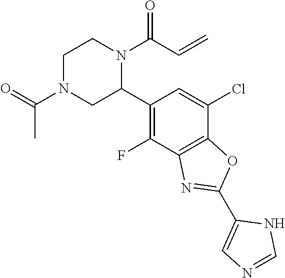 [SMILES: C=CC(=O)N1CCN(C(C)=O)CC1c1cc(Cl)c2oc(-c3cnc[nH]3)nc2c1F]

D10

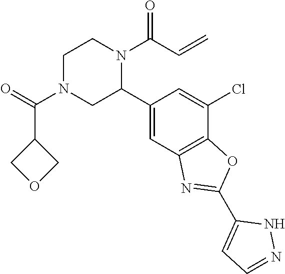 [SMILES: C=CC(=O)N1CCN(C(=O)C2COC2)CC1c1cc(Cl)c2oc(-c3ccn[nH]3)nc2c1]

D11

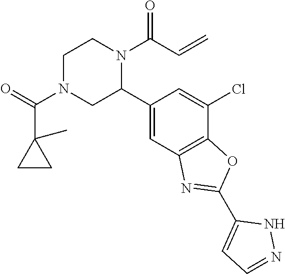 [SMILES: C=CC(=O)N1CCN(C(=O)C2(C)CC2)CC1c1cc(Cl)c2oc(-c3ccn[nH]3)nc2c1]

D12

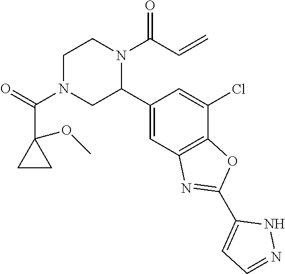 [SMILES: C=CC(=O)N1CCN(C(=O)C2(OC)CC2)CC1c1cc(Cl)c2oc(-c3ccn[nH]3)nc2c1]

D13

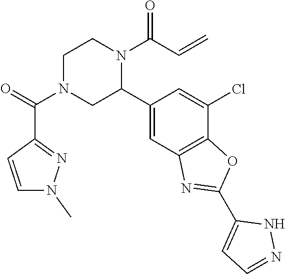 [SMILES: C=CC(=O)N1CCN(C(=O)c2ccn(C)n2)CC1c1cc(Cl)c2oc(-c3ccn[nH]3)nc2c1]

D14

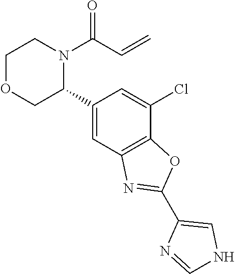 [SMILES: C=CC(=O)N1CCOC[C@H]1c1cc(Cl)c2oc(-c3c[nH]cn3)nc2c1]

D7A
(absolute
configuration
assumed)

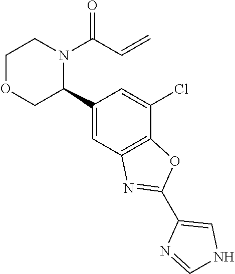 [SMILES: C=CC(=O)N1CCOC[C@@H]1c1cc(Cl)c2oc(-c3c[nH]cn3)nc2c1]

D7B
(absolute
configuration
assumed)

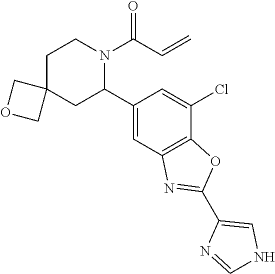 [SMILES: C=CC(=O)N1CCC2(COC2)CC1c1cc(Cl)c2oc(-c3c[nH]cn3)nc2c1]

D15

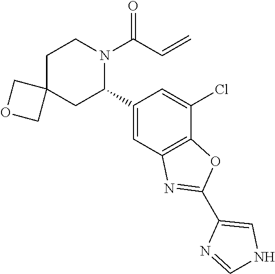 [SMILES: C=CC(=O)N1CCC2(COC2)C[C@H]1c1cc(Cl)c2oc(-c3c[nH]cn3)nc2c1]

D15A
(absolute
configuration
assumed)

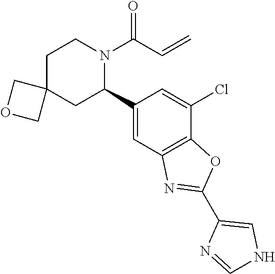 [SMILES: C=CC(=O)N1CCC2(COC2)C[C@@H]1c1cc(Cl)c2oc(-c3c[nH]cn3)nc2c1]

D15B
(absolute
configuration
assumed)

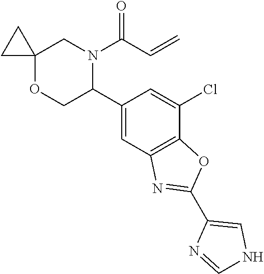 [SMILES: C=CC(=O)N1CC2(CC2)OCC1c1cc(Cl)c2oc(-c3c[nH]cn3)nc2c1]

D16

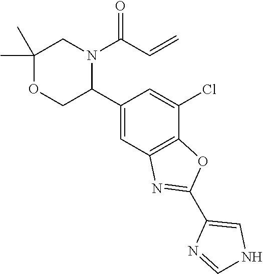 [SMILES: C=CC(=O)N1CC(C)(C)OCC1c1cc(Cl)c2oc(-c3c[nH]cn3)nc2c1]

D17

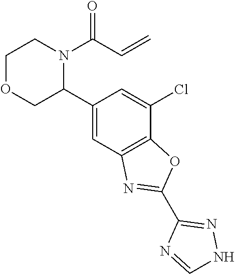 [SMILES: C=CC(=O)N1CCOCC1c1cc(Cl)c2oc(-c3nc[nH]n3)nc2c1]

D18

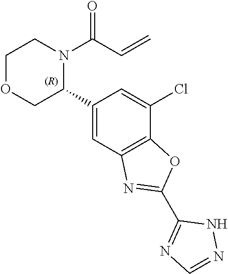

D18A
(absolute
configuration
assumed)

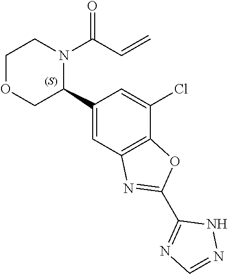

D18B
(absolute
configuration
assumed)

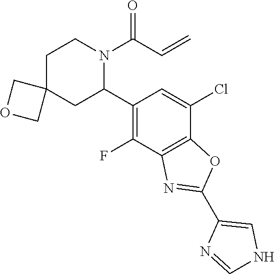 [SMILES: C=CC(=O)N1CCC2(COC2)CC1c1cc(Cl)c2oc(-c3c[nH]cn3)nc2c1F]

D19

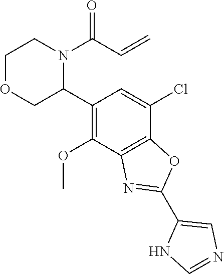 [SMILES: C=CC(=O)N1CCOCC1c1cc(Cl)c2oc(-c3cnc[nH]3)nc2c1OC]

D20

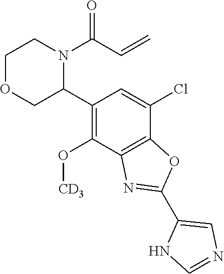 [SMILES: [2H]C([2H])([2H])Oc1c(C2COCCN2C(=O)C=C)cc(Cl)c2oc(-c3cnc[nH]3)nc12]

D21

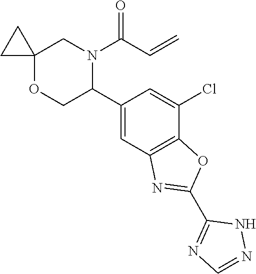 [SMILES: C=CC(=O)N1CC2(CC2)OCC1c1cc(Cl)c2oc(-c3ncn[nH]3)nc2c1]

D22

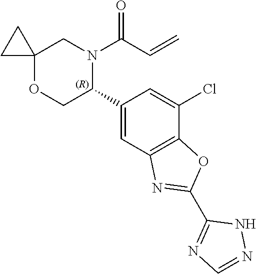

D22A
(absolute
configuration
assumed)

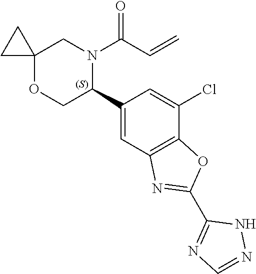

D22B
(absolute
configuration
assumed)

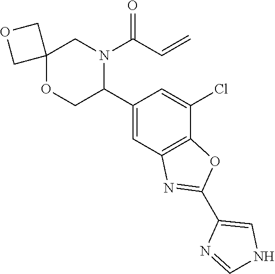 [SMILES: C=CC(=O)N1CC2(COC2)OCC1c1cc(Cl)c2oc(-c3c[nH]cn3)nc2c1]

D23

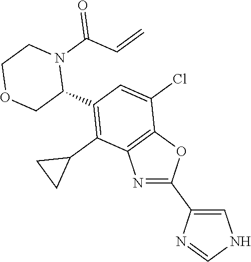 [SMILES: C=CC(=O)N1CCOC[C@H]1c1cc(Cl)c2oc(-c3c[nH]cn3)nc2c1C1CC1]

D24A
(absolute
configuration
assumed)

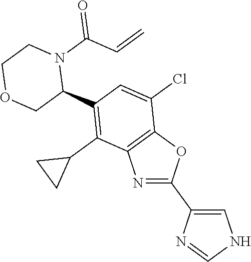 [SMILES: C=CC(=O)N1CCOC[C@@H]1c1cc(Cl)c2oc(-c3c[nH]cn3)nc2c1C1CC1]

D24B
(absolute
configuration
assumed)

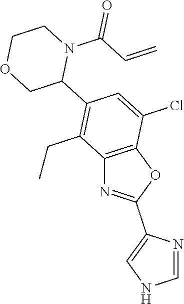 [SMILES: C=CC(=O)N1CCOCC1c1cc(Cl)c2oc(-c3c[nH]cn3)nc2c1CC]

D25

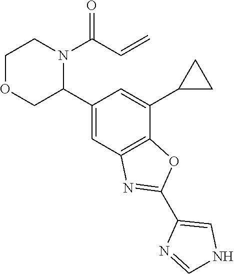 [SMILES: C=CC(=O)N1CCOCC1c1cc(C2CC2)c2oc(-c3c[nH]cn3)nc2c1]

D26

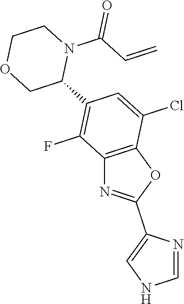 [SMILES: C=CC(=O)N1CCOC[C@H]1c1cc(Cl)c2oc(-c3c[nH]cn3)nc2c1F]

D27

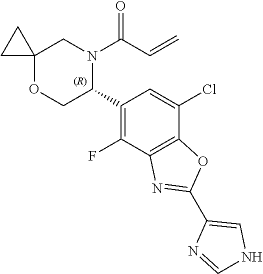

D28A
(absolute
configuration
assumed)

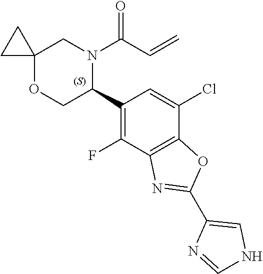

D28B
(absolute
configuration
assumed)

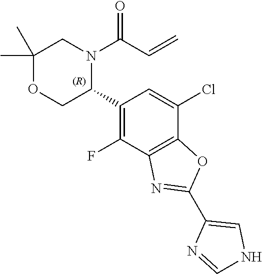

D29A
(absolute
configuration
assumed)

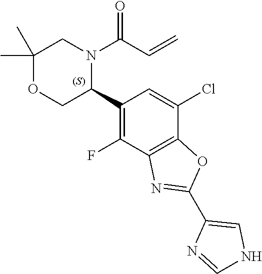

D29B
(absolute
configuration
assumed)

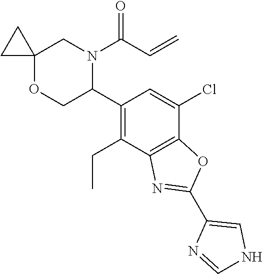 [SMILES: C=CC(=O)N1CC2(CC2)OCC1c1cc(Cl)c2oc(-c3c[nH]cn3)nc2c1CC]

D30

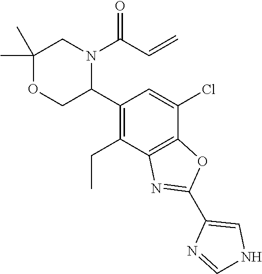 [SMILES: C=CC(=O)N1CC(C)(C)OCC1c1cc(Cl)c2oc(-c3c[nH]cn3)nc2c1CC]

D31

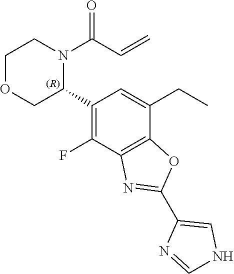

D32A
(absolute
configuration
assumed)

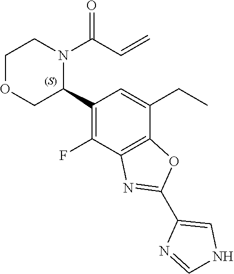

D32B
(absolute
configuration
assumed)

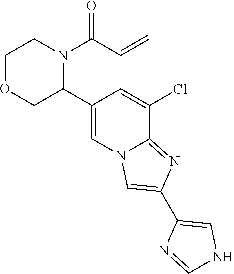 [SMILES: C=CC(=O)N1CCOCC1c1cc(Cl)c2nc(-c3c[nH]cn3)cn2c1]

E1

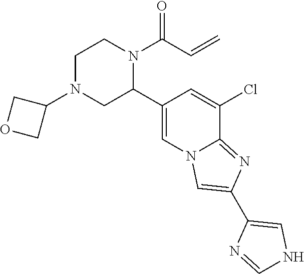 [SMILES: C=CC(=O)N1CCN(C2COC2)CC1c1cc(Cl)c2nc(-c3c[nH]cn3)cn2c1]

E2

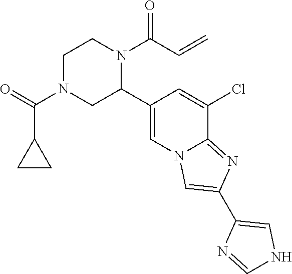 [SMILES: C=CC(=O)N1CCN(C(=O)C2CC2)CC1c1cc(Cl)c2nc(-c3c[nH]cn3)cn2c1]

E3

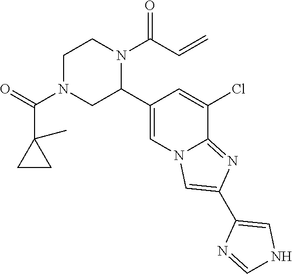 [SMILES: C=CC(=O)N1CCN(C(=O)C2(C)CC2)CC1c1cc(Cl)c2nc(-c3c[nH]cn3)cn2c1]

E4

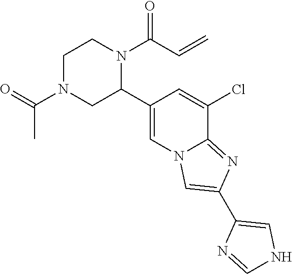 [SMILES: C=CC(=O)N1CCN(C(C)=O)CC1c1cc(Cl)c2nc(-c3c[nH]cn3)cn2c1]

E5

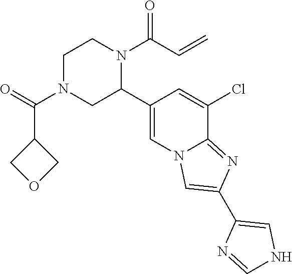 [SMILES: C=CC(=O)N1CCN(C(=O)C2COC2)CC1c1cc(Cl)c2nc(-c3c[nH]cn3)cn2c1]

E6

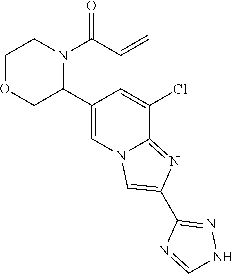 [SMILES: C=CC(=O)N1CCOCC1c1cc(Cl)c2nc(-c3nc[nH]n3)cn2c1]

E7

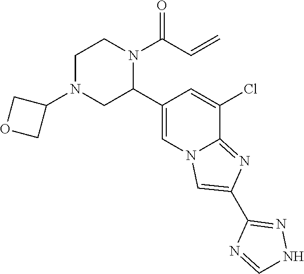 [SMILES: C=CC(=O)N1CCN(C2COC2)CC1c1cc(Cl)c2nc(-c3nc[nH]n3)cn2c1]

E8

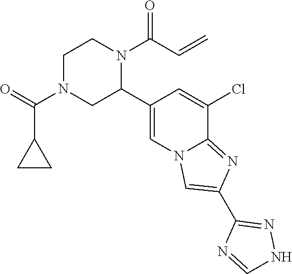 [SMILES: C=CC(=O)N1CCN(C(=O)C2CC2)CC1c1cc(Cl)c2nc(-c3nc[nH]n3)cn2c1]

E9

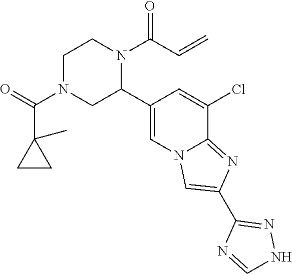 [SMILES: C=CC(=O)N1CCN(C(=O)C2(C)CC2)CC1c1cc(Cl)c2nc(-c3nc[nH]n3)cn2c1]

E10

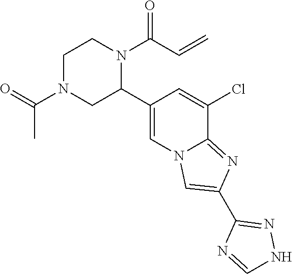 [SMILES: C=CC(=O)N1CCN(C(C)=O)CC1c1cc(Cl)c2nc(-c3nc[nH]n3)cn2c1]

E11

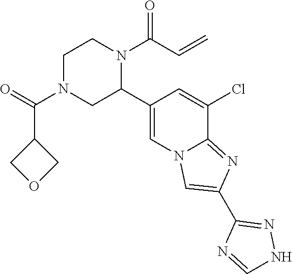 [SMILES: C=CC(=O)N1CCN(C(=O)C2COC2)CC1c1cc(Cl)c2nc(-c3nc[nH]n3)cn2c1]

E12

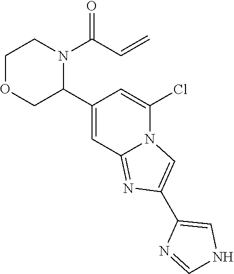 [SMILES: C=CC(=O)N1CCOCC1c1cc(Cl)n2cc(-c3c[nH]cn3)nc2c1]

F1

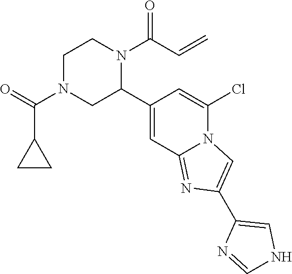 [SMILES: C=CC(=O)N1CCN(C(=O)C2CC2)CC1c1cc(Cl)n2cc(-c3c[nH]cn3)nc2c1]

F2

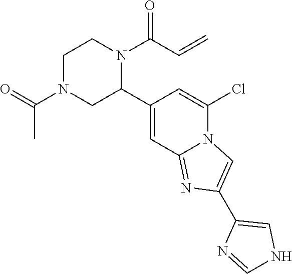 [SMILES: C=CC(=O)N1CCN(C(C)=O)CC1c1cc(Cl)n2cc(-c3c[nH]cn3)nc2c1]

F3

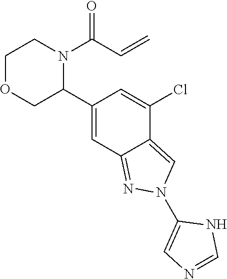 [SMILES: C=CC(=O)N1CCOCC1c1cc(Cl)c2cn(-c3cnc[nH]3)nc2c1]

G1

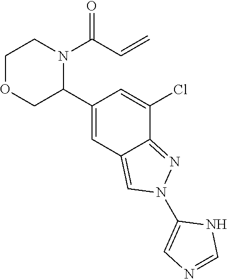 [SMILES: C=CC(=O)N1CCOCC1c1cc(Cl)c2nn(-c3cnc[nH]3)cc2c1]

G2

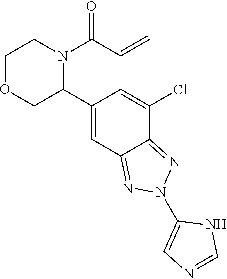 [SMILES: C=CC(=O)N1CCOCC1c1cc(Cl)c2nn(-c3cnc[nH]3)nc2c1]

G3

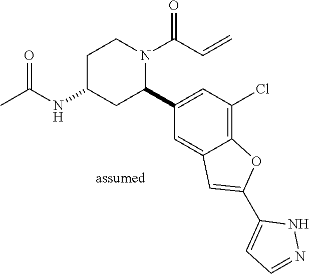

H1

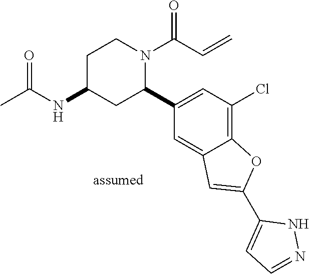 [SMILES: C=CC(=O)N1CC[C@H](NC(C)=O)C[C@@H]1c1cc(Cl)c2oc(-c3ccn[nH]3)cc2c1]

H2

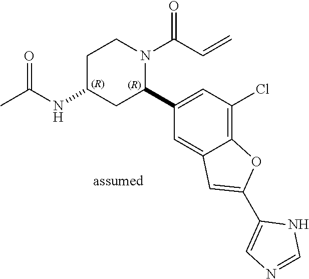

H3

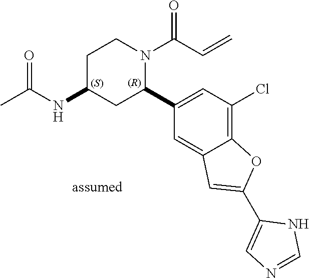

H4

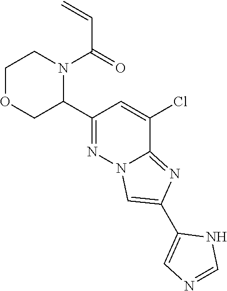 [SMILES: C=CC(=O)N1CCOCC1c1cc(Cl)c2nc(-c3cnc[nH]3)cn2n1]

I1

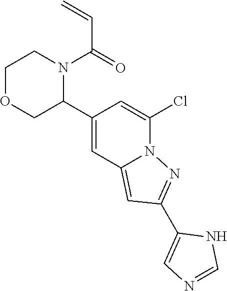 [SMILES: C=CC(=O)N1CCOCC1c1cc(Cl)n2nc(-c3cnc[nH]3)cc2c1]

J1

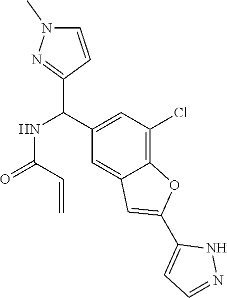 [SMILES: C=CC(=O)NC(c1cc(Cl)c2oc(-c3ccn[nH]3)cc2c1)c1ccn(C)n1]

K1

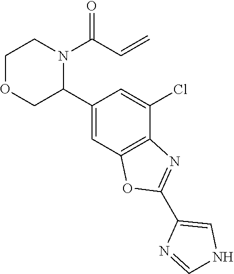 [SMILES: C=CC(=O)N1CCOCC1c1cc(Cl)c2nc(-c3c[nH]cn3)oc2c1]

L1

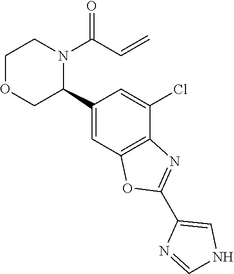 [SMILES: C=CC(=O)N1CCOC[C@@H]1c1cc(Cl)c2nc(-c3c[nH]cn3)oc2c1]

L1A
(absolute
configuration
assumed)

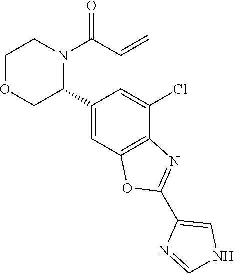 [SMILES: C=CC(=O)N1CCOC[C@H]1c1cc(Cl)c2nc(-c3c[nH]cn3)oc2c1]

L1B
(absolute
configuration
assumed)

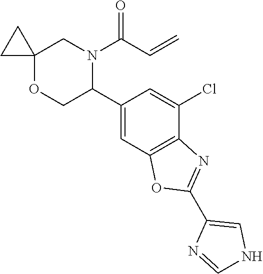 [SMILES: C=CC(=O)N1CC2(CC2)OCC1c1cc(Cl)c2nc(-c3c[nH]cn3)oc2c1]

L2

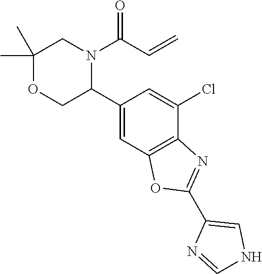 [SMILES: C=CC(=O)N1CC(C)(C)OCC1c1cc(Cl)c2nc(-c3c[nH]cn3)oc2c1]

L3

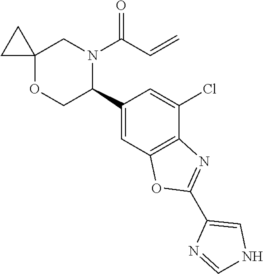 [SMILES: C=CC(=O)N1CC2(CC2)OC[C@@H]1c1cc(Cl)c2nc(-c3c[nH]cn3)oc2c1]

L2A
(absolute
configuration
assumed)

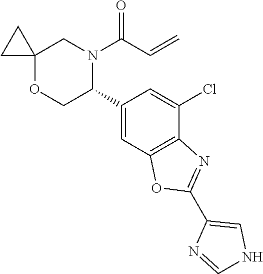 [SMILES: C=CC(=O)N1CC2(CC2)OC[C@H]1c1cc(Cl)c2nc(-c3c[nH]cn3)oc2c1]

L2B
(absolute
configuration
assumed)

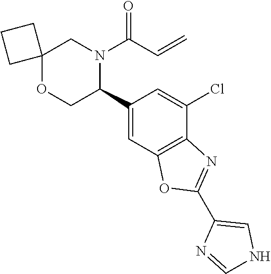 [SMILES: C=CC(=O)N1CC2(CCC2)OC[C@@H]1c1cc(Cl)c2nc(-c3c[nH]cn3)oc2c1]

L4A
(absolute
configuration
assumed)

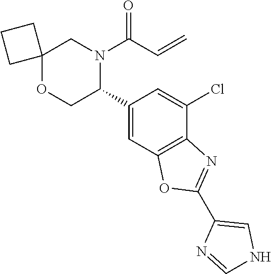 [SMILES: C=CC(=O)N1CC2(CCC2)OC[C@H]1c1cc(Cl)c2nc(-c3c[nH]cn3)oc2c1]

L4B
(absolute
configuration
assumed)

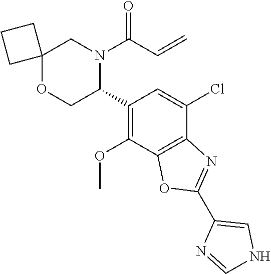 [SMILES: C=CC(=O)N1CC2(CCC2)OC[C@H]1c1cc(Cl)c2nc(-c3c[nH]cn3)oc2c1OC]

L5A
(absolute
configuration
assumed)

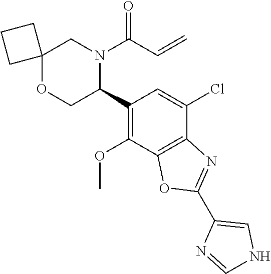 [SMILES: C=CC(=O)N1CC2(CCC2)OC[C@@H]1c1cc(Cl)c2nc(-c3c[nH]cn3)oc2c1OC]

L5B
(absolute
configuration
assumed)

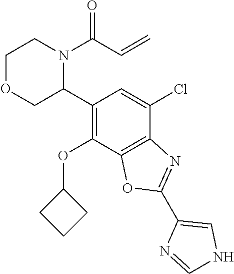 [SMILES: C=CC(=O)N1CCOCC1c1cc(Cl)c2nc(-c3c[nH]cn3)oc2c1OC1CCC1]

L6

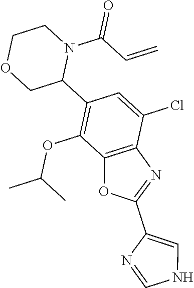 [SMILES: C=CC(=O)N1CCOCC1c1cc(Cl)c2nc(-c3c[nH]cn3)oc2c1OC(C)C]

L7

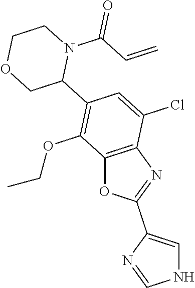 [SMILES: C=CC(=O)N1CCOCC1c1cc(Cl)c2nc(-c3c[nH]cn3)oc2c1OCC]

L8

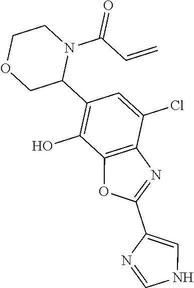 [SMILES: C=CC(=O)N1CCOCC1c1cc(Cl)c2nc(-c3c[nH]cn3)oc2c1O]

L9

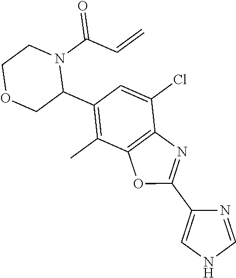 [SMILES: C=CC(=O)N1CCOCC1c1cc(Cl)c2nc(-c3c[nH]cn3)oc2c1C]

L10

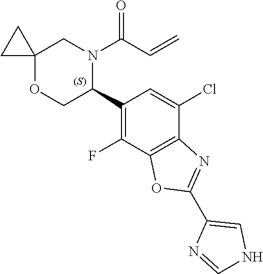

L11A
(absolute
configuration
assumed)

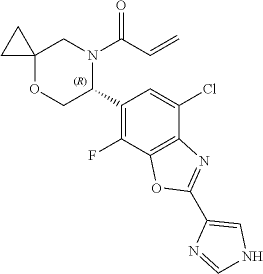

L11B
(absolute
configuration
assumed)

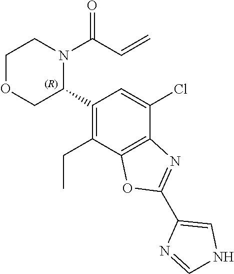

L12A
(absolute
configuration
assumed)

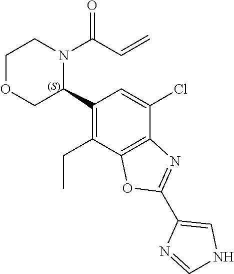

L12B
(absolute
configuration
assumed)

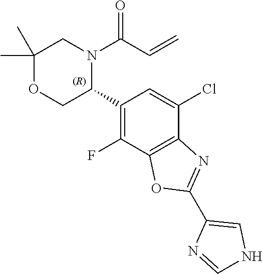

L13A
(absolute
configuration
assumed)

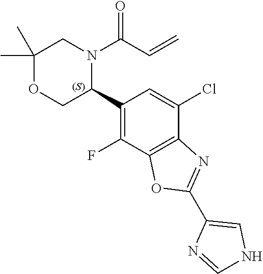

L13B
(absolute
configuration
assumed)

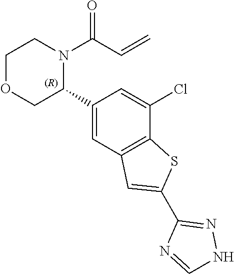

M1A
(absolute
configuration
assumed)

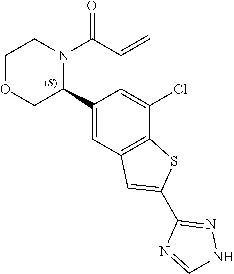

M1B
(absolute
configuration
assumed)

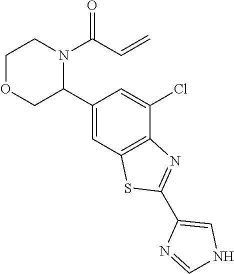 [SMILES: C=CC(=O)N1CCOCC1c1cc(Cl)c2nc(-c3c[nH]cn3)sc2c1]

N1

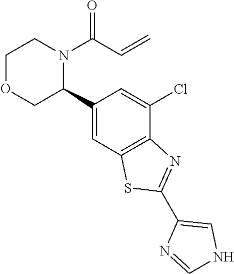 [SMILES: C=CC(=O)N1CCOC[C@@H]1c1cc(Cl)c2nc(-c3c[nH]cn3)sc2c1]

N1A
(absolute
configuration
assumed)

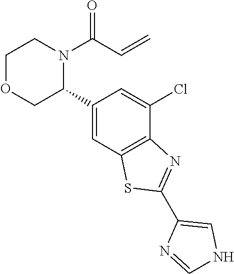 [SMILES: C=CC(=O)N1CCOC[C@H]1c1cc(Cl)c2nc(-c3c[nH]cn3)sc2c1]

N1B
(absolute
configuration
assumed)

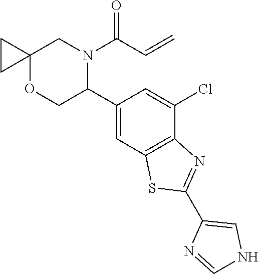 [SMILES: C=CC(=O)N1CC2(CC2)OCC1c1cc(Cl)c2nc(-c3c[nH]cn3)sc2c1]

N2

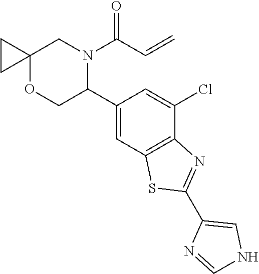 [SMILES: C=CC(=O)N1CC2(CC2)OCC1c1cc(Cl)c2nc(-c3c[nH]cn3)sc2c1]

N3

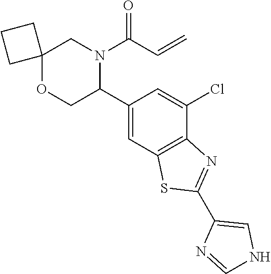 [SMILES: C=CC(=O)N1CC2(CCC2)OCC1c1cc(Cl)c2nc(-c3c[nH]cn3)sc2c1]

N4

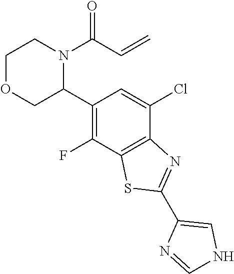 [SMILES: C=CC(=O)N1CCOCC1c1cc(Cl)c2nc(-c3c[nH]cn3)sc2c1F]

N5

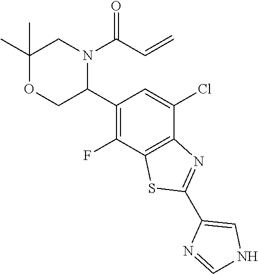 [SMILES: C=CC(=O)N1CC(C)(C)OCC1c1cc(Cl)c2nc(-c3c[nH]cn3)sc2c1F]

N6

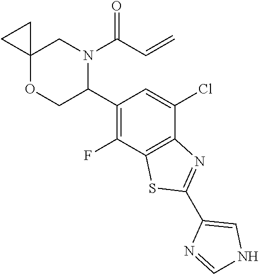 [SMILES: C=CC(=O)N1CC2(CC2)OCC1c1cc(Cl)c2nc(-c3c[nH]cn3)sc2c1F]

N7

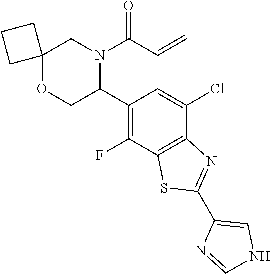 [SMILES: C=CC(=O)N1CC2(CCC2)OCC1c1cc(Cl)c2nc(-c3c[nH]cn3)sc2c1F]

N8

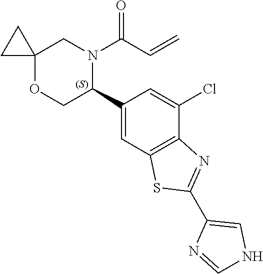

N2A
(absolute
configuration
assumed)

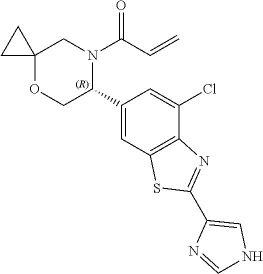

N2B
(absolute
configuration
assumed)

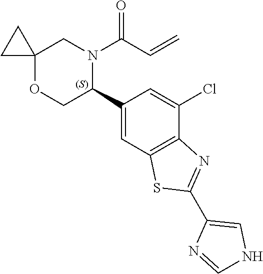

N3A
(absolute
configuration
assumed)

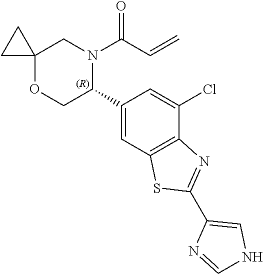

N3B
(absolute
configuration
assumed)

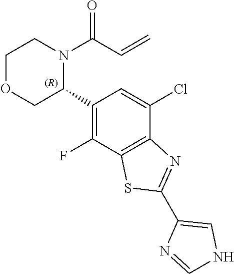

N5A
(absolute
configuration
assumed)

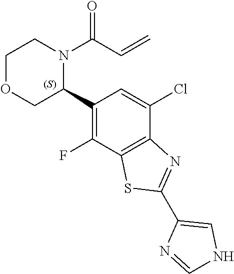

N5B
(absolute
configuration
assumed)

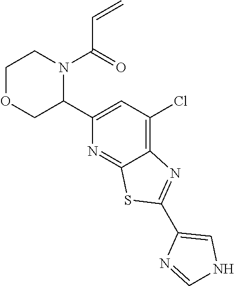 [SMILES: C=CC(=O)N1CCOCC1c1cc(Cl)c2nc(-c3c[nH]cn3)sc2n1]

N11

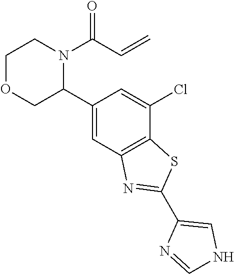 [SMILES: C=CC(=O)N1CCOCC1c1cc(Cl)c2sc(-c3c[nH]cn3)nc2c1]

O1

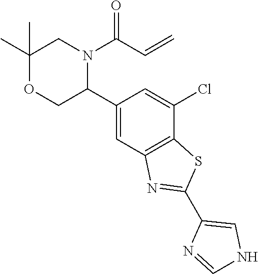 [SMILES: C=CC(=O)N1CC(C)(C)OCC1c1cc(Cl)c2sc(-c3c[nH]cn3)nc2c1]

O2

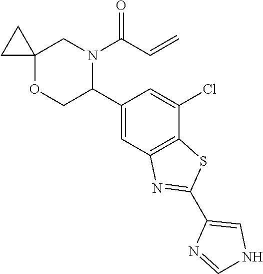 [SMILES: C=CC(=O)N1CC2(CC2)OCC1c1cc(Cl)c2sc(-c3c[nH]cn3)nc2c1]

O3

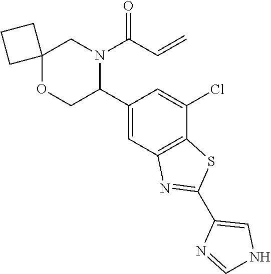 [SMILES: C=CC(=O)N1CC2(CCC2)OCC1c1cc(Cl)c2sc(-c3c[nH]cn3)nc2c1]

O4

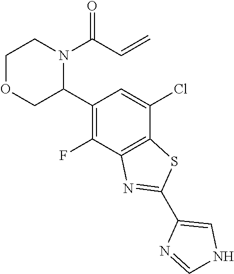 [SMILES: C=CC(=O)N1CCOCC1c1cc(Cl)c2sc(-c3c[nH]cn3)nc2c1F]

O5

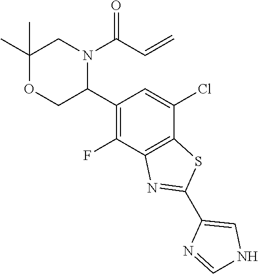 [SMILES: C=CC(=O)N1CC(C)(C)OCC1c1cc(Cl)c2sc(-c3c[nH]cn3)nc2c1F]

O6

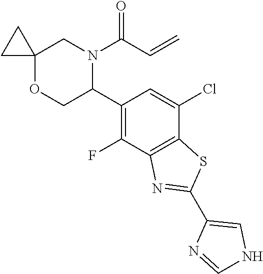 [SMILES: C=CC(=O)N1CC2(CC2)OCC1c1cc(Cl)c2sc(-c3c[nH]cn3)nc2c1F]

O7

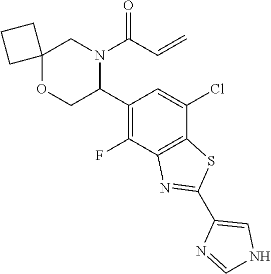 [SMILES: C=CC(=O)N1CC2(CCC2)OCC1c1cc(Cl)c2sc(-c3c[nH]cn3)nc2c1F]

O8

49. The compound of Embodiment 48, or a pharmaceutically acceptable salt thereof, or a hydrate thereof, or a solvate thereof, wherein the compound has one or more hydrogens replaced by deuterium, or wherein the compound has one or more hydrogens on the morpholine moiety replaced by deuterium.

50. A pharmaceutical composition comprising a compound of any of Embodiments 1-49 or a pharmaceutically acceptable salt thereof, or a hydrate thereof, or a solvate thereof, and a pharmaceutically acceptable excipient.

51. A method of inhibiting or degrading NRF2 by the activation of KEAP1, comprising administering to a subject in need thereof a therapeutically effective amount of a compound of any of Embodiments 1-49 or a pharmaceutically acceptable salt thereof, or a hydrate thereof, or a solvate thereof.

52. A method of treating a disease that is treatable by inhibition or degradation of NRF2 or by activation of KEAP1, comprising administering to a subject in need thereof a therapeutically effective amount of a compound of any of Embodiments 1-49 or a pharmaceutically acceptable salt thereof, or a hydrate thereof, or a solvate thereof.

53. The method of Embodiment 52, wherein the disease is cancer.

54. A compound of Formula (II),

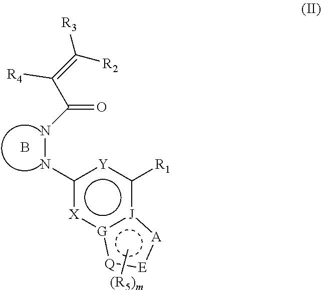

or a pharmaceutically acceptable salt thereof, or a hydrate thereof, or a solvate thereof,
wherein
each of A, E, and Q is independently O, S, N, NH, CH₂, or CH (for clarification, if R₅ substitutes at the corresponding position, NH is N, CH₂ is CH or C, and CH is C. In other words, the options for A, E, and Q as listed here do not take substitutions into consideration),
each of G and J is independently C or N,
X is N or CR₁″,
Y is N or CR₁′, provided that the ring formed by A, E, Q, G, and J is heterocyclyl or heteroaryl, and the ring formed by J, G, X, Y and the two carbons to which Y is aromatic,
R₁, R₁′, and R₁″ are independently H, halogen, CN, C₁₋₆ alkoxyl, C₃₋₈ cycloalkyl, —O—C₃₋₈ cycloalkyl, —OH, —O-4 to 7 membered heterocyclyl, or C₁₋₆ alkyl, wherein each of C₁₋₆ alkoxyl, C₃₋₈ cycloalkyl, —O—C₃₋₈ cycloalkyl, —O-4 to 7 membered heterocyclyl, and C₁₋₆ alkyl is optionally substituted with one or more groups independently selected from halogen, C₁₋₆ alkoxyl, CN, NH₂, NHC₁₋₆ alkyl, N(C₁₋₆ alkyl)₂, OH, —O—C₃₋₈ cycloalkyl, and 4 to 7 membered heterocyclyl that is optionally substituted with one or two groups independently selected from C₁₋₆ alkyl and halogen,
R₂ and R₃ are H,
R₄ is H, F, Cl, CN, NO₂, or C₁₋₆ alkyl substituted with one or more halogen,
R₅ substitutes at A, E, and/or Q, each R₅ is independently (1) oxo or C₁₋₆ alkyl optionally substituted with one or more groups independently selected from halogen, C₁₋₆ alkoxyl, OH, NH₂, NHC₁₋₆ alkyl, N(C₁₋₆ alkyl)₂, and CN, (2) CONR₅ₐR_{5b}, (3) 5-10 membered heteroaryl optionally substituted with one or more R_{5c} independently, (4) C₃₋₈ cycloalkyl optionally substituted with one or more R_{5c}′ independently, or (5) 4-10 membered heterocyclyl optionally substituted with one or more R_{5c}″ independently,
R_{5c}, R_{5c}′, and R_{5c}″ are independently C₁₋₆ alkyl, C₁₋₆ alkoxyl, oxo, CN, OH, halogen, CONR_{5d}R₅ₑ, NHCONR_{5d}R₅ₑ, SO₂NR_{5d}R₅ₑ, SO(NR_{5f})C₁₋₆alkyl, NR_{5d}R₅ₑ, C₃₋₈ cycloalkyl, 5-10 membered heteroaryl, or 4-10 membered heterocyclyl, wherein each of C₁₋₆ alkoxyl, C₁₋₆ alkyl, C₃₋₈ cycloalkyl, 5-10 membered heteroaryl, and 4-10 membered heterocyclyl is optionally substituted with one or more groups independently selected from halogen, C₁₋₆ alkyl, C₁₋₆ alkoxyl, CN, NH₂, NHC₁₋₆ alkyl, N(C₁₋₆ alkyl)₂, oxo, and OH,
R_{5f} is H, C₁₋₆ alkyl, or C₃₋₈ cycloalkyl,
m is 0, 1, or 2,
ring B is 5, 6, or 7 membered heterocyclic optionally substituted with one or two C₁₋₆ alkyl, wherein the one or two C₁₋₆ alkyl are optionally substituted with OH, NH₂, NH(C₁₋₆ alkyl), N(C₁₋₆ alkyl)₂, or C₁₋₆ alkoxy,
R₅ₐ, R_{5b}, R₅ₐ, and R₅ₑ are independently H, SO₂C₁₋₆ alkyl, OH, C₁₋₆ alkoxy, or C₁₋₆ alkyl optionally substituted with one or more groups independently selected from halogen, C₁₋₆ alkoxyl, CN, NH₂, NHC₁₋₆ alkyl, N(C₁₋₆ alkyl)₂, and OH,
or R₅ₐ and R_{5b}, R₅ₐ and R₅ₑ, together with the nitrogen to which they are attached independently form a 4-6 membered heterocyclyl optionally substituted with one or more groups independently selected from halogen, CN, OH, C₁₋₆ alkoxyl, or C₁₋₆ alkyl, wherein each of C₁₋₆ alkoxyl and C₁₋₆ alkyl is optionally independently substituted with one or more groups independently selected from halogen, C₁₋₆ alkoxyl, CN, NH₂, NHC₁₋₆ alkyl, N(C₁₋₆ alkyl)₂, and OH.

55. The compound of Embodiment 54, or a pharmaceutically acceptable salt thereof, or a hydrate thereof, or a solvate thereof, wherein ring B is

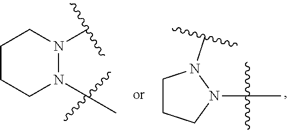 [SMILES: *N1CCCCN1* ; *N1CCCN1*]

each of which is optionally substituted with one or two C₁₋₆ alkyl.

56. The compound of Embodiment 54, or a pharmaceutically acceptable salt thereof, or a hydrate thereof, or a solvate thereof, wherein ring B is

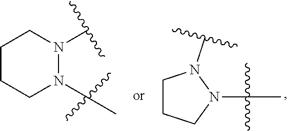 [SMILES: *N1CCCCN1* ; *N1CCCN1*]

each of which optionally has one or more hydrogens replaced with deuterium.

57. The compound of any of Embodiments 54-56, or a pharmaceutically acceptable salt thereof, or a hydrate thereof, or a solvate thereof, wherein R₁ is a halogen.

58. The compound of any of Embodiments 54-56, or a pharmaceutically acceptable salt thereof, or a hydrate thereof, or a solvate thereof, wherein R₁ is F or Cl.

59. The compound of any of Embodiments 54-56, or a pharmaceutically acceptable salt thereof, or a hydrate thereof, or a solvate thereof, wherein R₁ is C₁₋₆ alkyl or C₁₋₆ alkoxyl or C₃₋₈ cycloalkyl.

60. The compound of any of Embodiments 54-56, or a pharmaceutically acceptable salt thereof, or a hydrate thereof, or a solvate thereof, wherein R₁ is methyl or methoxyl or cyclopropyl.

61. The compound of any of Embodiments 54-60, or a pharmaceutically acceptable salt thereof, or a hydrate thereof, or a solvate thereof, wherein Y is CR₁′ and R′ is H.

62. The compound of any of Embodiments 54-60, or a pharmaceutically acceptable salt thereof, or a hydrate thereof, or a solvate thereof, wherein Y is N or CR₁′ and R₁′ is a halogen.

63. The compound of any of Embodiments 54-60, or a pharmaceutically acceptable salt thereof, or a hydrate thereof, or a solvate thereof, wherein Y is CR₁′ and R₁′ is F or Cl.

64. The compound of any of Embodiments 54-60, or a pharmaceutically acceptable salt thereof, or a hydrate thereof, or a solvate thereof, wherein Y is CR₁′ and R₁′ is C₁₋₆ alkyl.

65. The compound of any of Embodiments 54-60, or a pharmaceutically acceptable salt thereof, or a hydrate thereof, or a solvate thereof, wherein Y is CR₁′ and R₁′ is methyl.

66. The compound of any of Embodiments 54-65, or a pharmaceutically acceptable salt thereof, or a hydrate thereof, or a solvate thereof, wherein X is CH.

67. The compound of any of Embodiments 54-65, or a pharmaceutically acceptable salt thereof, or a hydrate thereof, or a solvate thereof, wherein X is (1)N, C—CN, C—C₁₋₆alkoxyl, C—C₁₋₆alkyl, C—F, or C—Cl, or (2) N, C—CN, C—OCH₃, C—OCD₃, C—CH₃, C—F, or C—Cl.

68. The compound of any of Embodiments 54-65, or a pharmaceutically acceptable salt thereof, or a hydrate thereof, or a solvate thereof, wherein X is CR₁″, and R₁″ is H, OH, CN, —C₁₋₆ alkyl, halogen, C₁₋₆ alkoxyl, C₃₋₈ cycloalkyl, —O—C₃₋₈ cycloalkyl, or —O-4 to 7 membered heterocyclyl, wherein each of the —C₁₋₆ alkyl, C₁₋₆ alkoxyl, C₃₋₈ cycloalkyl, —O—C₃₋₈ cycloalkyl, and —O-4 to 7 membered heterocyclyl is optionally substituted with —N(C₁₋₆ alkyl)₂, C₁₋₆ alkoxyl, —O—C₃₋₈ cycloalkyl, or 4 to 7 membered heterocyclyl that is optionally substituted with one or two groups independently selected from C₁₋₆alkyl and halogen.

69. The compound of any of Embodiments 54-65, or a pharmaceutically acceptable salt thereof, or a hydrate thereof, or a solvate thereof, wherein X is CR₁″, and R₁″ is H, OH, CN, methyl, ethyl, F, Cl, methoxy, ethoxy, —OCH(CH₃)₂, —OCH₂CH₂CH₃, —OCD₃, —O-cyclobutyl, cyclopropyl, —OCH₂CH₂N(CH₃)₂, —OCH₂CH₂OCH₃,

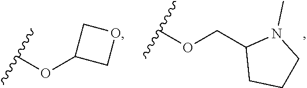

or —OCH₂CH₂O-cyclopropyl.

70. The compound of any of Embodiments 54-65, or a pharmaceutically acceptable salt thereof, or a hydrate thereof, or a solvate thereof, wherein X is N.

71. The compound of any of Embodiments 54-70, or a pharmaceutically acceptable salt thereof, or a hydrate thereof, or a solvate thereof, wherein either A or Q is O or S so that

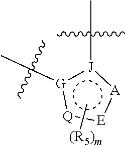

is

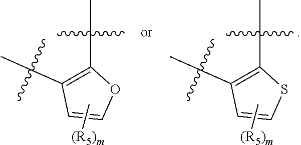

In other words,

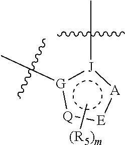

is

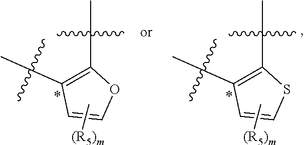

wherein * indicates the position of G or J.

72. The compound of any of Embodiments 54-70, or a pharmaceutically acceptable salt thereof, or a hydrate thereof, or a solvate thereof, wherein

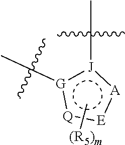

is

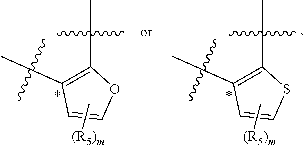

* indicates the position of G.

73. The compound of any of Embodiments 54-70, or a pharmaceutically acceptable salt thereof, or a hydrate thereof, or a solvate thereof, wherein A or Q is O or S so that

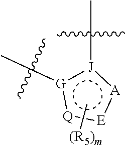

is

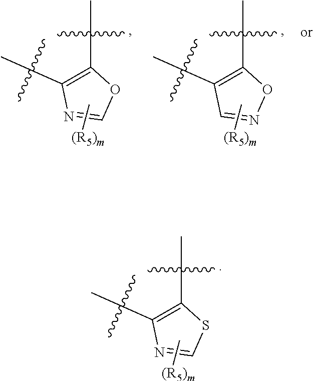

In other words,

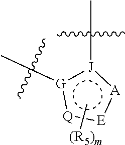

is

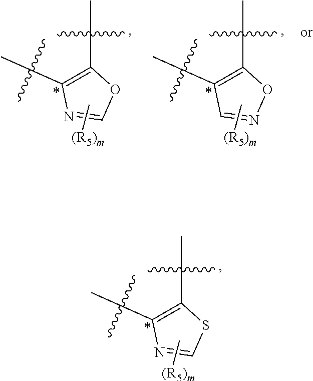

wherein * indicates the position of G or J.

74. The compound of any of Embodiments 54-70, or a pharmaceutically acceptable salt thereof, or a hydrate thereof, or a solvate thereof, wherein G is N so that

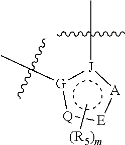

is

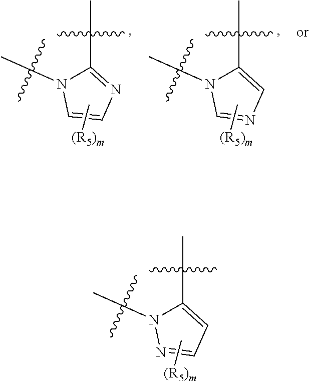

75. The compound of any of Embodiments 54-70, or a pharmaceutically acceptable salt thereof, or a hydrate thereof, or a solvate thereof, wherein J is N so that

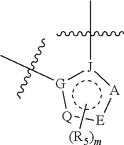

is

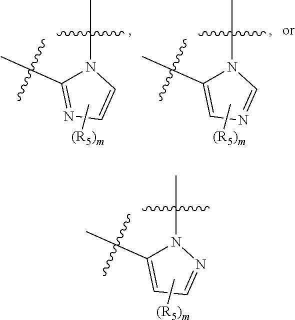

76. The compound of any of Embodiments 54-70, or a pharmaceutically acceptable salt thereof, or a hydrate thereof, or a solvate thereof, wherein either Q or A is N so that

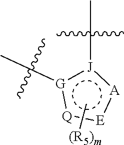

is

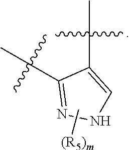

77. The compound of any of Embodiments 54-70, or a pharmaceutically acceptable salt thereof, or a hydrate thereof, or a solvate thereof, wherein

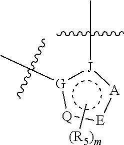

is

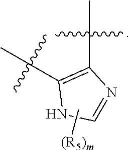

78. The compound of any of Embodiments 54-70, or a pharmaceutically acceptable salt thereof, or a hydrate thereof, or a solvate thereof, wherein

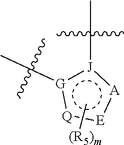

is

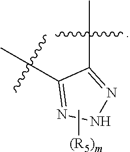

79. The compound of any of Embodiments 54-70, or a pharmaceutically acceptable salt thereof, or a hydrate thereof, or a solvate thereof, wherein

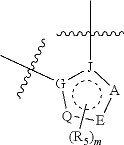

is

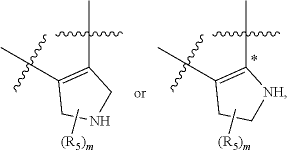

* represents G or J.

80. The compound of any of Embodiments 54-70, or a pharmaceutically acceptable salt thereof, or a hydrate thereof, or a solvate thereof, wherein

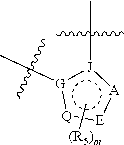

is

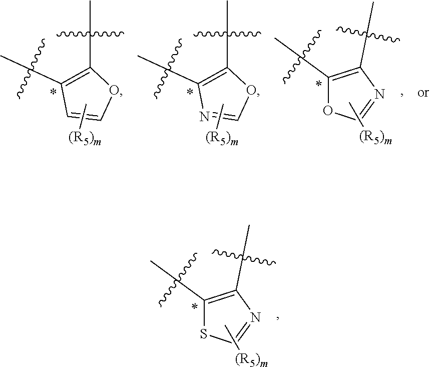

* indicates the position of G.

81. The compound of any of Embodiments 54-80, or a pharmaceutically acceptable salt thereof, or a hydrate thereof, or a solvate thereof, wherein m is 1, R₅ is a 5-10 membered heteroaryl optionally substituted with one or more R_{5c} independently or is a 4-10 membered heterocyclyl optionally substituted with one or more R_{5c}″ independently, with R_{5c} and R_{5c}″ being the same as in Embodiment 54.

82. The compound of any of Embodiments 54-80, or a pharmaceutically acceptable salt thereof, or a hydrate thereof, or a solvate thereof, wherein m is 1, R₅ is pyridinyl, pyrazinyl, pyridazinyl, pyrimidinyl, 1,3,5-triazinyl, indazolyl, pyrazolyl, triazolyl, thiophenyl, pyrrolyl, imidazolyl, furanyl, isothiazolyl, thiazolyl, pyrrolidinyl, piperidinyl, tetrahydrofuranyl, or 1,2,3,6-tetrahydropyridinyl, wherein each of the pyridinyl, pyrazinyl, pyridazinyl, pyrimidinyl, 1,3,5-triazinyl, indazolyl, pyrazolyl, triazolyl, thiophenyl, pyrrolyl, imidazolyl, furanyl, isothiazolyl, and thiazolyl is optionally substituted with one or more R_{5c} independently, wherein each of the pyrrolidinyl, piperidinyl, tetrahydrofuranyl, and 1,2,3,6-tetrahydropyridinyl is optionally substituted with one or more R_{5c}″ independently, with R_{5c} and R_{5c}″ being the same as in Embodiment 54.

83. The compound of any of Embodiments 54-80, or a pharmaceutically acceptable salt thereof, or a hydrate thereof, or a solvate thereof, wherein m is 1, R₅ is (1) pyridinyl, pyrazinyl, pyridazinyl, pyrimidinyl, 1,3,5-triazinyl, indazolyl, pyrazolyl, triazolyl, thiophenyl, pyrrolyl, imidazolyl, furanyl, isothiazolyl, or thiazolyl, each of which is optionally substituted with one or more groups independently selected from CONHC₁₋₆ alkoxyl, CONHOH, CONR_{5d}′R₅ₑ′, NR_{5d}′R₅ₑ′, C₁₋₆ alkyl optionally substituted with CN or C₁₋₆ alkoxyl, C₁₋₆ alkoxyl, CHF₂, CF₃, halogen, OH, CN, SO₂NH₂, SO(NH)C₁₋₆ alkyl, SO(NCH₃)C₁₋₆ alkyl, CONHSO₂C₁₋₆alkyl, NHCONR_{5d}′R₅ₑ′, and 4-6 membered heterocyclyl optionally substituted with C₁₋₆ alkyl, with R_{5d}′ and R₅ₑ′ independently being H or C₁₋₆ alkyl optionally substituted with CN, or R_{5d}′ and R₅ₑ′ together with the nitrogen to which they are attached form a pyrrolidine, piperidine or morpholine, or (2) pyrrolidinyl, piperidinyl, tetrahydrofuranyl, or 1,2,3,6-tetrahydropyridinyl, each of which is optionally substituted with one or more groups independently selected from C₁₋₆ alkyl and CONR_{5d}′R₅ₑ′, with R_{5d}′ and R₅ₑ′ independently being H or C₁₋₆ alkyl optionally substituted with CN, or R_{5d}′ and R₅ₑ′ together with the nitrogen to which they are attached form a pyrrolidine, piperidine or morpholine.

84. The compound of any of Embodiments 54-80, or a pharmaceutically acceptable salt thereof, or a hydrate thereof, or a solvate thereof, wherein m is 1, R₅ is pyridinyl, pyrazolyl, imidazolyl, or triazolyl, each of which is optionally substituted with one or more groups independently selected from C₁₋₆ alkyl optionally substituted with CN or C₁₋₆ alkoxyl, C₁₋₆ alkoxyl, CHF₂, CF₃, halogen, OH, CN, CONR_{5d}′R₅ₑ′, NR_{5d}′R₅ₑ′, and 4-6 membered heterocyclyl optionally substituted with C₁₋₆ alkyl, with R_{5d}′ and R₅ₑ′ independently being H or C₁₋₆ alkyl.

85. The compound of any of Embodiments 54-80, or a pharmaceutically acceptable salt thereof, or a hydrate thereof, or a solvate thereof, wherein m is 1, and R₅ is pyridinyl, pyrazolyl, imidazolyl, or triazolyl, each of which is optionally substituted with one or more groups independently selected from NH₂, methyl, F, Cl, CF₃, methoxyl, —CONH₂, CHF₂, CN, OH, oxetyl optionally substituted with C₁₋₆ alkyl, pyrrolidinyl optionally substituted with C₁₋₆ alkyl, and tetrahydrofuranyl optionally substituted with C₁₋₆ alkyl.

86. The compound of any of Embodiments 54-85, or a pharmaceutically acceptable salt thereof, or a hydrate thereof, or a solvate thereof, wherein m is 1 and R₅ substitutes at E.

87. The compound of any of Embodiments 54-80, or a pharmaceutically acceptable salt thereof, or a hydrate thereof, or a solvate thereof, wherein m is 1, and R₅ is

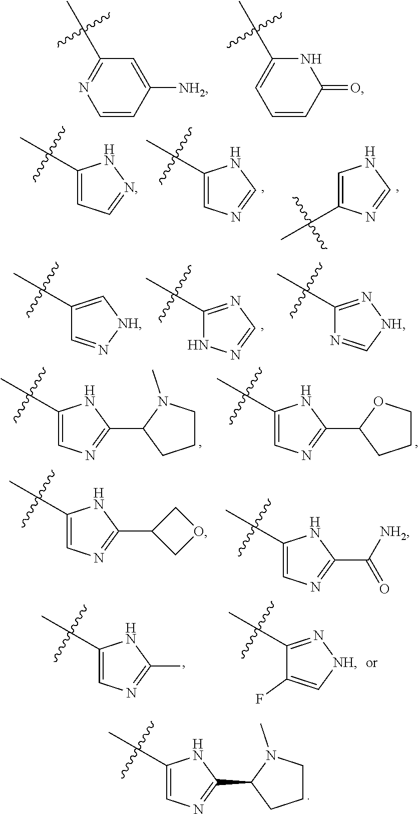

88. The compound of any of Embodiments 54-80, or a pharmaceutically acceptable salt thereof, or a hydrate thereof, or a solvate thereof, wherein m is 1, and R₅ is

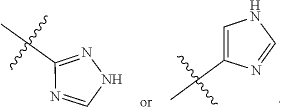 [SMILES: *c1nc[nH]n1 ; "or" ; *c1c[nH]cn1]

89. The compound of Embodiment 87 or 88, or a pharmaceutically acceptable salt thereof, or a hydrate thereof, or a solvate thereof, wherein R₅ substitutes at E.

90. The compound of any of Embodiments 54-89, or a pharmaceutically acceptable salt thereof, or a hydrate thereof, or a solvate thereof, wherein R₄ is H or D.

91. The compound of any of Embodiments 54-89, or a pharmaceutically acceptable salt thereof, or a hydrate thereof, or a solvate thereof, wherein R₄ is F.

92. The compound of any of Embodiments 54-89, or a pharmaceutically acceptable salt thereof, or a hydrate thereof, or a solvate thereof, wherein R₄ is CN or NO₂.

93. The compound of any of Embodiments 54-92, or a pharmaceutically acceptable salt thereof, or a hydrate thereof, or a solvate thereof, wherein the compound has one or more hydrogens replaced by deuterium.

94. The compound of any of Embodiments 54-92, or a pharmaceutically acceptable salt thereof, or a hydrate thereof, or a solvate thereof, wherein at least one of R₂ and R₃ is deuterium.

95. The compound of Embodiment 54, or a pharmaceutically acceptable salt thereof, or a hydrate thereof, or a solvate thereof, wherein the compound is selected from the compounds in Table 1B below, or a pharmaceutically acceptable salt thereof, or a hydrate thereof, or a solvate thereof.

TABLE 1B

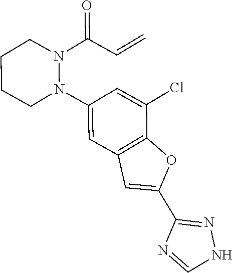 [SMILES: C=CC(=O)N1CCCCN1c1cc(Cl)c2oc(-c3nc[nH]n3)cc2c1]

B43

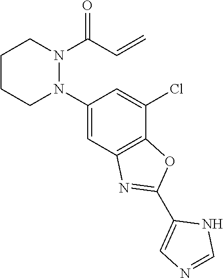 [SMILES: C=CC(=O)N1CCCCN1c1cc(Cl)c2oc(-c3cnc[nH]3)nc2c1]

D33

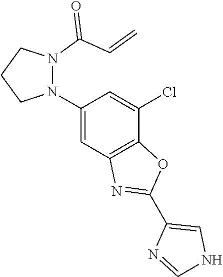 [SMILES: C=CC(=O)N1CCCN1c1cc(Cl)c2oc(-c3c[nH]cn3)nc2c1]

D34

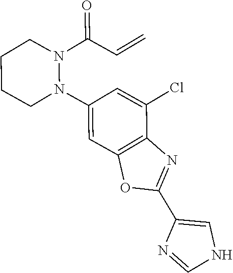 [SMILES: C=CC(=O)N1CCCCN1c1cc(Cl)c2nc(-c3c[nH]cn3)oc2c1]

L14

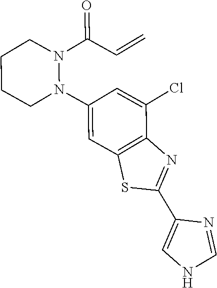 [SMILES: C=CC(=O)N1CCCCN1c1cc(Cl)c2nc(-c3c[nH]cn3)sc2c1]

N9

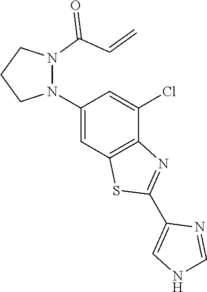 [SMILES: C=CC(=O)N1CCCN1c1cc(Cl)c2nc(-c3c[nH]cn3)sc2c1]

N10

96. The compound of Embodiment 95, or a pharmaceutically acceptable salt thereof, or a hydrate thereof, or a solvate thereof, wherein the compound has one or more hydrogens replaced by deuterium.

97. A pharmaceutical composition comprising a compound of any of Embodiments 54-96 or a pharmaceutically acceptable salt thereof, or a hydrate thereof, or a solvate thereof, and a pharmaceutically acceptable excipient.

98. A method of inhibiting or degrading NRF2 by the activation of KEAP1, comprising administering to a subject in need thereof a therapeutically effective amount of a compound of any of Embodiments 54-96 or a pharmaceutically acceptable salt thereof, or a hydrate thereof, or a solvate thereof.

99. A method of treating a disease that is treatable by inhibition or degradation of NRF2 or by activation of KEAP1, comprising administering to a subject in need thereof a therapeutically effective amount of a compound of any of Embodiments 54-99 or a pharmaceutically acceptable salt thereof, or a hydrate thereof, or a solvate thereof.

100. The method of Embodiment 99, wherein the disease is cancer.

The compound disclosed herein may contain asymmetric or chiral centers, and therefore, exist in different stereoisomeric forms, such as enantiomers, diastereomers, or atropisomers. All stereoisomeric forms of the compounds disclosed herein as well as mixtures thereof, including racemic mixtures, form part of the present invention.

It should also be noted that the compound disclosed herein can include E and Z isomers, or a mixture thereof, and cis and trans isomers or a mixture thereof.

It is also possible that the compound disclosed herein may exist in different tautomeric forms, and all such forms are within the scope of the present invention.

In the compound disclosed herein, the atoms may exhibit their natural abundances, or one or more of the atoms may be artificially enriched in a particular isotope having the same atomic number, but an atomic mass or mass number different from the atomic mass or mass number predominantly found in nature. All suitable isotopic variations of the compound of Formula (I) or Formula (II) are within the scope of the present invention. For example, different isotopic forms of hydrogen (H) include protium (¹H) and deuterium (²H).

As further examples, compounds of Formula (I) or Formula (II) include a compound of Formula (I) or Formula (II) (such as a compound of Table 1A or Table 1B) where one or more atoms are replaced with an atom having the same atomic number, but an atomic mass or mass number different from the atomic mass or mass number predominantly found in nature (also known as isotopically or radio-labeled compound). Exemplary isotopically or radio-labeled compound of Formula (I) or Formula (II) include a compound of Formula (I) or Formula (II) (such as a compound in Table 1A or Table 1B) wherein one or more hydrogen atoms are replaced by deuterium, for example, the hydrogen at R₂ and/or R₃ can be replaced by deuterium. Synthetic techniques for introducing isotopes into organic compounds are well established in the field, and a person of ordinary skill in the art will readily identify the appropriate methods applicable to the compounds of the invention.

### Methods for Making Compounds

The Compounds can be made using conventional organic syntheses and commercially available starting materials. It should be noted that one skilled in the art would know how to modify the procedures set forth in the illustrative schemes and examples to arrive at the desired products.

### EXAMPLES

The examples below are intended to be purely exemplary and should not be considered to be limiting in any way. Unless otherwise specified, the experimental methods in the Examples described below are conventional methods. Unless indicated otherwise, temperature is in degrees Centigrade. Reagents were purchased from commercial suppliers such as Sigma-Aldrich, Alfa Aesar, or TCI, and were used without further purification unless indicated otherwise. ¹H NMR spectra were recorded on a Varian instrument operating at 400 MHz.

In the following examples, the following abbreviations are used:

| | |
|---|---|
| AcOH | Acetic acid |
| Aq | Aqueous |
| Brine | Saturated aqueous sodium chloride solution |
| Bn | Benzyl |
| BnBr | Benzyl Bromide |
| (Boc)₂O | di-tert-butyl dicarbonate |
| Dppf | 1,1″-bis(diphenylphosphino)ferrocene |
| DBU | 1,8-diazabicyclo[5.4.0]undec-7-ene |
| DIEA or DIPEA | N-ethyl-N-isopropylpropan-2-amine |
| DMAP | 4-N,N-dimethylaminopyridine |
| DMF | N,N-dimethylformamide |
| DMF-DMA | N,N-dimethyl formamide dimethyl acctel |
| DMP | Dess-Martin Periodinane |
| DMSO | Dimethyl sulfoxide |
| EA | Ethyl acetate |
| EtOH | Ethanol |
| Et₂O or ether | Diethyl ether |
| Et₃N | Triethyl amine |
| HATU | O-(7-Azabenzotriazol-1-yl)-N,N,N′,N′- |
| | tetramethyluronium |
| | hexafluorophosphate |
| HBpin | 4,4,5,5-Tetramethyl-1,3,2-dioxaborolane |
| HPLC | High-performance liquid chromatography |
| IPA | 2-propanol |
| i-PrOH | Isopropyl alcohol |
| ms or MS | Mass spectrum |
| MTBE | Methyl tert-butyl ether |
| NaHMDS | Sodium Hexamethylenedisilazane |
| PE | petroleum ether |
| PPA | Polyphosphoric acid |
| p-TSA | p-Tolunesulfonic acid |
| Rt | Retention time |
| RT or rt | Room temperature |
| TBAF | Tetra-butyl ammonium fluoride |
| TBSCl | tert-Butyldimethylsilyl chloride |
| TFA | Trifluoroacetic acid |
| THF | tetrahydrofuran |
| TLC | thin layer chromatography |
| STAB | Sodium triacetoxyborohydride |
| dtbpf | 1,1′-Bis(di-tert-butylphosphino)ferrocene |

### Example 1

### Synthesis of Intermediates

### Example 1-1: Synthesis of 5-(5-bromo-7-chlorobenzofuran-2-yl)-1-((2-(trimethylsilyl)ethoxy) methyl)-1H-pyrazole

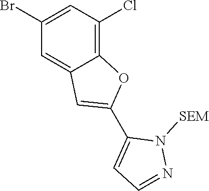 [SMILES: C[Si](C)(C)CCOCn1nccc1-c1cc2cc(Br)cc(Cl)c2o1]

### Step 1: 1-(5-bromo-7-chlorobenzofuran-2-yl)ethan-1-one

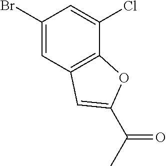

To a solution of 5-bromo-3-chloro-2-hydroxybenzaldehyde (10 g, 42.7 mmol) in acetone (150 mL) was added K₂CO₃ (8.9 g, 64.1 mmol) and 1-chloropropan-2-one (5.9 g, 64.1 mmol). The resulting mixture was heated to 55° C. with stirring for 16 hrs. After cooled to rt, the reaction mixture was quenched by water/ice and extracted with EA. The organic layer was washed with brine, dried over Na₂SO₄ and concentrated. The residue was purified by silica gel column chromatography (PE/EA=2:1) to give the desired product (8.4 g, 72%). MS (ESI) m/z [M+H]⁺ 273, 275.

### Step 2: 1-(5-bromo-7-chlorobenzofuran-2-yl)-3-(dimethylamino)prop-2-en-1-one

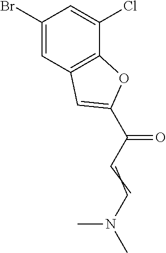 [SMILES: CN(C)C=CC(=O)c1cc2cc(Br)cc(Cl)c2o1]

To a solution of 1-(5-bromo-7-chlorobenzofuran-2-yl)ethan-1-one (8.4 g, 30.9 mmol) in toluene (150 mL) was added DMF-DMA (3.7 g, 30.9 mmol). The resulting mixture was heated to 120° C. with stirring for 2 hrs. Upon completion of the reaction, the resulting reaction was cooled to room temperature. The resulting mixture was concentrated. This resulted in 13 g of crude desired product. MS (ESI) m/z [M+H]⁺ 328, 330.

### Step 3: 5-(5-bromo-7-chlorobenzofuran-2-yl)-1H-pyrazole

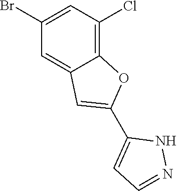 [SMILES: Clc1cc(Br)cc2cc(-c3ccn[nH]3)oc12]

To a solution of 1-(5-bromo-7-chlorobenzofuran-2-yl)-3-(dimethylamino)prop-2-en-1-one (13 g, crude) in AcOH (150 mL) was added hydrazinium hydroxide solution (1.8 g (85% by weight), 30.9 mmol). The resulting mixture was heated to 80° C. with stirring for 2 hrs. The resulting reaction was cooled to room temperature. The resulting reaction was diluted by DCM. Solids were filtered out. The filtration was concentrated. To the reaction solution was added EA. The solids were filtered out. The residue was purified by silica gel column chromatography (DCM/MeOH=20:1) to give the desired product (3.6 g, 40% for 2 steps). MS (ESI) m/z [M+H]⁺ 297, 299.

### Step 4: 5-(5-bromo-7-chlorobenzofuran-2-yl)-1-((2-(trimethylsilyl)ethoxy)methyl)-1H-pyrazole

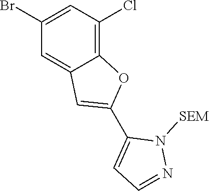 [SMILES: C[Si](C)(C)CCOCn1nccc1-c1cc2cc(Br)cc(Cl)c2o1]

A solution of 5-(5-bromo-7-chlorobenzofuran-2-yl)-1H-pyrazole (670 mg (impure), 2.3 mmol) in THF (12 mL) was cooled to 0° C. under nitrogen. To this was added NaH (60% in mineral oil, 100 mg, 2.5 mmol) at 0° C. The resulting mixture was stirred below 0° C. for 10 min. To this was then added a solution of SEM-Cl (416 mg, 2.5 mmol) in THF (3 mL) below 0° C. The resulting mixture was stirred at 10° C. for 30 min. The resulting reaction was warmed to rt with stirring for 30 min. Upon completion of the reaction, the reaction was quenched by NH₄Cl (aq.) and extracted by EA. The organic layer was washed with brine, dried over Na₂SO₄ and concentrated. The residue was purified by silica gel column chromatography (EA/PE=1:6) to give the desired product (150 mg, 16%). MS (ESI) m/z [M+H]⁺ 427, 429.

### Example 1-2: Synthesis of (5-(4-(tert-butoxycarbonyl)morpholin-3-yl)-7-chlorobenzofuran-2-yl)boronic Acid

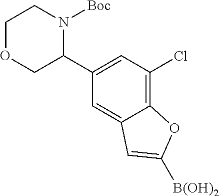 [SMILES: CC(C)(C)OC(=O)N1CCOCC1c1cc(Cl)c2oc(B(O)O)cc2c1]

### Step 1: 2-(7-chlorobenzofuran-5-yl)-4,4,5,5-tetramethyl-1,3,2-dioxaborolane

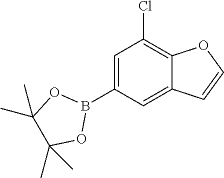 [SMILES: CC1(C)OB(c2cc(Cl)c3occc3c2)OC1(C)C]

To a round bottom flask was placed a mixture of 5-bromo-7-chlorobenzofuran (6 g, 26.1 mmol), 4,4,4′,4′,5,5,5′,5′-octamethyl-2,2′-bi(1,3,2-dioxaborolane) (8.6 g, 33.9 mmol), Pd(dppf)Cl2 (953 mg, 1.3 mmol), KOAc (3.8 g, 39.1 mmol) and dioxane (100 mL). The resulting mixture was charged into nitrogen and heated to 90° C. with stirring for 16 hrs. After cooled to rt, EA was added and the solids were filtered out. The filtrate was concentrated in vacuo and the residue was purified with silica gel column chromatography (EA/PE=1:15) to give the desired product (6.6 g, 91%). MS (ESI) m/z [M+H]⁺ 279.

### Step 2: tert-butyl 5-(7-chlorobenzofuran-5-yl)-2,3-dihydro-4H-1,4-oxazine-4-carboxylate

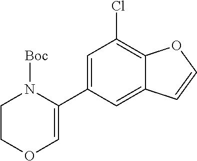 [SMILES: CC(C)(C)OC(=O)N1CCOC=C1c1cc(Cl)c2occc2c1]

A mixture of 2-(7-chlorobenzofuran-5-yl)-4,4,5,5-tetramethyl-1,3,2-dioxaborolane (6.6 g, 23.7 mmol), tert-butyl 5-((diphenoxyphosphoryl)oxy)-2,3-dihydro-4H-1,4-oxazine-4-carboxylate (10.3 g, 23.7 mmol), Pd(dppf)Cl₂ (870 mg, 1.2 mmol) and K₂CO₃ (6.5 g, 47.5 mmol) were added into dioxane (100 mL) and H₂O (25 mL). The resulting mixture was charged into nitrogen and heated to 80° C. with stirring for 16 hrs. After cooled to rt, the resulting reaction was diluted with EA. The organic layer was washed with brine, dried over Na₂SO₄ and concentrated. The residue was applied onto a silica gel column chromatography (EA/PE=1:5) to give the desired product (6.1 g, 77%). MS (ESI) m/z [M+H]⁺ 336.

### Step 3: 3-(7-chlorobenzofuran-5-yl)morpholine

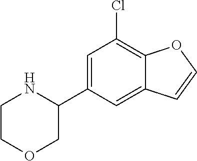 [SMILES: Clc1cc(C2COCCN2)cc2ccoc12]

A solution of tert-butyl 5-(7-chlorobenzofuran-5-yl)-2,3-dihydro-4H-1,4-oxazine-4-carboxylate (6.1 g, 18.2 mmol) in DCM (40 mL) was cooled to 0° C. To this was added TFA (20 mL) at 0° C. The resulting reaction was warmed to rt with stirring for 30 min. The reaction mixture was re-cooled to 0° C. To this was added STAB (15.4 g, 72.8 mmol) in several portions and stirred below 10° C. for 30 min. The resulting reaction was warmed to rt with stirring for 1 h. Upon completion of the reaction, the solvent was removed. The residue was dissolved into DCM/MeOH (10:1). The organic layer was washed with NaHCO₃ (aq.) and brine, dried over Na2SO4 and concentrated to give the desired product (5.8 g, crude). MS (ESI) m/z [M+H]⁺ 238.

### Step 4: tert-butyl 3-(7-chlorobenzofuran-5-yl)morpholine-4-carboxylate

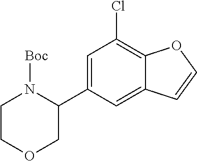 [SMILES: CC(C)(C)OC(=O)N1CCOCC1c1cc(Cl)c2occc2c1]

To a solution of 3-(7-chlorobenzofuran-5-yl)morpholine (5.8 g, crude) in DCM (30 mL) and NaHCO₃ (aq) (30 mL) was added a solution of Boc₂O (7.9 g, 36.4 mmol) at 0° C. The mixture was stirred at rt for 16 hrs. Upon completion of the reaction, the reaction was diluted by EA. The organic layer was washed with brine and concentrated. The residue was applied onto a silica gel column chromatography (EA/PE=1:6) to give the desired product (5.5 g, 89% for 2 steps). MS (ESI) m/z [M+H]⁺=338.

### Step 5: (5-(4-(tert-butoxycarbonyl)morpholin-3-yl)-7-chlorobenzofuran-2-yl)boronic Acid

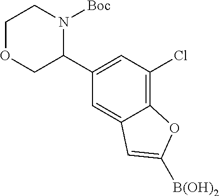 [SMILES: CC(C)(C)OC(=O)N1CCOCC1c1cc(Cl)c2oc(B(O)O)cc2c1]

To a solution of tert-butyl 3-(7-chlorobenzofuran-5-yl)morpholine-4-carboxylate (3.5 g, 10.4 mmol) in THF (60 mL) was added Ir(OMe)(1,5-cod)]2 (103 mg, 0.16 mmol) and dtbpy (84 mg, 0.3 mmol). The resulting mixture was degassed with nitrogen. To this was added HBpin (4 g, 31.2 mmol). The resulting reaction was stirred at rt for 3 hrs. The resulting reaction was quenched by MeOH. The solvents were removed in vacuo. The residue was applied onto C18 column with MeCN/H₂O (0.1% FA) to give the desired product (2.7 g, 68%). MS (ESI) m/z [M+H]⁺ 382.

### Example 1-3: Synthesis of (5-(4-(tert-butoxycarbonyl)piperazin-2-yl)-7-chlorobenzofuran-2-yl)boronic Acid

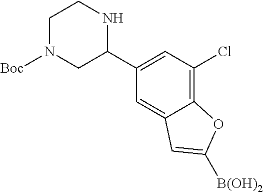 [SMILES: CC(C)(C)OC(=O)N1CCNC(c2cc(Cl)c3oc(B(O)O)cc3c2)C1]

### Step 1: 2-(7-chlorobenzofuran-5-yl)pyrazine

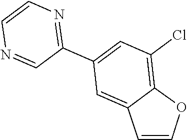 [SMILES: Clc1cc(-c2cnccn2)cc2ccoc12]

To a round bottom flask was placed 2-bromopyrazine (4.7 g, 30 mmol), 2-(7-chlorobenzofuran-5-yl)-4,4,5,5-tetramethyl-1,3,2-dioxaborolane (8.2 g, 29 mmol), Pd(dppf)Cl₂ (1.1 g, 1.5 mmol) and K₂CO₃ (8.1 g, 59 mmol). To this was added 1,4-dioxane (120 mL) and H₂O (20 mL). The resulting mixture was charged into nitrogen and heated to 80° C. with stirring for 16 hrs. Upon completion of the reaction, the resulting reaction was cooled to rt and EA was added. The organic layer was washed with brine, dried over Na₂SO₄ and concentrated. The residue was applied onto a silica gel column chromatography (EA/PE=1:2) to give the desired product (6.2 g, 91%). MS (ESI) m/z [M+H]⁺ 231.

### Step 2: 2-(7-chlorobenzofuran-5-yl)piperazine

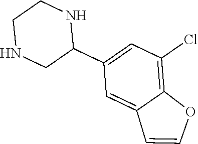 [SMILES: Clc1cc(C2CNCCN2)cc2ccoc12]

2-(7-Chlorobenzofuran-5-yl)pyrazine (6.2 g, 27 mmol), N-phenylaniline (10.3 g, 60.7 mmol) and 4,4,5,5-tetramethyl-1,3,2-dioxaborolane (19.4 g, 151.7 mmol) were added into toluene (100 mL). N₂ was charged into the resulting mixture. To this was added tris(2,3,4,5,6-pentafluorophenyl)borane (1.6 g, 3.0 mmol). The resulting mixture was heated to 110° C. with stirring for 16 hrs. After cooled to rt, toluene was removed in vacuo. The residue was added into HCl (0.5 N) and the aqueous was extracted with MTBE for two times. The pH value was then adjusted to 12 by NaOH (1N) and extracted with DCM/MeOH (10:1). The organic layer was washed with brine, dried over Na2SO4 and concentrated. to give the title product (8.9 g, purity: 65%, yield: 91%). MS (ESI) m/z [M+H]⁺ 237.

### Step 3: tert-butyl 3-(7-chlorobenzofuran-5-yl)piperazine-1-carboxylate

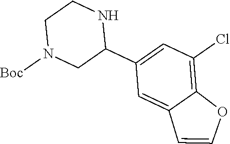 [SMILES: CC(C)(C)OC(=O)N1CCNC(c2cc(Cl)c3occc3c2)C1]

To a round bottom flask was added a solution of 2-(7-chlorobenzofuran-5-yl)piperazine (8.9 g, 24 mmol, 65% purity) in THF (100 mL). To this was added sat. NaHCO₃ (aq.) (100 mL). The resulting mixture was cooled to 0° C. To this was then added tert-butoxycarbonyl tert-butyl carbonate (5.2 g, 24 mmol) at 0° C. The resulting reaction was stirred below 10° C. for 1 h. The reaction was diluted with water and extracted by EA. The organic layer was washed with brine, dried over Na₂SO₄ and concentrated. The residue was applied onto a silica gel column chromatography with DCM/MeOH (20:1) to give the title product (9.7 g, 23 mmol, purity: 80%, yield: 94%). MS (ESI) m/z [M+H]⁺ 337.

### Step 4: (5-(4-(tert-butoxycarbonyl)piperazin-2-yl)-7-chlorobenzofuran-2-yl)boronic Acid

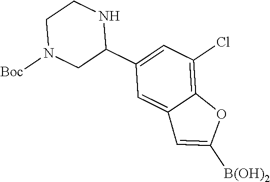 [SMILES: CC(C)(C)OC(=O)N1CCNC(c2cc(Cl)c3oc(B(O)O)cc3c2)C1]

To a round bottom flask was placed tert-butyl 3-(7-chlorobenzofuran-5-yl)piperazine-1-carboxylate (4.1 g, 9.7 mmol, purity: 80%), Ir(OMe)(1,5-cod)]2 (0.16 g, 0.24 mmol) and 4,4′-di-tert-butyl-2,2′-bipyridine (128 mg, 0.48 mmol) were added into THF (60 mL). The resulting mixture was charged into nitrogen. This was then added 4,4,5,5-tetramethyl-1,3,2-dioxaborolane (4.7 g, 37 mmol) at rt. The resulting reaction was stirred at R.T. for 16 hrs. Upon completion of the reaction, the solvent was removed in vacuo and the residue was applied onto C18 column with MeCN/H2O (0.1 FA) to give the title product (3.1 g, 84% yield). MS (ESI) m/z [M+H]⁺ 381.

### Example 1-4: Synthesis of 7-chloro-5-(piperazin-2-yl)-2-(1-((2-(trimethylsilyl) ethoxy)methyl)-1H-pyrazol-5-yl)benzo[d]oxazole

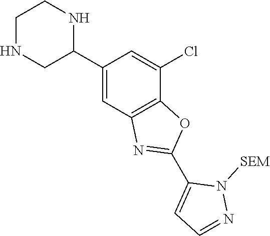 [SMILES: C[Si](C)(C)CCOCn1nccc1-c1nc2cc(C3CNCCN3)cc(Cl)c2o1]

### Step 1: 5-bromo-7-chloro-2-(1H-pyrazol-5-yl)benzo[d]oxazole

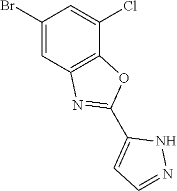 [SMILES: Clc1cc(Br)cc2nc(-c3ccn[nH]3)oc12]

A solution of 2-amino-4-bromo-6-chlorophenol (9.8 g, 44.1 mmol) and 5-(trifluoromethyl)-1H-pyrazole (5.0 g, 36.8 mmol) in NaOH/H₂O (1M, 110 mL) was stirred at 80° C. for overnight. After cooled to rt, the reaction mixture was filtered, the filter cake was washed with 1N HCl, water and the solid was dried in vacuo to give the title compound (7.0 g, 53%). MS (ESI) m/z [M+H]⁺ 298/300.

### Step 2: 5-bromo-7-chloro-2-(1-((2-(trimethylsilyl)ethoxy)methyl)-1H-pyrazol-5-yl)benzo[d]oxazole

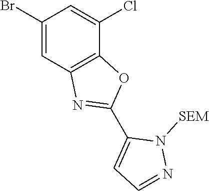 [SMILES: C[Si](C)(C)CCOCn1nccc1-c1nc2cc(Br)cc(Cl)c2o1]

To a solution of 5-bromo-7-chloro-2-(1H-pyrazol-5-yl)benzo[d]oxazole (2.0 g, 6.73 mmol) in THF (30 mL) and DMF (30 mL) was added NaH (323 mg, 60% in oil, 8.08 mmol) at 0° C. and the mixture was stirred at 0° C. for 1 hour. Then SEM-Cl (1.46 mg, 8.75 mmol) was added at 0° C. The resulting mixture was stirred at 0° C. for 1 hour. Upon completion of the reaction, the reaction mixture was quenched with water and extracted with EA, the organic layer was washed with brine, dried over with Na₂SO₄, filtered, concentrated and residue was purified by silica gel column (PE/EA=20/1 to 17/3) to give the title compound (2.8 g, 97%). MS (ESI) m/z [M+H]⁺ 428/430.

### Step 3: 7-chloro-5-(4,4,5,5-tetramethyl-1,3,2-dioxaborolan-2-yl)-2-(1-((2-(trimethylsilyl)ethoxy)methyl)-1H-pyrazol-5-yl)benzo[d]oxazole

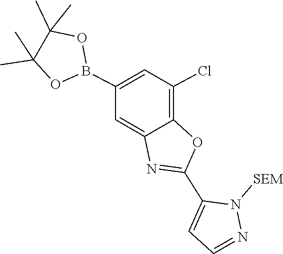 [SMILES: CC1(C)OB(c2cc(Cl)c3oc(-c4ccnn4COCC[Si](C)(C)C)nc3c2)OC1(C)C]

A mixture of 5-bromo-7-chloro-2-(1-((2-(trimethylsilyl)ethoxy)methyl)-1H-pyrazol-5-yl)benzo[d]oxazole (1.4 g, 3.3 mmol), B₂Pin₂ (1.2 g, 4.7 mmol), Pd(dppf)Cl₂ (0.24 g, 0.33 mmol) and KOAc (0.63 g, 6.6 mmol) in 1,4-dioxane (40 mL) was stirred at 90° C. for 2 hours. The solvent was removed in vacuo to give the crude product and it was used directly for the next step without purification. MS (ESI) m/z [M+H]⁺ 476.

### Step 4: 7-chloro-5-(pyrazin-2-yl)-2-(1-((2-(trimethylsilyl)ethoxy)methyl)-1H-pyrazol-5-yl)benzo[d]oxazole

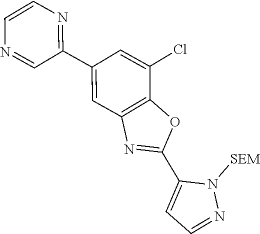 [SMILES: C[Si](C)(C)CCOCn1nccc1-c1nc2cc(-c3cnccn3)cc(Cl)c2o1]

A solution of 7-chloro-5-(4,4,5,5-tetramethyl-1,3,2-dioxaborolan-2-yl)-2-(1-((2-(trimethylsilyl)ethoxy)methyl)-1H-pyrazol-5-yl)benzo[d]oxazole (1.6 g, 3.4 mmol), 2-bromopyrazine (0.53 g, 3.3 mmol), Pd(dppf)Cl₂ (0.25 g, 0.34 mmol) and K₂CO₃ (0.93 g, 6.7 mmol) in 1,4-dioxane (40 mL) and H₂O (10 mL) was stirred at 90° C. for 2 hours. After cooled to rt, the reaction mixture was concentrated and EA was added, the mixture was washed with brine, dried over Na₂SO₄, filtered, concentrated under reduced pressure. The residue was purified by silica gel column chromatography (PE/EtOAc 5/1 to 1/1) to give the title compound (1.26 g, 90% for two steps). MS (ESI) m/z [M+H]⁺ 428.

### Step 5: 7-chloro-5-(piperazin-2-yl)-2-(1-((2-(trimethylsilyl)ethoxy)methyl)-1H-pyrazol-5-yl)benzo[d]oxazole

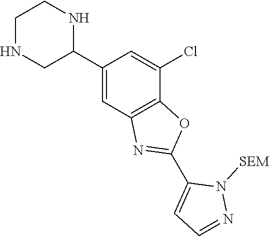 [SMILES: C[Si](C)(C)CCOCn1nccc1-c1nc2cc(C3CNCCN3)cc(Cl)c2o1]

A solution of 7-chloro-5-(pyrazin-2-yl)-2-(1-((2-(trimethylsilyl)ethoxy)methyl)-1H-pyrazol-5-yl)benzo[d]oxazole (1.2 g, 2.8 mmol), 4,4,5,5-tetramethyl-1,3,2-dioxaborolane (2.9 g, 23 mmol), N-phenylaniline (1.9 g, 11 mmol) and tris(2,3,4,5,6-pentafluorophenyl)borane (0.29 g, 0.57 mmol) in toluene (20 mL) was stirred at 110° C. for overnight. The reaction was concentrated in vacuo and the residue was purified by silica gel column chromatography (MeOH/DCM 10/1 to 2/3) to give the title compound (1.06 g, 87.1%). MS (ESI) m/z [M+H]⁺ 434.

### Example 1-5: Synthesis of 1-(5-(4-acetamido-1-tosylpiperidin-2-yl)-7-chlorobenzofuran-2-yl)boronic Acid

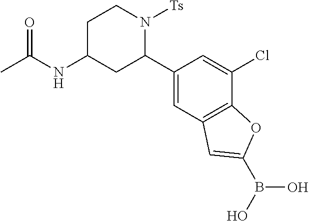 [SMILES: CC(=O)NC1CCN(S(=O)(=O)c2ccc(C)cc2)C(c2cc(Cl)c3oc(B(O)O)cc3c2)C1]

### Step 1: (7-chlorobenzofuran-5-yl)methanol

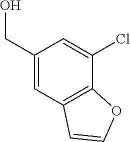 [SMILES: OCc1cc(Cl)c2occc2c1]

A solution of 5-bromo-7-chloro-benzofuran (2.0 g, 8.6 mmol), tributylstannylmethanol (4.2 g, 13 mmol) and Pd-XPhos-G4 (370 mg, 0.43 mmol) in 1,4-dioxane (20 mL) was stirred at 100° C. for 1 hour. After cooled to rt, the reaction mixture was concentrated in vacuo and the residue was purified by silica gel column chromatography (PE/EtOAc 10/1 to 2/3) to give the title compound (1.5 g, 94.8%). MS (ESI) m/z [M+H]⁺ 183.

### Step 2: 7-chlorobenzofuran-5-carbaldehyde

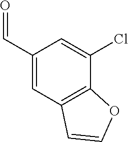 [SMILES: O=Cc1cc(Cl)c2occc2c1]

To a solution of (7-chlorobenzofuran-5-yl) methanol (1.5 g, 8.2 mmol) in DCM (50 mL) was added DMP (7 g, 16.5 mmol) and the resulting mixture was stirred at rt for 1 hour. Upon completion of the reaction, the reaction mixture was filtered and the filtrate was concentrated, the residue was purified by silica gel column chromatography (PE/EtOAc 10/1 to 1/3) to give the title compound (1.29 g, 86.9%). MS (ESI) m/z [M+H]⁺ 181.

### Step 3: N-(2-(7-chlorobenzofuran-5-yl)-1-tosylpiperidin-4-yl)acetamide

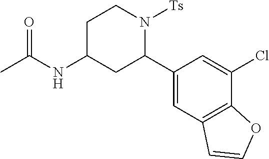 [SMILES: CC(=O)NC1CCN(S(=O)(=O)c2ccc(C)cc2)C(c2cc(Cl)c3occc3c2)C1]

To a solution of 7-chlorobenzofuran-5-carbaldehyde (1.24 g, 6.87 mmol) and N-but-3-enyl-4-methyl-benzenesulfonamide (1.55 g, 6.88 mmol) in ACN (30 mL) was added TfOH (1.54 g, 10.3 mmol) at 0° C. and the resulting mixture was stirred at 0° C. for 1 hours. The reaction solution was concentrated in vacuo and the residue was diluted with DCM, washed with brine, dried over Na₂SO₄, filtered, concentrated and the residue was purified by silica gel column chromatography (MeOH/DCM 1/100 to 1/20) to give the title compound (2.2 g, 71.6%). MS (ESI) m/z [M+H]⁺ 447.

### Step 4: (5-(4-acetamido-1-tosylpiperidin-2-yl)-7-chlorobenzofuran-2-yl)boronic Acid

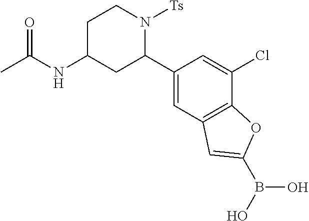 [SMILES: CC(=O)NC1CCN(S(=O)(=O)c2ccc(C)cc2)C(c2cc(Cl)c3oc(B(O)O)cc3c2)C1]

A solution of N-[2-(7-chlorobenzofuran-5-yl)-1-(p-tolylsulfonyl)-4-piperidyl]acetamide (500 mg, 1.12 mmol), HBPin (430 mg, 3.36 mmol), Ir[(OMe)(1,5-cod)]2 (37 mg, 0.06 mmol) and dtbpy (30 mg, 0.11 mmol) in THF (20 mL) was stirred at rt for overnight. The reaction was concentrated and the residue was purified by C18 column [H₂O(0.1% FA):ACN=10% to 90%] to give the title compound (440 mg, 80.1%). MS (ESI) m/z [M+H]⁺ 491.

### Example 1-6: Synthesis of 7-chloro-5-(4,4,5,5-tetramethyl-1,3,2-dioxaborolan-2-yl)-2-(1-((2-(trimethylsilyl)ethoxy)methyl)-1H-imidazol-4-yl)benzo[d]oxazole

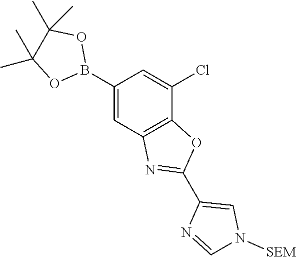 [SMILES: CC1(C)OB(c2cc(Cl)c3oc(-c4cn(COCC[Si](C)(C)C)cn4)nc3c2)OC1(C)C]

A solution of 2-[[4-(5-bromo-7-chloro-1,3-benzoxazol-2-yl)imidazol-1-yl]methoxy]ethyl-trimethyl-silane (1.0 g, 2.3 mmol) (it was synthesized using similar procedure as example 1-4, step 1-2), B₂Pin₂ (890 mg, 3.50 mmol), Pd(dppf)Cl₂ (170 mg, 0.23 mmol) and KOAc (460 mg, 4.69 mmol) in 1,4-dioxane (20 mL) was stirred at 90° C. for overnight under N2 atmosphere. After cooled to rt, the reaction was concentrated in vacuo to give the crude product. The product was used directly for next step without purification. MS (ESI) m/z [M+H]⁺ 476.

### Example 1-7: Synthesis of tert-butyl 6-((diphenoxyphosphoryl)oxy)-4-oxa-7-azaspiro[2.5]oct-5-ene-7-carboxylate

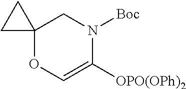

### Step 1: 2-chloro-N-((1-hydroxycyclopropyl)methyl)acetamide

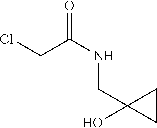

To a solution of 1-(aminomethyl)cyclopropan-1-ol (25 g, 286.9 mmol) in DCM (250 mL) was added TEA (63880 mg, 631.3 mmol, 87.8 mL) and 2-chloroacetyl chloride (35650 mg, 315.6 mmol, 25.1 mL) at 0° C. and the resulting mixture was stirred at rt for 1 h. Upon completion of the reaction, the mixture was filtered and the filtrate was concentrated in vacuum to give the product (65 g, crude). ¹H NMR (400 MHz, CDCl3) δ=7.07 (br s, 1H), 4.11 (s, 2H), 3.47 (d, J=5.6 Hz, 2H), 0.88-0.84 (m, 2H), 0.66-0.62 (m, 2H).

### Step 2: 4-oxa-7-azaspiro[2.5]octan-6-one

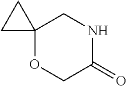

To a solution of t-BuOK (57960 mg, 516.5 mmol) in t-BuOH (600 mL) was added dropwise a solution of 2-chloro-N-[(1-hydroxycyclopropyl)methyl]acetamide (65 g, 397.3 mmol) in t-BuOH (50 mL) at rt. The mixture was stirred at rt for 0.5 h. Upon completion of the reaction, the mixture was concentrated in vacuum and the residue was purified by silica gel column chromatography (n-hexane:ethyl acetate=1:0 to 0:1) to give the product (28 g, 33% yield, 60% purity). ¹H NMR (400 MHz, DMSO-d6) δ=8.07 (br s, 1H), 3.95 (s, 2H), 3.23 (d, J=2.0 Hz, 2H), 0.85-0.81 (m, 2H), 0.65-0.62 (m, 2H).

### Step 3: tert-butyl 6-oxo-4-oxa-7-azaspiro[2.5]octane-7-carboxylate

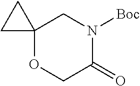 [SMILES: CC(C)(C)OC(=O)N1CC2(CC2)OCC1=O]

To a solution of 4-oxa-7-azaspiro[2.5]octan-6-one (31 g, 243.8 mmol) in DCM (300 mL) was added TEA (29610 mg, 292.5 mmol, 40.7 mL), DMAP (1490 mg, 12.1 mmol) and Boc₂O (58540 mg, 268.2 mmol, 61.6 mL) at 25° C. The mixture was stirred at 25° C. for 0.5 h. Upon completion of the reaction, the mixture was concentrated in vacuum and the residue was purified by silica gel column chromatography (n-hexane:ethyl acetate=1:0 to 5:1) to give the product (28 g, 42% yield, 85% purity). ¹H NMR (400 MHz, CDCl3) δ=4.21 (s, 2H), 3.68 (s, 2H), 1.55 (s, 9H), 1.01-0.98 (m, 2H), 0.74-0.71 (m, 2H).

### Step 4: tert-butyl 6-((diphenoxyphosphoryl)oxy)-4-oxa-7-azaspiro[2.5]oct-5-ene-7-carboxylate

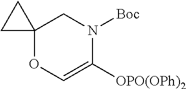

To a solution of tert-butyl 6-oxo-4-oxa-7-azaspiro[2.5]octane-7-carboxylate (28 g, 123.2 mmol) and [chloro(phenoxy)phosphoryl]oxybenzene (36410 mg, 135.5 mmol, 28.0 mL) in THF (280 mL) was added dropwise LiHMDS (1 M in THF, 147.8 mL) over 1 min at 25° C. under N₂. Upon completion of the reaction, the reaction mixture was poured into sat. NH₄Cl (200 mL, aq.) and the aqueous phase was extracted with ethyl acetate (200 mL×3). The combined organic phase was washed with brine, dried over anhydrous Na₂SO₄, filtered and concentrated in vacuum. The residue was purified by silica gel column chromatography (n-hexanes:Ethyl acetate=1:0 to 3:1) to give the product (39500 mg, 66% yield, 96% purity). MS(ESI) m/z [M-Boc+1]360.0. ¹H NMR (400 MHz, CDCL3) δ=7.39-7.33 (m, 4H), 7.28-7.18 (m, 6H), 6.33 (d, J=3.6 Hz, 1H), 3.59 (s, 2H), 1.45 (s, 9H), 0.93-0.88 (m, 2H), 0.75-0.71 (m, 2H)

### Example 1-8: Synthesis of tert-butyl 7-diphenoxyphosphoryloxy-5-oxa-8-azaspiro [3.5]non-6-ene-8-carboxylate

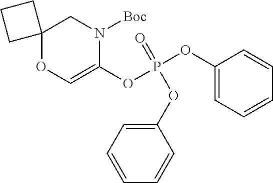 [SMILES: CC(C)(C)OC(=O)N1CC2(CCC2)OC=C1OP(=O)(Oc1ccccc1)Oc1ccccc1]

### Step 1: 2-chloro-N-[(1-hydroxycyclobutyl)methyl]acetamide

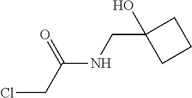 [SMILES: O=C(CCl)NCC1(O)CCC1]

To a solution of 1-(aminomethyl)cyclobutanol (1) (50 g, 494 mmol, 1 eq) and DIEA (95.8 g, 742 mmol, 129 mL, 1.5 eq) in DCM (500 mL) was added 2-chloroacetyl chloride (61.4 g, 544 mmol, 43.3 mL, 1.1 eq) in DCM (100 mL) under N₂ at 0° C. The mixture was stirred at 25° C. for 16 h. Upon completion of the reaction, H₂O (500 mL) was added and the resulting mixture was extracted with DCM. The combined organic layer was dried over Na₂SO₄, concentrated in vacuo to give a residue. The residue was purified by silica gel column chromatography (n-hexanes/ethyl acetate=3/1 to 1/1) to give the product (150 g, 85.4% yield, 50% purity). ¹H NMR (400 MHz, CDCl₃) δ 7.09 (br s, 1H), 4.03 (s, 2H), 3.43 (d, J=5.8 Hz, 2H), 3.03 (q, J=7.5 Hz, 3H), 2.04-1.99 (m, 2H), 1.72-1.67 (m, 1H), 1.53-1.50 (m, 1H).

### Step 2: 5-oxa-8-azaspiro[3.5]nonan-7-one

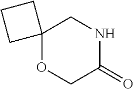

To a solution oft-BuOK (30.8 g, 274 mmol, 1.5 eq) in i-PrOH (1137 mL) at 50° C. was added dropwise a solution of 2-chloro-N-[(1-hydroxycyclobutyl)methyl]acetamide (2) (65 g, 183 mmol, 1 eq) in THF (812 mL) with stirring over for 0.5 h at 60° C. under N₂. After cooled to rt, saturated NH₄Cl (2000 mL) was added at 0° C. under N₂ and the resulting solution was extracted with DCM. The combined organic phase was concentrated in vacuo and the residue was purified by silica gel column chromatography (n-hexanes/ethyl acetate=1/1 to 0/1) to give the product (35 g, 67.8% yield). LCMS: [M+H], 142.2. ¹H NMR: (400 MHz, CDCL3) δ 7.02 (br s, 1H), 4.09 (d, J=1.1 Hz, 2H), 3.34 (s, 2H), 2.14 (q, J=10.4 Hz, 2H), 2.01-1.95 (m, 2H), 1.89-1.80 (m, 1H), 1.62 (td, J=9.3, 18.5 Hz, 1H)

### Step 3: tert-butyl 7-oxo-5-oxa-8-azaspiro[3.5]nonane-8-carboxylate

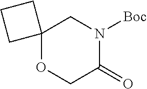 [SMILES: CC(C)(C)OC(=O)N1CC2(CCC2)OCC1=O]

To a solution of 5-oxa-8-azaspiro[3.5]nonan-7-one (40 g, 283 mmol, 1 eq) in DCM (340 mL) was added DMAP (34.6 g, 283 mmol, 1 eq) and Boc₂O (92.8 g, 425 mmol, 97.6 mL, 1.5 eq). The mixture was stirred at 25° C. for 16 h. Upon completion of the reaction, H₂O (500 mL) was added and the resulting mixture was extracted with DCM. The combined organic layers was washed with brine and the organic layer was dried over Na₂SO₄, concentrated in vacuo. The residue was purified by silica gel column chromatography (n-hexanes/ethyl acetate=10/1 to 0/1) to give the product (58 g, 84.8% yield). LCMS: [M−55]⁺, 1862. ¹H NMR (400 MHz, CDCL3) δ 4.12 (s, 2H), 3.66 (s, 2H), 2.21-2.07 (m, 2H), 2.04-1.98 (m, 2H), 1.90-1.77 (m, 1H), 1.72-1.62 (m, 1H), 1.49 (s, 9H).

### Step 4: tert-butyl 7-diphenoxyphosphoryloxy-5-oxa-8-azaspiro[3.5]non-6-ene-8-carboxylate

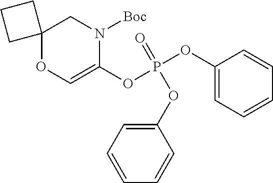 [SMILES: CC(C)(C)OC(=O)N1CC2(CCC2)OC=C1OP(=O)(Oc1ccccc1)Oc1ccccc1]

To a solution of tert-butyl 7-oxo-5-oxa-8-azaspiro[3.5]nonane-8-carboxylate (58 g, 240 mmol, 1 eq) in THF (580 mL) was added dropwise LiHMDS (1 M, 361 mL, 1.5 eq) at −40° C. under N₂. After addition, the mixture was stirred at this temperature for 1 h, and then [chloro(phenoxy)phosphoryl]oxybenzene (96.9 g, 361 mmol, 74.7 mL, 1.5 eq) in THF (116 mL) was added dropwise at −40° C. The resulting mixture was stirred at −40° C. for 2 h under N₂. Upon completion of the reaction, saturated NH₄Cl solution was added under N₂ to quench the reaction and the resulting solution was extracted with EtOAc. The combined organic layers were washed with brine (1000 mL), dried over Na₂SO₄, concentrated in vacuo to give a residue. The residue was purified by silica gel column chromatography (n-hexane/Ethyl acetate=100/1 to 10/1) to give the product (47 g, 40.6% yield, 98.35% purity). LCMS: [M+H]⁺, 474.2. H NMR: (400 MHz, CDCl₃) δ 7.31-7.24 (m, 4H), 7.19-7.09 (m, 6H), 6.16 (d, J=3.9 Hz, 1H), 3.52 (s, 2H), 2.13-2.03 (m, 2H), 1.96-1.87 (m, 2H), 1.81-1.73 (m, 1H), 1.64-1.58 (m, 1H), 1.37 (s, 9H).

### Example 1-9: Synthesis of tert-butyl 5-((diphenoxyphosphoryl)oxy)-2,2-dimethyl-2,3-dihydro-4H-1,4-oxazine-4-carboxylate

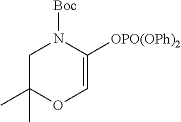

### Step 1: tert-butyl 2,2-dimethyl-5-oxomorpholine-4-carboxylate

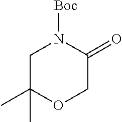 [SMILES: CC(C)(C)OC(=O)N1CC(C)(C)OCC1=O]

To a solution of 6,6-dimethylmorpholin-3-one (4.5 g, 34.84 mmol) in THF (50 mL) was added Boc₂O (15.21 g, 69.68 mmol, 16.01 mL), TEA (10.58 g, 104.52 mmol, 14.55 mL) and DMAP (851.29 mg, 6.97 mmol). The resulting mixture was stirred at 25° C. for 1 hr. Upon completion of the reaction, the reaction mixture was filtered and the filtrate was concentrated under reduced pressure to give a residue. The residue was purified by silica gel column chromatography (n-hexanes:Ethyl acetate=10/1 to 1/1) to give the product (7 g, 87% yield).

### Step 2: tert-butyl 5-((diphenoxyphosphoryl)oxy)-2,2-dimethyl-2,3-dihydro-4H-1,4-oxazine-4-carboxylate

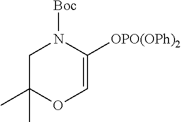

To a solution of tert-butyl 5-hydroxy-2,2-dimethyl-3H-1,4-oxazine-4-carboxylate (7 g, 30.53 mmol) in THF (70 mL) was added dropwise LiHMDS (1 M, 33.58 mL) at −20° C. under N2. The resulting mixture was stirred at −20° C. for 1 h under N2. Then [chloro(phenoxy)phosphoryl]oxybenzene (8.61 g, 32.06 mmol, 6.64 mL) was added and stirred at 25° C. for 1 h. Upon completion of the reaction, the reaction mixture was poured into sat. NH₄Cl (50 mL) to quench the reaction, and the aqueous phase was extracted with ethyl acetate. The combined organic phase was washed with brine, dried over anhydrous Na₂SO₄, filtered and concentrated in vacuum. The residue was purified by silica gel column chromatography (n-hexanes:Ethyl acetate=20/1 to 10/1) to give the product (6.1 g, 13.22 mmol, 43.30% yield).

¹H NMR (400 MHz, DMSO-d₆) δ=7.49-7.40 (m, 4H), 7.33-7.19 (m, 6H), 6.39 (d, J=4.0 Hz, 1H), 3.36 (s, 2H), 1.36 (s, 9H), 1.16 (s, 6H).

### Example 1-10: Synthesis of 6-bromo-4-chloro-2-(1-((2-(trimethylsilyl)ethoxy)methyl)-1H-imidazol-4-yl)benzo[d]thiazole

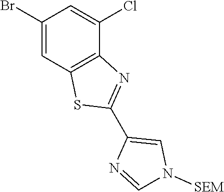 [SMILES: C[Si](C)(C)CCOCn1cnc(-c2nc3c(Cl)cc(Br)cc3s2)c1]

### Step 1: 6-bromo-4-chlorobenzo[d]thiazol-2-amine

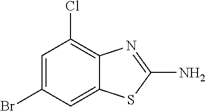 [SMILES: Nc1nc2c(Cl)cc(Br)cc2s1]

To a solution of 4-bromo-2-chloroaniline (200.0 g, 968.7 mmol) in AcOH (2000 mL) was added KSCN (392.2 g, 387.5 mmol), and the resulting mixture was stirred at rt for 30 min. After cooled to 10° C., a solution of Br₂ (309.6 g, 193.7 mmol) in AcOH (500 mL) was added, the resulting mixture was stirred at rt for 12 h. Upon completion of the reaction, water was added and the pH value was adjusted to 8-9 with aq. NaOH (4N), the precipitated solid was collected by filtration. This filter cake was redissolved in EA (2000 mL) and the resulting mixture was filtered to remove solid. This filtrate was concentrated in vacuo to give the title compound (70.0 g, 27.4%). MS (ESI) m/z [M+H]⁺ 263, 265.

### Step 2: 2-amino-5-bromo-3-chlorobenzenethiol

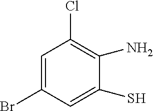

To a solution of 6-bromo-4-chlorobenzo[d]thiazol-2-amine (70.0 g, 265.6 mmol) in ethylene glycol (150 mL) was added KOH (10 N, 300 mL), and the resulting mixture was stirred at 160° C. for 8 h. After cooling to rt, the reaction mixture was filtered through a pad of Celite, and the pH value of the filtrated was adjusted to 4-5 with aq. HCl (4N), the precipitated solid was collected by filtration, further dried in vacuo to give the title compound (30.0 g, 47.4%). MS (ESI) m/z [M+H]⁺ 238, 240.

### Step 3: 6-bromo-4-chloro-2-(1H-imidazol-5-yl)benzo[d]thiazole

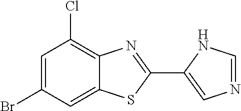 [SMILES: Clc1cc(Br)cc2sc(-c3cnc[nH]3)nc12]

To a solution of 2-amino-5-bromo-3-chlorobenzenethiol (30.0 g, 125.8 mmol) in AcOH (150 mL) was added 1H-imidazole-4-carbaldehyde (12.1 g, 125.8 mmol), and the resulting mixture was stirred at 120° C. for 48 h. After cooling to rt, the reaction mixture was poured into ice water (500 mL). The pH value of this solution was adjusted to 8-9 with aq. Na₂CO₃, and the mixture was extracted with EA. The organic layer was washed with brine, dried over anhydrous Na₂SO₄ and concentrated to give the title compound (20.0 g, 50.6%). MS (ESI) m/z [M+H]⁺ 314, 316.

### Step 4: 6-bromo-4-chloro-2-(1-((2-(trimethylsilyl)ethoxy)methyl)-1H-imidazol-4-yl)benzo[d]thiazole (Assumed SEM Position)

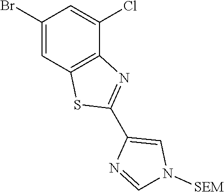 [SMILES: C[Si](C)(C)CCOCn1cnc(-c2nc3c(Cl)cc(Br)cc3s2)c1]

To a solution of 6-bromo-4-chloro-2-(1H-imidazol-5-yl)benzo[d]thiazole (2.0 g, 6.4 mmol) in DMF (20 mL) was added NaH (304 mg, 7.6 mmol, 60% in mineral oil), and the resulting mixture was stirred at rt for 10 min, followed by SEMCl (1.27 g, 7.6 mmol). The resulting mixture was stirred at rt for 1 h. Upon completion of the reaction, the mixture was poured into ice water and extracted with EA. The organic phase was washed with brine, dried over anhydrous Na₂SO₄ and concentrated. The residue was purified by column chromatography (PE/EA 10/1 to 4/1) to give the title compound (0.92 g, 32.4%). MS (ESI) m/z [M+H]⁺ 444, 446.

### Example 2

### Synthesis of Compounds

### Example 2-1: Synthesis of 1-(3-(7-chloro-2-(1H-pyrazol-5-yl)benzofuran-5-yl)morpholino)prop-2-en-1-one (B1)

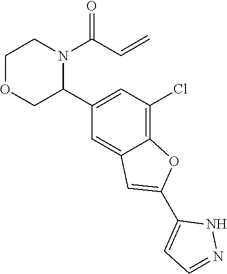 [SMILES: C=CC(=O)N1CCOCC1c1cc(Cl)c2oc(-c3ccn[nH]3)cc2c1]

### Step 1: 5-(7-chloro-5-(4,4,5,5-tetramethyl-1,3,2-dioxaborolan-2-yl)benzofuran-2-yl)-1-((2-(trimethylsilyl)ethoxy)methyl)-1H-pyrazole

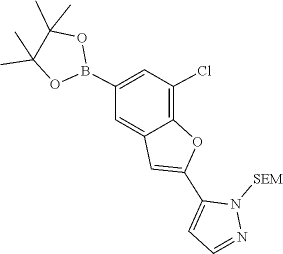 [SMILES: CC1(C)OB(c2cc(Cl)c3oc(-c4ccnn4COCC[Si](C)(C)C)cc3c2)OC1(C)C]

To a round bottom flask was placed a mixture of 5-(5-bromo-7-chlorobenzofuran-2-yl)-1-((2-(trimethylsilyl)ethoxy)methyl)-1H-pyrazole (150 mg, 0.35 mmol), 4,4,4′,4′,5,5,5′,5′-octamethyl-2,2′-bi(1,3,2-dioxaborolane) (107 mg, 0.42 mmol), Pd(dppf)Cl2 (26 mg, 0.04 mmol), KOAc (69 mg, 0.7 mmol) and 1,4-dioxane (8 mL). The resulting mixture was introduced into nitrogen and heated at 100° C. with stirring for 3 hrs. The resulting reaction was cooled to room temperature. To the reaction solution was added EA. The solids were filtered out. The filtration was concentrated to give the desired crude product (280 mg crude). MS (ESI) m/z [M+H]⁺ 475.

### Step 2: tert-butyl 5-(7-chloro-2-(1-((2-(trimethylsilyl)ethoxy)methyl)-1H-pyrazol-5-yl)benzofuran-5-yl)-2,3-dihydro-4H-1,4-oxazine-4-carboxylate

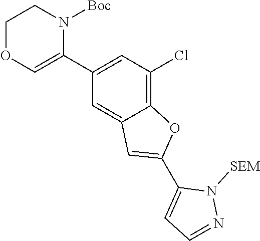 [SMILES: CC(C)(C)OC(=O)N1CCOC=C1c1cc(Cl)c2oc(-c3ccnn3COCC[Si](C)(C)C)cc2c1]

A mixture of (5-(7-chloro-5-(4,4,5,5-tetramethyl-1,3,2-dioxaborolan-2-yl)benzofuran-2-yl)-1-((2-(trimethylsilyl)ethoxy)methyl)-1H-pyrazole (280 mg, crude), 4H-1,4-Oxazine-4-carboxylic acid, 5-[(diphenoxyphosphinyl)oxy]-2,3-dihydro-, 1,1-dimethylethyl ester (152 mg, 0.35 mmol), Pd(dppf)Cl₂ (26 mg, 0.04 mmol) and K₂CO₃ (97 mg, 0.7 mmol) were added into dioxane (10 mL) and water (2.5 mL). The resulting mixture was introduced into nitrogen and heated at 90° C. with stirring for 3 hrs. After cooled to room temperature, the resulting reaction mixture was diluted with EA. The organic layer was washed with brine, dried over Na₂SO₄ and concentrated. The residue was purified on Prep-TLC (EA/PE=1:4) to give the desired product (90 mg, 48% for 2 steps). MS (ESI) m/z [M+H]⁺ 532.

### Step 3: 3-(7-chloro-2-(1-((2-(trimethylsilyl)ethoxy)methyl)-1H-pyrazol-5-yl)benzofuran-5-yl)morpholine

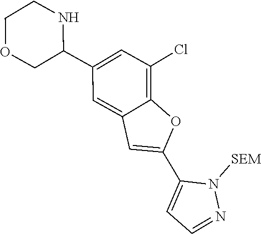 [SMILES: C[Si](C)(C)CCOCn1nccc1-c1cc2cc(C3COCCN3)cc(Cl)c2o1]

A solution of tert-butyl 5-(7-chloro-2-(1-((2-(trimethylsilyl)ethoxy)methyl)-1H-pyrazol-5-yl)benzofuran-5-yl)-2,3-dihydro-4H-1,4-oxazine-4-carboxylate (90 mg, 0.17 mmol) in DCM (3 mL) was stirred at 0° C. To this was added TFA (3 mL) at 0° C. The resulting reaction was warmed to rt with stirring for 30 min. The reaction mixture was re-cooled to 0° C. To this was added STAB (360 mg, 1.7 mmol) and stirred at 0° C. for 30 min. Upon completion of the reaction, the solvent was removed. The residue was dissolved into DCM/MeOH (10:1). The organic layer was washed with NaHCO₃ (aq.) and brine, dried over Na₂SO₄ and concentrated. The residue was purified on Prep-TLC with DCM/MeOH (20:1) to give the desired product (30 mg, 41%). MS (ESI) m/z [M+H]⁺ 434.

### Step 4: 1-(3-(7-chloro-2-(1-((2-(trimethylsilyl)ethoxy)methyl)-1H-pyrazol-5-yl)benzofuran-5-yl)morpholino)prop-2-en-1-one

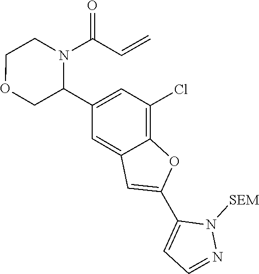 [SMILES: C=CC(=O)N1CCOCC1c1cc(Cl)c2oc(-c3ccnn3COCC[Si](C)(C)C)cc2c1]

To a solution of 3-(7-chloro-2-(1-((2-(trimethylsilyl)ethoxy)methyl)-1H-pyrazol-5-yl)benzofuran-5-yl)morpholine (30 mg, 0.07 mmol) in DCM (2 mL) and NaHCO₃ (aq) (3 mL) was added a solution of acryloyl chloride (6.3 mg, 0.07 mmol) in DCM (1 mL) dropwise at 0° C. The mixture was stirred at 0° C. for 10 min. Upon completion of the reaction, the reaction was quenched by DCM/MeOH (10:1). The organic layer was washed with brine and concentrated. The residue was purified on Prep-TLC with DCM/MeOH (20:1) to give the desired product (25 mg, 74%). MS (ESI) m/z [M+H]⁺=488.

### Step 5: 1-(3-(7-chloro-2-(1H-pyrazol-5-yl)benzofuran-5-yl)morpholino)prop-2-en-1-one

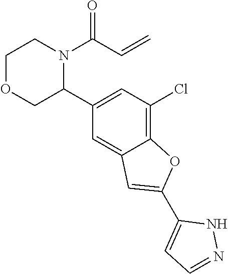 [SMILES: C=CC(=O)N1CCOCC1c1cc(Cl)c2oc(-c3ccn[nH]3)cc2c1]

To a solution of 1-(3-(7-chloro-2-(1-((2-(trimethylsilyl)ethoxy)methyl)-1H-pyrazol-5-yl)benzofuran-5-yl)morpholino)prop-2-en-1-one (25 mg, 0.05 mmol) in DCM (3 mL) was added TFA (3 mL) at 0° C. The mixture was warmed to rt with stirring for 1 hr. Upon completion of the reaction, the reaction mixture was concentrated. The residue was applied onto Prep-TLC with DCM/MeOH (20:1) for purification. The product was repurified with C18 column with MeCN/H2O (0.1% FA) to give the desired product (6.61 mg, 37%). ¹H NMR (400 MHz, DMSO-d₆) δ 13.31 (s, 1H), 7.90 (s, 1H), 7.62 (s, 1H), 7.38 (s, 1H), 7.28 (s, 1H), 6.92-6.83 (m, 1H), 6.77 (s, 1H), 6.23 (d, J=16.5 Hz, 1H), 5.76 (d, J=10.5 Hz, 1H), 5.67-5.28 (m, 1H), 4.45 (d, J=12.2 Hz, 1H), 4.33-3.78 (m, 3H), 3.61-2.98 (m, 2H). MS (ESI) m/z [M+H]⁺=358.

### Example 2-2: Synthesis of 1-(3-(7-chloro-2-(1H-imidazol-5-yl)benzofuran-5-yl)morpholino)prop-2-en-1-one (B2)

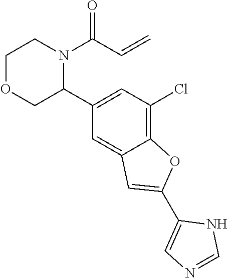 [SMILES: C=CC(=O)N1CCOCC1c1cc(Cl)c2oc(-c3cnc[nH]3)cc2c1]

### Step 1: tert-butyl 3-(7-chloro-2-(1H-imidazol-5-yl)benzofuran-5-yl)morpholine-4-carboxylate

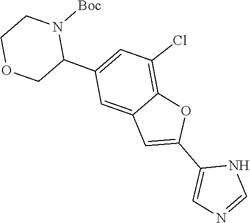 [SMILES: CC(C)(C)OC(=O)N1CCOCC1c1cc(Cl)c2oc(-c3cnc[nH]3)cc2c1]

A mixture of (5-(4-(tert-butoxycarbonyl)morpholin-3-yl)-7-chlorobenzofuran-2-yl)boronic acid (150 mg, 0.39 mmol), tert-butyl 4-bromo-1H-imidazole-1-carboxylate (126 mg, 0.39 mmol), Pd(dtbpf)Cl₂ (26 mg, 0.04 mmol) and K₂CO₃ (108 mg, 0.79 mmol) were added into dioxane (12 mL) and H₂O (3 mL). The resulting mixture was degassed with nitrogen and heated at 80° C. with stirring for 1 hr. After cooled to rt, the solvents were removed under vacuum. The residue was purified on Prep-TLC (DCM/MeOH=20:1) to give the desired product (130 mg, 81%). MS (ESI) m/z [M+H]⁺ 404.

### Step 2: 3-(7-chloro-2-(1H-imidazol-5-yl)benzofuran-5-yl)morpholine

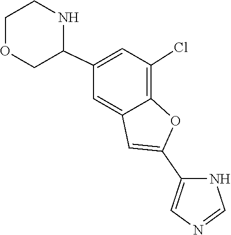 [SMILES: Clc1cc(C2COCCN2)cc2cc(-c3cnc[nH]3)oc12]

A solution of tert-butyl 3-(7-chloro-2-(1H-imidazol-5-yl)benzofuran-5-yl)morpholine-4-carboxylate (130 mg, 0.32 mmol) in DCM (7 mL) was cooled to 0° C. To this was added HCl (4N, in EA) (7 mL). The resulting reaction was warmed to rt with stirring for 1 h. The resulting mixture was concentrated. This resulted in 120 mg of the crude desired product which was used for next step directly. MS (ESI) m/z [M+H]⁺ 304.

### Step 3:1-(3-(7-chloro-2-(1H-imidazol-5-yl)benzofuran-5-yl)morpholino)prop-2-en-1-one

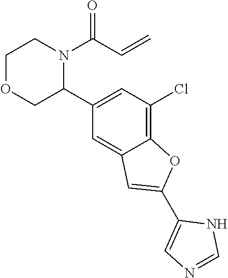 [SMILES: C=CC(=O)N1CCOCC1c1cc(Cl)c2oc(-c3cnc[nH]3)cc2c1]

To a solution of 3-(7-chloro-2-(1H-imidazol-5-yl)benzofuran-5-yl)morpholine (120 mg, crude) in DCM (7 mL) and NaHCO₃ (aq) (8 mL) was added a solution of acryloyl chloride (29 mg, 0.32 mmol) in DCM (1 mL) dropwise at 0° C. The mixture was stirred at 0° C. for 10 min. Upon completion of the reaction, the reaction was quenched by DCM/MeOH (10:1). The organic layer was washed with brine and concentrated. The residue was applied onto Prep-TLC with DCM/MeOH (15:1) for purification. The product was repurified on Prep-HPLC (column: Sunfire C18 (19×150 mm, 5 μm); mobile phase: [H₂O (0.1% FA)-ACN]; gradient: 35%-55% B over 11.0 min) to give the title compound (12.54 mg, 11%). ¹H NMR (400 MHz, DMSO-d₆) δ 12.54 (s, 1H), 7.83 (s, 1H), 7.69 (s, 1H), 7.58 (s, 1H), 7.32 (s, 1H), 7.07 (s, 1H), 6.95-6.81 (m, 1H), 6.23 (d, J=16.6 Hz, 1H), 5.76 (d, J=10.5 Hz, 1H), 5.66-5.30 (m, 1H), 4.44 (d, J=12.2 Hz, 1H), 4.30-3.78 (m, 3H), 3.51 (t, J=11.0 Hz, 1H), 3.25-2.97 (m, 1H). MS (ESI) m/z [M+1]⁺ 358.

### Step 4: Synthesis of Example B5 and B6: 1-(3-(7-chloro-2-(1H-imidazol-5-yl)benzofuran-5-yl)morpholino)prop-2-en-1-one (Unknown Absolute Isomer1) and 1-(3-(7-chloro-2-(1H-imidazol-5-yl)benzofuran-5-yl)morpholino)prop-2-en-1-one (Unknown Absolute Isomer2)

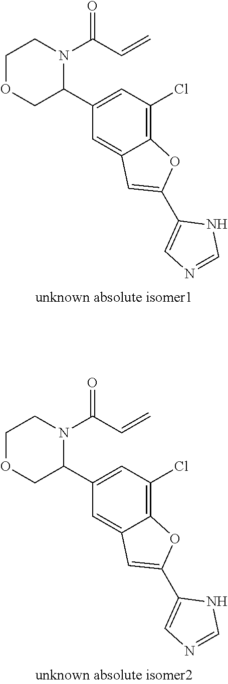 [SMILES: C=CC(=O)N1CCOCC1c1cc(Cl)c2oc(-c3cnc[nH]3)cc2c1 ; C=CC(=O)N1CCOCC1c1cc(Cl)c2oc(-c3cnc[nH]3)cc2c1]

1-(3-(7-chloro-2-(1H-imidazol-5-yl)benzofuran-5-yl)morpholino)prop-2-en-1-one was applied onto SFC: column: CHIRALPAK AD-H, 20 mm×250 mm, 5 um; fluent: 45% MeOH/C02 give the title compounds:

1-(3-(7-chloro-2-(1H-imidazol-5-yl)benzofuran-5-yl)morpholino)prop-2-en-1-one (unknown absolute isomer1). ¹H NMR (400 MHz, DMSO-d₆) δ 12.52 (s, 1H), 7.83 (s, 1H), 7.71 (s, 1H), 7.58 (s, 1H), 7.32 (s, 1H), 7.07 (s, 1H), 6.94-6.82 (m, 1H), 6.23 (d, J=16.5 Hz, 1H), 5.76 (d, J=10.4 Hz, 1H), 5.68-5.20 (m, 1H), 4.44 (d, J=12.1 Hz, 1H), 4.35-3.76 (m, 3H), 3.51 (t, J=11.4 Hz, 1H), 3.30-2.93 (m, 1H). MS (ESI) m/z [M+1]⁺ 358.

1-(3-(7-chloro-2-(1H-imidazol-5-yl)benzofuran-5-yl)morpholino)prop-2-en-1-one (unknown absolute isomer2) ¹H NMR (400 MHz, DMSO-d₆) δ 12.53 (s, 1H), 7.83 (s, 1H), 7.70 (s, 1H), 7.58 (s, 1H), 7.32 (s, 1H), 7.07 (s, 1H), 6.92-6.83 (m, 1H), 6.23 (d, J=16.5 Hz, 1H), 5.76 (d, J=10.4 Hz, 1H), 5.68-5.38 (m, 1H), 4.44 (d, J=12.2 Hz, 1H), 4.32-3.76 (m, 3H), 3.51 (t, J=11.2 Hz, 1H), 3.29-2.91 (m, 1H). MS (ESI) m/z [M+1]⁺ 358.

Compounds B3-B8, and B10 in Table 2-1 below were synthesized by using corresponding starting materials according to procedures similar to those described for the synthesis of compounds B1 and B2

TABLE 2-1

| | | | ¹H NMR data LC/MS m/z |
|---|---|---|---|
| Compound | Structure | Chemical Name | (M + H) |
| B3 | 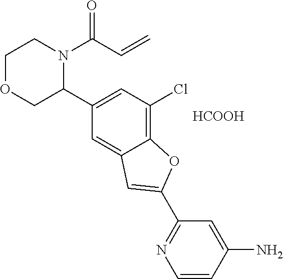 [SMILES: C=CC(=O)N1CCOCC1c1cc(Cl)c2oc(-c3cc(N)ccn3)cc2c1 ; "HCOOH"] | 1-(3-(2-(4-aminopyridin-2-yl)- 7-chlorobenzofuran-5- yl)morpholino)prop-2-en-1- one formate | ¹H NMR (400 MHz, DMSO-d₆) δ 8.09 (d, J = 5.5 Hz, 1H), 7.68 (s, 1H), 7.47 (s, 1H), 7.43 (s, 1H), 7.15 (d, J = 1.6 Hz, 1H), 6.92- 6.80 (m, 1H), 6.53-6.47 (m, 1H), 6.36 (s, 2H), 6.29-6.20 (m, 1H), 5.80-5.73 (m, 1H), 5.70-5.29 (m, 1H), 4.45 (d, J = 12.2 Hz, 1H), 4.33-3.75 (m, 3H), 3.52 (t, J = 10.7 Hz, 1H), 3.31-2.90 (m, 1H). MS (ESI) m/z [M + 1]⁺ 384. |
| B4 | 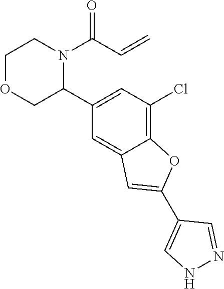 [SMILES: C=CC(=O)N1CCOCC1c1cc(Cl)c2oc(-c3cn[nH]c3)cc2c1] | 1-(3-(7-chloro-2-(1H-pyrazol- 4-yl)benzofuran-5- yl)morpholino)prop-2-en-1- one | ¹H NMR (400 MHz, DMSO-d₆) δ 13.32 (s, 1H), 8.38 (s, 1H), 8.00 (s, 1H), 7.55 (s, 1H), 7.31 (s, 1H), 7.09 (s, 1H), 6.93-6.76 (m, 1H), 6.30-6.17 (m, 1H), 5.84-5.71 (m, 1H), 5.69-5.31 (m, 1H), 4.44 (d, J = 12.2 Hz, 1H), 4.24-3.77 (m, 3H), 3.51 (t, J = 10.6 Hz, 1H), 3.22-2.92 (m, 1H). MS (ESI) m/z [M + 1]⁺ 358. |
| B5 | 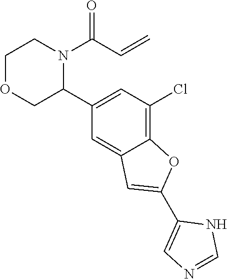 [SMILES: C=CC(=O)N1CCOCC1c1cc(Cl)c2oc(-c3cnc[nH]3)cc2c1] | 1-(3-(7-chloro-2-(1H- imidazol-5-yl)benzofuran-5- yl)morpholino)prop-2-en-1- one (unknow absolute isomer1) | ¹H NMR (400 MHz, DMSO-d₆) δ 12.52 (s, 1H), 7.83 (s, 1H), 7.71 (s, 1H), 7.58 (s, 1H), 7.32 (s, 1H), 7.07 (s, 1H), 6.94-6.82 (m, 1H), 6.23 (d, J = 16.5 Hz, 1H), 5.76 (d, J = 10.4 Hz, 1H), 5.68-5.20 (m, 1H), 4.44 (d, J = 12.1 Hz, 1H), 4.35-3.76 (m, 3H), 3.51 (t, J = 11.4 Hz, 1H), 3.30-2.93 (m, 1H). MS (ESI) m/z [M + 1]⁺ 358. |
| B6 | 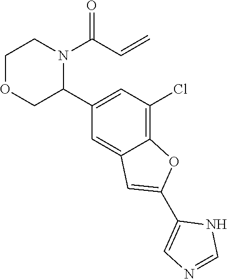 [SMILES: C=CC(=O)N1CCOCC1c1cc(Cl)c2oc(-c3cnc[nH]3)cc2c1] | 1-(3-(7-chloro-2-(1H- imidazol-5-yl)benzofuran-5- yl)morpholino)prop-2-en-1- one (unknow absolute isomer1) | ¹H NMR (400 MHz, DMSO-d₆) δ 12.53 (s, 1H), 7.83 (s, 1H), 7.70 (s, 1H), 7.58 (s, 1H), 7.32 (s, 1H), 7.07 (s, 1H), 6.92-6.83 (m, 1H), 6.23 (d, J = 16.5 Hz, 1H), 5.76 (d, J = 10.4 Hz, 1H), 5.68-5.38 (m, 1H), 4.44 (d, J = 12.2 Hz, 1H), 4.32-3.76 (m, 3H), 3.51 (t, J = 11.2 Hz, 1H), 3.29-2.91 (m, 1H). MS (ESI) m/z [M + 1]⁺ 358. |
| B7 | 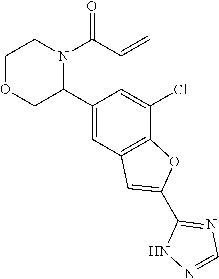 [SMILES: C=CC(=O)N1CCOCC1c1cc(Cl)c2oc(-c3ncn[nH]3)cc2c1] | 1-(3-(7-chloro-2-(1H-1,2,4- triazol-5-yl)benzofuran-5- yl)morpholino)prop-2-en-1- one | ¹H NMR (400 MHz, DMSO-d₆) δ 8.65 (s, 1H), 7.67 (s, 1H), 7.48 (s, 1H), 7.43 (s, 1H), 6.84 (dd, J = 16.6, 10.5 Hz, 1H), 6.20 (d, J = 16.6 Hz, 1H), 5.73 (d, J = 10.4 Hz, 1H), 5.67-5.29 (m, 1H), 4.42 (d, J = 12.2 Hz, 1H), 4.27-3.73 (m, 3H), 3.49 (t, J = 10.8 Hz, 1H), 3.12- 2.89 (m, 1H). MS (ESI) m/z [M + H]⁺ = 359. |
| B8 | 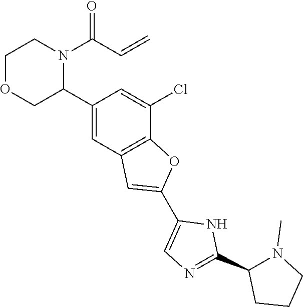 [SMILES: C=CC(=O)N1CCOCC1c1cc(Cl)c2oc(-c3cnc([C@@H]4CCCN4C)[nH]3)cc2c1] | 1-(3-(7-chloro-2-(2-((S)-1- methylpyrrolidin-2-yl)-1H- imidazol-5-yl)benzofuran-5- yl)morpholino)prop-2-en-1- one | ¹H NMR (400 MHz, DMSO-d₆) δ 12.31 (s, 1H), 8.27 (s, 1H), 7.53 (s, 1H), 7.27 (s, 1H), 7.00 (s, 1H), 6.83 (dd, J = 16.5, 10.6 Hz, 1H), 6.20 (d, J = 16.7 Hz, 1H), 5.73 (d, J = 10.4 Hz, 1H), 5.62-5.29 (m, 1H), 4.41 (d, J = 12.1 Hz, 1H), 3.91-3.72 (m, 3H), 3.51-3.46 (m, 2H), 3.09-3.06 (m, 1H), 2.25- 2.20 (m, 1H), 2.13 (s, 3H), 2.11- 2.06 (m, 1H), 1.90-1.70 (m, 4H). MS (ESI) m/z [M + H]⁺ = 441. |
| B10 | 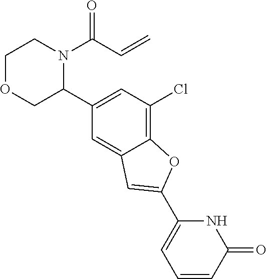 [SMILES: C=CC(=O)N1CCOCC1c1cc(Cl)c2oc(-c3cccc(=O)[nH]3)cc2c1] | 6-(5-(4-acryloylmorpholin-3- yl)-7-chlorobenzofuran-2- yl)pyridin-2(1H)-one | 1H NMR (400 MHz, DMSO-d6) δ 11.73 (s, 1H), 7.78-7.60 (m, 3H), 7.47 (s, 1H), 7.09 (s, 1H), 6.98- 6.81 (m, 1H), 6.55 (d, J = 8.7 Hz, 1H), 6.30-6.17 (m, 1H), 5.76 (d, J = 10.4 Hz, 1H), 5.70-5.30 (m, 1H), 4.46 (d, J = 12.1 Hz, 1H), 4.32-3.74 (m, 3H), 3.52 (t, J = 10.9 Hz, 1H), 3.28-2.96 (m, 1H). MS (ESI) m/z [M + 1]+ 385. |

### Example 2-3: Synthesis of 1-(4-acetyl-2-(7-chloro-2-(1H-imidazol-5-yl)benzofuran-5-yl)piperazin-1-yl)prop-2-en-1-one (C1)

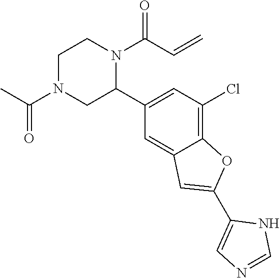 [SMILES: C=CC(=O)N1CCN(C(C)=O)CC1c1cc(Cl)c2oc(-c3cnc[nH]3)cc2c1]

### Step 1: tert-butyl 3-(7-chloro-2-(1-((2-(trimethylsilyl)ethoxy)methyl)-1H-imidazol-4-yl)benzofuran-5-yl)piperazine-1-carboxylate

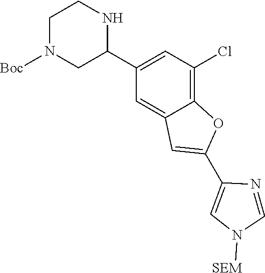 [SMILES: CC(C)(C)OC(=O)N1CCNC(c2cc(Cl)c3oc(-c4cn(COCC[Si](C)(C)C)cn4)cc3c2)C1]

A mixture of (5-(4-(tert-butoxycarbonyl)piperazin-2-yl)-7-chlorobenzofuran-2-yl)boronic acid (2 g, 5.3 mmol), 2-[(4-bromoimidazol-1-yl)methoxy]ethyl-trimethyl-silane (1.46 g, 5.27 mmol), Pd(dtbpf)Cl₂ (171.82 mg, 0.3 mmol) and Na2CO₃ (1.1 g, 10.5 mmol) were added into 1,4-dioxane (50 mL) and H₂O (10 mL). The resulting reaction was degassed with nitrogen and sealed into a tube. The reaction was heated at 80° C. with stirring for 1 h. After cooled to rt, the reaction was diluted with EA. The organic layer was washed with water and brine, dried over Na₂SO₄. The residue was purified onto C18 column with MeCN/H₂O (0.1% FA) to give the title product (1.3 g, 2.4 mmol, 46% yield). MS (ESI) m/z [M+H]⁺ 533.

### Step 2: tert-butyl 4-acryloyl-3-(7-chloro-2-(1-((2-(trimethylsilyl)ethoxy)methyl)-1H-imidazol-4-yl)benzofuran-5-yl)piperazine-1-carboxylate

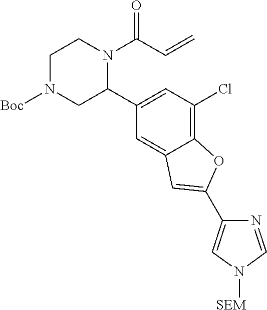 [SMILES: C=CC(=O)N1CCN(C(=O)OC(C)(C)C)CC1c1cc(Cl)c2oc(-c3cn(COCC[Si](C)(C)C)cn3)cc2c1]

A mixture of tert-butyl 3-(7-chloro-2-(1-((2-(trimethylsilyl)ethoxy)methyl)-1H-imidazol-4-yl)benzofuran-5-yl)piperazine-1-carboxylate (200 mg, 0.37 mmol) in DCM (7 mL) and sat.NaHCO₃ (aq.) (8 mL) was cooled to 0° C. To this was added prop-2-enoyl chloride (34 mg, 0.38 mmol) dropwise with stirring at 0° C. The reaction was stirred below 10° C. for 10 min. The reaction was diluted with DCM/MeOH (10:1). The organic layer was washed with brine and concentrated. The residue was purified on Prep-TLC with DCM/ZA (1:1) to give the title product (180 mg, 82% yield). MS (ESI) m/z [M+H]⁺ 587.

### Step 3:1-(2-(7-chloro-2-(1H-imidazol-4-yl)benzofuran-5-yl)piperazin-1-yl)prop-2-en-1-one

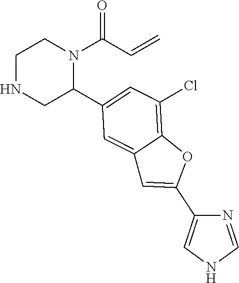 [SMILES: C=CC(=O)N1CCNCC1c1cc(Cl)c2oc(-c3c[nH]cn3)cc2c1]

To a round bottom flask was place a solution of tert-butyl 4-acryloyl-3-(7-chloro-2-(1-((2-(trimethylsilyl)ethoxy)methyl)-1H-imidazol-4-yl)benzofuran-5-yl)piperazine-1-carboxylate (180 mg, 0.31 mmol) in DCM (8 mL). To this was added TFA (8 mL) at rt. The resulting reaction was stirred at R.T. for 1 h. The resulting reaction was concentrated. This resulted in 1-[2-[7-chloro-2-(1H-imidazol-4-yl)benzofuran-5-yl]piperazin-1-yl]prop-2-en-1-one (160 mg, purity=65%, 95% Yield) which was used for next step directly. MS (ESI) m/z [M+H]⁺ 357.

### Step 4: 1-(4-acetyl-2-(7-chloro-2-(1H-imidazol-4-yl)benzofuran-5-yl)piperazin-1-yl)prop-2-en-1-one

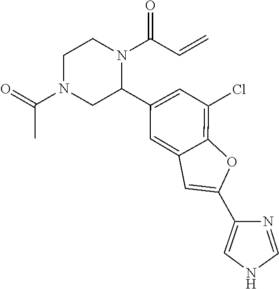 [SMILES: C=CC(=O)N1CCN(C(C)=O)CC1c1cc(Cl)c2oc(-c3c[nH]cn3)cc2c1]

To a mixture of 1-(2-(7-chloro-2-(1H-imidazol-4-yl)benzofuran-5-yl)piperazin-1-yl)prop-2-en-1-one (160 mg, 0.36 mmol, P=80%) in DCM (12 mL) was added TEA (0.14 g, 1.4 mmol). The resulting mixture was cooled to 0° C. To this was added acetyl acetate (0.04 g, 0.4 mmol) dropwise with stirring at 0° C. The resulting reaction was stirred at R.T. for 30 min. Upon completion of the reaction, DCM/MeOH (10:1) (20 mL) was added. The organic layer was washed with brine and then concentrated. The residue was purified on Prep-TLC with DCM/MeOH(12:1). The product was repurified on Prep-HPLC (column: Sunfire C18 (19×150 mm, 5 m); mobile phase: [H₂O (0.1% FA)-ACN]; gradient: 30%-45% B over 11.0 min) to give the title compound (18.28 mg, 13%). ¹H NMR (400 MHz, DMSO-d₆) δ 12.68 (s, 1H), 7.87 (s, 1H), 7.70 (s, 1H), 7.53-7.18 (m, 2H), 7.11-7.03 (m, 1H), 6.98-6.48 (m, 1H), 6.28-6.12 (m, 1H), 5.84-5.43 (m, 2H), 4.76-4.02 (m, 2H), 3.88-3.56 (m, 2H), 3.37-3.17 (m, 2H), 2.02-1.88 (m, 3H). MS (ESI) m/z [M+1]⁺ 399.

Compounds C2 in Table 2-2 below were synthesized by using corresponding starting materials according to procedures similar to those described for the synthesis of compounds C1

TABLE 2-2

| Compound | Structure | Chemical Name | ¹H NMR data LC/MS m/z (M + H) |
|---|---|---|---|
| C2 | 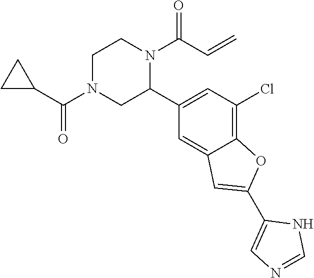 [SMILES: C=CC(=O)N1CCN(C(=O)C2CC2)CC1c1cc(Cl)c2oc(-c3cnc[nH]3)cc2c1] | 1-(2-(7-chloro-2-(1H- imidazol-5- yl)benzofuran-5-yl)-4- (cyclopropanecarbonyl) piperazin-1-yl)prop-2- en-1-one | ¹H NMR (400 MHz, DMSO-d₆) δ 12.56 (s, 1H), 7.83 (s, 1H), 7.70 (s, 1H), 7.50-7.17 (m, 2H), 7.10-6.54 (m, 2H), 6.29-6.16 (m, 1H), 5.87- 5.42 (m, 2H), 4.77-3.81 (m, 4H), 3.67-3.47 (m, 1H), 3.21-2.95 (m, 1H), 2.18-1.82 (m, 1H), 0.82-0.28 (m, 4H). MS (ESI) m/z [M + 1]⁺ 425. |

### Example 2-4: Synthesis of 1-(2-(7-chloro-2-(1H-pyrazol-5-yl)benzo[d]oxazol-5-yl)-4-(oxetane-3-carbonyl)piperazin-1-yl)prop-2-en-1-one (D11)

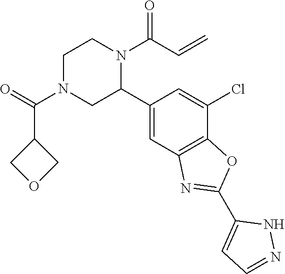 [SMILES: C=CC(=O)N1CCN(C(=O)C2COC2)CC1c1cc(Cl)c2oc(-c3ccn[nH]3)nc2c1]

### Step 1: tert-butyl 3-(7-chloro-2-(1-((2-(trimethylsilyl)ethoxy)methyl)-1H-pyrazol-5-yl)benzo[d]oxazol-5-yl)piperazine-1-carboxylate

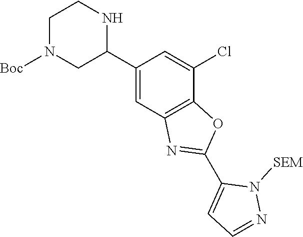 [SMILES: CC(C)(C)OC(=O)N1CCNC(c2cc(Cl)c3oc(-c4ccnn4COCC[Si](C)(C)C)nc3c2)C1]

To a solution of 7-chloro-5-(piperazin-2-yl)-2-(1-((2-(trimethylsilyl)ethoxy)methyl)-1H-pyrazol-5-yl)benzo[d]oxazole (458 mg, 1.1 mmol) in THF (10 mL) and sat.NaHCO₃ (10 mL) was added Boc₂O (207 mg, 0.95 mmol) at 0° C. and the resulting mixture was stirred at rt for 1 hour. The reaction was diluted with DCM, was washed with brine, dried over Na₂SO₄, filtered and concentrated to give the product (560 mg, crude). MS (ESI) m/z [M+H]⁺ 534.

### Step 2: tert-butyl 4-acryloyl-3-(7-chloro-2-(1-((2-(trimethylsilyl)ethoxy)methyl)-1H-pyrazol-5-yl)benzo[d]oxazol-5-yl)piperazine-1-carboxylate

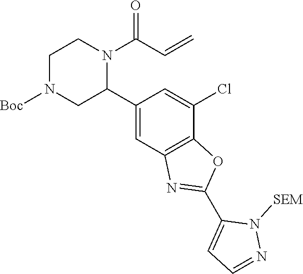 [SMILES: C=CC(=O)N1CCN(C(=O)OC(C)(C)C)CC1c1cc(Cl)c2oc(-c3ccnn3COCC[Si](C)(C)C)nc2c1]

To a solution of tert-butyl 3-(7-chloro-2-(1-((2-(trimethylsilyl)ethoxy)methyl)-1H-pyrazol-5-yl)benzo[d]oxazol-5-yl)piperazine-1-carboxylate (560 mg, 1.05 mmol) in DCM (20 mL) was added TEA (318 mg, 3.15 mmol) and prop-2-enoyl chloride (190 mg, 2.1 mmol) and the resulting mixture was stirred at rt for 10 mins. The reaction mixture was washed with brine, dried over Na₂SO₄, filtered, concentrated and the residue was purified by column chromatography (PE/EtOAc 5/1 to 3/2) to give the title compound (375 mg, 60% for two steps). MS (ESI) m/z [M+H]⁺ 588.

### Step 3:1-(2-(7-chloro-2-(1H-pyrazol-5-yl)benzo[d]oxazol-5-yl)piperazin-1-yl)prop-2-en-1-one

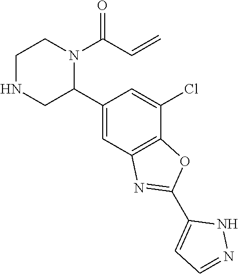 [SMILES: C=CC(=O)N1CCNCC1c1cc(Cl)c2oc(-c3ccn[nH]3)nc2c1]

A solution of tert-butyl 4-acryloyl-3-(7-chloro-2-(1-((2-(trimethylsilyl)ethoxy)methyl)-1H-pyrazol-5-yl)benzo[d]oxazol-5-yl)piperazine-1-carboxylate (375 mg, 0.64 mmol) in DCM (5 mL) and TFA (5 mL) was stirred at rt for 2 hours. The solution was concentrated to give the title compound (228 mg, crude). MS (ESI) m/z [M+H]⁺ 358.

### Step 4: 1-(2-(7-chloro-2-(1H-pyrazol-5-yl)benzo[d]oxazol-5-yl)-4-(oxetane-3-carbonyl)piperazin-1-yl)prop-2-en-1-one

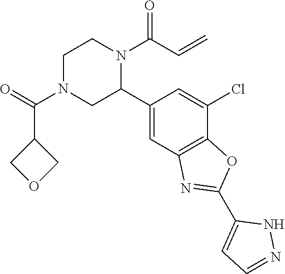 [SMILES: C=CC(=O)N1CCN(C(=O)C2COC2)CC1c1cc(Cl)c2oc(-c3ccn[nH]3)nc2c1]

A solution of 1-(2-(7-chloro-2-(1H-pyrazol-5-yl)benzo[d]oxazol-5-yl)piperazin-1-yl)prop-2-en-1-one (32 mg, 0.09 mmol), oxetane-3-carboxylic acid (9 mg, 0.09 mmol), HATU (51 mg, 0.13 mmol) and DIEA (58 mg, 0.45 mmol) in DMF (5 mL) was stirred at rt for 1 hour. The reaction solution was concentrated in vacuo and the residue was purified by Prep-TLC (MeOH:DCM=1:20) to give crude product. The crude product was purified by Prep-HPLC to give the title compound (5.2 mg, 13.2%). ¹H NMR (400 MHz, DMSO-d₆) δ 13.68 (s, 1H), 7.99 (s, 1H), 7.65-7.54 (m, 1H), 7.48-7.28 (m, 1H), 7.02-6.95 (m, 1H), 6.94-6.67 (m, 1H), 6.25-6.08 (m, 1H), 5.83-5.43 (m, 2H), 4.85-4.54 (m, 3H), 4.50-4.37 (m, 1H), 4.23-4.01 (m, 2H), 3.98-3.61 (m, 2H), 3.41-3.30 (m, 2H), 3.21-3.11 (m, 1H). MS (ESI) m/z [M+H]⁺=442.

Compounds D1-D2, D12-D14 in Table 2-3 below were synthesized by using corresponding starting materials according to procedures similar to those described for the synthesis of compounds D1

TABLE 2-3

| Compound | Structure | Chemical Name | ¹H NMR data LC/MS m/z (M + H) |
|---|---|---|---|
| D1 | 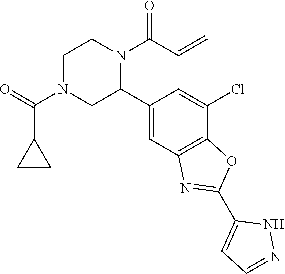 [SMILES: C=CC(=O)N1CCN(C(=O)C2CC2)CC1c1cc(Cl)c2oc(-c3ccn[nH]3)nc2c1] | 1-(2-(7-chloro-2-(1H-pyrazol- 5-yl)benzo[d]oxazol-5-yl)-4- (cyclopropanecarbonyl) piperazin-1-yl) prop-2-en-1-one | ¹H NMR (400 MHz, DMSO-d₆) δ 13.69 (s, 1H), 7.99 (s, 1H), 7.59 (d, J = 14.8 Hz, 1H), 7.52-7.29 (m,1H), 6.98 (s, 1H), 6.95-6.51 (m, 1H), 6.18 (t, J = 15.4 Hz, 1H), 5.84-5.47 (m, 2H), 4.71- 4.56 (m, 1H), 4.53-3.80 (m, 3H), 3.57-3.32 (m, 2H), 2.05-1.81 (m, 1H), 0.76-0.20 (m, 4H). MS (ESI) m/z [M + H]⁺ = 426. |
| D2 | 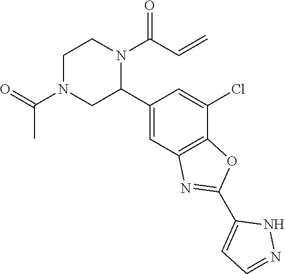 [SMILES: C=CC(=O)N1CCN(C(C)=O)CC1c1cc(Cl)c2oc(-c3ccn[nH]3)nc2c1] | 1-(4-acetyl-2-(7-chloro-2-(1H- pyrazol-5-yl)benzo[d]oxazol- 5-yl)piperazin-1-yl)prop-2-en- 1-one | ¹H NMR (400 MHz, DMSO-d₆) δ 13.69 (s, 1H), 7.99 (s, 1H), 7.67-7.55 (m, 1H), 7.51-7.28 (m, 1H), 6.99 (s, 1H), 6.95-6.60 (m, 1H), 6.25-6.08 (m, 1H), 5.82-5.44 (m, 2H), 4.86-4.23 (m, 1H), 4.20-3.50 (m, 3H), 3.29- 3.06 (m, 2H), 2.00-1.82 (m, 3H). MS (ESI) m/z [M + H]⁺ = 400. |
| D12 | 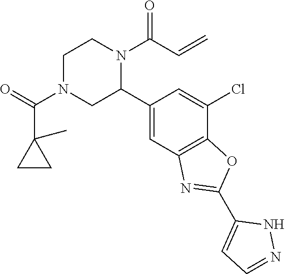 [SMILES: C=CC(=O)N1CCN(C(=O)C2(C)CC2)CC1c1cc(Cl)c2oc(-c3ccn[nH]3)nc2c1] | 1-(2-(7-chloro-2-(1H-pyrazol- 5-yl)benzo[d]oxazol-5-yl)-4- (1-methylcyclopropane-1- carbonyl)piperazin-1-yl)prop- 2-en-1-one | ¹H NMR (400 MHz, DMSO-d₆) δ 13.69 (s, 1H), 7.98 (s, 1H), 7.57 (s, 1H), 7.37 (s, 1H), 6.99 (s, 1H), 6.94-6.58 (m, 1H), 6.19 (d, J = 16.7 Hz, 1H), 5.84- 5.46 (m, 2H), 4.72-4.37 (m, 1H), 4.32- 3.78 (m, 2H), 3.59-3.30 (m, 2H), 3.29-3.22 (m, 1H), 1.06 (s, 3H), 0.71- 0.40 (m, 4H). MS (ESI) m/z [M + H]⁺ = 440. |
| D13 | 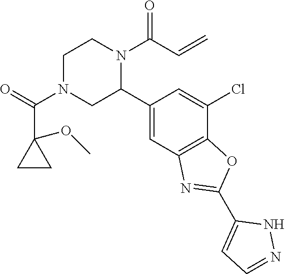 [SMILES: C=CC(=O)N1CCN(C(=O)C2(OC)CC2)CC1c1cc(Cl)c2oc(-c3ccn[nH]3)nc2c1] | 1-(2-(7-chloro-2-(1H-pyrazol- 5-yl)benzo[d]oxazol-5-yl)-4- (1-methoxycyclopropane-1- carbonyl)piperazin-1-yl)prop- 2-en-1-one | ¹H NMR (400 MHz, DMSO-d₆) δ 13.68 (s, 1H), 7.99 (s, 1H), 7.58 (s, 1H), 7.50- 7.25 (m,1H), 7.10-6.71 (m, 2H), 6.29-6.11 (m, 1H), 5.94-5.52 (m, 2H), 4.86-4.61 (m, 1H), 4.29-3.70 (m, 3H), 3.46-3.34 (m, 1H), 3.10- 2.69 (m, 4H), 1.04-0.61 (m, 4H). MS (ESI) m/z [M + H]⁺ = 456. |
| D14 | 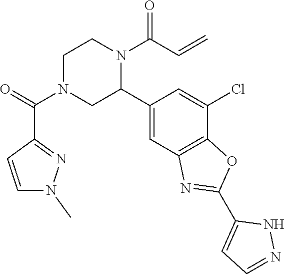 [SMILES: C=CC(=O)N1CCN(C(=O)c2ccn(C)n2)CC1c1cc(Cl)c2oc(-c3ccn[nH]3)nc2c1] | 1-(2-(7-chloro-2-(1H-pyrazol- 5-yl)benzo[d]oxazol-5-yl)-4- (1-methyl-1H-pyrazole-3- carbonyl)piperazin-1-yl)prop- 2-en-1-one | ¹H NMR (400 MHz, DMSO-d₆) δ 13.67 (s, 1H), 7.98 (s, 1H), 7.71 (s, 1H), 7.66- 7.41 (m, 1H), 7.35 (s, 1H), 7.07- 6.70 (m, 2H), 6.60-6.30 (m, 1H), 6.28- 6.12 (m, 1H), 5.93-5.50 (m, 2H), 5.28-4.80 (m, 1H), 4.68-4.00 (m, 2H), 3.98-3.74 (m, 3H), 3.63-3.36 (m, 2H), 3.27-3.14 (m, 1H). MS (ESI) m/z [M + H]⁺ = 466. |

### Example 2-5: Synthesis of N-(1-acryloyl-2-(7-chloro-2-(1H-imidazol-5-yl)benzofuran-5-yl)piperidin-4-yl)acetamide (H3 and H4)

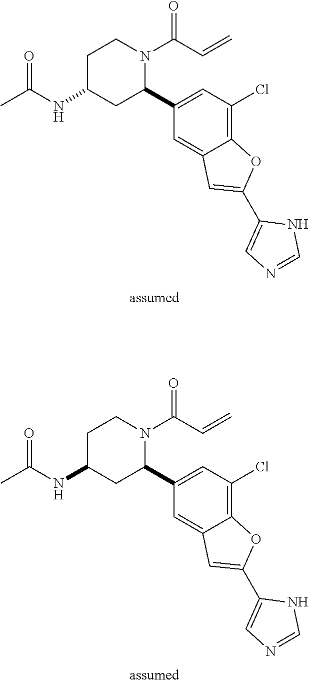

### Step 1: N-(2-(7-chloro-2-(1-((2-(trimethylsilyl)ethoxy)methyl)-1H-imidazol-4-yl)benzofuran-5-yl)-1-tosylpiperidin-4-yl)acetamide

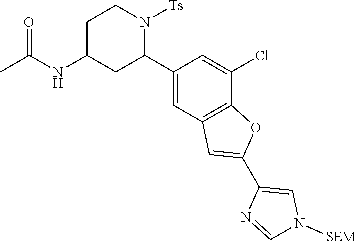 [SMILES: CC(=O)NC1CCN(S(=O)(=O)c2ccc(C)cc2)C(c2cc(Cl)c3oc(-c4cn(COCC[Si](C)(C)C)cn4)cc3c2)C1]

A solution of (5-(4-acetamido-1-tosylpiperidin-2-yl)-7-chlorobenzofuran-2-yl)boronic acid (200 mg, 0.41 mmol), 2-[(4-bromoimidazol-1-yl)methoxy]ethyl-trimethyl-silane (147 mg, 0.53 mmol), Pd(dtbpf)Cl₂ (27 mg, 0.04 mmol) and K₂CO₃ (113 mg, 0.82 mmol) in 1,4-dioxane (10 mL) and H₂O (2 mL) was stirred at 80° C. for 1 hour. After cooled to rt, the reaction was diluted with DCM, washed with brine, dried over Na₂SO₄, filtered, concentrated and the residue was purified on silica gel column chromatography (MeOH/DCM 1/100 to 1/20) to give the title compound (220 mg, 83.9%). MS (ESI) m/z [M+H]⁺ 643.

### Step 2: N-(2-(7-chloro-2-(1H-imidazol-4-yl)benzofuran-5-yl)piperidin-4-yl)acetamide

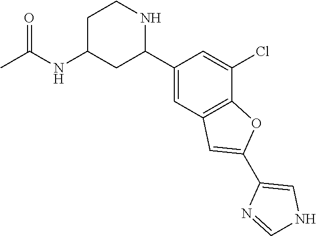 [SMILES: CC(=O)NC1CCNC(c2cc(Cl)c3oc(-c4c[nH]cn4)cc3c2)C1]

A solution of N-(2-(7-chloro-2-(1-((2-(trimethylsilyl)ethoxy)methyl)-1H-imidazol-4-yl)benzofuran-5-yl)-1-tosylpiperidin-4-yl)acetamide (105 mg, 0.16 mmol) in HBr/AcOH (10 mL) was stirred at 80° C. for 1 hour. The reaction solution was concentrated and the residue was purified on C18 column [H₂O (0.1% FA):ACN=10% to 90%] to give the title compound (50 mg, 85.3%). MS (ESI) m/z [M+H]⁺ 359.

### Step 3: N-(1-acryloyl-2-(7-chloro-2-(1H-imidazol-4-yl)benzofuran-5-yl)piperidin-4-yl)acetamide

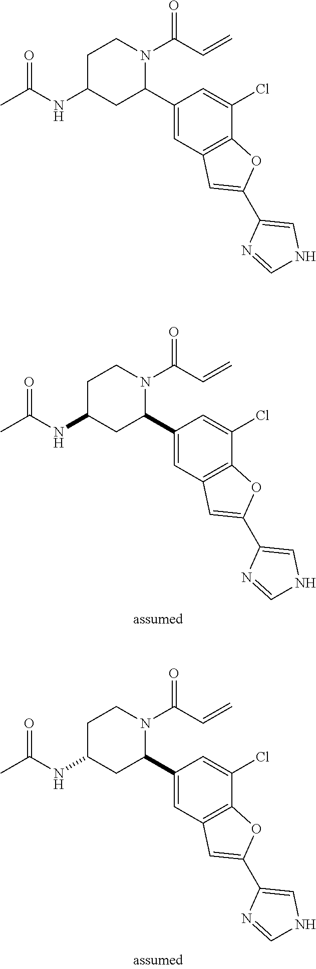

To a solution of N-(2-(7-chloro-2-(1H-imidazol-4-yl)benzofuran-5-yl)piperidin-4-yl)acetamide (50 mg, 0.14 mmol) in DCM (10 mL) and sat.NaHCO₃ (10 mL) was added prop-2-enoyl chloride (12 mg, 0.13 mmol) at 0° C. and the resulting mixture was stirred at 0° C. for 10 mins. The reaction mixture was diluted with DCM, washed with brine, dried over Na₂SO₄, filtered, concentrated and the residue was purified on Prep-TLC(M:D=1:20) to give the crude product. The crude was purified on Prep-HPLC to give the compound H3 (4.25 mg, 7.4%) and the compound H4 (0.4 mg, 0.7%). MS (ESI) m/z [M+H]⁺ 413.

| Compound | Structure | Chemical Name | ¹H NMR data LC/MS m/z (M + H) |
|---|---|---|---|
| H3 | 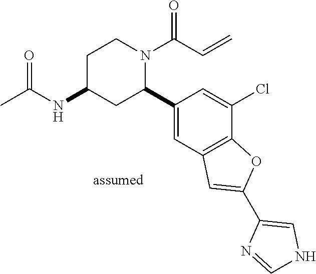 [SMILES: C=CC(=O)N1CC[C@H](NC(C)=O)C[C@@H]1c1cc(Cl)c2oc(-c3c[nH]cn3)cc2c1] | A mixture of N-((cis)-1- acryloyl-2-(7-chloro-2-(1H- imidazol-4-yl)benzofuran-5- yl)piperidin-4-yl)acetamide or N-((trans)-1-acryloyl-2- (7-chloro-2-(1H-imidazol-4- yl)benzofuran-5- yl)piperidin-4-yl)acetamide, assumed | ¹H NMR (400 MHz, DMSO-d₆) δ 12.49 (s, 1H), 7.93-7.84 (m, 1H), 7.79 (s, 1H), 7.67 (s, 1H), 7.48-7.35 (m, 1H), 7.13 (s, 1H), 7.05-6.70 (m, 2H), 6.19 (m, 1H), 6.00-5.51 (m, 2H), 4.55- 3.96 (m, 1H), 3.87-3.62 (m, 1H), 2.59- 2.54 (m, 1H), 1.80 (s, 3H), 1.73- 1.49 (m, 2H), 1.38-1.13 (m, 2H). MS (ESI) m/z [M + H]⁺ = 413. |
| H4 | 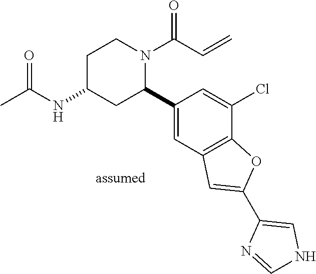 | A mixture of N-((cis)-1- acryloyl-2-(7-chloro-2-(1H- imidazol-4-yl)benzofuran-5- yl)piperidin-4-yl)acetamide or N-((trans)-1-acryloyl-2- (7-chloro-2-(1H-imidazol-4- yl)benzofuran-5- yl)piperidin-4-yl)acetamide, assumed | ¹H NMR (400 MHz, DMSO-d₆) δ 12.47 (s, 1H), 7.78 (s, 1H), 7.66 (s, 1H), 7.60 (d, J = 6.7 Hz, 1H), 7.30 (s, 1H), 7.09 (s, 1H), 7.00 (s, 1H), 6.80-6.60 (m, 1H), 6.11 (m, 1H), 5.63 (m, 1H), 5.43- 5.32 (m, 1H), 4.26-4.12 (m, 1H), 3.82- 3.75 (m, 1H), 2.32-2.24 (m, 1H), 2.08-1.88 (m, 2H), 1.58 (s, 3H), 1.51- 1.43 (m, 1H), 1.28-1.12 (m, 1H). MS (ESI) m/z [M+ H]⁺ = 413. |

Compounds H1/2 in Table 2-4 below were synthesized by using corresponding starting materials according to procedures similar to those described for the synthesis of compounds H3/4.

TABLE 2-4

| Compound | Structure | Chemical Name | ¹H NMR data LC/MS m/z (M + H) |
|---|---|---|---|
| H1 | 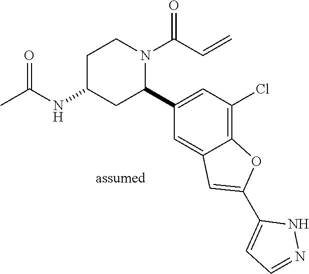 | N-(-1-acryloyl-2-(7-chloro- 2-(1H-pyrazol-5- yl)benzofuran-5- yl)piperidin-4-yl)acetamide (assumed) | ¹H NMR (400 MHz, DMSO-d₆) δ 13.25 (s, 1H), 7.87 (s, 1H), 7.60 (d, J = 6.5 Hz, 1H), 7.35 (s, 1H), 7.22 (s, 1H), 7.15 (s, 1H), 6.79-6.60 (m, 2H), 6.15- 6.06 (m, 1H), 5.64 (d, J = 9.4 Hz, 1H), 5.43-5.34 (m, 1H), 4.24-4.13 (m, 1H), 3.85-3.70 (m, 2H), 2.06-2.01 (m, 1H), 1.97-1.91 (m, J = 6.7 Hz, 1H), 1.54 (s, 3H), 1.50-1.45 (m, 1H), 1.22-1.18 (m, 1H). MS (ESI) m/z [M + H]⁺ = 413. |
| H2 | 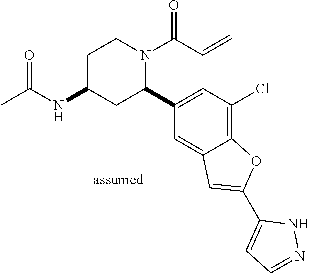 [SMILES: C=CC(=O)N1CC[C@H](NC(C)=O)C[C@@H]1c1cc(Cl)c2oc(-c3ccn[nH]3)cc2c1] | N-(-1-acryloyl-2-(7-chloro- 2-(1H-pyrazol-5- yl)benzofuran-5- yl)piperidin-4-yl)acetamide (assumed) | ¹H NMR (400 MHz, DMSO-d₆) δ 13.26 (s, 1H), 7.94-7.82 (m, 2H), 7.45 (s, 1H), 7.27-7.14 (m, 2H), 7.03-6.77 (m, 1H), 6.74 (s, 1H), 6.24-6.13 (m, 1H), 6.03-5.50 (m, 2H), 4.54-3.70 (m, 1H), 3.24-2.88 (m, 1H), 2.67- 2.55 (m, 1H), 1.77 (s, 3H), 1.71-1.49 (m, 2H), 1.37-1.11 (m, 2H). MS (ESI) m/z [M + H]⁺ = 413. |

### Example 2-6: Synthesis of (R)-1-(3-(7-chloro-2-(1H-1,2,4-triazol-5-yl)benzofuran-5-yl)morpholino)prop-2-en-1-one (Assumed) (B37A) and (S)-1-(3-(7-chloro-2-(1H-1,2,4-triazol-5-yl)benzofuran-5-yl)morpholino)prop-2-en-1-one (Assumed) (B37B)

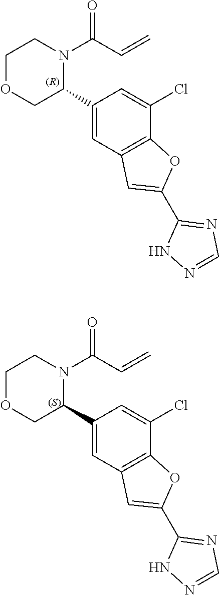

### Step 1: tert-butyl 3-(7-chloro-2-(1-((2-(trimethylsilyl)ethoxy)methyl)-1H-1,2,4-triazol-5-yl)benzofuran-5-yl)morpholine-4-carboxylate (Mixture of SEM Regio-Isomers)

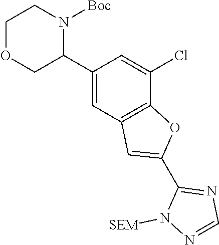 [SMILES: CC(C)(C)OC(=O)N1CCOCC1c1cc(Cl)c2oc(-c3ncnn3COCC[Si](C)(C)C)cc2c1]

A solution of 2-[(5-bromo-1,2,4-triazol-1-yl)methoxy]ethyl-trimethyl-silane (two regio-isomers, ratio 7:1) (200 mg, 0.72 mmol), [5-(4-tert-butoxycarbonylmorpholin-3-yl)-7-chloro-benzofuran-2-yl]boronic acid (275 mg, 0.72 mmol), Pd(dtbpf)Cl₂ (47 mg, 0.072 mmol) and K₂CO₃ (199 mg, 1.44 mmol) in 1,4-dioxane (10 mL) and H₂O (2 mL) was stirred at 80° C. for 1 hour under N2 atmosphere. After cooled to rt, the reaction was quenched with water and extracted with EA, the mixture was washed with brine, dried over Na₂SO₄, filtered and concentrated under reduced pressure and the residue was purified by silica gel column chromatography (PE/EtOAc 10/1 to 3/2) to give the title compound (mixture of SEM regio-isomers, 162 mg, 42%). MS (ESI) m/z [M+H]⁺ 535.

### Step 2: 3-(7-chloro-2-(1H-1,2,4-triazol-5-yl)benzofuran-5-yl)morpholine

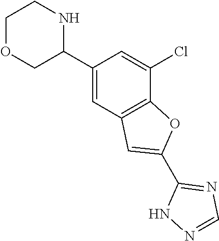 [SMILES: Clc1cc(C2COCCN2)cc2cc(-c3ncn[nH]3)oc12]

A solution of tert-butyl 3-[7-chloro-2-[2-(2-trimethylsilylethoxymethyl)-1,2,4-triazol-3-yl]benzofuran-5-yl]morpholine-4-carboxylate (mixture of SEM regio-isomers, 162 mg, 0.30 mmol) in DCM (5 mL) and TFA (5 mL) was stirred at rt for 2 hours. Upon completion of the reaction, the solvent was removed under reduced pressure to give the crude product. MS (ESI) m/z [M+H]⁺ 305.

### Step 3:1-(3-(7-chloro-2-(1H-1,2,4-triazol-5-yl)benzofuran-5-yl)morpholino)prop-2-en-1-one (B7)

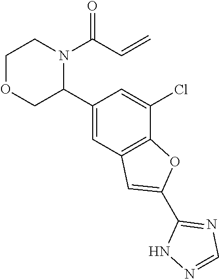 [SMILES: C=CC(=O)N1CCOCC1c1cc(Cl)c2oc(-c3ncn[nH]3)cc2c1]

To a solution of 3-[7-chloro-2-(1H-1,2,4-triazol-5-yl)benzofuran-5-yl]morpholine (92 mg, 0.30 mmol) in DCM (5 mL) and sat.NaHCO₃ (5 mL) was added acryloyl chloride (28 mg, 0.31 mmol) in DCM (1 mL) at 0° C., the resulting mixture was stirred at 0° C. for 10 min. Upon completion of the reaction, the reaction was extracted with DCM, the organic phase was dried over Na₂SO₄ and concentrated. The residue was purified by C18 column [ACN:H₂O (0.1% FA)=2:3] to give the product (25 mg, 23%). MS (ESI) m/z [M+H]⁺ 359.

### Step 4: (R)-1-(3-(7-chloro-2-(1H-1,2,4-triazol-5-yl)benzofuran-5-yl)morpholino)prop-2-en-1-one (Assumed) and (S)-1-(3-(7-chloro-2-(1H-1,2,4-triazol-5-yl)benzofuran-5-yl)morpholino)prop-2-en-1-one (Assumed)

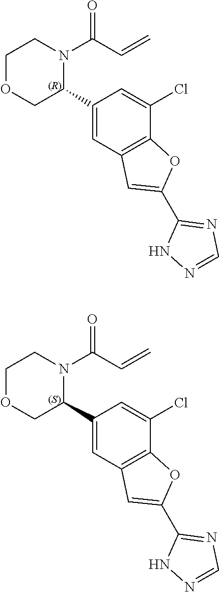

Racemic 1-(3-(7-chloro-2-(1H-1,2,4-triazol-5-yl)benzofuran-5-yl)morpholino)prop-2-en-1-one was separated by using the following conditions:

Column: CHIRALPAK AD-H 20 mm×250 mm, 5 m; 35% MeOH(0.3% 2M NH₃ MeOH), isomer 1, retention time=6.72 min, isomer 2, retention time=10.28 min.

Isomer 1:

¹H NMR (400 MHz, DMSO-d₆) δ 8.65 (s, 1H), 7.67 (s, 1H), 7.50-7.38 (m, 2H), 6.88-6.79 (m, 1H), 6.20 (d, J=16.6 Hz, 1H), 5.73 (d, J=10.4 Hz, 1H), 5.68-5.28 (m, 1H), 4.42 (d, J=12.1 Hz, 1H), 4.31-3.90 (m, 1H), 3.89-3.73 (m, 2H), 3.49 (t, J=10.9 Hz, 1H), 3.15-2.94 (m, 1H). MS (ESI) m/z [M+H]⁺=359.

Isomer 2:

¹H NMR (400 MHz, DMSO-d₆) δ 8.66 (s, 1H), 7.67 (s, 1H), 7.51-7.36 (m, 2H), 6.90-6.76 (m, 1H), 6.21 (d, J=16.6 Hz, 1H), 5.73 (d, J=9.6 Hz, 1H), 5.66-5.30 (m, 1H), 4.42 (d, J=12.2 Hz, 1H), 4.30-3.91 (m, 1H), 3.90-3.75 (m, 2H), 3.53-3.45 (m, 1H), 3.16-2.96 (m, 1H). MS (ESI) m/z [M+H]⁺=359.

### Example 2-7: Synthesis of 1-(3-(3,7-dichloro-2-(1H-1,2,4-triazol-3-yl)benzofuran-5-yl)morpholino)prop-2-en-1-one (B34)

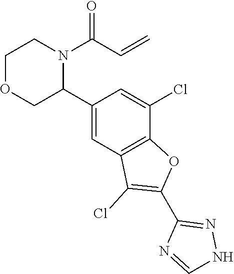 [SMILES: C=CC(=O)N1CCOCC1c1cc(Cl)c2oc(-c3nc[nH]n3)c(Cl)c2c1]

### Step 1: 3-(5-bromo-3,7-dichlorobenzofuran-2-yl)-1-((2-(trimethylsilyl)ethoxy)methyl)-1H-1,2,4-triazole

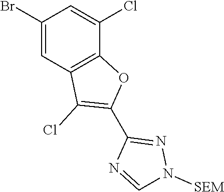 [SMILES: C[Si](C)(C)CCOCn1cnc(-c2oc3c(Cl)cc(Br)cc3c2Cl)n1]

To a solution of 3-(5-bromo-7-chlorobenzofuran-2-yl)-1-((2-(trimethylsilyl)ethoxy)methyl)-1H-1,2,4-triazole (450 mg, 1.05 mmol) in MeCN (5 mL) was added N-chlorosuccinimide (160 mg, 1.2 mmol) at room temperature. The reaction mixture was then stirred for 3 h at 65° C. Upon completion of the reaction, the volatiles were removed under reduced pressure and the resulting residue was diluted with water and extracted with ethyl acetate for three times. The combined organic layers were washed with brine, dried over Na₂SO₄, filtered and concentrated under reduced pressure to afford a residue, which was purified by silica gel column chromatography (EA/PE 1/10 to 1/2) to give the title compound (170 mg, 35%). MS (ESI) m/z [M+H]⁺ 462, 464.

### Step 2: tert-butyl 5-(3,7-dichloro-2-(1-((2-(trimethylsilyl)ethoxy)methyl)-1H-1,2,4-triazol-3-yl)benzofuran-5-yl)-2,3-dihydro-4H-1,4-oxazine-4-carboxylate

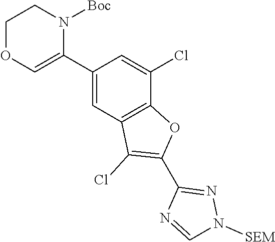 [SMILES: CC(C)(C)OC(=O)N1CCOC=C1c1cc(Cl)c2oc(-c3ncn(COCC[Si](C)(C)C)n3)c(Cl)c2c1]

A solution of 3-(5-bromo-3,7-dichlorobenzofuran-2-yl)-1-((2-(trimethylsilyl)ethoxy)methyl)-1H-1,2,4-triazole (170 mg, 0.36 mmol), bis(pinacolato)diboron (140 mg, 0.55 mmol), Pd(dppf)Cl₂ (36 mg, 0.05 mmol) and KOAc (70 mg, 0.72 mmol) in 1,4-dioxane (6 mL) was stirred at 90° C. under N₂ atmosphere for 2 h. After completion of the reaction, the reaction mixture was cooled to room temperature, tert-butyl 5-((diphenoxyphosphoryl)oxy)-2,3-dihydro-4H-1,4-oxazine-4-carboxylate (156 mg, 0.36 mmol), K₂CO₃ (99 mg, 0.72 mmol), Pd(dppf)Cl₂ (36 mg, 0.05 mmol) and water (1.5 mL) were added successively. The mixture was then stirred at 90° C. under N₂ atmosphere for 2 h before it was diluted with water and extracted with ethyl acetate for three times. The combined organic layers were washed with brine, dried over Na₂SO₄, filtered and concentrated under reduced pressure to afford a residue, which was purified by silica gel column chromatography (EA/PE 1/10 to 1/1) to give the title compound (60 mg, 29%). MS (ESI) m/z [M+H]⁺ 567.

### Step 3:1-(3-(3,7-dichloro-2-(1H-1,2,4-triazol-3-yl)benzofuran-5-yl)morpholino)prop-2-en-1-one

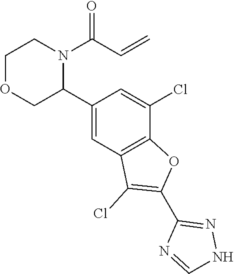 [SMILES: C=CC(=O)N1CCOCC1c1cc(Cl)c2oc(-c3nc[nH]n3)c(Cl)c2c1]

To a solution of tert-butyl 5-(3,7-dichloro-2-(1-((2-(trimethylsilyl)ethoxy)methyl)-1H-1,2,4-triazol-3-yl)benzofuran-5-yl)-2,3-dihydro-4H-1,4-oxazine-4-carboxylate (60 mg, 0.11 mmol) in DCM (2 mL) was added trifluoroacetic acid (2 mL) at rt. The mixture was stirred at rt for 1 h, then it was cooled to 0° C. and sodium triacetoxyborohydride (117 mg, 0.55 mmol) was added in one portion. The resulting reaction mixture was allowed to warm up to rt and stirred for 3 h before it was concentrated under reduced pressure to remove the volatiles. Then the resulting residue was redissolved in 3 mL THF and 3 mL sat. NaHCO₃ solution, followed by the addition of acryloyl chloride (10 mg, 0.11 mmol) at room temperature. The reaction was stirred at rt for 1 h and diluted with water. The mixture was then extracted with ethyl acetate. The combined organic layers were washed with brine, dried over Na2SO4, filtered and concentrated under reduced pressure to give a residue, which was purified by C18 column (mobile phase, ACN in water (0.1% FA), 25% to 45%) to give the title compound (8.2 mg, 20%). 1H NMR (500 MHz, DMSO-d₆) δ 8.79 (s, 1H), 7.59 (s, 2H), 6.89 (dd, J=16.6, 10.4 Hz, 1H), 6.25 (dd, J=16.6, 2.1 Hz, 1H), 5.78 (dd, J=10.4, 2.3 Hz, 1H), 5.66 (s, 1H), 4.50 (d, J=12.3 Hz, 1H), 4.45-3.82 (m, 3H), 3.54 (t, J=10.4 Hz, 1H), 3.23-2.73 (m, 1H). MS (ESI) m/z [M+H]⁺=393.

### Example 2-8: Synthesis of 1-(3-(7-chloro-3-methyl-2-(1H-1,2,4-triazol-3-yl)benzofuran-5-yl)morpholino)prop-2-en-1-one (B35)

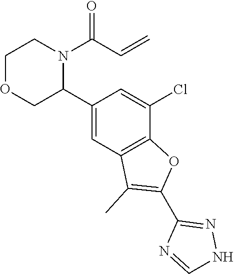 [SMILES: C=CC(=O)N1CCOCC1c1cc(Cl)c2oc(-c3nc[nH]n3)c(C)c2c1]

### Step 1: ethyl 7-chloro-3-methylbenzofuran-2-carboxylate

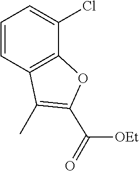 [SMILES: CCOC(=O)c1oc2c(Cl)cccc2c1C]

To a solution of 1-(3-chloro-2-hydroxyphenyl)ethan-1-one (5 g, 29.4 mmol) in DMF (50 mL) was added ethyl bromoacetate (5.89 g, 35.3 mmol) and K₂CO₃ (8.11 g, 58.8 mmol). The resulting reaction mixture was stirred for 2 h at 50° C. before it was diluted with water (250 mL) and extracted with ethyl acetate. The combined organic layers were washed with brine, dried over N₂SO₄, filtered and concentrated under reduced pressure to afford a residue, which was used for the next step directly without further purification. The residue was redissolved in 50 mL toluene and to this resulting solution was added DBU (8.96 g, 58.8 mmol) at room temperature. Then it was stirred at 100° C. for 1 h. After completion of the reaction, it was quenched with a cold aq. solution of 1N HCl (60 mL) and extracted with ethyl acetate three times. The combined organic layers were washed with brine, dried over N₂SO₄, filtered and concentrated under reduced pressure. The residue was purified by silica gel column chromatography (EA/PE 1/10 to 1/1) to give the title compound (3.6 g, 51%). MS (ESI) m/z [M+H]⁺ 239.

### Step 2: ethyl 7-chloro-3-methyl-5-(4,4,5,5-tetramethyl-1,3,2-dioxaborolan-2-yl)benzofuran-2-carboxylate

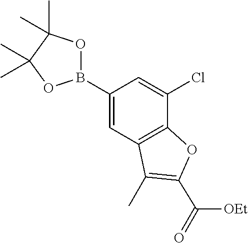 [SMILES: CCOC(=O)c1oc2c(Cl)cc(B3OC(C)(C)C(C)(C)O3)cc2c1C]

To a solution of ethyl 7-chloro-3-methylbenzofuran-2-carboxylate (830 mg, 3.49 mmol) in tetrahydrofuran (20 mL) was added 4,4′-di-tert-butyl-2,2′-bipyridyl (dtbbpy) (187 mg, 0.70 mmol) and (1,5-cyclooctadiene)(methoxy)iridium(I) dimer (231.2 mg, 0.35 mmol) at rt under N₂ atmosphere. Then pinacolborane (HBpin) (2.23 g, 17.5 mmol) was added to this solution dropwise via syringe. The resulting reaction mixture was stirred at 60° C. for 1 h. Then it was cooled to rt and quenched with MeOH (15 mL). After stirring at rt for 5 min, the reaction mixture was diluted with water and extracted with ethyl acetate. The combined organic layers were washed with brine, dried over N₂SO₄, filtered and concentrated under reduced pressure to give a residue, which was purified by silica gel column chromatography (EA/PE 1/10 to 1/2) to give the title compound (1.06 g, 85%). MS (ESI) m/z [M+H]⁺=365.

### Step 3: tert-butyl 5-(7-chloro-2-(ethoxycarbonyl)-3-methylbenzofuran-5-yl)-2,3-dihydro-4H-1,4-oxazine-4-carboxylate

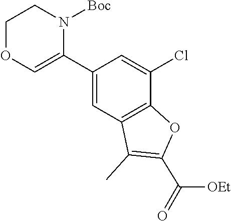 [SMILES: CCOC(=O)c1oc2c(Cl)cc(C3=COCCN3C(=O)OC(C)(C)C)cc2c1C]

To a solution of ethyl 7-chloro-3-methyl-5-(4,4,5,5-tetramethyl-1,3,2-dioxaborolan-2-yl)benzofuran-2-carboxylate (360 mg, 0.99 mmol) in 1,4-dioxane (10 mL) and water (2 mL) was added Pd(dppf)Cl₂ (73.1 mg, 0.1 mmol), K₂CO₃ (205 mg, 1.50 mmol) and tert-butyl 5-((diphenoxyphosphoryl)oxy)-2,3-dihydro-4H-1,4-oxazine-4-carboxylate (511 mg, 1.18 mmol) at rt under N₂ atmosphere. Then the resulting reaction mixture was stirred at 90° C. for 1.5 h. Upon completion of the reaction monitored by LC-MS, the reaction was diluted with water and extracted with ethyl acetate. The combined organic layers were washed with brine, dried over Na2SO4, filtered and concentrated under reduced pressure to give a residue, which was purified by silica gel column chromatography (EA/PE 1/10 to 1/1) to give the title compound (310 mg, 75%). MS (ESI) m/z [M+H]⁺=422.

### Step 4: tert-butyl 3-(7-chloro-2-(ethoxycarbonyl)-3-methylbenzofuran-5-yl)morpholine-4-carboxylate

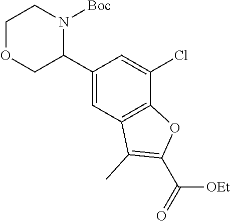 [SMILES: CCOC(=O)c1oc2c(Cl)cc(C3COCCN3C(=O)OC(C)(C)C)cc2c1C]

To a solution of tert-butyl 5-(7-chloro-2-(ethoxycarbonyl)-3-methylbenzofuran-5-yl)-2,3-dihydro-4H-1,4-oxazine-4-carboxylate (310 mg, 0.73 mmol) in DCM (5 mL) was added trifluoroacetic acid (2.5 mL) at rt. The mixture was stirred at rt for 1 h, then it was cooled to 0° C. and sodium triacetoxyborohydride (775 mg, 3.66 mmol) was added in one portion. The resulting reaction mixture was allowed to warm up to rt and stirred for 3 h before it was concentrated under reduced pressure to remove the volatiles. Then the resulting residue was redissolved in 5 mL THF and 5 mL sat. aq. NaHCO₃ solution, followed by the addition of Boc₂O (192 mg, 0.88 mmol) at room temperature. The reaction was stirred for 2 h and diluted with water. The mixture was then extracted with ethyl acetate. The combined organic layers were washed with brine, dried over Na2SO4, filtered and concentrated under reduced pressure to give a residue, which was purified by silica gel column chromatography (EA/PE 1/10 to 1/2) to give the title compound (180 mg, 58%). MS (ESI) m/z [M+H]⁺=424.

### Step 5: tert-butyl 3-(2-carbamoyl-7-chloro-3-methylbenzofuran-5-yl)morpholine-4-carboxylate

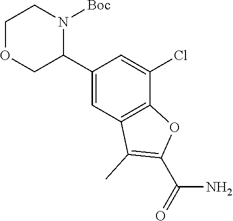 [SMILES: Cc1c(C(N)=O)oc2c(Cl)cc(C3COCCN3C(=O)OC(C)(C)C)cc12]

Tert-butyl 3-(7-chloro-2-(ethoxycarbonyl)-3-methylbenzofuran-5-yl)morpholine-4-carboxylate (180 mg, 0.43 mmol) was dissolved in 7 M NH₃/MeOH (10 mL), the mixture was stirred at 55° C. overnight. Upon completion of the reaction, it was concentrated under reduced pressure to remove the volatiles to afford the crude title compound (166 mg) which was used for the next step without further purification. MS (ESI) m/z [M+H]⁺=395.

### Step 6: tert-butyl 3-(7-chloro-3-methyl-2-(1H-1,2,4-triazol-3-yl)benzofuran-5-yl)morpholine-4-carboxylate

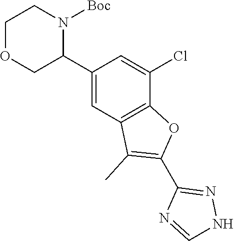 [SMILES: Cc1c(-c2nc[nH]n2)oc2c(Cl)cc(C3COCCN3C(=O)OC(C)(C)C)cc12]

To a solution of crude tert-butyl 3-(2-carbamoyl-7-chloro-3-methylbenzofuran-5-yl)morpholine-4-carboxylate (166 mg, 0.43 mmol) in DMF (3 mL) was added DMF-DMA (3 mL). The reaction mixture was stirred at 120° C. for 2 h. Then the volatiles were removed under vacuum to give the crude tert-butyl-3-(7-chloro-2-(((dimethylamino)methylene)carbamoyl)-3-methylbenzofuran-5-yl)morpholine-4-carboxylate (193 mg), which was used for the next step without further purification. MS (ESI) m/z [M+H]⁺ 450.

To a solution of the above described crude tert-butyl-3-(7-chloro-2-(((dimethylamino)methylene)carbamoyl)-3-methylbenzofuran-5-yl)morpholine-4-carboxylate (193 mg, 0.43 mmol) in AcOH (2 mL) and EtOH (6 mL) was added hydrazine hydrate (0.25 mL, 4.30 mmol, 85% w % in water) at room temperature. The reaction mixture was stirred at 90° C. for 1 h. After completion of the reaction, the volatiles were removed under vacuum. The resulting residue was dispersed in 5 mL water and stirred for 10 min at rt before it was filtered. The solid was collected to give the crude tert-butyl 3-(7-chloro-3-methyl-2-(1H-1,2,4-triazol-3-yl)benzofuran-5-yl)morpholine-4-carboxylate (125 mg, 0.30 mmol), which was used for the next step without further purification. MS (ESI) m/z [M+H]⁺ 419.

### Step 7: 1-(3-(7-chloro-3-methyl-2-(1H-1,2,4-triazol-3-yl)benzofuran-5-yl)morpholino)prop-2-en-1-one

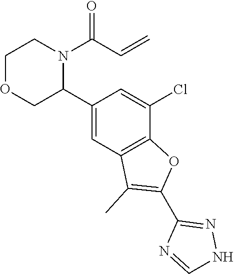 [SMILES: C=CC(=O)N1CCOCC1c1cc(Cl)c2oc(-c3nc[nH]n3)c(C)c2c1]

To a solution of tert-butyl 3-(7-chloro-3-methyl-2-(1H-1,2,4-triazol-3-yl)benzofuran-5-yl)morpholine-4-carboxylate (125 mg, 0.30 mmol) in DCM (5 mL) was added trifluoroacetic acid (2.5 mL). The mixture was stirred at rt for 1 h, then it was concentrated under reduced pressure to remove the volatiles. The resulting residue was redissolved in 5 mL THF and 5 mL sat. aq. NaHCO₃ solution, followed by the addition of acryloyl chloride (27 mg, 0.30 mmol) at room temperature. The reaction was stirred for 1 h and diluted with water. The mixture was then extracted with ethyl acetate. The combined organic layers were washed with brine, dried over Na2SO4, filtered and concentrated under reduced pressure to give a residue, which was purified by C18 column (mobile phase, ACN in water (0.1% FA), 25% to 45%) to give the title compound (36.6 mg, 33%).

1H NMR (400 MHz, DMSO-d₆) δ 8.62 (s, 1H), 7.56 (s, 1H), 7.35 (s, 1H), 6.80 (dd, J=16.6, 10.5 Hz, 1H), 6.16 (d, J=16.6 Hz, 1H), 5.69 (d, J=10.4 Hz, 1H), 5.51 (s, 1H), 4.41 (d, J=12.2 Hz, 1H), 4.05-3.68 (m, 3H), 3.45 (t, J=11.3 Hz, 1H), 3.13-2.86 (m, 1H), 2.42 (s, 3H). MS (ESI) m/z [M+H]⁺=373.

### Example 2-9: Synthesis of 1-(3-(3-bromo-7-chloro-2-(1H-1,2,4-triazol-5-yl)benzofuran-5-yl)morpholino)prop-2-en-1-one (B36)

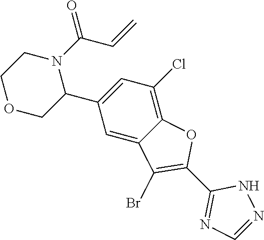 [SMILES: C=CC(=O)N1CCOCC1c1cc(Cl)c2oc(-c3ncn[nH]3)c(Br)c2c1]

### Step 1: tert-butyl 5-(7-chloro-2-(ethoxycarbonyl)benzofuran-5-yl)-2,3-dihydro-4H-1,4-oxazine-4-carboxylate

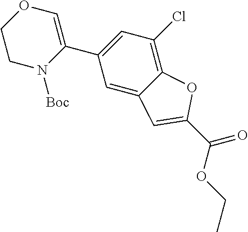 [SMILES: CCOC(=O)c1cc2cc(C3=COCCN3C(=O)OC(C)(C)C)cc(Cl)c2o1]

Ethyl 5-bromo-7-chloro-benzofuran-2-carboxylate (5.6 g, 18.5 mmol), 4,4,5,5-tetramethyl-2-(4,4,5,5-tetramethyl-1,3,2-dioxaborolan-2-yl)-1,3,2-dioxaborolane (5.2 g, 20.5 mmol), Pd(dppf)Cl₂ (0.8 g, 1.1 mmol) and KOAc (3.8 g, 38.7 mmol) were added into 1,4-dioxane (100 mL). The resulting reaction was charged into nitrogen and heated to 90° C. with stirring for 16 h. After cooled to rt, tert-butyl 5-diphenoxyphosphoryloxy-2,3-dihydro-1,4-oxazine-4-carboxylate (6.9 g, 16.0 mmol), Pd(dppf)Cl₂ (583.8 mg, 0.8 mmol), K₂CO₃ (4.4 g, 31.9 mmol) and H₂O (25 mL) were added into above reaction. The resulting reaction was charged into nitrogen and heated to 80° C. with stirring for 3 h. After cooled to rt, dioxane was removed in vacuo. The residue was diluted with EA/H₂O. The organic layer was washed with brine, dried over Na₂SO₄ and concentrated. The residue was applied onto a silica gel column chromatography with (EA/PE=1:7) to give the desired product (2.8 g, 90% purity, 38% yield). MS (ESI) m/z [M+H]⁺ 408.

### Step 2: tert-butyl 3-(7-chloro-2-(ethoxycarbonyl)benzofuran-5-yl)morpholine-4-carboxylate

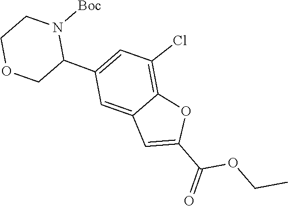 [SMILES: CCOC(=O)c1cc2cc(C3COCCN3C(=O)OC(C)(C)C)cc(Cl)c2o1]

Tert-butyl 5-(7-chloro-2-ethoxycarbonyl-benzofuran-5-yl)-2,3-dihydro-1,4-oxazine-4-carboxylate (2.8 g, 6.1 mmol, 90% purity) was added into DCM (30 mL). To this was added TFA (10 mL). The resulting reaction was stirred at rt for 1 h. The resulting mixture was cooled to 0° C. To this was added STAB (7.8 g, 36.7 mmol7) in several portions. The resulting reaction was warmed to rt with stirring for 3 h. The reaction mixture was concentrated. The residue was dissolved into DCM/MeOH (10:1). The organic layer was washed with sat. NaHCO₃ (aq.) and brine, dried over Na₂SO₄ and concentrated. The residue was added into sat. NaHCO₃ (aq.) (20 mL) and THF (20 mL). To this was added Boc₂O (0.4 g, 1.9 mmol) and the resulting mixture was stirred at rt for 2 h. The reaction was diluted with EA. The organic layer was washed with water and brine, dried over Na₂SO₄ and concentrated. The residue was applied onto a silica gel column chromatography (EA/PE=1:2) to give the desired product (2.2 g). MS (ESI) m/z [M+H]⁺ 410.

### Step 3: tert-butyl 3-(7-chloro-2-(ethoxycarbonyl)-3-(4,4,5,5-tetramethyl-1,3,2-dioxaborolan-2-yl)benzofuran-5-yl)morpholine-4-carboxylate

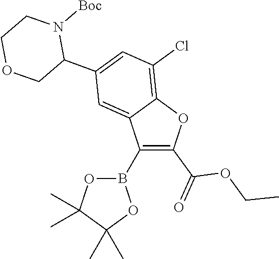 [SMILES: CCOC(=O)c1oc2c(Cl)cc(C3COCCN3C(=O)OC(C)(C)C)cc2c1B1OC(C)(C)C(C)(C)O1]

Tert-butyl 3-(7-chloro-2-ethoxycarbonyl-benzofuran-5-yl)morpholine-4-carboxylate (2 g, 4.9 mmol), [Ir(μ-OMe)(cod)₂](164.8 mg, 0.25 mmol), 4-tert-butyl-2-(4-tert-butyl-2-pyridyl)pyridine (144 mg, 0.5 mmol) and 4,4,5,5-tetramethyl-2-(4,4,5,5-tetramethyl-1,3,2-dioxaborolan-2-yl)-1,3,2 -dioxaborolane (1.8 g, 7.1 mmol) were added into n-hexane (50 mL). The resulting mixture was charged into nitrogen and heated to 70° C. with stirring for 1 h. After cooled to rt, the reaction was concentrated. The residue was applied onto a silica gel column chromatography (EA/PE=1:4 to 1:1) to give the desired product (3.3 g, 70% purity, 88% yield). MS (ESI) m/z [M+H]⁺ 536.

### Step 4: ethyl 3-bromo-7-chloro-5-(morpholin-3-yl)benzofuran-2-carboxylate

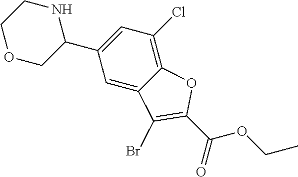 [SMILES: CCOC(=O)c1oc2c(Cl)cc(C3COCCN3)cc2c1Br]

Tert-butyl 3-[7-chloro-2-ethoxycarbonyl-3-(4,4,5,5-tetramethyl-1,3,2-dioxaborolan-2-yl)benzofuran-5-yl]morpholine-4-carboxylate (3.3 g, 4.3 mmol, 70 mass %) was dissolved into MeOH (50 mL). To this was added CuBr₂ (2 g, 8.6 mmol). The resulting mixture was charged into nitrogen and heated to 50° C. with stirring for 18 h. After cooled to rt, MeOH was removed in vacuo. The residue was applied onto a silica gel column chromatography (EA/DCM=1:3) to give the desired product (2 g, 80% purity, 95% yield). MS (ESI) m/z [M+H]⁺ 388, 390.

### Step 5: tert-butyl 3-(3-bromo-7-chloro-2-(ethoxycarbonyl)benzofuran-5-yl)morpholine-4-carboxylate

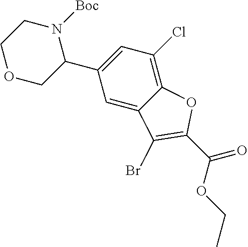 [SMILES: CCOC(=O)c1oc2c(Cl)cc(C3COCCN3C(=O)OC(C)(C)C)cc2c1Br]

Ethyl 3-bromo-7-chloro-5-morpholin-3-yl-benzofuran-2-carboxylate (2 g, 5.1 mmol, 80% purity) was added into sat. NaHCO₃ (aq.) (30 mL) and THF (30 mL). To this was added Boc₂O (2.8 g, 12.8 mmol). The resulting mixture was stirred at rt for 2 h. Upon completion of the reaction, EA was added and the organic layer was separated out, then it was washed with water and brine, dried over Na₂SO₄ and concentrated. The residue was applied onto a silica gel column chromatography (EA/PE=1:2) to give the desired product (1.5 g, 75% yield). MS (ESI) m/z [M+H]⁺ 488, 490.

### Step 6: tert-butyl 3-(3-bromo-2-carbamoyl-7-chlorobenzofuran-5-yl)morpholine-4-carboxylate

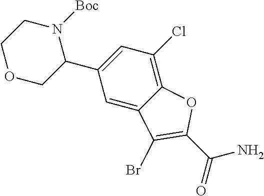 [SMILES: CC(C)(C)OC(=O)N1CCOCC1c1cc(Cl)c2oc(C(N)=O)c(Br)c2c1]

Tert-butyl 3-(3-bromo-7-chloro-2-ethoxycarbonyl-benzofuran-5-yl)morpholine-4-carboxylate (1.5 g, 3.1 mmol) was added into NH₃ in MeOH (7M) (30 mL). The resulting mixture was sealed into a tube and heated to 50° C. with stirring for 1 h. After cooled to rt, the reaction was concentrated to give the crude desired product (1 g, 71% yield). MS (ESI) m/z [M+H]⁺ 459, 461.

### Step 7: tert-butyl 3-(3-bromo-7-chloro-2-(1H-1,2,4-triazol-5-yl)benzofuran-5-yl)morpholine-4-carboxylate

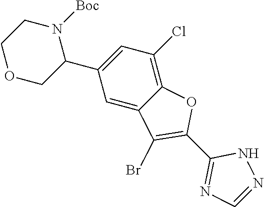 [SMILES: CC(C)(C)OC(=O)N1CCOCC1c1cc(Cl)c2oc(-c3ncn[nH]3)c(Br)c2c1]

Tert-butyl 3-(3-bromo-2-carbamoyl-7-chloro-benzofuran-5-yl)morpholine-4-carboxylate (1 g, 2.2 mmol) and DMF-DMA (1 mL) were added into DMF (10 mL). The resulting reaction was heated to 110° C. with stirring for 2 h. After cooled to rt, the reaction was concentrated. The residue was dissolved into EtOH (12 mL) and acetic acid (0.5 mL). To this was added hydrazine hydrate (530 mg, 10.6 mmol, 85% aq.) at rt. The resulting mixture was stirred at 90° C. for 1 h. After cooled to rt, the solids were collected by filtration and dried in vacuo to give the desired product (810 mg, 78% yield). MS (ESI) m/z [M+H]⁺ 483, 485.

### Step 8: tert-butyl 3-(3-bromo-7-chloro-2-(1-((2-(trimethylsilyl)ethoxy)methyl)-1H-1,2,4-triazol-5-yl)benzofuran-5-yl)morpholine-4-carboxylate (Mixture of SEM Regio-Isomers)

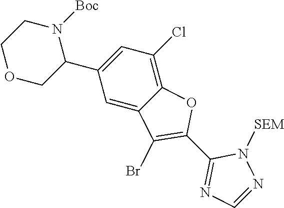 [SMILES: CC(C)(C)OC(=O)N1CCOCC1c1cc(Cl)c2oc(-c3ncnn3COCC[Si](C)(C)C)c(Br)c2c1]

Tert-butyl 3-[3-bromo-7-chloro-2-(1H-1,2,4-triazol-5-yl)benzofuran-5-yl]morpholine-4-carboxylate (810 mg, 1.7 mmol) was added into DMF (15 mL). To this was added TEA (507 mg, 5.0 mmol) and followed by addition of 2-(chloromethoxy)ethyl-trimethyl-silane (363 mg, 2.2 mmol at rt. The resulting reaction was stirred at rt for 18 h. Upon completing reaction, the reaction mixture was added into ice/water and extracted with EA. The organic layer was washed with water and brine, dried over Na₂SO₄ and concentrated. The residue was applied onto a silica gel column chromatography (EA/PE=1:3) to give the desired product (910 mg, 88% yield). MS (ESI) m/z [M+H]⁺ 613, 615.

### Step 9: 3-(3-bromo-7-chloro-2-(1H-1,2,4-triazol-5-yl)benzofuran-5-yl)morpholine

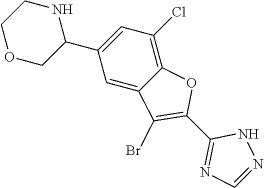 [SMILES: Clc1cc(C2COCCN2)cc2c(Br)c(-c3ncn[nH]3)oc12]

Tert-butyl 3-[3-bromo-7-chloro-2-(1H-1,2,4-triazol-5-yl)benzofuran-5-yl]morpholine-4-carboxylate (mixture of SEM regio-isomers, 89 mg, 0.2 mmol) was added into DCM (9 mL). To this was added TFA (3 mL). The resulting mixture was stirred at rt for 1 h. The reaction was concentrated to give the desired product (80 mg, 90% yield). MS (ESI) m/z [M+H]⁺ 383, 385.

### Step 10: 1-(3-(3-bromo-7-chloro-2-(1H-1,2,4-triazol-5-yl)benzofuran-5-yl)morpholino)prop-2-en-1-one

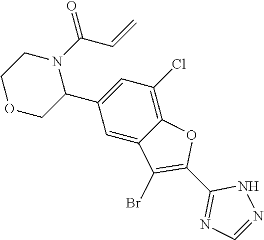 [SMILES: C=CC(=O)N1CCOCC1c1cc(Cl)c2oc(-c3ncn[nH]3)c(Br)c2c1]

3-[3-Bromo-7-chloro-2-(1H-1,2,4-triazol-5-yl)benzofuran-5-yl]morpholine (80 mg, 0.2 mmol) was added into sat. NaHCO₃ (10 mL) and DCM (10 mL). The resulting mixture was cooled to 0° C. To this was added acryloyl chloride (20 mg, 0.2 mmol) dropwise with stirring at 0° C. The resulting reaction was stirred below 10° C. for 10 min. Upon completion of the reaction, DCM/MeOH (10:1) (30 mL) was added. The organic layer was washed with brine and then concentrated. The residue was applied onto Prep-TLC with DCM/MeOH (20:1). And then repurified onto Prep-HPLC with MeCN/water (0.1% FA) to give the desired product (43 mg, 59% yield). ¹H NMR (400 MHz, DMSO-d₆) δ 8.80 (s, 1H), 7.66-7.49 (m, 2H), 6.89 (dd, J=16.4, 10.6 Hz, 1H), 6.25 (d, J=16.7 Hz, 1H), 5.78 (d, J=10.4 Hz, 1H), 5.63 (s, 1H), 4.49 (d, J=12.3 Hz, 1H), 4.40-3.78 (m, 3H), 3.54 (t, J=11.3 Hz, 1H), 3.17-2.83 (m, 1H). MS (ESI) m/z [M+H]⁺ 437, 439.

### Example 2-10: Synthesis of (R)-1-(6-(7-chloro-2-(1H-1,2,4-triazol-3-yl)benzofuran-5-yl)-4-oxa-7-azaspiro[2.5]octan-7-yl)prop-2-en-1-one (Assumed) (B26A) and (S)-1-(6-(7-chloro-2-(1H-1,2,4-triazol-3-yl)benzofuran-5-yl)-4-oxa-7-azaspiro[2.5]octan-7-yl)prop-2-en-1-one (Assumed) (B26B)

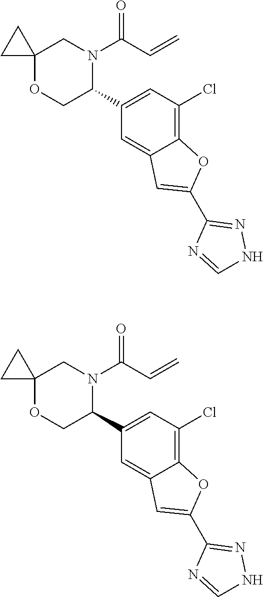 [SMILES: C=CC(=O)N1CC2(CC2)OC[C@H]1c1cc(Cl)c2oc(-c3nc[nH]n3)cc2c1 ; C=CC(=O)N1CC2(CC2)OC[C@@H]1c1cc(Cl)c2oc(-c3nc[nH]n3)cc2c1]

Racemic 1-(6-(7-chloro-2-(1H-1,2,4-triazol-3-yl)benzofuran-5-yl)-4-oxa-7-azaspiro[2.5]octan-7-yl)prop-2-en-1-one (synthesized according to procedures for compound B7 using corresponding starting materials) was separated using the following conditions:

SFC: column: YMC i-Amylose-3, 30 mm×250 mm, 5 um; fluent: 45% MeOH (0.3% 2M NH3 MeOH)/CO2; isomer 1, retention time=4.60 min; isomer 2, retention time=7.93 min.

Isomer 1:

¹H NMR (500 MHz, DMSO-d6) δ 8.76 (s, 1H), 7.77 (s, 1H), 7.53-7.50 (m, 2H), 6.85 (s, 1H), 6.27-6.24 (m, 1H), 5.78-5.76 (m, 2H), 4.45 (d, J=10.0 Hz, 1H), 3.96-3.94 (m, 1H), 3.66-3.60 (m, 2H), 0.84-0.82 (m, 1H), 0.75-0.71 (m, 2H), 0.55-0.53 (m, 1H). MS (ESI) m/z [M+1]⁺ 385.

Isomer2:

¹H NMR (500 MHz, DMSO-d6) δ 8.76 (s, 1H), 7.77 (s, 1H), 7.55-7.50 (m, 2H), 6.86 (s, 1H), 6.27-6.23 (m, 1H), 5.78-5.75 (m, 2H), 4.45 (d, J=10.0 Hz, 1H), 3.96-3.94 (m, 1H), 3.66-3.60 (m, 2H), 0.86-0.84 (s, 1H), 0.75-0.71 (m, 2H), 0.55-0.53 (m, 1H). MS (ESI) m/z [M+1]⁺ 385.

### Example 2-11: Synthesis of (R)-1-(5-(7-chloro-2-(1H-1,2,4-triazol-3-yl)benzofuran-5-yl)-2,2-dimethylmorpholino)prop-2-en-1-one (Assumed) (B27A) and (S)-1-(5-(7-chloro-2-(1H-1,2,4-triazol-3-yl)benzofuran-5-yl)-2,2-dimethylmorpholino)prop-2-en-1-one (Assumed) (B27B)

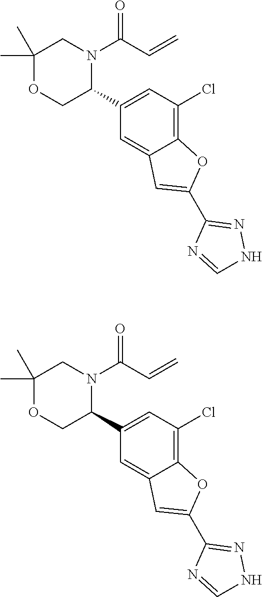 [SMILES: C=CC(=O)N1CC(C)(C)OC[C@H]1c1cc(Cl)c2oc(-c3nc[nH]n3)cc2c1 ; C=CC(=O)N1CC(C)(C)OC[C@@H]1c1cc(Cl)c2oc(-c3nc[nH]n3)cc2c1]

Racemic 1-(5-(7-chloro-2-(1H-1,2,4-triazol-3-yl)benzofuran-5-yl)-2,2-dimethylmorpholino) prop-2-en-1-one (synthesized according to procedures for compound B7 using corresponding starting materials) was separated using the following conditions:

Chiral separation: column: AD-H, 25 mm×250 mm, 5 um; fluent: 43% EtOH/Hex (0.2% 2M NH3 MeOH); isomer 1, retention time=12.80 min, isomer 2, retention time=9.80 min.

Isomer1:

¹H NMR (500 MHz, DMSO-d6) δ 8.68 (s, 1H), 7.71 (s, 1H), 7.50-7.45 (m, 2H), 6.84 (s, 1H), 6.25-6.22 (m, 1H), 5.75-5.73 (m, 1H), 5.58-5.32 (m, 1H), 4.18-3.96 (m, 2H), 3.85-3.51 (m, 1H), 3.08-2.91 (m, 1H), 1.17-1.15 (m, 6H). MS (ESI) m/z [M+1]⁺ 387.

Isomer2:

¹H NMR (500 MHz, DMSO-d6) δ 8.69 (s, 1H), 7.71 (s, 1H), 7.51-7.48 (m, 2H), 6.85 (s, 1H), 6.26-6.22 (m, 1H), 5.75-5.73 (m, 1H), 5.58-5.32 (m, 1H), 4.18-3.96 (m, 2H), 3.81-3.51 (m, 1H), 3.08-2.89 (m, 1H), 1.18-1.15 (m, 6H). MS (ESI) m/z [M+1]⁺ 387.

### Example 2-12: Synthesis of (R)-1-(3-(7-chloro-4-methoxy-2-(4H-1,2,4-triazol-3-yl)benzofuran-5-yl)morpholino)prop-2-en-1-one (Assumed) (B32A) and (S)-1-(3-(7-chloro-4-methoxy-2-(4H-1,2,4-triazol-3-yl)benzofuran-5-yl)morpholino)prop-2-en-1-one (Assumed) (B32B)

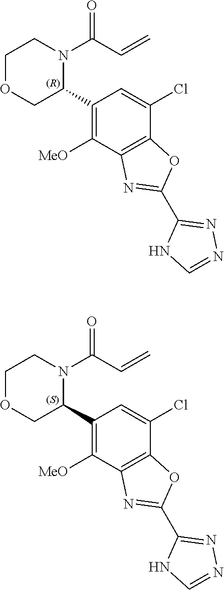

### Step 1: 4-bromo-2-chloro-5-methoxyphenol

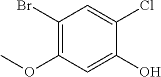

To a solution of 4-bromo-3-methoxyphenol (50 g, 246.2 mmol) in HFIP (500 mL) was added NCS (36 g, 270.9 mmol). The resulting mixture was stirred at 40° C. for 12 h. Upon completion of the reaction, the mixture was cooled to rt. The reaction mixture was concentrated under reduced pressure to give the product (67 g, crude). ¹H NMR (400 MHz, CDCL3-d) δ=7.47 (s, 1H), 6.62 (s, 1H), 3.87 (s, 3H).

### Step 2: 1-bromo-5-chloro-4-(2,2-dimethoxyethoxy)-2-methoxybenzene

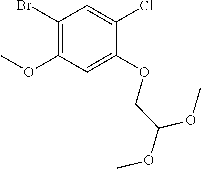

To a solution of 4-bromo-2-chloro-5-methoxyphenol (67 g, 282.1 mmol) and 2-bromo-1,1-dimethoxy-ethane (57 g, 338.6 mmol, 39.7 mL) in NMP (600 mL) was added K₂CO₃ (58490 mg, 423.2 mmol). The resulting mixture was stirred at 130° C. for 12 h. After cooled to rt, water (1000 mL) was added and the resulting solution was extracted with EA. The combined organic layer was washed with water and brine, dried over anhydrous Na₂SO₄, filtered and concentrated in vacuo. The residue was purified by silica gel column chromatography (hexanes:EA=1:0 to 5:1) to give the product (67 g, 72% yield). ¹H NMR (400 MHz, CDCL3-d) δ=7.51 (s, 1H), 6.56 (s, 1H), 4.75-4.72 (m, 1H), 4.06 (d, J=5.2 Hz, 2H), 3.88 (s, 3H), 3.51 (s, 6H)

### Step 3: 5-bromo-7-chloro-4-methoxybenzofuran

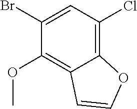

To a solution of PPA (72 g, 184.2 mmol) in toluene (200 mL) was added 1-bromo-5-chloro-4-(2,2-dimethoxyethoxy)-2-methoxybenzene (20 g, 61.4 mmol) and the resulting mixture was stirred at 110° C. for 3 h. After cooled to rt, water (1000 mL) was added and the aqueous phase was extracted with EA for several times. The combined organic layer was washed with brine, dried over anhydrous Na₂SO₄, filtered and concentrated in vacuo. The residue was purified by silica gel column chromatography (hexanes:EA=1:0 to 5:1) to the product (12.8 g, 78% yield). ¹H NMR (400 MHz, CDCL3-d) δ=7.65 (d, J=2.0 Hz, 1H), 7.48 (s, 1H), 6.97 (d, J=2.0 Hz, 1H), 4.08 (s, 3H)

### Step 4: tert-butyl 5-(7-chloro-4-methoxybenzofuran-5-yl)-2,3-dihydro-4H-1,4-oxazine-4-carboxylate

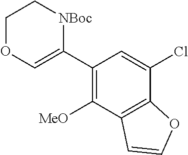 [SMILES: COc1c(C2=COCCN2C(=O)OC(C)(C)C)cc(Cl)c2occc12]

A mixture of 5-bromo-7-chloro-4-methoxybenzofuran (1500 mg, 5.7 mmol), bis(pinacolato)diboron (3.6 g, 14.1 mmol), Pd(dppf)Cl₂ (420 mg, 0.6 mmol) and KOAc (1001 mg, 11.4 mmol) in 1,4-dioxane (60 mL) was stirred at 90° C. for 6 hours. The reaction mixture was cooled to rt and concentrated under reduced pressure. The crude product was purified by silica gel column chromatography (EA/PE 1/100 to 2/3) to give 2-(7-chloro-4-methoxybenzofuran-5-yl)-4,4,5,5-tetramethyl-1,3,2-dioxaborolane (3.0 g, crude). MS (ESI) m/z [M+H]⁺ 309.

A mixture of 2-(7-chloro-4-methoxybenzofuran-5-yl)-4,4,5,5-tetramethyl-1,3,2-dioxaborolane (3.0 g, ca. 5.7 mmol, crude), tert-butyl 5-((diphenoxyphosphoryl)oxy)-2,3-dihydro-4H-1,4-oxazine-4-carboxylate (2.7 g, 6.3 mmol), K₂CO₃ (1.6 g, 11.4 mmol), Pd(dppf)Cl₂ (0.4 g, 0.6 mmol) in 1,4-dioxane (60 mL) and water (12 mL) was stirred at 90° C. for 4 h under N₂ atmosphere. After cooled to rt, the mixture was extracted with EA. The organic layers were washed with brine, dried over anhydrous Na₂SO₄ and filtered. The solvent was removed in vacuo, and the residue was purified by silica gel column chromatography (EA/PE 1/100 to 1/8) to give the title compound (1.13 g, 54% over 2 steps). MS (ESI) m/z [M+H−56]⁺ 310.

### Step 5: tert-butyl 3-(7-chloro-4-methoxybenzofuran-5-yl)morpholine-4-carboxylate

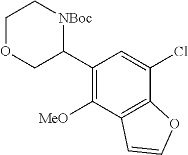 [SMILES: COc1c(C2COCCN2C(=O)OC(C)(C)C)cc(Cl)c2occc12]

To a solution of tert-butyl 5-(7-chloro-4-methoxybenzofuran-5-yl)-2,3-dihydro-4H-1,4-oxazine-4-carboxylate (1.2 g, 3.3 mmol) in DCM (20 mL) was added TFA (10 mL) at 0° C., and the resulting mixture was stirred at rt for 1 h. Upon completion of the reaction, the solvent was removed in vacuo to give crude 5-(7-chloro-4-methoxybenzofuran-5-yl)-3,4-dihydro-2H-1,4-oxazine. MS (ESI) m/z [M+H]⁺ 266.

To a solution of crude 5-(7-chloro-4-methoxybenzofuran-5-yl)-3,4-dihydro-2H-1,4-oxazine (ca. 3.3 mmol, crude) in MeOH (30 mL) was added NaBH₃CN (0.4 g, 6.7 mmol) at 0° C., and the resulting mixture was stirred at rt for 2 hours. Upon completion of the reaction, the solvent was removed in vacuo to give crude 3-(7-chloro-4-methoxybenzofuran-5-yl)morpholine. MS (ESI) m/z [M+H]⁺ 268.

To a solution of crude 3-(7-chloro-4-methoxybenzofuran-5-yl)morpholine (ca. 3.3 mmol) in 1,4-dioxane (30 mL) and water (30 mL) was added Na₂CO₃ (13 g, 122.6 mmol) and (Boc)₂O (2.2 g, 10 mmol) at 0° C. The resulting mixture was stirred at rt for 2 hours. Upon completion of the reaction, the mixture was extracted with EA. The organic layers were washed with brine, dried over anhydrous Na₂SO₄ and filtered. The solvent was removed in vacuo, and the residue was purified by silica gel column chromatography (EA/PE 1/100 to 3/7) to give the title compound (0.75 g, 61% over 3 steps). MS (ESI) m/z [M+H−100]⁺ 268.

### Step 6: (5-(4-(tert-butoxycarbonyl)morpholin-3-yl)-7-chloro-4-methoxybenzofuran-2-yl)boronic Acid

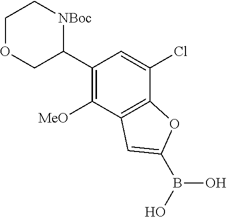 [SMILES: COc1c(C2COCCN2C(=O)OC(C)(C)C)cc(Cl)c2oc(B(O)O)cc12]

To a mixture of tert-butyl 3-(7-chloro-4-methoxybenzofuran-5-yl)morpholine-4-carboxylate (650 mg, 1.8 mmol), [Ir(μ-OMe)(cod)₂](58.5 mg, 0.09 mmol) and 4-tert-butyl-2-(4-tert-butyl-2-pyridyl)pyridine (47.4 mg, 0.18 mmol) in 1,4-dioxane was added 4,4,5,5-tetramethyl-1,3,2-dioxaborolane (1.2 g, 9.0 mmol) at rt under N₂ atmosphere. The resulting mixture was stirred at rt for 2 hours. Upon completion of the reaction, the solvent was removed in vacuo, and the residue was purified by C18 column (mobile phase, ACN in water, 20% to 80%) to give the title compound (615 mg, 85% yield).

### Step 7: tert-butyl 3-(7-chloro-4-methoxy-2-(1-((2-(trimethylsilyl)ethoxy)methyl)-1H-1,2,4-triazol-5-yl)benzofuran-5-yl)morpholine-4-carboxylate

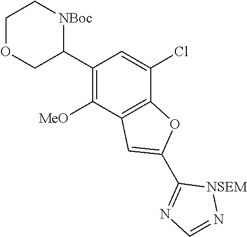 [SMILES: COc1c(C2COCCN2C(=O)OC(C)(C)C)cc(Cl)c2oc(-c3ncnn3COCC[Si](C)(C)C)cc12]

A mixture of (5-(4-(tert-butoxycarbonyl)morpholin-3-yl)-7-chloro-4-methoxybenzofuran-2-yl)boronic acid (610 mg, 1.5 mmol), 2-[(5-bromo-1,2,4-triazol-1-yl)methoxy]ethyl-trimethyl-silane (54 mg, 1.6 mmol), Na₂CO₃ (314 mg, 3.0 mmol) and Pd(dtbpf)Cl₂ (204 mg, 0.3 mmol) in 1,4-dioxane (20 mL)/water (4 mL) was stirred at 70° C. for 2 hours under N₂ atmosphere. After cooled to rt, water was added and the mixture was extracted with EA. The organic layers were washed with brine and dried over anhydrous Na₂SO₄. The solvent was removed in vacuo, and the residue was purified by silica gel column chromatography (EA/PE 1/100 to 1/2) to give the title compound (210 mg, 25%). MS (ESI) m/z [M+H]⁺ 565.

### Step 8: 1-(3-(7-chloro-4-methoxy-2-(4H-1,2,4-triazol-3-yl)benzofuran-5-yl)morpholino)prop-2-en-1-one

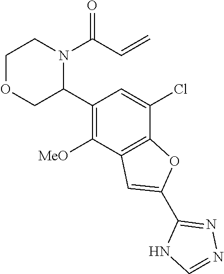 [SMILES: C=CC(=O)N1CCOCC1c1cc(Cl)c2oc(-c3nnc[nH]3)cc2c1OC]

To a solution of tert-butyl 3-(7-chloro-4-methoxy-2-(1-((2-(trimethylsilyl)ethoxy)methyl)-1H-1,2,4-triazol-5-yl)benzofuran-5-yl)morpholine-4-carboxylate (210 mg, 0.4 mmol) in DCM (20 mL) was added TFA (10 mL) at 0° C. The resulting mixture was stirred at rt for 3.5 hours. Upon completion of the reaction, the solvent was removed in vacuo, and the residue was re-dissolved with THF (15 mL) and sat.aq. NaHCO₃ (15 mL), then acryloyl chloride (0.24 g, 2.7 mmol) in CH₂Cl₂ (24 mL) was added dropwise at 0° C. The resulting mixture was stirred at 0° C. for 20 min. Upon completion of the reaction, the mixture was extracted with MeOH/DCM (1/10). The organic layers were washed with brine and dried over anhydrous Na₂SO₄. The solvent was removed in vacuo, and the residue was purified by C18 column (mobile phase, ACN in water (0.1% FA), 25% to 40%) to give the title compound (60 mg, 41% over 2 steps).

¹H NMR (500 MHz, DMSO-d₆) δ 8.71 (s, 1H), 7.73 (s, 1H), 7.51 (brs, 1H), 6.85 (dd, J=16.7, 10.4 Hz, 1H), 6.16 (dd, J=16.7, 2.2 Hz, 1H), 5.72 (dd, J=10.4, 2.3 Hz, 1H), 5.49 (brs, 1H), 4.31-4.13 (m, 2H), 4.11 (s, 3H), 4.01-3.95 (m, 1H), 3.82 (dd, J=12.0, 3.5 Hz, 1H), 3.51 (td, J=11.5, 3.5 Hz, 1H), 3.26-2.96 (m, 1H). MS (ESI) m/z [M+H]⁺=389.

### Step 9: (S)-1-(3-(7-chloro-4-methoxy-2-(4H-1,2,4-triazol-3-yl)benzofuran-5-yl)morpholino)prop-2-en-1-one (Assumed) and (R)-1-(3-(7-chloro-4-methoxy-2-(4H-1,2,4-triazol-3-yl)benzofuran-5-yl)morpholino)prop-2-en-1-one (Assumed)

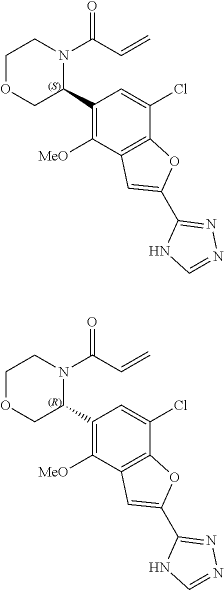

Racemic 1-(3-(7-chloro-4-methoxy-2-(4H-1,2,4-triazol-3-yl)benzofuran-5-yl)morpholino)prop-2-en-1-one was separated using the following conditions:

SFC: column: YMC i-Amylose-3, 30 mm×250 mm, 5 um; fluent: 32% MeOH (0.3% 2M NH₃ MeOH)/CO2 isomer 1, retention time=6.35 min, isomer 2, retention time=8.75 min;

Isomer 1:

¹H NMR (500 MHz, DMSO-d₆) δ 8.71 (s, 1H), 7.74 (s, 1H), 7.51 (brs, 1H), 6.85 (dd, J=16.5, 10.5 Hz, 1H), 6.16 (d, J=16.6 Hz, 1H), 5.73 (d, J=10.5 Hz, 1H), 5.50 (brs, 1H), 4.27-4.15 (m, 2H), 4.12 (s, 3H), 4.00-3.93 (m, 1H), 3.82 (d, J=11.5 Hz, 1H), 3.51 (t, J=1.0 Hz, 1H), 3.29-3.10 (m, 1H). MS (ESI) m/z [M+H]⁺=389.

Isomer 2:

¹H NMR (500 MHz, DMSO-d₆) δ 8.71 (s, 1H), 7.74 (s, 1H), 7.50 (brs, 1H), 6.85 (dd, J=16.5, 10.5 Hz, 1H), 6.16 (d, J=16.9 Hz, 1H), 5.72 (dd, J=10.3 Hz, 1H), 5.50 (brs, 1H), 4.30-4.15 (m, 2H), 4.12 (s, 3H), 4.01-3.94 (m, 1H), 3.82 (d, J=11.3 Hz, 1H), 3.51 (t, J=10.4 Hz, 1H), 3.28-3.11 (m, 1H). MS (ESI) m/z [M+H]⁺=389.

Compounds B15-B18, and B33 in Table 2-5 below were synthesized by using corresponding starting materials according to procedures similar to those described for the synthesis of compounds B1, B2, B32, and D15.

TABLE 2-5

| Compound | Structure | Chemical Name | ¹H NMR data LC/MS m/z (M + H) |
|---|---|---|---|
| B15A | 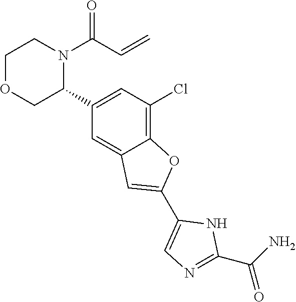 [SMILES: C=CC(=O)N1CCOC[C@H]1c1cc(Cl)c2oc(-c3cnc(C(N)=O)[nH]3)cc2c1] | (R)-5-(5-(4- acryloylmorpholin-3-yl)-7- chlorobenzofuran-2-yl)-1H- imidazole-2-carboxamide (assumed) | ¹H NMR (400 MHz, DMSO-d₆) δ 13.45 (s, 1H), 7.95 (s, 1H), 7.82 (s, 1H), 7.62 (s, 2H), 7.35 (s, 1H), 7.16 (s, 1H), 6.93- 6.82 (m, 1H), 6.23 (d, J = 15.0 Hz, 1H), 5.76 (d, J = 10.3 Hz, 1H), 5.69- 5.36 (m, 1H), 4.45 (d, J = 12.2 Hz, 1H), 4.36-3.73 (m, 3H), 3.52 (t, J = 11.0 Hz, 1H), 3.24-2.87 (m, 1H). MS (ESI) m/z [M + H]⁺ 401. |
| B15B | 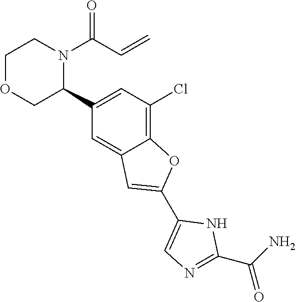 [SMILES: C=CC(=O)N1CCOC[C@@H]1c1cc(Cl)c2oc(-c3cnc(C(N)=O)[nH]3)cc2c1] | (S)-5-(5-(4- acryloylmorpholin-3-yl)-7- chlorobenzofuran-2-yl)-1H- imidazole-2-carboxamide (assumed) | ¹H NMR (400 MHz, DMSO-d₆) δ13.45 (s, 1H), 7.95 (s, 1H), 7.83 (s, 1H), 7.63 (s, 2H), 7.35 (s, 1H), 7.16 (s, 1H), 6.93- 6.82 (m, 1H), 6.23 (d, J = 16.6 Hz, 1H), 5.76 (d, J = 10.4 Hz, 1H), 5.68- 5.40 (m, 1H), 4.45 (d, J = 12.2 Hz, 1H), 4.37-3.75 (m, 3H), 3.52 (t, J = 10.9 Hz, 1H), 3.25-2.94 (m, 1H). MS (ESI) m/z [M + H]⁺ 401. |
| B16A | 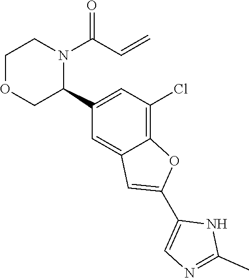 [SMILES: C=CC(=O)N1CCOC[C@@H]1c1cc(Cl)c2oc(-c3cnc(C)[nH]3)cc2c1] | (S)-1-(3-(7-chloro-2-(2- methyl-1H-imidazol-5- yl)benzofuran-5- yl)morpholino)prop-2-en-1- one (assumed) | ¹H NMR (400 MHz, DMSO-d₆) δ 12.29 (s, 1H), 7.55 (m, 2H), 7.30 (s, 1H), 7.00 (s, 1H), 6.92-6.82 (m, 1H), 6.23 (d, J = 16.6 Hz, 1H), 5.76 (d, J = 10.7 Hz, 1H), 5.68-5.27 (m, 1H), 4.44 (d, J = 12.1 Hz, 1H), 4.33-3.73 (m, 3H), 3.51 (t, J = 10.9 Hz, 1H), 3.25-2.91 (m, 1H), 2.34 (s, 3H). MS (ESI) m/z [M + H]⁺ 372. |
| B16B | 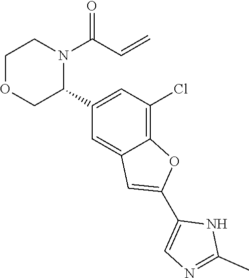 [SMILES: C=CC(=O)N1CCOC[C@H]1c1cc(Cl)c2oc(-c3cnc(C)[nH]3)cc2c1] | (R)-1-(3-(7-chloro-2-(2- methyl-1H-imidazol-5- yl)benzofuran-5- yl)morpholino)prop-2-en-1- one (assumed) | ¹H NMR (400 MHz, DMSO-d₆) δ 12.17 (s, 1H), 7.56 (m, 2H), 7.30 (s, 1H), 6.99 (s, 1H), 6.91-6.81 (m, 1H), 6.23 (d, J = 16.6 Hz, 1H), 5.76 (d, J = 10.4 Hz, 1H), 5.67-5.30 (m, 1H), 4.43 (d, J = 12.2 Hz, 1H), 4.33-3.73 (m, 3H), 3.51 (t, J = 11.0 Hz, 1H), 3.28-2.94 (m, 1H), 2.33 (s, 3H). MS (ESI) m/z [M + H]⁺ 372. |
| B17 | 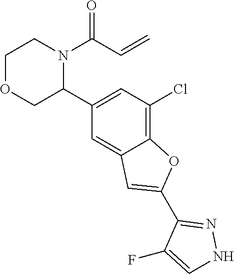 [SMILES: C=CC(=O)N1CCOCC1c1cc(Cl)c2oc(-c3n[nH]cc3F)cc2c1] | 1-(3-(7-chloro-2-(4-fluoro- 1H-pyrazol-3-yl)benzofuran- 5-yl)morpholino)prop-2-en- 1-one | ¹H NMR (400 MHz, DMSO-d₆) δ 13.33 (s, 1H), 8.00 (s, 1H), 7.63 (s, 1H), 7.39 (s, 1H), 7.22 (s, 1H), 6.88-6.78 (m, 1H), 6.20 (d, J = 16.6 Hz, 1H), 5.73 (d, J = 10.4 Hz, 1H), 5.66-5.23 (m, 1H), 4.41 (d, J = 12.2 Hz, 1H), 4.30-3.73 (m, 3H), 3.49 (t, J = 10.9 Hz, 1H), 3.22- 2.85 (m, 1H). MS (ESI) m/z [M + H]⁺ 376. |
| B18 | 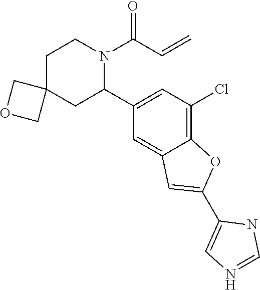 [SMILES: C=CC(=O)N1CCC2(COC2)CC1c1cc(Cl)c2oc(-c3cnc[nH]3)cc2c1] | 1-(6-(7-chloro-2-(1H- imidazol-4-yl)benzofuran-5- yl)-2-oxa-7- azaspiro[3.5]nonan-7- yl)prop-2-en-1-one | ¹H NMR (400 MHz, DMSO-d₆) δ 12.75 (s, 1H), 7.89 (s, 1H), 7.71 (s, 1H), 7.39 (s, 1H), 7.24 (s, 1H), 7.06 (s, 1H), 6.94- 6.60 (m, 1H), 6.14 (d, J = 16.6 Hz, 1H), 5.75-5.52 (m, 2H), 4.45-4.08 (m, 3H), 4.00-3.87 (m, 2H), 3.50- 3.14 (m, 1H), 2.81 (d, J = 13.7 Hz, 1H), 2.14-1.99 (m, 2H), 1.78-1.66 (m, 1H). MS (ESI) m/z [M + 1]⁺ 398. |
| B33 | 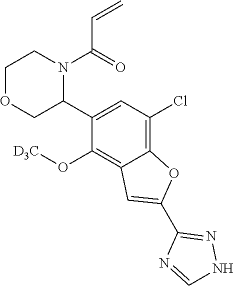 [SMILES: [2H]C([2H])([2H])Oc1c(C2COCCN2C(=O)C=C)cc(Cl)c2oc(-c3nc[nH]n3)cc12] | 1-(3-(7-chloro-4-(methoxy- d3)-2-(1H-1,2,4-triazol-3- yl)benzofuran-5- yl)morpholino)prop-2-en-1- one | ¹H NMR (400 MHz, DMSO-d₆) δ 8.71 (s, 1H), 7.73 (s, 1H), 7.51 (s, 1H), 6.85 (dd, J = 16.6, 10.4 Hz, 1H), 6.17 (d, J = 16.6 Hz, 1H), 5.72 (d, J = 10.4 Hz, 1H), 5.53 (s, 1H), 4.29-3.94 (m, 3H), 3.87- 3.80 (m, 1H), 3.55-3.48 (m, 1H), 3.18-2.96 (m, 1H). MS (ESI) m/z [M + 1]⁺ 392. |

### Example 2-13: Synthesis of (R)-1-(3-(7-chloro-2-(1H-imidazol-4-yl)benzo[d]oxazol-5-yl)morpholino)prop-2-en-1-one (Assumed) (D7A) and (S)-1-(3-(7-chloro-2-(1H-imidazol-4-yl)benzo[d]oxazol-5-yl)morpholino)prop-2-en-1-one (Assumed) (D7B)

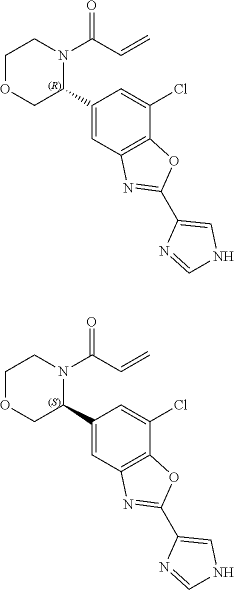

### Step 1: tert-butyl 5-(7-chloro-2-(1-((2-(trimethylsilyl)ethoxy)methyl)-1H-imidazol-4-yl)benzo[d]oxazol-5-yl)-2,3-dihydro-4H-1,4-oxazine-4-carboxylate

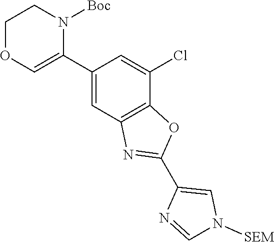 [SMILES: CC(C)(C)OC(=O)N1CCOC=C1c1cc(Cl)c2oc(-c3cn(COCC[Si](C)(C)C)cn3)nc2c1]

A solution of 2-[[4-[7-chloro-5-(4,4,5,5-tetramethyl-1,3,2-dioxaborolan-2-yl)-1,3-benzoxazol-2-yl]imidazol-1-yl]methoxy]ethyl-trimethyl-silane (1.0 g, 2.1 mmol), tert-butyl 5-diphenoxyphosphoryloxy-2,3-dihydro-1,4-oxazine-4-carboxylate (1.4 g, 3.2 mmol), Pd(dppf)Cl₂ (150.00 mg, 0.21 mmol) and K₂CO₃ (580 mg, 4.20 mmol) in THF (10 mL)/ACN (10 mL)/H₂O (5 mL) was stirred at 90° C. for overnight under N2 atmosphere. After cooled to rt, the reaction was concentrated in vacuo and EA was added, the mixture was washed with brine, dried over Na₂SO₄, filtered and concentrated. The residue was purified by silica gel column chromatography (MeOH/DCM 1/100 to 1/20) to give the title compound (1.3 g, 52%). MS (ESI) m/z [M+H]⁺ 533.

### Step 2: 7-chloro-2-(1H-imidazol-4-yl)-5-(morpholin-3-yl)benzo[d]oxazole

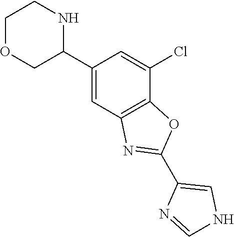 [SMILES: Clc1cc(C2COCCN2)cc2nc(-c3c[nH]cn3)oc12]

To a solution of tert-butyl 5-[7-chloro-2-[1-(2-trimethylsilylethoxymethyl)imidazol-4-yl]-1,3-benzoxazol-5-yl]-2,3-dihydro-1,4-oxazine-4-carboxylate (500 mg, 0.94 mmol) in 1,1,1,3,3,3-hexafluoropropan-2-ol (10 mL) was added MsOH (0.2 mL), the mixture was stirred at rt for 10 min. The reaction solution was diluted with MeOH (20 mL) and added NaBH₃CN (169 mg, 2.68 mmol) in portions. The mixture was stirred at rt for 1 hour. Upon completion of the reaction, the reaction was concentrated in vacuo and the residue was purified by C18 column (ACN/H2O (0.1% FA) 1/100 to 1/9) to give the title compound (200 mg, 36%). MS (ESI) m/z [M+H]⁺ 305.

### Step 3:1-(3-(7-chloro-2-(1H-imidazol-4-yl)benzo[d]oxazol-5-yl)morpholino)prop-2-en-1-one

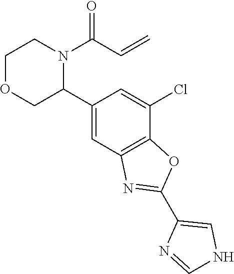 [SMILES: C=CC(=O)N1CCOCC1c1cc(Cl)c2oc(-c3c[nH]cn3)nc2c1]

To a solution of 7-chloro-2-(1H-imidazol-4-yl)-5-morpholin-3-yl-1,3-benzoxazole (180 mg, 0.44 mmol) in THF (10 mL)/sat.NaHCO₃ (10 mL) was added acryloyl chloride (40 mg, 0.44 mmol), the mixture was stirred at rt for 10 min. Upon completion of the reaction, the reaction was concentrated and the residue was purified by C18 column (ACN/H₂O (0.1% FA) 1/100 to 3/7) to give the title compound (29 mg, 6%). ¹H NMR (400 MHz, DMSO-d₆) δ 12.86 (s, 1H), 8.10 (s, 1H), 7.92 (s, 1H), 7.61 (s, 1H), 7.39 (s, 1H), 6.90-6.79 (m, 1H), 6.20 (d, J=16.6, 1H), 5.73 (d, J=10.4, 1H), 5.66-5.32 (m, 1H), 4.44 (d, J=12.2 Hz, 1H), 4.30-3.90 (m, 1H), 3.89-3.74 (m, 2H), 3.54-3.45 (m, 1H), 3.12-2.94 (m, 1H). MS (ESI) m/z [M+H]⁺=359.

### Step 4: (R)-1-(3-(7-chloro-2-(1H-imidazol-4-yl)benzo[d]oxazol-5-yl)morpholino)prop-2-en-1-one (Assumed) and (S)-1-(3-(7-chloro-2-(1H-imidazol-4-yl)benzo[d]oxazol-5-yl)morpholino)prop-2-en-1-one (Assumed)

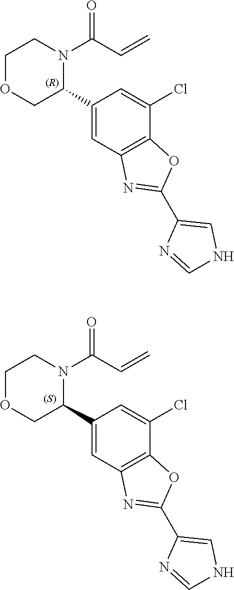

Racemic 1-(3-(7-chloro-2-(1H-imidazol-4-yl)benzo[d]oxazol-5-yl)morpholino)prop-2-en-1-one was separated by using the following conditions:

SFC: column: CHIRALPAK AD-H, 20 mm×250 mm, 5 um; fluent: 38% MeOH (0.3% 2M NH₃ MeOH)/CO2, flow rate: 40.0 mL/min, isomer 1, retention time=6.28 min, isomer 2, retention time=9.25 min;

Isomer 1:

¹H NMR (400 MHz, CDCl₃) δ 8.35 (s, 1H), 7.99 (s, 1H), 7.74 (s, 1H), 7.46 (s, 1H), 6.64-6.50 (m, 1H), 6.45-6.32 (m, 1H), 5.85-5.35 (m, 2H), 4.45 (d, J=11.9 Hz, 1H), 4.01-3.85 (m, 2H), 3.80-3.53 (m, 2H), 3.49-3.21 (m, 1H). MS (ESI) m/z [M+H]⁺=359.

Isomer 2:

¹H NMR (400 MHz, CDCl₃) δ 8.36 (s, 1H), 7.97 (s, 1H), 7.76 (s, 1H), 7.56-7.36 (m, 1H), 6.65-6.52 (m, 1H), 6.45-6.34 (m, 1H), 5.83-5.49 (m, 2H), 4.47 (d, J=11.9 Hz, 1H), 4.04-3.87 (m, 2H), 3.82-3.54 (m, 2H), 3.50-3.25 (m, 1H). MS (ESI) m/z [M+H]⁺=359.

### Example 2-14: Synthesis of (R)-1-(6-(7-chloro-2-(1H-imidazol-4-yl)benzo[d]oxazol-5-yl)-2-oxa-7-azaspiro[3.5]nonan-7-yl)prop-2-en-1-one (Assumed) (D15A) and (S)-1-(6-(7-chloro-2-(1H-imidazol-4-yl)benzo[d]oxazol-5-yl)-2-oxa-7-azaspiro[3.5]nonan-7-yl)prop-2-en-1-one (Assumed) (D15B)

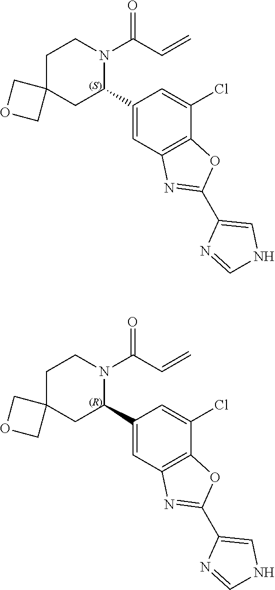

### Step 1: tert-butyl 6-(7-chloro-2-(1-((2-(trimethylsilyl)ethoxy)methyl)-1H-imidazol-4-yl)benzo[d]oxazol-5-yl)-2-oxa-7-azaspiro[3.5]non-5-ene-7-carboxylate

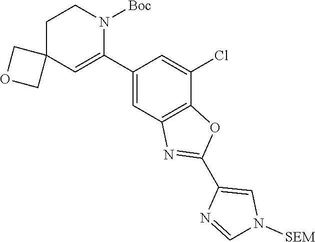 [SMILES: CC(C)(C)OC(=O)N1CCC2(C=C1c1cc(Cl)c3oc(-c4cn(COCC[Si](C)(C)C)cn4)nc3c1)COC2]

A solution of 2-[[4-[7-chloro-5-(4,4,5,5-tetramethyl-1,3,2-dioxaborolan-2-yl)-1,3-benzoxazol-2-yl]imidazol-1-yl]methoxy]ethyl-trimethyl-silane (1.0 g, 2.1 mmol), tert-butyl 6-(trifluoromethylsulfonyloxy)-2-oxa-7-azaspiro[3.5]non-5-ene-7-carboxylate (1.2 g, 3.2 mmol), Pd(dtbpf)Cl₂ (140 mg, 0.21 mmol) and K₃PO₄ (890 mg, 4.20 mmol) in 1,4-dioxane (20 mL) and H₂O (5 mL) was stirred at 80° C. for 1 hour. After cooled to rt, the reaction was concentrated and EA was added, the mixture was washed with brine, dried over Na₂SO₄, filtered and concentrated. The residue was purified by silica gel column chromatography (EA/P1 1/10 to 7/3) to give the title compound (800 mg, 66%). MS (ESI) m/z [M+H]⁺ 573.

### Step 2: 1-(6-(7-chloro-2-(1H-imidazol-4-yl)benzo[d]oxazol-5-yl)-2-oxa-7-azaspiro[3.5]nonan-7-yl)prop-2-en-1-one

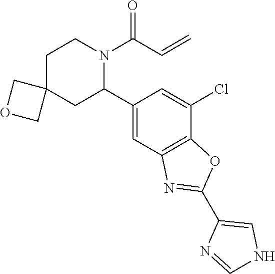 [SMILES: C=CC(=O)N1CCC2(COC2)CC1c1cc(Cl)c2oc(-c3c[nH]cn3)nc2c1]

To a solution of tert-butyl 6-[7-chloro-2-[1-(2-trimethylsilylethoxymethyl)imidazol-4-yl]-1,3-benzoxazol-5-yl]-2-oxa-7-azaspiro[3.5]non-5-ene-7-carboxylate (400 mg, 0.70 mmol) in 1,1,1,3,3,3-hexafluoropropan-2-ol (20 mL) was added MsOH (0.5 mL), the mixture was stirred at rt for 10 mins and MeOH (50 mL) was added. To the solution was added NaBH₃CN (147 mg, 2.33 mmol), the mixture was stirred at rt for 1 hour. The reaction was concentrated and the residue was dissolved in ACN (10 mL) and sat.NaHCO₃ (10 mL) and then acryloyl chloride (105 mg, 1.16 mmol) in ACN (1 mL) was added. The mixture was stirred at rt for 10 min. Upon completion of the reaction, the reaction was concentrated in vacuo, and the residue was purified by silica gel column chromatography (MeOH/DCM 1/100 to 1/20) to give the title compound (51 mg, 11%). ¹H NMR (400 MHz, DMSO-d₆) δ 12.84 (s, 1H), 8.09 (s, 1H), 7.91 (s, 1H), 7.43 (s, 1H), 7.31 (s, 1H), 6.78 (br, 1H), 6.10 (d, J=16.6, 1H), 5.78-5.53 (m, 2H), 4.37 (d, J=5.6 Hz, 1H), 4.35-4.15 (m, 1H), 4.13 (d, J=5.6 Hz, 1H), 3.96-3.91 (m, 1H), 3.90-3.84 (m, 1H), 3.21-2.96 (m, 1H), 2.78 (d, J=13.2 Hz, 1H), 2.04 (d, J=12.4 Hz, 2H), 1.78-1.66 (m, 1H). MS (ESI) m/z [M+H]⁺=399.

### Step 3: (R)-1-(6-(7-chloro-2-(1H-imidazol-4-yl)benzo[d]oxazol-5-yl)-2-oxa-7-azaspiro[3.5]nonan-7-yl)prop-2-en-1-one (Assumed) and (S)-1-(6-(7-chloro-2-(1H-imidazol-4-yl)benzo[d]oxazol-5-yl)-2-oxa-7-azaspiro[3.5]nonan-7-yl)prop-2-en-1-one (Assumed)

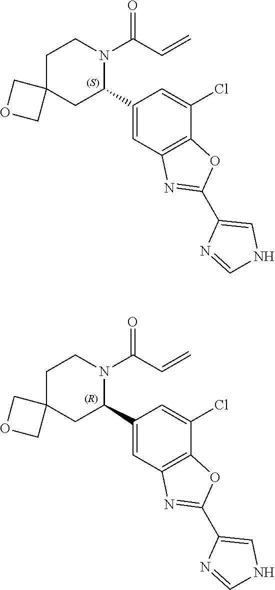

Racemic 1-(6-(7-chloro-2-(1H-imidazol-4-yl)benzo[d]oxazol-5-yl)-2-oxa-7-azaspiro[3.5]nonan-7-yl)prop-2-en-1-one was separated by using the following conditions:

SFC: column: YMC Cellulose-SB, 20 mm×250 mm, 5 um; fluent: 45% MeOH (0.3% 2M NH₃ MeOH)/CO₂, flow rate: 40.0 mL/min, isomer 1, retention time=3.88 min, isomer 2, retention time=5.82 min;

Isomer 1:

¹H NMR (400 MHz, DMSO-d₆) δ 12.85 (s, 1H), 8.09 (s, 1H), 7.91 (s, 1H), 7.43 (s, 1H), 7.31 (s, 1H), 6.78 (br, 1H), 6.11 (d, J=16.6 Hz, 1H), 5.79-5.47 (m, 2H), 4.37 (d, J=5.5 Hz, 1H), 4.35-4.15 (m, 1H), 4.13 (d, J=5.5 Hz, 1H), 3.97-3.91 (m, 1H), 3.90-3.84 (m, 1H), 3.20-3.00 (m, 1H), 2.79 (d, J=13.7 Hz, 1H), 2.04 (d, J=10.1 Hz, 2H), 1.76-1.66 (m, 1H). MS (ESI) m/z [M+H]⁺=399.

Isomer 2:

¹H NMR (400 MHz, DMSO-d₆) δ 12.84 (s, 1H), 8.09 (s, 1H), 7.91 (s, 1H), 7.43 (s, 1H), 7.31 (s, 1H), 6.77 (br, 1H), 6.10 (d, J=16.7 Hz, 1H), 5.75-5.51 (m, 2H), 4.37 (d, J=5.6 Hz, 1H), 4.35-4.15 (m, 1H), 4.13 (d, J=5.6 Hz, 1H), 3.96-3.91 (m, 1H), 3.90-3.84 (m, 1H), 3.20-2.98 (m, 1H), 2.78 (d, J=13.3 Hz, 1H), 2.04 (d, J=10.0 Hz, 2H), 1.78-1.64 (m, 1H). MS (ESI) m/z [M+H]⁺=399.

Compounds D16, D17, and D23 in Table 2-6 below were synthesized by using corresponding starting materials according to procedures similar to those described for the synthesis of compounds D7.

TABLE 2-6

| Compound | Structure | Chemical Name | ¹H NMR data LC/MS m/z (M + H) |
|---|---|---|---|
| D16 | 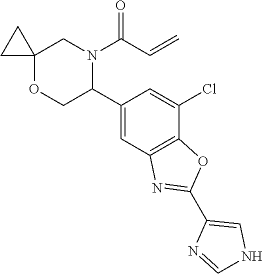 [SMILES: C=CC(=O)N1CC2(CC2)OCC1c1cc(Cl)c2oc(-c3c[nH]cn3)nc2c1] | 1-(6-(7-chloro-2-(1H- imidazol-4- yl)benzo[d]oxazol-5- yl)-4-oxa-7- azaspiro[2.5]octan-7- yl)prop-2-en-1-one | ¹H NMR (400 MHz, DMSO-d₆) δ 12.84 (s, 1H), 8.11 (s, 1H), 7.91 (s, 1H), 7.65 (s, 1H), 7.41 (s, 1H), 6.94-6.73 (m, 1H), 6.26-6.15 (m, 1H), 5.77-5.40 (m, 2H), 4.43 (d, J = 12.2 Hz, 1H), 3.94-3.87 (m, 1H), 3.72-3.57 (m, 2H), 0.83- 0.78 (m, 1H), 0.70-0.62 (m, 2H), 0.55-0.47 (m, 1H). MS (ESI) m/z [M + H]⁺ = 385 |
| D17 | 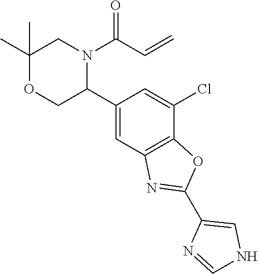 [SMILES: C=CC(=O)N1CC(C)(C)OCC1c1cc(Cl)c2oc(-c3c[nH]cn3)nc2c1] | 1-(5-(7-chloro-2-(1H- imidazol-4- yl)benzo[d]oxazol-5- yl)-2,2- dimethylmorpholino) prop-2-en-1-one | ¹H NMR (400 MHz, DMSO-d6) δ 12.84 (s, 1H), 8.10 (s, 1H), 7.91 (s, 1H), 7.64 (s, 1H), 7.38 (s, 1H), 6.91-6.68 (m, 1H), 6.19 (d, J = 16.5 Hz, 1H), 5.71 (d, J = 9.4 Hz, 1H), 5.59-5.23 (m, 1H), 4.12-4.04 (m, 1H), 4.02-3.94 (m, 1H), 3.89- 3.69 (m, 1H), 2.91-2.81 (m, 1H), 1.16-1.09 (m, 6H). MS (ESI) m/z [M + H]+ = 387. |
| D23 | 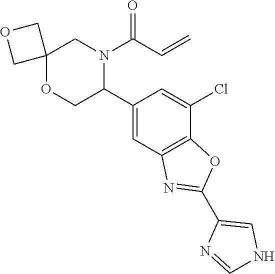 [SMILES: C=CC(=O)N1CC2(COC2)OCC1c1cc(Cl)c2oc(-c3c[nH]cn3)nc2c1] | 1-(7-(7-chloro-2-(1H- imidazol-4- yl)benzo[d]oxazol-5- yl)-2,5-dioxa-8- azaspiro[3.5]nonan-8- yl)prop-2-en-1-one | ¹H NMR (400 MHz, DMSO-D6-d₆) δ 12.84 (s, 1H), 8.10 (s, 1H), 7.91 (s, 1H), 7.60 (s, 1H), 7.36 (s, 1H), 7.10-6.70 (m, 1H), 6.24 (dd, J = 16.6, 1.9 Hz, 1H), 5.78 (d, J = 10.0 Hz, 1H), 5.63-5.31 (m, 1H), 4.54 (d, J = 6.6 Hz, 1H), 4.51-4.15 (m, 5H), 3.82 (d, J = 9.9 Hz, 1H), 3.24-3.10 (m, 1H). MS (ESI) m/z [M + H]⁺ = 401. |

### Example 2-15: (R)-1-(6-(7-chloro-2-(1H-imidazol-4-yl)benzo[d]oxazol-5-yl)-4-oxa-7-azaspiro[2.5]octan-7-yl)prop-2-en-1-one (D16A, Assumed) and (S)-1-(6-(7-chloro-2-(1H-imidazol-4-yl)benzo[d]oxazol-5-yl)-4-oxa-7-azaspiro[2.5]octan-7-yl)prop-2-en-1-one (D16B, Assumed)

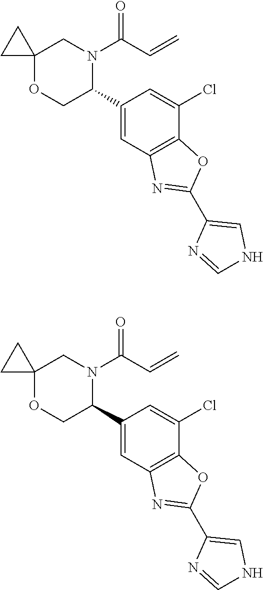 [SMILES: C=CC(=O)N1CC2(CC2)OC[C@H]1c1cc(Cl)c2oc(-c3c[nH]cn3)nc2c1 ; C=CC(=O)N1CC2(CC2)OC[C@@H]1c1cc(Cl)c2oc(-c3c[nH]cn3)nc2c1]

Racemic1-(6-(7-chloro-2-(1H-imidazol-4-yl)benzo[d]oxazol-5-yl)-4-oxa-7-azaspiro[2.5]octan-7-yl)prop-2-en-1-one (D16) was separated by using the following conditions:

Column: YMC i-Amylose-3 30 mm×250 mm, 5 μm; 45% MeOH(0.3% 2M NH₃ MeOH), isomer 1, retention time=6.57 min, isomer 2, retention time=9.70 min;

Isomer 1: (R)-1-(6-(7-chloro-2-(1H-imidazol-4-yl)benzo[d]oxazol-5-yl)-4-oxa-7-azaspiro[2.5]octan-7-yl)prop-2-en-1-one (D16A, assumed)

1H NMR (400 MHz, DMSO-d6) 1H NMR (400 MHz, DMSO-d6) δ 12.87 (br, 1H), 8.09 (s, 1H), 7.92 (s, 1H), 7.66 (s, 1H), 7.42 (s, 1H), 6.83 (s, 1H), 6.22 (d, J=16.5 Hz, 1H), 5.79-5.40 (m, 2H), 4.44 (d, J= 12.1 Hz, 1H), 4.01-3.83 (m, 1H), 3.81-3.41 (m, 2H), 0.85-0.76 (m, 1H), 0.73-0.61 (m, 2H), 0.58-0.44 (m, 1H). MS (ESI) m/z [M+H]⁺=385.

Isomer 2: (S)-1-(6-(7-chloro-2-(1H-imidazol-4-yl)benzo[d]oxazol-5-yl)-4-oxa-7-azaspiro[2.5]octan-7-yl)prop-2-en-1-one (D16B, assumed)

1H NMR (399 MHz, DMSO-d6) δ 12.86 (s, 1H), 8.09 (s, 1H), 7.92 (s, 1H), 7.66 (s, 1H), 7.42 (s, 1H), 6.83 (s, 1H), 6.22 (d, J=16.5 Hz, 1H), 5.79-5.38 (m, 2H), 4.43 (d, J=12.2 Hz, 1H), 4.01-3.84 (m, 1H), 3.80-3.43 (m, 2H), 0.86-0.77 (m, 1H), 0.73-0.60 (m, 2H), 0.58-0.43 (m, 1H). m/z [M+H]⁺=385.

### Example 2-16: (R)-1-(5-(7-chloro-2-(1H-imidazol-4-yl)benzo[d]oxazol-5-yl)-2,2-dimethylmorpholino)prop-2-en-1-one (D17A, Assumed) and (S)-1-(5-(7-chloro-2-(1H-imidazol-4-yl)benzo[d]oxazol-5-yl)-2,2-dimethylmorpholino)prop-2-en-1-one (D17B, Assumed)

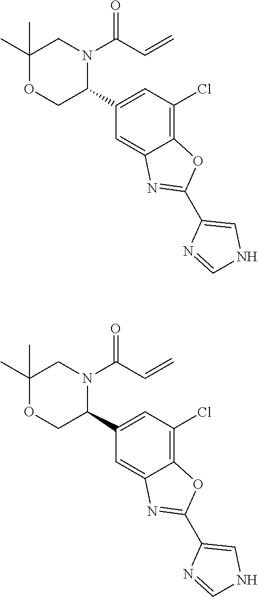 [SMILES: C=CC(=O)N1CC(C)(C)OC[C@H]1c1cc(Cl)c2oc(-c3c[nH]cn3)nc2c1 ; C=CC(=O)N1CC(C)(C)OC[C@@H]1c1cc(Cl)c2oc(-c3c[nH]cn3)nc2c1]

Racemic1-(5-(7-chloro-2-(1H-imidazol-4-yl)benzo[d]oxazol-5-yl)-2,2-dimethylmorpholino)prop-2-en-1-one (D17) was separated by using the following conditions:

Column: YMC i-Amylose-3 30 mm×250 mm, 5 μm; 45% MeOH(0.3% 2M NH₃ MeOH).

isomer 1, retention time=4.32 min, isomer 2, retention time=7.60 min;

Isomer 1: (R)-1-(5-(7-chloro-2-(1H-imidazol-4-yl)benzo[d]oxazol-5-yl)-2,2-dimethylmorpholino)prop-2-en-1-one (D17A, assumed)

¹H NMR (400 MHz, DMSO-d6) 1H NMR (400 MHz, DMSO-d6) δ 12.89 (br, 1H), 8.08 (s, 1H), 7.91 (s, 1H), 7.63 (s, 1H), 7.37 (s, 1H), 6.90-6.69 (m, 1H), 6.19 (dd, J=16.6, 1.9 Hz, 1H), 5.71 (d, J=9.8 Hz, 1H), 5.61-5.26 (m, 1H), 4.10-3.94 (m, 2H), 3.91-3.69 (m, 1H), 3.13-3.00 (m, 1H), 1.18-1.06 (m, 6H). MS (ESI) m/z [M+H]⁺=387.

Isomer 2: (S)-1-(5-(7-chloro-2-(1H-imidazol-4-yl)benzo[d]oxazol-5-yl)-2,2-dimethylmorpholino)prop-2-en-1-one (D17B, assumed)

¹H NMR (399 MHz, DMSO-d6) δ 12.86 (br, 1H), 8.08 (s, 1H), 7.91 (s, 1H), 7.64 (s, 1H), 7.38 (s, 1H), 6.80 (s, 1H), 6.20 (d, J=16.5 Hz, 1H), 5.71 (d, J=8.7 Hz, 1H), 5.61-5.21 (m, 1H), 4.26-3.72 (m, 3H), 3.14-2.84 (m, 1H), 1.20-1.06 (m, 6H). m/z [M+H]⁺=387.

### Example 2-17: Synthesis of (R)-1-(3-(7-chloro-2-(1H-1,2,4-triazol-5-yl)benzo[d]oxazol-5-yl)morpholino)prop-2-en-1-one (D18A, Assumed) and (S)-1-(3-(7-chloro-2-(1H-1,2,4-triazol-5-yl)benzo[d]oxazol-5-yl)morpholino)prop-2-en-1-one (D18B, Assumed)

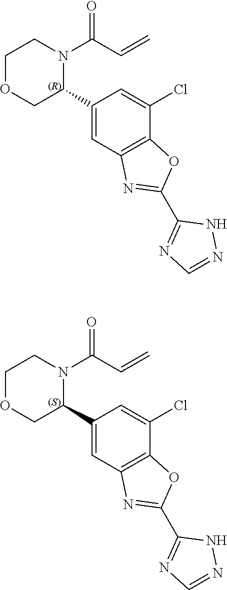

### Step 1: 5-bromo-7-chloro-2-(1H-1,2,4-triazol-5-yl)benzo[d]oxazole

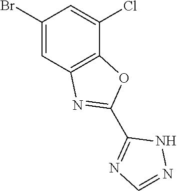 [SMILES: Clc1cc(Br)cc2nc(-c3ncn[nH]3)oc12]

To a solution of 1H-1,2,4-triazole-5-carboxylic acid (10.0 g, 88.4 mmol) in toluene (125 mL) was added oxalyl dichloride (15 mL, 176.9 mmol) at 0° C., followed by cat. DMF (0.25 mL) slowly at 0° C. The mixture was stirred at rt for 22 h. Upon completion of the reaction, the reaction was concentrated in vacuo directly to give the crude 1H-1,2,4-triazole-5-carbonyl chloride (12.0 g, crude), which could be used directly in the next step.

To a solution of 2-amino-4-bromo-6-chlorophenol (9.2 g, 41.6 mmol) and 1H-1,2,4-triazole-5-carbonyl chloride (10.9 g, 83.3 mmol) in dry DCM (120 mL) was added pyridine (14.6 mL, 181.2 mmol) slowly at 0° C. The mixture was stirred at 0° C. to rt for 16 h. Upon completion of the reaction, DCM (100 mL) was added and the precipitate was collected by filtration to give the crude N-(5-bromo-3-chloro-2-hydroxyphenyl)-1H-1,2,4-triazole-5-carboxamide (18.8 g). MS (ESI) m/z [M+H]⁺ 317, 319.

A mixture of N-(5-bromo-3-chloro-2-hydroxyphenyl)-1H-1,2,4-triazole-5-carboxamide (18.8 g) and polyphosphoric acid (85% purity, 150.0 g) was stirred at 130° C. for 24 h. Upon completion of the reaction, the mixture was poured into ice cold water and stirred for several hours at rt until the polyphosphoric acid was dissolved. The mixture was filtered, and the filter cake was washed with water. The filter cake was dried in vacuo to give the crude 5-bromo-7-chloro-2-(1H-1,2,4-triazol-5-yl)benzo[d]oxazole (17.4 g). MS (ESI) m/z [M+H]⁺ 299, 301.

### Step 2: 5-bromo-7-chloro-2-(1-((2-(trimethylsilyl)ethoxy)methyl)-1H-1,2,4-triazol-5-yl)benzo[d]oxazole (Assumed) and 5-bromo-7-chloro-2-(1-((2-(trimethylsilyl)ethoxy)methyl)-1H-1,2,4-triazol-3-yl)benzo[d]oxazole (Assumed)

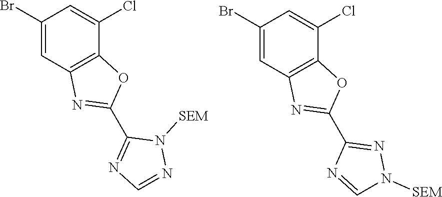 [SMILES: C[Si](C)(C)CCOCn1ncnc1-c1nc2cc(Br)cc(Cl)c2o1 ; C[Si](C)(C)CCOCn1cnc(-c2nc3cc(Br)cc(Cl)c3o2)n1]

To a solution of crude 5-bromo-7-chloro-2-(1H-1,2,4-triazol-5-yl)benzo[d]oxazole above (17.4 g, ca. 41.6 mmol) in dry THF (300 mL) was added NaH (60% purity, 5.0 g, 124.8 mmol) quickly at 0° C. The mixture was stirred at 0° C. for 30 min before SEMCl (22.1 mL, 124.8 mmol) was added at 0° C. The mixture was stirred at rt for 16 h. Upon completion of the reaction, the mixture was slowly added to sat.aq. NH₄Cl at 0° C. and extracted with EA. The organic layers were washed with brine, dried over Na₂SO₄, filtered and concentrated. The residue was purified by silica gel column chromatography (EA/PE 1/100 to 2/3) to give the title compounds 5-bromo-7-chloro-2-(1-((2-(trimethylsilyl)ethoxy)methyl)-1H-1,2,4-triazol-5-yl)benzo[d]oxazole (assumed, 3.9 g, 21.9%) and 5-bromo-7-chloro-2-(1-((2-(trimethylsilyl)ethoxy)methyl)-1H-1,2,4-triazol-3-yl)benzo[d]oxazole (assumed, 9.2 g, 51.6%). MS (ESI) m/z [M+H]⁺ 429, 431.

### Step 3: tert-butyl 5-(7-chloro-2-(1-((2-(trimethylsilyl)ethoxy)methyl)-1H-1,2,4-triazol-3-yl)benzo[d]oxazol-5-yl)-2,3-dihydro-4H-1,4-oxazine-4-carboxylate (Assumed)

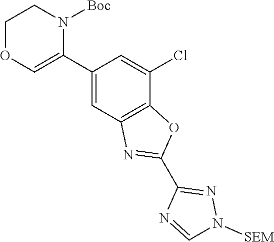 [SMILES: CC(C)(C)OC(=O)N1CCOC=C1c1cc(Cl)c2oc(-c3ncn(COCC[Si](C)(C)C)n3)nc2c1]

A solution of 5-bromo-7-chloro-2-(1-((2-(trimethylsilyl)ethoxy)methyl)-1H-1,2,4-triazol-3-yl)benzo[d]oxazole (assumed, 280 mg, 0.65 mmol), Bis(pinacolato)diboron (217 mg, 0.85 mmol), Pd(dppf)Cl₂ (48.5 mg, 0.07 mmol) and KOAc (129 mg, 1.34 mmol) in 1,4-dioxane (10 mL) was stirred at 90° C. for 1.5 h. After cooled to rt, tert-butyl 5-((diphenoxyphosphoryl)oxy)-2,3-dihydro-4H-1,4-oxazine-4-carboxylate (312 mg, 0.72 mmol), K₂CO₃ (181 mg, 1.3 mmol), Pd(dppf)Cl₂ (49 mg, 0.07 mmol) and water (2 mL) were added at rt. The mixture was stirred at 90° C. for 2 h. After completion of the reaction, EA was added and extracted with EA×3. The organic layers were washed with brine and dried over with anhydrous Na₂SO₄. The solvent was removed in vacuo, and the residue was purified by silica gel column chromatography (EA/PE 1/100 to 3/7) to give the title compound (147 mg, 42%). MS (ESI) m/z [M+H]⁺ 534.

### Step 4: 1-(3-(7-chloro-2-(1H-1,2,4-triazol-5-yl)benzo[d]oxazol-5-yl)morpholino)prop-2-en-1-one

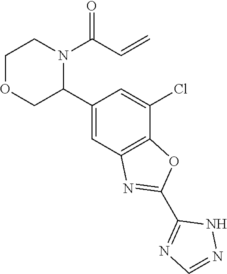 [SMILES: C=CC(=O)N1CCOCC1c1cc(Cl)c2oc(-c3ncn[nH]3)nc2c1]

To a solution of tert-butyl 5-(7-chloro-2-(1-((2-(trimethylsilyl)ethoxy)methyl)-1H-1,2,4-triazol-3-yl)benzo[d]oxazol-5-yl)-2,3-dihydro-4H-1,4-oxazine-4-carboxylate (assumed, 147 mg, 0.28 mmol) in DCM (20 mL) was added TFA (10 mL) at 0° C., the mixture was stirred at rt for 4 h. To the solution was added sodium triacetoxyborohydride (585 mg, 2.8 mmol) in several portions at 0° C., the mixture was stirred at rt for 5 h. Upon completion of the reaction, the solvent was removed in vacuo, and the residue was dissolved in THF (24 mL) and sat.aq. NaHCO₃ (24 mL) and then acryloyl chloride (17.7 mg, 0.20 mmol) in CH₂Cl₂ (1.8 mL) was added dropwise at 0° C. The mixture was stirred at 0° C. for 10 minutes. Upon completion of the reaction, a solution of MeOH/DCM (1/10, 30 mL) was added at 0° C. and extracted with MeOH/DCM (1/10)×3. The organic layers were washed with brine and dried over with anhydrous Na₂SO₄. The solvent was removed in vacuo, and the residue was purified by C18 column (mobile phase, ACN in water (0.1% FA), 20% to 35%) to give the title compound (12.6 mg, 12.7%). ¹H NMR (400 MHz, DMSO-d₆) δ 8.85 (s, 1H), 7.80 (s, 1H), 7.56 (s, 1H), 6.89 (dd, J=16.5, 10.4 Hz, 1H), 6.24 (d, J=16.5 Hz, 1H), 5.78 (d, J=10.6 Hz, 1H), 5.63 (brs, 1H), 4.49 (d, J=12.2 Hz, 1H), 4.18 (brs, 1H), 3.90 (d, J=11.6 Hz, 1H), 3.83 (d, J=11.6 Hz, 1H), 3.60-3.48 (m, 1H), 3.21-2.88 (m, 1H), 1.23 (s, 1H). MS (ESI) m/z [M+H]⁺=360.

### Step 5: (R)-1-(3-(7-chloro-2-(1H-1,2,4-triazol-5-yl)benzo[d]oxazol-5-yl)morpholino)prop-2-en-1-one (Assumed) (D18A) and (S)-1-(3-(7-chloro-2-(1H-1,2,4-triazol-5-yl)benzo[d]oxazol-5-yl)morpholino)prop-2-en-1-one (Assumed) (D18B)

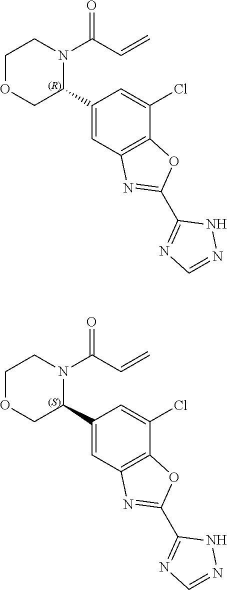

Racemic 1-(3-(7-chloro-2-(1H-1,2,4-triazol-5-yl)benzo[d]oxazol-5-yl)morpholino)prop-2-en-1-one was separated by using the following conditions:

SFC: column: YMC Amylose-SA, 30 mm×250 mm, 5 um; fluent: 45% MeOH (0.3% 2M NH₃ MeOH)/CO₂, flow rate: 90.0 mL/min; isomer 1, retention time=4.60 min, isomer 2, retention time=14.33 min;

Isomer 1:

¹H NMR (400 MHz, DMSO-d₆) δ 8.85 (s, 1H), 7.80 (s, 1H), 7.56 (s, 1H), 6.89 (dd, J=16.6, 10.4 Hz, 1H), 6.24 (d, J=16.7 Hz, 1H), 5.77 (d, J=10.3 Hz, 1H), 5.62 (brs, 1H), 4.49 (d, J=12.3 Hz, 1H), 4.18 (brs, 1H), 3.91 (d, J=10.0 Hz, 1H), 3.83 (d, J=10.9 Hz, 1H), 3.54 (t, J=10.9 Hz, 1H), 3.18-2.80 (m, 1H), 1.23 (s, 1H). MS (ESI) m/z [M+H]⁺=360.

Isomer 2:

¹H NMR (400 MHz, DMSO-d₆) δ 8.84 (s, 1H), 7.79 (s, 1H), 7.56 (s, 1H), 6.89 (dd, J=16.6, 10.5 Hz, 1H), 6.24 (d, J=16.2 Hz, 1H), 5.77 (d, J=10.6 Hz, 1H), 5.62 (brs, 1H), 4.49 (d, J=12.4 Hz, 1H), 4.18 (brs, 1H), 3.91 (d, J=10.0 Hz, 1H), 3.83 (d, J=10.8 Hz, 1H), 3.54 (t, J=11.4 Hz, 1H), 3.16-2.89 (m, 1H), 1.23 (s, 1H). MS (ESI) m/z [M+H]⁺=360.

### Example 2-18: Synthesis of (R)-1-(6-(7-chloro-2-(1H-1,2,4-triazol-5-yl)benzo[d]oxazol-5-yl)-4-oxa-7-azaspiro[2.5]octan-7-yl)prop-2-en-1-one (D22A, Assumed) and (S)-1-(6-(7-chloro-2-(1H-1,2,4-triazol-5-yl)benzo[d]oxazol-5-yl)-4-oxa-7-azaspiro[2.5]octan-7-yl)prop-2-en-1-one (D22B, Assumed)

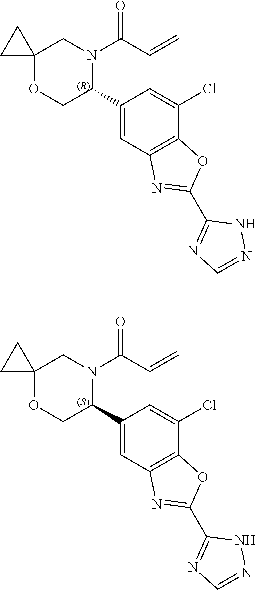

### Step 1: tert-butyl 6-(7-chloro-2-(1-((2-(trimethylsilyl)ethoxy)methyl)-1H-1,2,4-triazol-3-yl)benzo[d]oxazol-5-yl)-4-oxa-7-azaspiro[2.5]oct-5-ene-7-carboxylate (Assumed)

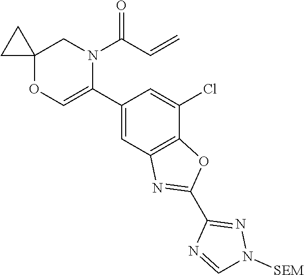 [SMILES: C=CC(=O)N1CC2(CC2)OC=C1c1cc(Cl)c2oc(-c3ncn(COCC[Si](C)(C)C)n3)nc2c1]

A solution of 5-bromo-7-chloro-2-(1-((2-(trimethylsilyl)ethoxy)methyl)-1H-1,2,4-triazol-3-yl)benzo[d]oxazole (assumed, 140 mg, 0.33 mmol), Bis(pinacolato)diboron (109 mg, 0.43 mmol), Pd(dppf)Cl₂ (24.3 mg, 0.04 mmol) and KOAc (65 mg, 0.67 mmol) in 1,4-dioxane (5 mL) was stirred at 90° C. for 1.5 h. After cooled to rt, tert-butyl 6-((diphenoxyphosphoryl)oxy)-4-oxa-7-azaspiro[2.5]oct-5-ene-7-carboxylate (165 mg, 0.36 mmol), K₂CO₃ (90 mg, 0.65 mmol), Pd(dppf)Cl₂ (24.3 mg, 0.04 mmol) and water (1 mL) were added at rt. The mixture was stirred at 90° C. for 4 h. After completion of the reaction, EA was added and extracted with EA×3. The organic layers were washed with brine and dried over with anhydrous Na₂SO₄. The solvent was removed in vacuo, and the residue was purified by silica gel column chromatography (EA/PE 1/100 to 1/2) to give the title compound (87 mg, 48%). MS (ESI) m/z [M+H]⁺ 560.

### Step 2: 1-(6-(7-chloro-2-(1H-1,2,4-triazol-5-yl)benzo[d]oxazol-5-yl)-4-oxa-7-azaspiro[2.5]octan-7-yl)prop-2-en-1-one

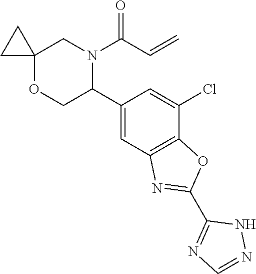 [SMILES: C=CC(=O)N1CC2(CC2)OCC1c1cc(Cl)c2oc(-c3ncn[nH]3)nc2c1]

To a solution of tert-butyl 6-(7-chloro-2-(1-((2-(trimethylsilyl)ethoxy)methyl)-1H-1,2,4-triazol-3-yl)benzo[d]oxazol-5-yl)-4-oxa-7-azaspiro[2.5]oct-5-ene-7-carboxylate (assumed, 87 mg, 0.16 mmol) in DCM (12 mL) was added TFA (6 mL) at 0° C., the mixture was stirred at rt for 2 h. To the solution was added sodium triacetoxyborohydride (329 mg, 1.6 mmol) in several portions at 0° C., the mixture was stirred at rt for 2 h. The reaction was concentrated, and the residue was dissolved in THF (24 mL) and sat.aq. NaHCO₃ (24 mL) and then acryloyl chloride (9.8 mg, 0.11 mmol) in CH₂Cl₂ (1.0 mL) was added dropwise at 0° C. The mixture was stirred at 0° C. for 10 minutes. Upon completion of the reaction, a solution of MeOH/DCM (1/10, 30 mL) was added at 0° C. and extracted with MeOH/DCM (1/10)×3. The organic layers were washed with brine and dried over with anhydrous Na₂SO₄. The solvent was removed in vacuo, and the residue was purified by C18 column (mobile phase, ACN in water (0.1% FA), 30% to 45%) to give the title compound (11.2 mg, 18.7%).

¹H NMR (400 MHz, DMSO-d₆) δ 8.90 (s, 1H), 7.85 (s, 1H), 7.59 (s, 1H), 6.86 (brs, 1H), 6.26 (d, J=16.7 Hz, 1H), 5.77 (d, J=9.0 Hz, 2H), 4.49 (d, J=12.0 Hz, 1H), 3.96 (s, 1H), 3.64 (s, 3H), 0.85 (s, 1H), 0.70 (d, J=6.6 Hz, 2H), 0.56 (s, 1H). MS (ESI) m/z [M+H]⁺ 386.

### Step 3: Synthesis of (R)-1-(6-(7-chloro-2-(1H-1,2,4-triazol-5-yl)benzo[d]oxazol-5-yl)-4-oxa-7-azaspiro[2.5]octan-7-yl)prop-2-en-1-one (Assumed) (D22A) and (S)-1-(6-(7-chloro-2-(1H-1,2,4-triazol-5-yl)benzo[d]oxazol-5-yl)-4-oxa-7-azaspiro[2.5]octan-7-yl)prop-2-en-1-one (Assumed) (D22B)

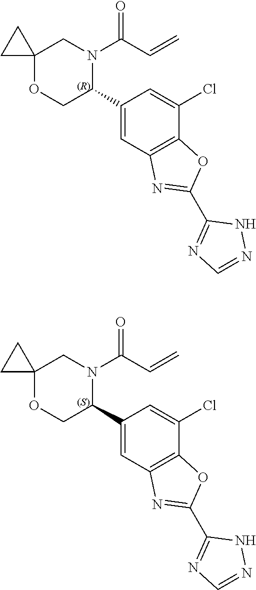

Racemic 1-(6-(7-chloro-2-(1H-1,2,4-triazol-5-yl)benzo[d]oxazol-5-yl)-4-oxa-7-azaspiro[2.5]octan-7-yl)prop-2-en-1-one was separated by using the following conditions:

SFC: column: Lux Cellulose-4, 30 mm×250 mm, 5 um; fluent: 42% MeOH (0.3% 2M NH₃ MeOH)/CO₂, flow rate: 90.0 mL/min; isomer 1, retention time=7.60 min, isomer 2, retention time=10.57 min;

Isomer 1:

¹H NMR (500 MHz, DMSO-d₆) δ 8.88 (s, 1H), 7.85 (s, 1H), 7.59 (s, 1H), 6.87 (brs, 1H), 6.26 (d, J=16.5 Hz, 1H), 5.77 (d, J=10.3 Hz, 1H), 4.49 (d, J=12.3 Hz, 1H), 3.96 (s, 1H), 3.84-3.36 (m, 3H), 0.85 (s, 1H), 0.70 (d, J=8.0 Hz, 2H), 0.56 (s, 1H). MS (ESI) m/z [M+H]⁺ 386.

Isomer 2:

¹H NMR (500 MHz, DMSO-d₆) δ 8.87 (s, 1H), 7.85 (s, 1H), 7.59 (s, 1H), 6.87 (brs, 1H), 6.26 (d, J=16.6 Hz, 1H), 5.77 (d, J=10.1 Hz, 1H), 4.49 (d, J=12.2 Hz, 1H), 3.96 (s, 1H), 3.88-3.35 (m, 3H), 0.85 (s, 1H), 0.71 (d, J=8.4 Hz, 2H), 0.56 (s, 1H). MS (ESI) m/z [M+H]⁺ 386.

### Example 2-19: Synthesis of (S)-1-(3-(4-chloro-2-(1H-imidazol-4-yl)benzo[d]oxazol-6-yl)morpholino)prop-2-en-1-one (L1A, Assumed) and (R)-1-(3-(4-chloro-2-(1H-imidazol-4-yl)benzo[d]oxazol-6-yl)morpholino)prop-2-en-1-one (L1B, Assumed)

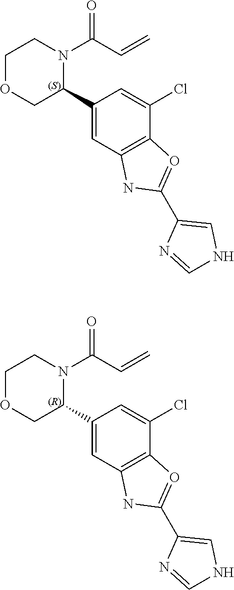

### Step 1: 2-amino-5-bromo-3-chlorophenol

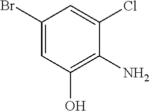

To a solution of 2-amino-3-chloro-phenol (1.0 g, 7.0 mmol) in DCM (20 mL) was added Br2 (1.7 g, 10.76 mmol) at 0° C., the mixture was stirred at 0° C. for 2 hours. Upon completion of the reaction, the solid was collected by filtration and the filter cake was dissolved in EA, the resulting solution was washed with brine, dried over Na₂SO₄, filtered and concentrated under reduced pressure to give a residue. The residue was purified by column chromatography (PE/EtOAc 10/1 to 2/1) to give the title compound (1.4 g, 90%). MS (ESI) m/z [M+H]⁺ 222/224.

### Step 2: 6-bromo-4-chloro-2-(1H-imidazol-4-yl)benzo[d]oxazole

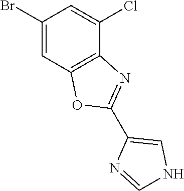 [SMILES: Clc1cc(Br)cc2oc(-c3c[nH]cn3)nc12]

2-amino-5-bromo-3-chloro-phenol (1.23 g, 5.53 mmol) and 4-(trifluoromethyl)-1H-imidazole (750 mg, 5.51 mmol) were dissolved in NaOH (50 mL, 30 mmol, 0.5 mol/L), the resulting mixture was stirred at 80° C. for 1 hour. After cooled to rt, the solid was collected by filtration and the filter cake was washed with water and dried in vacuo to give the title compound (1.26 g, 76.7%). MS (ESI) m/z [M+H]⁺ 298/300.

### Step 3: 6-bromo-4-chloro-2-(1-((2-(trimethylsilyl)ethoxy)methyl)-1H-imidazol-4-yl)benzo[d]oxazole (SEM Position Assumed)

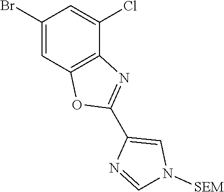 [SMILES: C[Si](C)(C)CCOCn1cnc(-c2nc3c(Cl)cc(Br)cc3o2)c1]

To a solution of 6-bromo-4-chloro-2-(1H-imidazol-4-yl)-1,3-benzoxazole (1.26 g, 4.22 mmol) in DMF (50 mL) was added NaH (253.00 mg, 6.33 mmol, 60% in mineral oil) at 0° C., and the resulting mixture was stirred at 0° C. for 1 hour. Then SEMCl (1.41 g, 8.44 mmol) was added at 0° C., the mixture was stirred at 0° C. for 1 hour. Upon completion of the reaction, the reaction was quenched with water and extracted with EA, the combined organic layer was washed with brine, dried over Na₂SO₄, filtered and concentrated under reduced pressure to give a residue. The residue was purified by column chromatography (PE/EtOAc 10/1 to 3/2) to give the title compound (SEM position assumed, 532 mg, 29.4%). MS (ESI) m/z [M+H]⁺ 428/430.

### Step 4: 4-chloro-6-(4,4,5,5-tetramethyl-1,3,2-dioxaborolan-2-yl)-2-(1-((2-(trimethylsilyl)ethoxy)methyl)-1H-imidazol-4-yl)benzo[d]oxazole (SEM Position Assumed)

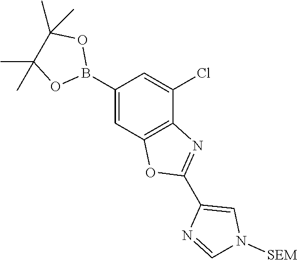 [SMILES: CC1(C)OB(c2cc(Cl)c3nc(-c4cn(COCC[Si](C)(C)C)cn4)oc3c2)OC1(C)C]

A solution of 2-[[4-(6-bromo-4-chloro-1,3-benzoxazol-2-yl)imidazol-1-yl]methoxy]ethyl-trimethyl-silane (SEM position assumed, 532 mg, 1.24 mmol), B2Pin2 (473 mg, 1.86 mmol), Pd(dppf)Cl2 (91 mg, 0.12 mmol) and KOAc (243 mg, 2.48 mmol) in 1,4-dioxane (10 mL) was stirred at 90° C. for overnight. After cooled to rt, water was added and the resulting mixture was extracted with EA. The combined organic layer was washed with brine, dried over Na2SO4, filtered and concentrated under reduced pressure to give a residue. The residue was purified by column chromatography (PE/EtOAc 10/1 to 1/1) to give the title compound (SEM position assumed, 578 mg, 97.9%). MS (ESI) m/z [M+H]⁺ 476.

### Step 5: tert-butyl 5-(4-chloro-2-(1-((2-(trimethylsilyl)ethoxy)methyl)-1H-imidazol-4-yl)benzo[d]oxazol-6-yl)-2,3-dihydro-4H-1,4-oxazine-4-carboxylate (SEM Position Assumed)

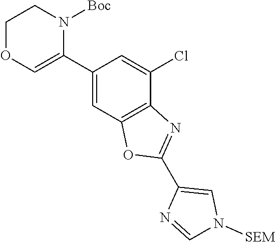 [SMILES: CC(C)(C)OC(=O)N1CCOC=C1c1cc(Cl)c2nc(-c3cn(COCC[Si](C)(C)C)cn3)oc2c1]

A solution of 2-[[4-[4-chloro-6-(4,4,5,5-tetramethyl-1,3,2-dioxaborolan-2-yl)-1,3-benzoxazol-2-yl]imidazol-1-yl]methoxy]ethyl-trimethyl-silane (SEM position assumed, 578 mg, 1.21 mmol), tert-butyl 5-diphenoxyphosphoryloxy-2,3-dihydro-1,4-oxazine-4-carboxylate (790 mg, 1.82 mmol), Pd(dtbpf)Cl2 (79 mg, 0.12 mmol) and K3PO4 (515 mg, 2.43 mmol) in 1,4-dioxane (20 mL) and H2O (5 mL) was stirred at 90° C. for 2 hours. After cooled to rt, water was added and the resulting mixture was extracted with EA. The combined organic layer was washed with brine, dried over Na2SO4, filtered and concentrated under reduced pressure to give a residue. The residue was purified by column chromatography (DCM/MeOH 100/1 to 20/1) to give the title compound (SEM position assumed, 600 mg, 92.7%). MS (ESI) m/z [M+H]⁺ 533.

### Step 6: 4-chloro-6-(5,6-dihydro-2H-1,4-oxazin-3-yl)-2-(1H-imidazol-4-yl)benzo[d]oxazole

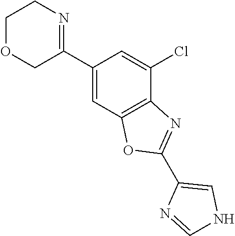 [SMILES: Clc1cc(C2=NCCOC2)cc2oc(-c3c[nH]cn3)nc12]

To a solution of tert-butyl 5-[4-chloro-2-[1-(2-trimethylsilylethoxymethyl)imidazol-4-yl]-1,3-benzoxazol-6-yl]-2,3-dihydro-1,4-oxazine-4-carboxylate (SEM position assumed, 200 mg, 0.38 mmol) in HFIP (5 mL) was added MsOH (0.2 mL), the mixture was stirred at rt for 10 min. The reaction solution was used directly for the next step without concentration.

### Step 7: 4-chloro-2-(1H-imidazol-4-yl)-6-(morpholin-3-yl)benzo[d]oxazole

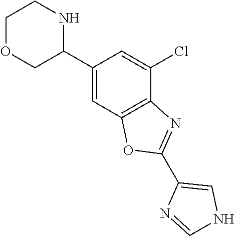 [SMILES: Clc1cc(C2COCCN2)cc2oc(-c3c[nH]cn3)nc12]

To a crude HFIP solution of 4-chloro-6-(3,6-dihydro-2H-1,4-oxazin-5-yl)-2-(1H-imidazol-4-yl)-1,3-benzoxazole (200 mg, 0.66 mmol) from previous step was added MeOH (30 mL), and to this mixture was added NaBH3CN (83 mg, 1.32 mmol), the resulting mixture was stirred at rt for 20 mins. Upon completion of the reaction, the solvent was removed in vacuo to give the crude product and it was used directly for next step. MS (ESI) m/z [M+H]⁺ 305.

### Step 8: 1-(3-(4-chloro-2-(1H-imidazol-4-yl)benzo[d]oxazol-6-yl)morpholino)prop-2-en-1-one

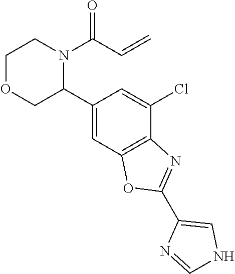 [SMILES: C=CC(=O)N1CCOCC1c1cc(Cl)c2nc(-c3c[nH]cn3)oc2c1]

To a solution of 4-chloro-2-(1H-imidazol-4-yl)-6-morpholin-3-yl-1,3-benzoxazole (200 mg, 0.66 mmol) in ACN (20 mL) and sat. NaHCO₃ (20 mL) was added acryloyl chloride (59 mg, 0.65 mmol) in ACN, the mixture was stirred at rt for 10 mins. Upon completion of the reaction, the reaction was extracted with MeOH:DCM (v:v=1:20), the combined organic layer was dried over Na2SO4, and concentrated in vacuo. The residue was purified by silica gel column chromatography (MeOH/DCM 1/100 to 1/20) to give the crude product. The crude product was purified by C18 column chromatography (ACN/H2O 1/20 to 1/1) to give the product. 1H NMR (400 MHz, DMSO-d6) δ 12.84 (br, 1H), 8.20-8.05 (m, 1H), 7.92 (s, 1H), 7.66 (s, 1H), 7.38 (s, 1H), 6.90-6.77 (m, 1H), 6.20 (dd, J=16.6, 2.1 Hz, 1H), 5.73 (dd, J=10.4, 2.0 Hz, 1H), 5.67-5.32 (m, 1H), 4.43 (d, J=12.2 Hz, 1H), 3.87-3.76 (m, 2H), 3.52-3.45 (m, 2H), 3.19-3.11 (m, 1H). MS (ESI) m/z [M+H]⁺ 359.

### Step 9: (S)-1-(3-(4-chloro-2-(1H-imidazol-4-yl)benzo[d]oxazol-6-yl)morpholino)prop-2-en-1-one (L1A assumed) and (R)-1-(3-(4-chloro-2-(1H-imidazol-4-yl)benzo[d]oxazol-6-yl)morpholino)prop-2-en-1-one (L1B Assumed)

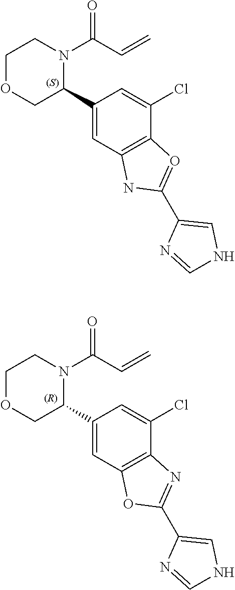

Racemic 1-(3-(4-chloro-2-(1H-imidazol-4-yl)benzo[d]oxazol-6-yl)morpholino)prop-2-en-1-one was separated by using the following conditions:

Column: Lux Cellulose-4 30 mm×250 mm, 5 μm; 38% MeOH (0.3% 2M NH₃ MeOH); isomer 1, retention time=9.23 min, isomer 2, retention time=11.12 min;

Isomer 1: (S)-1-(3-(4-chloro-2-(1H-imidazol-4-yl)benzo[d]oxazol-6-yl)morpholino)prop-2-en-1-one (L1A, assumed)

¹H NMR (400 MHz, DMSO-d₆) δ 12.85 (br, 1H), 8.11 (s, 1H), 7.92 (s, 1H), 7.65 (s, 1H), 7.38 (s, 1H), 6.89-6.76 (m, 1H), 6.20 (d, J=16.6 Hz, 1H), 5.73 (d, J=10.4 Hz, 1H), 5.66-5.33 (m, 1H), 4.43 (d, J=12.2 Hz, 1H), 3.92-3.72 (m, 3H), 3.52-3.44 (m, 1H), 3.05-2.97 (m, 1H). MS (ESI) m/z [M+H]⁺=359.

Isomer 2: (R)-1-(3-(4-chloro-2-(1H-imidazol-4-yl)benzo[d]oxazol-6-yl)morpholino)prop-2-en-1-one (L1B, assumed)

¹H NMR (399 MHz, DMSO-d₆) δ 12.84 (br, 1H), 8.11 (s, 1H), 7.91 (s, 1H), 7.66 (s, 1H), 7.38 (s, 1H), 6.90-6.77 (m, 1H), 6.20 (d, J=16.3 Hz, 1H), 5.73 (d, J=10.2 Hz, 1H), 5.66-5.42 (m, 1H), 4.43 (d, J=12.1 Hz, 1H), 4.00-3.72 (m, 3H), 3.54-3.44 (m, 1H), 2.96-2.88 (m, 1H). m/z [M+H]⁺=359.

Compounds L2 in Table 2-7 below was synthesized by using corresponding starting materials according to procedures similar to those described for the synthesis of compounds E1.

TABLE 2-7

| Compound | Structure | Chemical Name | ¹H NMR data LC/MS m/z (M + H) |
|---|---|---|---|
| L2 | 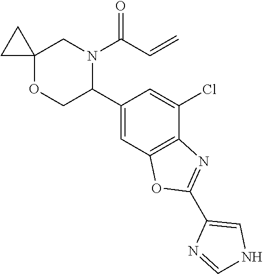 [SMILES: C=CC(=O)N1CC2(CC2)OCC1c1cc(Cl)c2nc(-c3c[nH]cn3)oc2c1] | 1-(6-(4-chloro-2-(1H- imidazol-4- yl)benzo[d]oxazol-6- yl)-4-oxa-7- azaspiro[2.5]octan-7- yl)prop-2-en-1-one | ¹H NMR (400 MHz, DMSO-d6) δ 12.86 (s, 1H), 8.17 (s, 1H), 7.95 (s, 1H), 7.73 (s, 1H), 7.41 (s, 1H), 6.94-6.78 (m, 1H), 6.25 (d, J = 16.6 Hz, 1H), 5.81-5.39 (m, 2H), 4.47 (d, J = 12.2 Hz, 1H), 4.03-3.88 (m, 1H), 3.86-3.39 (m, 2H), 0.87-0.80 (m, 1H), 0.77-0.64 (m, 2H), 0.61-0.48 (m, 1H). MS (ESI) m/z [M + H]⁺ = 385. |

### Example 2-20: (S)-1-(6-(4-chloro-2-(1H-imidazol-4-yl)benzo[d]oxazol-6-yl)-4-oxa-7-azaspiro[2.5]octan-7-yl)prop-2-en-1-one (Assumed) (L2A) and (R)-1-(6-(4-chloro-2-(1H-imidazol-4-yl)benzo[d]oxazol-6-yl)-4-oxa-7-azaspiro[2.5]octan-7-yl)prop-2-en-1-one (Assumed) (L2B)

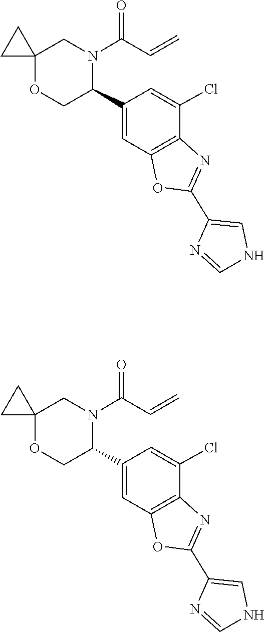 [SMILES: C=CC(=O)N1CC2(CC2)OC[C@@H]1c1cc(Cl)c2nc(-c3c[nH]cn3)oc2c1 ; C=CC(=O)N1CC2(CC2)OC[C@H]1c1cc(Cl)c2nc(-c3c[nH]cn3)oc2c1]

Racemic 1-(6-(4-chloro-2-(1H-imidazol-4-yl)benzo[d]oxazol-6-yl)-4-oxa-7-azaspiro[2.5]octan-7-yl)prop-2-en-1-one was separated by using the following conditions:

Column: YMC i-Amylose-3 30 mm×250 mm, 5 μm; 45% MeOH (0.3% 2M NH₃ MeOH); isomer 1, retention time=5.15 min, isomer 2, retention time=8.40 min;

Isomer 1:

¹H NMR (500 MHz, DMSO-d6) δ 12.87 (s, 1H), 8.19 (s, 1H), 7.98 (s, 1H), 7.73 (s, 1H), 7.45 (s, 1H), 6.86 (s, 1H), 6.25 (d, J=16.6 Hz, 1H), 5.81-5.41 (m, 2H), 4.47 (d, J=12.2 Hz, 1H), 4.03-3.87 (m, 1H), 3.83-3.39 (m, 2H), 0.90-0.78 (m, 1H), 0.76-0.65 (m, 2H), 0.62-0.48 (m, 1H). MS (ESI) m/z [M+H]⁺=385.

Isomer 2:

¹H NMR (500 MHz, DMSO-d6) δ 12.87 (s, 1H), 8.19 (s, 1H), 7.98 (s, 1H), 7.73 (s, 1H), 7.45 (s, 1H), 6.86 (s, 1H), 6.25 (d, J=16.2 Hz, 1H), 5.82-5.39 (m, 2H), 4.47 (d, J=12.2 Hz, 1H), 4.02-3.88 (m, 1H), 3.84-3.39 (m, 2H), 0.89-0.79 (m, 1H), 0.76-0.65 (m, 2H), 0.62-0.48 (m, 1H). m/z [M+H]⁺=385.

### Example 2-21: Synthesis of (R)-1-(3-(7-chloro-2-(1H-1,2,4-triazol-3-yl)benzo[b]thiophen-5-yl)morpholino)prop-2-en-1-one (M1A, Assumed) and (S)-1-(3-(7-chloro-2-(1H-1,2,4-triazol-3-yl)benzo[b]thiophen-5-yl)morpholino)prop-2-en-1-one (M1B, Assumed)

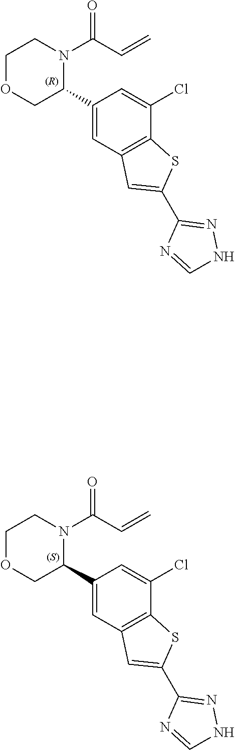

### Step 1: 3-(5-bromo-7-chlorobenzo[b]thiophen-2-yl)-1H-1,2,4-triazole

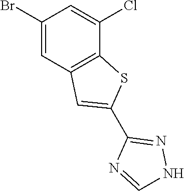 [SMILES: Clc1cc(Br)cc2cc(-c3nc[nH]n3)sc12]

To a solution of 5-bromo-3-chloro-2-fluorobenzaldehyde (10.0 g, 42.1 mmol) and K₂CO₃ (8.7 g, 63.2 mmol) in DMF (100 mL) was added dropwise methyl 2-mercaptoacetate (4.6 mL, 50.5 mmol) at 0° C. The mixture was stirred at rt for 1.5 h, and then stirred at 60° C. for 1 hour. The reaction mixture was treated with water and stirred at rt for 15 min. The resulting suspension was filtered, and the filter cake was washed with water and dried under high vacuum to give the crude methyl 5-bromo-7-chlorobenzo[b]thiophene-2-carboxylate (14.0 g).

To a solution of crude methyl 5-bromo-7-chlorobenzo[b]thiophene-2-carboxylate above (4.4 g, ca. 13.3 mmol) in dry DMF (60 mL) was added ammonia solution (60 mL, 25% to 30% in water) at rt. The reaction mixture was stirred at 50° C. for 12 h. After completion of the reaction, the solvent was removed in vacuo directly to give the crude 5-bromo-7-chlorobenzo[b]thiophene-2-carboxamide (5.0 g). MS (ESI) m/z [M+H]⁺ 290, 292.

To a solution of crude 5-bromo-7-chlorobenzo[b]thiophene-2-carboxamide (5.0 g, ca. 13.3 mmol) in dry DMF (80 mL) was added DMF-DMA (8 mL) at rt. The reaction mixture was stirred at 120° C. for 12 h. Upon completion of the reaction, the solvent was removed in vacuo directly to give the crude 5-bromo-7-chloro-N-((dimethylamino)methylene)benzo[b]thiophene-2-carboxamide, which was used directly in the next step without further purification. MS (ESI) m/z [M+H]⁺ 345, 347.

To a solution of crude 5-bromo-7-chloro-N-((dimethylamino)methylene) benzo[b]thiophene-2-carboxamide above in AcOH (120 mL) was added hydrazinium hydroxide (2.6 g, 43.9 mmol, 85%) at rt. The reaction mixture was stirred at 100° C. for 1 hour. After cooled to rt, the solvent was removed in vacuo, and the residue was purified by silica gel column chromatography (MeOH/DCM 1/20) to give the title compound (4.0 g, 96%). MS (ESI) m/z [M+H]⁺ 314, 316.

### Step 2: 5-(5-bromo-7-chlorobenzo[b]thiophen-2-yl)-1-((2-(trimethylsilyl)ethoxy)methyl)-1H-1,2,4-triazole (Assumed) and 3-(5-bromo-7-chlorobenzo[b]thiophen-2-yl)-1-((2-(trimethylsilyl)ethoxy)methyl)-1H-1,2,4-triazole (Assumed)

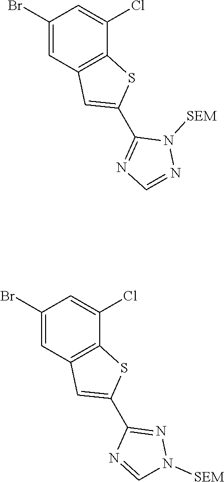 [SMILES: C[Si](C)(C)CCOCn1ncnc1-c1cc2cc(Br)cc(Cl)c2s1 ; C[Si](C)(C)CCOCn1cnc(-c2cc3cc(Br)cc(Cl)c3s2)n1]

To a solution of 3-(5-bromo-7-chlorobenzo[b]thiophen-2-yl)-1H-1,2,4-triazole (1.5 g, 4.8 mmol) in dry THF (40 mL) was added NaH (0.23 g, 5.8 mmol, 60% in mineral oil) slowly at 0° C. The mixture was stirred at 0° C. for 30 min, followed by (2-(chloromethoxy)ethyl) trimethylsilane (1.1 mL, 6.2 mmol). The resulting mixture was stirred at rt for 2.5 h. Upon completion of the reaction, sat.aq. NH₄Cl was slowed added at 0° C. and the resulting mixture was extracted with EA. The combined organic layers were washed with brine, dried over Na₂SO₄, filtered and concentrated. The residue was purified by silica gel column chromatography (EA/PE 1/100 to 3/2) to give the title compounds 5-(5-bromo-7-chlorobenzo[b]thiophen-2-yl)-1-((2-(trimethylsilyl)ethoxy)methyl)-1H-1,2,4-triazole (assumed, 420 mg, 19.8%) and 3-(5-bromo-7-chlorobenzo[b]thiophen-2-yl)-1-((2-(trimethylsilyl)ethoxy)methyl)-1H-1,2,4-triazole (assumed, 970 mg, 45.7%). MS (ESI) m/z [M+H]⁺ 444, 446.

### Step 3: tert-butyl 5-(7-chloro-2-(1-((2-(trimethylsilyl)ethoxy)methyl)-1H-1,2,4-triazol-3-yl)benzo[b]thiophen-5-yl)-2,3-dihydro-4H-1,4-oxazine-4-carboxylate

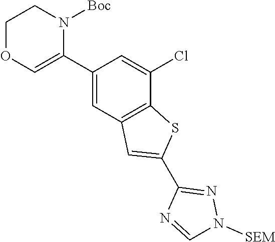 [SMILES: CC(C)(C)OC(=O)N1CCOC=C1c1cc(Cl)c2sc(-c3ncn(COCC[Si](C)(C)C)n3)cc2c1]

A solution of 3-(5-bromo-7-chlorobenzo[b]thiophen-2-yl)-1-((2-(trimethylsilyl)ethoxy)methyl)-1H-1,2,4-triazole (assumed, 600 mg, 1.35 mmol), bis(pinacolato)diboron (445 mg, 1.75 mmol), Pd(dppf)Cl₂ (99 mg, 0.14 mmol) and KOAc (260 mg, 2.70 mmol) in 1,4-dioxane (30 mL) was stirred at 90° C. for 1 hour. After completion of the reaction, it was cooled to rt, tert-butyl 5-((diphenoxyphosphoryl)oxy)-2,3-dihydro-4H-1,4-oxazine-4-carboxylate (643 mg, 1.48 mmol), K₂CO₃ (372 mg, 2.70 mmol), Pd(dppf)Cl₂ (99 mg, 0.14 mmol) and water (6 mL) were added at rt. The mixture was stirred at 90° C. for 4 h. After cooled to rt, the resulting mixture was extracted with EA. The organic layers were washed with brine and dried over with anhydrous Na₂SO₄. The solvent was removed in vacuo, and the residue was purified by silica gel column chromatography (EA/PE 1/100 to 3/2) to give the title compound (680 mg, 91%). MS (ESI) m/z [M+H]⁺ 549, 551.

### Step 4: 1-(3-(7-chloro-2-(1H-1,2,4-triazol-3-yl)benzo[b]thiophen-5-yl)morpholino)prop-2-en-1-one

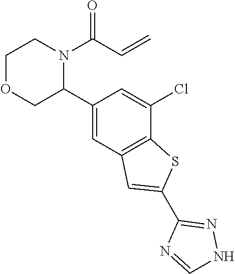 [SMILES: C=CC(=O)N1CCOCC1c1cc(Cl)c2sc(-c3nc[nH]n3)cc2c1]

To a solution of tert-butyl 5-(7-chloro-2-(1-((2-(trimethylsilyl)ethoxy)methyl)-1H-1,2,4-triazol-3-yl)benzo[b]thiophen-5-yl)-2,3-dihydro-4H-1,4-oxazine-4-carboxylate (680 mg, 1.24 mmol) in DCM (30 mL) was added TFA (30 mL) at 0° C., the mixture was stirred at rt for 3.5 h. Then to the solution was added sodium triacetoxyborohydride (5.15 mg, 24.3 mmol) in several portions at 0° C., the mixture was stirred at rt for 48 h. Upon completion of the reaction, the reaction was concentrated and the residue was re-dissolved in THF (60 mL) and sat.aq. NaHCO₃ (60 mL), then acryloyl chloride (65 mg, 0.72 mmol) in CH₂Cl₂ (6.5 mL) was added dropwise at 0° C. The mixture was stirred at 0° C. for 10 min. Upon completion of the reaction, the resulting mixture was extracted with MeOH/DCM (1/10). The organic layers were washed with brine and dried over with anhydrous Na₂SO₄. The solvent was removed in vacuo, and the residue was purified by C18 column (mobile phase, ACN in water (0.1% FA), 25% to 45%) to give the title compound (134.7 mg, 24.8%).

¹H NMR (400 MHz, DMSO-d₆) δ 8.66 (s, 1H), 8.05 (s, 1H), 7.93 (s, 1H), 7.49 (s, 1H), 6.86 (dd, J=16.1, 10.7 Hz, 1H), 6.24 (d, J=16.6 Hz, 1H), 5.76 (d, J=10.2 Hz, 1H), 5.63 (brs, 1H), 4.47 (d, J=12.2 Hz, 1H), 4.23 (brs, 1H), 3.89 (d, J=10.2 Hz, 1H), 3.83 (d, J=11.9 Hz, 1H), 3.53 (t, J=11.1 Hz, 1H), 3.31-2.91 (m, 1H). MS (ESI) m/z [M+H]⁺=375, 377.

### Step 5: (R)-1-(3-(7-chloro-2-(1H-1,2,4-triazol-3-yl)benzo[b]thiophen-5-yl)morpholino)prop-2-en-1-one (M1A, Assumed) and (S)-1-(3-(7-chloro-2-(1H-1,2,4-triazol-3-yl)benzo[b]thiophen-5-yl)morpholino)prop-2-en-1-one (M1B, Assumed)

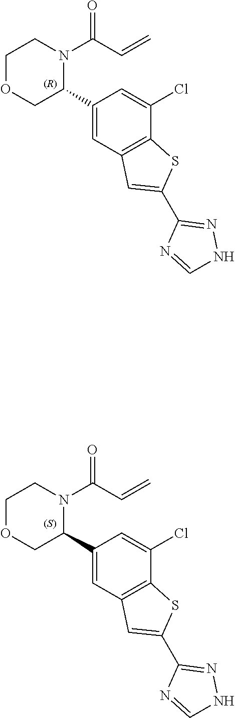

Racemic 1-(3-(7-chloro-2-(1H-1,2,4-triazol-3-yl)benzo[b]thiophen-5-yl)morpholino)prop-2-en-1-one was separated by using the following conditions:

SFC: column: CHIRALPAK AD-H, 20 mm×250 mm, 5 um; fluent: 45% MeOH(0.3% 2M NH₃ MeOH)/CO₂, flow rate: 40.0 mL/min; isomer 1, retention time=7.92 min, isomer 2, retention time=10.33 min;

Isomer 1:

¹H NMR (400 MHz, DMSO-d₆) δ 8.72 (s, 1H), 8.04 (s, 1H), 7.93 (s, 1H), 7.49 (s, 1H), 6.92-6.81 (m, 1H), 6.24 (d, J=16.6 Hz, 1H), 5.77 (d, J=10.1 Hz, 1H), 5.62 (brs, 1H), 4.47 (d, J=12.5 Hz, 1H), 4.23 (brs, 1H), 3.89 (d, J=10.3 Hz, 1H), 3.83 (d, J=12.0 Hz, 1H), 3.53 (t, J=11.4 Hz, 1H), 3.25-2.88 (m, 1H). MS (ESI) m/z [M+H]⁺=375.

Isomer 2:

¹H NMR (400 MHz, DMSO-d₆) δ 8.65 (s, 1H), 8.04 (s, 1H), 7.92 (s, 1H), 7.49 (s, 1H), 6.87 (dd, J=16.5, 10.5 Hz, 1H), 6.24 (d, J=16.5 Hz, 1H), 5.77 (d, J=10.3 Hz, 1H), 5.61 (brs, 1H), 4.47 (d, J=12.2 Hz, 1H), 4.23 (brs, 1H), 3.90 (d, J=10.1 Hz, 1H), 3.83 (d, J=11.5 Hz, 1H), 3.53 (t, J=10.4 Hz, 1H), 3.24-2.82 (m, 1H). MS (ESI) m/z [M+H]⁺=375.

### Example 2-22: Synthesis of (R)-1-(3-(7-chloro-4-fluoro-2-(4H-1,2,4-triazol-3-yl)benzofuran-5-yl)morpholino)prop-2-en-1-one (B29A, Assumed) and (S)-1-(3-(7-chloro-4-fluoro-2-(4H-1,2,4-triazol-3-yl)benzofuran-5-yl)morpholino)prop-2-en-1-one (B29B, Assumed)

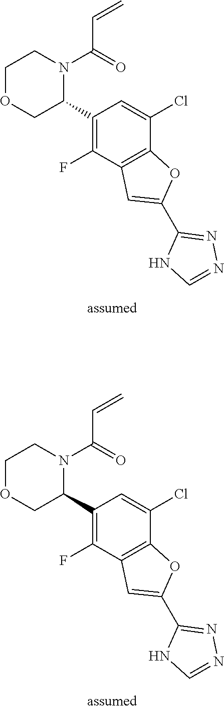 [SMILES: C=CC(=O)N1CCOC[C@H]1c1cc(Cl)c2oc(-c3nnc[nH]3)cc2c1F ; C=CC(=O)N1CCOC[C@@H]1c1cc(Cl)c2oc(-c3nnc[nH]3)cc2c1F]

### Step 1: 1-bromo-5-chloro-2-fluoro-4-methoxybenzene

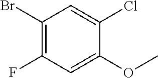

To a solution of 1-chloro-4-fluoro-2-methoxy-benzene (200 g, 1.2 mol) in DCM (2 L) was added Br₂ (298.5 g, 1.8 mol, 96.2 mL) dropwise at 0° C. The mixture was stirred at 0° C. for 12 hr. Upon completion of the reaction, the mixture was quenched by addition of Na₂S₂O₃ (1000 mL) and then extracted with DCM (1000 mL×3). The combined organic layers were washed with brine, dried over Na₂SO₄, filtered and concentrated under reduced pressure to give the residue. The residue was purified by silica gel column chromatography (n-hexane: EA=100/1 to 10/1) to give the product (200 g, 67% yield).

¹H NMR (400 MHz, DMSO-d₆) δ=7.79 (d, J=7.2 Hz, 1H), 7.30 (d, J=10.8 Hz, 1H), 3.87 (s, 3H).

### Step 2: 2-(5-chloro-2-fluoro-4-methoxyphenyl)-4,4,5,5-tetramethyl-1,3,2-dioxaborolane

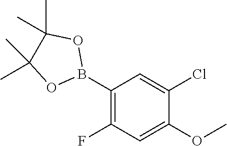

A mixture of 1-bromo-5-chloro-2-fluoro-4-methoxy-benzene (100 g, 417.6 mmol), 4,4,5,5-tetramethyl-2-(4,4,5,5-tetramethyl-1,3,2-dioxaborolan-2-yl)-1,3,2-dioxaborolane (127.2 g, 501.1 mmol), Pd(dppf)Cl₂ (15.3 g, 20.8 mmol) and KOAc (122.9 g, 1.2 mol) in dioxane (500 mL) was degassed and purged with N₂ for 3 times, and then the mixture was stirred at 110° C. for 6 hr under N₂ atmosphere. Upon completion of the reaction, the mixture was filtered and concentrated under reduced pressure to give a residue. The residue was purified by silica gel column chromatography (n-hexane: EA=100/1 to 10/1) to give the product (150 g, 75.2% yield, 60% purity).

### Step 3: tert-butyl 5-(5-chloro-2-fluoro-4-methoxyphenyl)-2,3-dihydro-4H-1,4-oxazine-4-carboxylate

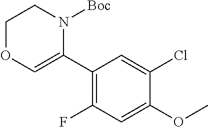 [SMILES: COc1cc(F)c(C2=COCCN2C(=O)OC(C)(C)C)cc1Cl]

A mixture of 2-(5-chloro-2-fluoro-4-methoxy-phenyl)-4,4,5,5-tetramethyl-1,3,2-dioxaborolane (50 g, 174.5 mmol), tert-butyl 5-diphenoxyphosphoryloxy-2,3-dihydro-1,4-oxazine-4-carboxylate (75.6 g, 174.50 mmol), K₂CO₃ (72.3 g, 523.5 mmol) and Pd(dppf)Cl₂ (6.4 g, 8.7 mmol) in dioxane (500 mL) and H₂O (100 mL) was degassed and purged with N₂ for 3 times, and then the mixture was stirred at 90° C. for 2 hr under N₂ atmosphere. Upon completion of the reaction, the mixture was filtered and concentrated under reduced pressure to give a residue. The residue was purified by silica gel column chromatography (n-hexane: EA=20/1 to 5/1) to give the product (50 g, 42% yield). MS (ESI) m/z [M+H]⁺=344. ¹H NMR (400 MHz, DMSO-d₆) δ=7.26 (d, J=8.0 Hz, 1H), 7.08 (d, J=12.4 Hz, 1H), 6.31 (s, 1H), 4.12 (t, J=4.4 Hz, 2H), 3.85 (s, 3H), 3.67 (t, J=4.4 Hz, 2H), 1.10 (s, 9H).

### Step 4: 2-chloro-4-(3,4-dihydro-2H-1,4-oxazin-5-yl)-5-fluorophenol

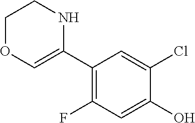 [SMILES: Oc1cc(F)c(C2=COCCN2)cc1Cl]

To a solution of tert-butyl 5-(5-chloro-2-fluoro-4-methoxy-phenyl)-2,3-dihydro-1,4-oxazine-4-carboxylate (50 g, 145.4 mmol) in DCM (500 mL) was added dropwise BBr₃ (145.44 mL, 290.9 mmol, 2 M) at −60° C. under N₂. The mixture was stirred at −60° C. for 0.5 h and 20° C. for 1 h. Upon completion of the reaction, the mixture was poured into ice water (500 mL) and stirred at 0° C. for 30 min. The solution was freeze-dried to give the title product (34 g, crude). MS (ESI) m/z [M+H]⁺=230.

### Step 5: 2-chloro-5-fluoro-4-(morpholin-3-yl)phenol

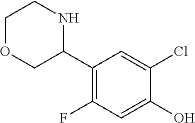 [SMILES: Oc1cc(F)c(C2COCCN2)cc1Cl]

To a solution of 2-chloro-4-(3,4-dihydro-2H-1,4-oxazin-5-yl)-5-fluorophenol (34 g, 148.0 mmol) in DCM (400 mL) was added dropwise NaBH₃CN (37.2 g, 592.2 mmol) at 0° C. under N₂. The mixture was stirred at 25° C. for 1 h. Upon completion of the reaction, the reaction mixture was filtered and concentrated under reduced pressure to give product (35 g, crude). MS (ESI) m/z [M+H]⁺=232.

### Step 6: tert-butyl 3-(5-chloro-2-fluoro-4-hydroxyphenyl)morpholine-4-carboxylate

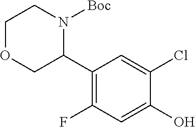 [SMILES: CC(C)(C)OC(=O)N1CCOCC1c1cc(Cl)c(O)cc1F]

The 2-chloro-5-fluoro-4-morpholin-3-yl-phenol (35 g, 151.1 mmol) was transferred to the reaction flask, and a solution of NH₃/MeOH (20 mL) was added dropwise at 0° C. The mixture was then stirred at 20° C. for 30 minutes. After that, the mixture was concentrated under reduced pressure. The mixture was added Boc₂O (49.4 g, 226.6 mmol, 52.0 mL) in EtOH (40 mL), and stirred at 80° C. for 1 hr. Upon completion of the reaction, the mixture was filtered and concentrated under reduced pressure to give a residue. The residue was purified by silica gel column chromatography (n-hexane: EA=20/1 to 5/1) to give the product (40 g, 79% yield). MS (ESI) m/z [M+H]⁺=332. ¹H NMR (400 MHz, DMSO-d6) δ=10.67 (s, 1H), 7.38 (d, J=8.0 Hz, 1H), 6.77 (d, J=12.0 Hz, 1H), 5.03 (d, J=2.8 Hz, 1H), 4.05-3.99 (m, 1H), 3.93-3.86 (m, 1H), 3.79-3.66 (m, 2H), 3.47 (dt, J=3.2, 11.6 Hz, 1H), 3.21-3.07 (m, 1H), 1.35 (s, 9H).

### Step 7: tert-butyl 3-(5-chloro-2-fluoro-4-hydroxy-3-iodophenyl)morpholine-4-carboxylate

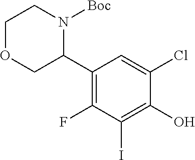 [SMILES: CC(C)(C)OC(=O)N1CCOCC1c1cc(Cl)c(O)c(I)c1F]

To a solution of tert-butyl 3-(5-chloro-2-fluoro-4-hydroxy-phenyl)morpholine-4-carboxylate (36 g, 108.5 mmol) in DCM (400 mL) was added NIS (24.41 g, 108.5 mmol). The resulting mixture was stirred at 25° C. for 1 hr. Upon completion of the reaction, the reaction mixture was filtered and concentrated under reduced pressure to give a residue. The residue was purified by silica gel column chromatography (n-hexane: EA=20/1 to 5/1) to give the title compound (30 g, 42% yield, 70% purity). MS (ESI) m/z [M+H]⁺=458. ¹H NMR (400 MHz, DMSO-d₆) δ=10.48 (s, 1H), 7.46 (d, J=8.0 Hz, 1H), 5.06 (d, J=2.4 Hz, 1H), 4.06-3.99 (m, 1H), 3.91 (dd, J=2.8, 11.4 Hz, 1H), 3.78-3.66 (m, 2H), 3.54-3.46 (m, 1H), 3.25-3.13 (m, 1H), 1.35 (s, 9H).

### Step 8: tert-butyl 3-(5-chloro-2-fluoro-4-hydroxy-3-((trimethylsilyl)ethynyl)phenyl) morpholine-4-carboxylate

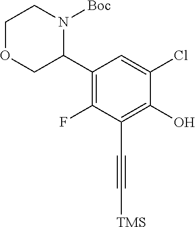 [SMILES: CC(C)(C)OC(=O)N1CCOCC1c1cc(Cl)c(O)c(C#C[Si](C)(C)C)c1F]

To a solution of tert-butyl 3-(5-chloro-2-fluoro-4-hydroxy-3-iodo-phenyl)morpholine-4-carboxylate (20 g, 43.7 mmol) and ethynyl(trimethyl)silane (8.6 g, 87.4 mmol, 12.1 mL) in dioxane (200 mL) was added TEA (13.3 g, 131.1 mmol, 18.3 mL), CuI (1.7 g, 8.7 mmol) and dichloropalladium triphenylphosphane (3.0 g, 4.4 mmol) at 25° C. under N₂. The resulting solution was stirred at 60° C. for 7 h. After cooled to rt, the mixture was filtered through celite. The filtrate was concentrated under reduced pressure to give the crude product (12 g).

### Step 9: tert-butyl 3-(7-chloro-4-fluoro-2-(trimethylsilyl)benzofuran-5-yl)morpholine-4-carboxylate

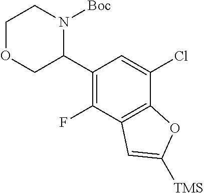 [SMILES: CC(C)(C)OC(=O)N1CCOCC1c1cc(Cl)c2oc([Si](C)(C)C)cc2c1F]

To a solution of tert-butyl 3-[5-chloro-2-fluoro-4-hydroxy-3-(2-trimethylsilylethynyl)phenyl]morpholine-4-carboxylate (12.5 g, 29.2 mmol) in dioxane (40 mL) were added CuI (1.1 g, 5.8 mmol), dichloropalladium triphenylphosphane (2.0 g, 2.9 mmol) and TEA (8.8 g, 87.6 mmol) at 25° C. under N₂. The solution was stirred at 90° C. for 12 hr. The mixture was filtered through celite, and the filtrate was concentrated under reduced pressure to give the product (8 g, 64% yield).

### Step 10: tert-butyl 3-(7-chloro-4-fluorobenzofuran-5-yl)morpholine-4-carboxylate

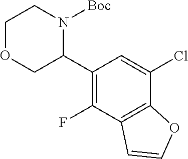 [SMILES: CC(C)(C)OC(=O)N1CCOCC1c1cc(Cl)c2occc2c1F]

To a solution of tert-butyl 3-(7-chloro-4-fluoro-2-trimethylsilyl-benzofuran-5-yl)morpholine-4-carboxylate (8 g, 18.7 mmol) in MeOH (100 mL) was added KF (2.2 g, 37.4 mmol) at 25° C. under N₂. The solution was stirred at 60° C. for 12 hr. After cooled to rt, the filtrate was concentrated in vacuo to give a residue. The residue was purified by column chromatography (n-hexane: EA=10/1 to 5/1) to give the product (2.7 g, 39% yield). MS (ESI) m/z [M+H]*=356. ¹H NMR (400 MHz, DMSO-d₆) δ=8.18 (d, J=2.0 Hz, 1H), 7.52 (d, J=6.0 Hz, 1H), 7.21 (d, J=2.0 Hz, 1H), 5.25 (d, J=2.4 Hz, 1H), 4.09 (d, J=12.0 Hz, 1H), 3.94 (dd, J=2.8, 11.2 Hz, 1H), 3.83 (dd, J=4.4, 12.4 Hz, 1H), 3.71 (dd, J=2.0, 13.6 Hz, 1H), 3.54 (dt, J=3.6, 11.6 Hz, 1H), 3.31-3.22 (m, 1H), 1.33 (s, 9H).

### Step 11: (5-(4-(tert-butoxycarbonyl)morpholin-3-yl)-7-chloro-4-fluorobenzofuran-2-yl)boronic Acid

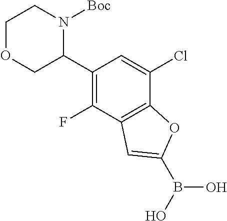 [SMILES: CC(C)(C)OC(=O)N1CCOCC1c1cc(Cl)c2oc(B(O)O)cc2c1F]

A solution of tert-butyl 3-(7-chloro-4-fluoro-benzofuran-5-yl)morpholine-4-carboxylate (200 mg, 0.56 mmol), bis(1,5-cyclooctadiene)dimethoxydiiridium (37 mg, 0.056 mmol), 4,4′-di-tert-butyl-2,2′-dipyridyl (30 mg, 0.11 mmol) and 4,4,5,5-tetramethyl-1,3,2-dioxaborolane (358 mg, 2.80 mmol) in THF (10 mL) was stirred at rt overnight under N₂. Upon completion of the reaction, the solvent was removed and the residue was used in next step without purification. MS (ESI) m/z [M+H]⁺=400.

### Step 12: tert-butyl 3-(7-chloro-4-fluoro-2-(1-(tetrahydro-2H-pyran-2-yl)-1H-1,2,4-triazol-3-yl)benzofuran-5-yl)morpholine-4-carboxylate (Assumed THP Position)

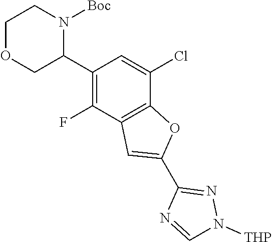

A solution of [5-(4-tert-butoxycarbonylmorpholin-3-yl)-7-chloro-4-fluoro-benzofuran-2-yl]boronic acid (200 mg, 0.50 mmol), 3-bromo-4-tetrahydropyran-2-yl-1,2,4-triazole (130 mg, 0.56 mmol), Xphos Pd G₄ (48.2 mg, 0.056 mmol) and K₃PO₄ (237.4 mg, 1.12 mmol) in dioxane (12 mL) and H₂O (3 mL) was stirred at 85° C. for 2 h under N₂. Upon completion of the reaction, the solvent was removed. The residue was purified by silica gel column chromatography to give the product (250 mg, 90% yield). MS (ESI) m/z [M+H]⁺=507.

### Step 13: 3-(7-chloro-4-fluoro-2-(1H-1,2,4-triazol-3-yl)benzofuran-5-yl)morpholine

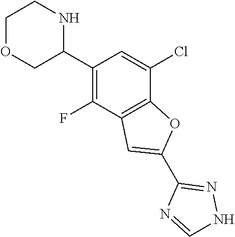 [SMILES: Fc1c(C2COCCN2)cc(Cl)c2oc(-c3nc[nH]n3)cc12]

A solution of tert-butyl 3-[7-chloro-4-fluoro-2-[4-(2-trimethylsilylethoxymethyl)-1,2,4-triazol-3-yl]benzofuran-5-yl]morpholine-4-carboxylate (250 mg, 0.45 mmol) in TFA (5 mL) and DCM (20 mL) was stirred at rt for 2 h. The solvent was removed, and the crude product was used for next step without purification. MS (ESI) m/z [M+H]⁺=323.

### Step 14: 1-(3-(7-chloro-4-fluoro-2-(4H-1,2,4-triazol-3-yl)benzofuran-5-yl)morpholino)prop-2-en-1-one

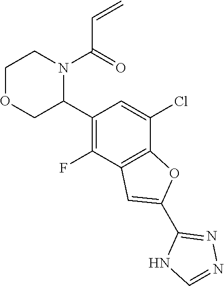 [SMILES: C=CC(=O)N1CCOCC1c1cc(Cl)c2oc(-c3nnc[nH]3)cc2c1F]

To a solution of 3-[7-chloro-4-fluoro-2-(4H-1,2,4-triazol-3-yl)benzofuran-5-yl]morpholine (140 mg, 0.43 mmol, TFA salt) and NaHCO₃ (100 mg, 1.19 mmol) in MeOH (10 mL) and H₂O (10 mL) was added prop-2-enoyl chloride (50 mg, 0.55 mmol) dropwise at 0° C. The mixture was stirred at 0° C. for 1 h. Upon completion of the reaction, the mixture was extracted with DCM. The organic layer was dried over Na₂SO₄, filtered and concentrated. The residue was purified by Prep-HPLC (column: Sunfire C18 (19×150 mm, 5 m); mobile phase: [H₂O (0.1% FA)-ACN]; gradient: 27%-43% B over 15.0 min) to give the racemic compound (24 mg, 0.064 mmol, 15% yield). MS (ESI) m/z [M+H]⁺=377.

### Step 15: Chiral separation of (R)-1-(3-(7-chloro-4-fluoro-2-(4H-1,2,4-triazol-3-yl)benzofuran-5-yl)morpholino)prop-2-en-1-one (Assumed) and (S)-1-(3-(7-chloro-4-fluoro-2-(4H-1,2,4-triazol-3-yl)benzofuran-5-yl)morpholino)prop-2-en-1-one (Assumed)

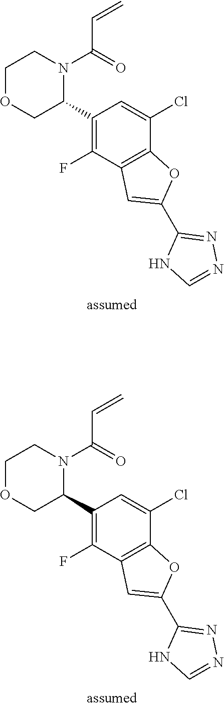 [SMILES: C=CC(=O)N1CCOC[C@H]1c1cc(Cl)c2oc(-c3nnc[nH]3)cc2c1F ; C=CC(=O)N1CCOC[C@@H]1c1cc(Cl)c2oc(-c3nnc[nH]3)cc2c1F]

The racemic 1-(3-(7-chloro-4-fluoro-2-(4H-1,2,4-triazol-3-yl)benzofuran-5-yl)morpholino)prop-2-en-1-one was separated by SFC: Column: CHIRALPAK IH; Eluent: 25% MeOH (0.3% 2M NH₃ MeOH)/CO2, flow rate: 40.0 mL/min.

Isomer 1: retention time=4.88 min. (6.89 mg, 28.7% Yield).

¹H NMR (600 MHz, DMSO-d6) δ 14.55 (s, 1H), 8.75 (s, 1H), 7.65 (d, J=5.3 Hz, 1H), 7.56 (s, 1H), 6.98-6.79 (m, 1H), 6.19 (d, J=16.3 Hz, 1H), 5.82-5.58 (m, 2H), 4.29 (d, J=11.4 Hz, 1H), 4.19-3.93 (m, 2H), 3.85 (d, J=9.7 Hz, 1H), 3.58-3.02 (m, 2H). MS (ESI) m/z [M+H]⁺=377.

Isomer 2: retention time=6.17 min; (6.52 mg, 27% yield).

¹H NMR (600 MHz, DMSO-d6) δ 14.49 (s, 1H), 8.73 (s, 1H), 7.65 (d, J=5.3 Hz, 1H), 7.56 (s, 1H), 6.93-6.79 (m, 1H), 6.19 (d, J=16.7 Hz, 1H), 5.82-5.61 (m, 2H), 4.29 (d, J=12.4 Hz, 1H), 4.18-3.91 (m, 2H), 3.85 (d, J=11.7 Hz, 1H), 3.58-3.02 (m, 2H). MS (ESI) m/z [M+H]⁺=377.

### Example 2-23: Synthesis of (R)-1-(3-(7-chloro-4-cyclopropyl-2-(1H-imidazol-4-yl)benzo[d]oxazol-5-yl)morpholino)prop-2-en-1-one (D24A, Assumed) and (S)-1-(3-(7-chloro-4-cyclopropyl-2-(1H-imidazol-4-yl)benzo[d]oxazol-5-yl)morpholino)prop-2-en-1-one (D24B, Assumed)

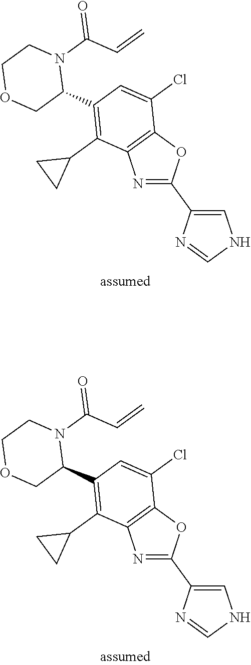 [SMILES: C=CC(=O)N1CCOC[C@H]1c1cc(Cl)c2oc(-c3c[nH]cn3)nc2c1C1CC1 ; C=CC(=O)N1CCOC[C@@H]1c1cc(Cl)c2oc(-c3c[nH]cn3)nc2c1C1CC1]

### Step 1: 1-chloro-4-cyclopropyl-2-methoxy-benzene

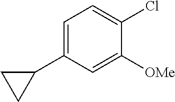 [SMILES: COc1cc(C2CC2)ccc1Cl]

A mixture of 4-bromo-1-chloro-2-methoxy-benzene (10 g, 45.15 mmol), cyclopropylboronic acid (5.82 g, 67.72 mmol), Pd(OAc)₂ (1.01 g, 4.51 mmol), PCy₃ (1.26 g, 4.52 mmol) and Cs₂CO₃ (29.44 g, 90.30 mmol) in toluene (100 mL) and H₂O (30 mL) was stirred at 85° C. for 16 h under nitrogen atmosphere. After cooled to rt, the mixture was diluted with water (100 mL). The resulting mixture was extracted with DCM. The combined organic layers were dried over anhydrous Na₂SO₄. After filtration, the filtrate was concentrated under reduced pressure. The residue was purified by silica gel column chromatography (10%-40% EA in PE) to give the product (7 g, 85% yield).

### Step 2: 1-bromo-5-chloro-2-cyclopropyl-4-methoxy-benzene

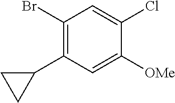 [SMILES: COc1cc(C2CC2)c(Br)cc1Cl]

To a stirred mixture of 1-chloro-4-cyclopropyl-2-methoxy-benzene (3.00 g, 16 mmol) in ACN (30 mL) was added NBS (2.9 g, 16 mmol) in portions at 0° C. The resulting mixture was stirred for 10 h at rt. Upon completion of the reaction, the mixture was concentrated in vacuo. The residue was purified by silica gel column chromatography (10%-60% EA in PE) to give the product (3.0 g, 70% yield).

### Step 3: 4-bromo-2-chloro-5-cyclopropyl-phenol

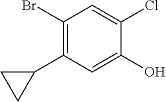 [SMILES: Oc1cc(C2CC2)c(Br)cc1Cl]

To a solution of 1-bromo-5-chloro-2-cyclopropyl-4-methoxy-benzene (1.5 g, 5.7 mmol) in DCM (15 mL) was added BBr₃ (8.6 mL, 8.6 mmol, 1 mol/L) dropwise at 0° C. The resulting mixture was stirred for 16 h at rt. Upon completion of the reaction, water was added to quench the reaction, and the resulting mixture was extracted with DCM. The combined organic layers were dried over anhydrous Na₂SO₄. After filtration, the filtrate was concentrated under reduced pressure. The residue was purified by silica gel column chromatography (10%-50% EA in PE) to the product (1.3 g, 92% yield).

### Step 4: 4-bromo-6-chloro-3-cyclopropyl-2-nitro-phenol

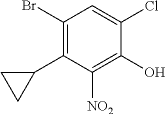 [SMILES: O=[N+]([O-])c1c(O)c(Cl)cc(Br)c1C1CC1]

To a stirred solution of 4-bromo-2-chloro-5-cyclopropyl-phenol (630 mg, 2.54 mmol) in Ac₂O (10 mL) was added HNO₃ (247 mg, 2.55 mmol, 65 mass %) dropwise at 0° C. The resulting mixture was stirred for 1 h at 0° C. Upon completion of the reaction, aq. NaHCO₃ was added to quench the reaction, and the resulting mixture was extracted with DCM. The combined organic layers were dried over anhydrous Na₂SO₄. After filtration, the filtrate was concentrated under reduced pressure to give the product (2 g, 2.05 mmol, crude).

### Step 5: 2-amino-4-bromo-6-chloro-3-cyclopropyl-phenol

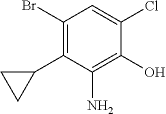 [SMILES: Nc1c(O)c(Cl)cc(Br)c1C1CC1]

A mixture of 4-bromo-6-chloro-3-cyclopropyl-2-nitro-phenol (2 g, 2.05 mmol, crude), Iron powder (574 mg, 10.25 mmol) and NH₄Cl (1.1 g, 21 mmol) in EtOH (20 mL)/H₂O (20 mL) was stirred at 80° C. for 2 h. After cooled to rt, the solid was filtrated off The filtrate was concentrated under reduced pressure. The residue was purified by silica gel column chromatography (10%-80% EA in PE) to give the product (300 mg, 56% yield). MS (ESI) m/z [M+H]⁺=262, 264.

### Step 6: 5-bromo-7-chloro-4-cyclopropyl-2-(1H-imidazol-4-yl)-1,3-benzoxazole

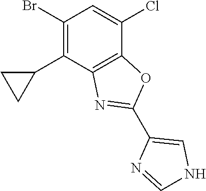 [SMILES: Clc1cc(Br)c(C2CC2)c2nc(-c3c[nH]cn3)oc12]

A mixture of 2-amino-4-bromo-6-chloro-3-cyclopropyl-phenol (300 mg, 1.14 mmol) and 4-(trifluoromethyl)-1H-imidazole (155 mg, 1.14 mmol) in aq. NaOH (10 mL, 5 mmol, 0.5 mol/L) was stirred at 80° C. for 16 h. After cooled to rt, the solid was collected by filtration to give the product (300 mg, 78% yield). MS (ESI) m/z [M+H]⁺=338, 340.

### Step 7: 2-[[4-(5-bromo-7-chloro-4-cyclopropyl-1,3-benzoxazol-2-yl)imidazol-1-yl]methoxy]ethyl-trimethyl-silane (Assumed SEM Position)

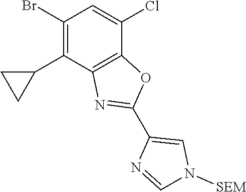 [SMILES: C[Si](C)(C)CCOCn1cnc(-c2nc3c(C4CC4)c(Br)cc(Cl)c3o2)c1]

To a solution of 5-bromo-7-chloro-4-cyclopropyl-2-(1H-imidazol-4-yl)-1,3-benzoxazole (270 mg, 0.79 mmol) in DMF (10 mL) was added NaH (48 mg, 1.2 mmol, 60% in mineral oil) portion wise at 0° C. The resulting mixture was stirred for 1 h at 0° C. To the above was added SEM-Cl (199 mg, 1.19 mmol) at 0° C., and the resulting mixture was stirred for 1 h at rt. Upon completion of the reaction, water was added to quench the reaction, and the resulting mixture was extracted with DCM. The combined organic layers were dried over anhydrous Na₂SO₄. After filtration, the filtrate was concentrated under reduced pressure. The residue was purified by silica gel column chromatography (10%-50% EA in PE) to give the product (330 mg, 88% yield). MS (ESI) m/z [M+H]⁺=468, 470.

### Step 8: 2-[[4-[7-chloro-4-cyclopropyl-5-(4,4,5,5-tetramethyl-1,3,2-dioxaborolan-2-yl)-1,3-benzoxazol-2-yl]imidazol-1-yl]methoxy]ethyl-trimethyl-silane (Assumed SEM Position)

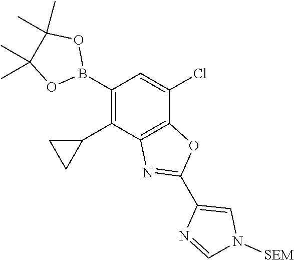 [SMILES: CC1(C)OB(c2cc(Cl)c3oc(-c4cn(COCC[Si](C)(C)C)cn4)nc3c2C2CC2)OC1(C)C]

A mixture of 2-[[4-(5-bromo-7-chloro-4-cyclopropyl-1,3-benzoxazol-2-yl)imidazol-1-yl]methoxy]ethyl-trimethyl-silane (270 mg, 0.58 mmol), 4,4,5,5-tetramethyl-2-(4,4,5,5-tetramethyl-1,3,2-dioxaborolan-2-yl)-1,3,2-dioxaborolane (219 mg, 0.86 mmol), Pd(dppf)Cl₂ (42 mg, 0.058 mmol) and KOAc (169 mg, 1.72 mmol) in 1,4-dioxane (15 mL) was stirred at 90° C. for 20 h under nitrogen atmosphere. After cooled to rt, the resulting mixture was concentrated under vacuum. The residue was purified by silica gel column chromatography (10%-80% EA in PE) to give the product (290 mg, 98% yield). MS (ESI) m/z [M+H]⁺=516.

### Step 9: tert-butyl 5-[7-chloro-4-cyclopropyl-2-[1-(2-trimethylsilylethoxymethyl) imidazol-4-yl]-1,3-benzoxazol-5-yl]-2,3-dihydro-1,4-oxazine-4-carboxylate (Assumed SEM Position)

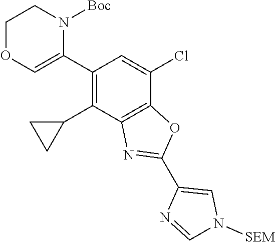 [SMILES: CC(C)(C)OC(=O)N1CCOC=C1c1cc(Cl)c2oc(-c3cn(COCC[Si](C)(C)C)cn3)nc2c1C1CC1]

A mixture of 2-[[4-[7-chloro-4-cyclopropyl-5-(4,4,5,5-tetramethyl-1,3,2-dioxaborolan-2-yl)-1,3-benzoxazol-2-yl]imidazol-1-yl]methoxy]ethyl-trimethyl-silane (260 mg, 0.50 mmol), tert-butyl 5-diphenoxyphosphoryloxy-2,3-dihydro-1,4-oxazine-4-carboxylate (218 mg, 0.50 mmol), Pd(dppf)Cl₂ (37 mg, 0.050 mmol) and K₂CO₃ (209 mg, 1.51 mmol) in 1,4-dioxane (15 mL) and H₂O (5 mL) was stirred at 90° C. for 2 h under nitrogen atmosphere. After cooled to rt, the resulting mixture was concentrated under vacuum. The residue was purified by silica gel column chromatography (10%-80% EA in PE) to give the product (120 mg, 42% yield). MS (ESI) m/z [M+H]⁺=573.

### Step 10: 7-chloro-4-cyclopropyl-2-(1H-imidazol-4-yl)-5-morpholin-3-yl-1,3-benzoxazole

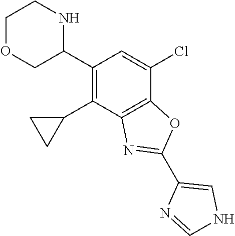 [SMILES: Clc1cc(C2COCCN2)c(C2CC2)c2nc(-c3c[nH]cn3)oc12]

To a stirred solution of tert-butyl 5-[7-chloro-4-cyclopropyl-2-[1-(2-trimethylsilylethoxymethyl)imidazol-4-yl]-1,3-benzoxazol-5-yl]-2,3-dihydro-1,4-oxazine-4-carboxylate (50 mg, 0.087 mmol) in HFIP (5 mL) was added MsOH (84 mg, 0.87 mmol) dropwise at rt and the resulting mixture was stirred for 1 h at rt. Upon completion of the reaction, to the above mixture was added MeOH (5 mL) and NaBH₃CN (9 mg, 0.15 mmol), and the mixture was stirred at rt for 1 h. Upon completion of the reaction, the solvent was concentrated under reduced pressure to give the product (40 mg, crude). MS (ESI) m/z [M+H]⁺=345.

### Step 11: 1-[3-[7-chloro-4-cyclopropyl-2-(1H-imidazol-4-yl)-1,3-benzoxazol-5-yl]morpholin-4-yl]prop-2-en-1-one

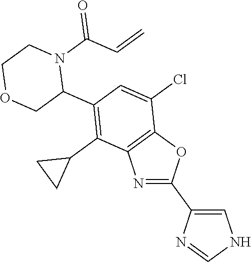 [SMILES: C=CC(=O)N1CCOCC1c1cc(Cl)c2oc(-c3c[nH]cn3)nc2c1C1CC1]

To a stirred mixture of 7-chloro-4-cyclopropyl-2-(1H-imidazol-4-yl)-5-morpholin-3-yl-1,3-benzoxazole (40 mg, 0.058 mmol, crude) in ACN (3 mL) and NaHCO₃ (3 mL, 2 mmol, 0.8 mol/L) was added prop-2-enoyl chloride (6 mg, 0.073 mmol) dropwise at rt. The resulting mixture was stirred at rt for 1 h. Upon completion of the reaction, water was added to quench the reaction and the resulting mixture was extracted with DCM. The combined organic layers were dried over anhydrous Na₂SO₄. After filtration, the filtrate was concentrated under reduced pressure. The residue was purified by Prep-HPLC (column: Sunfire C18 (19×150 mm, 5 m); mobile phase: [H₂O (0.1% FA)-ACN]; gradient: 20%-~40% B over 15.0 min) to give the product (4.1 mg, 18% yield). ¹H NMR (600 MHz, DMSO-d6) δ 12.84 (brs, 1H), 8.10 (s, 1H), 7.92 (s, 1H), 7.55 (s, 1H), 6.75 (s, 1H), 6.17-6.14 (m, 1H), 5.89-5.87 (m, 1H), 5.71 (d, J=9.5 Hz, 1H), 4.22 (d, J=11.9 Hz, 1H), 4.08-3.83 (m, 3H), 3.57-3.50 (m, 2H), 2.13-2.10 (m, 1H), 1.51-1.40 (m, 2H), 1.05-0.97 (m, 2H). MS (ESI) m/z [M+H]⁺=399.

Compounds D25-D27 in Table 2-8 below was synthesized by using corresponding starting materials according to procedures similar to those described for the synthesis of compounds D24.

TABLE 2-8

| | | | ¹H NMR data LC/MS m/z |
|---|---|---|---|
| Compound | Structure | Chemical Name | (M + H) |
| D25 | 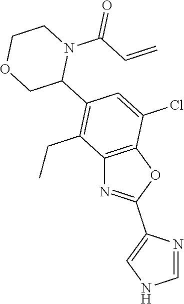 [SMILES: C=CC(=O)N1CCOCC1c1cc(Cl)c2oc(-c3c[nH]cn3)nc2c1CC] | 1-(3-(7-chloro-4-ethyl-2- (1H-imidazol-4- yl)benzo[d]oxazol-5- yl)morpholino)prop-2-en- 1-one | ¹H NMR (600 MHz, DMSO-d₆) δ 12.86 (s, 1H), 8.16 (s, 1H), 7.94 (s, 1H), 7.75 (s, 1H), 6.85-6.70 (m, 1H), 6.22-6.14 (m, 1H), 5.76- 6.66 (m, 2H), 4.25-4.15 (m, 1H), 4.05-3.98 (m, 1H), 3.97-3.79 (m, 2H), 3.58-3.50 (m, 1H), 3.46- 3.37 (m, 1H), 3.07-2.97 (m, 1H), 2.97-2.88 (m, 1H), 1.15 (t, J = 7.0 Hz, 3H). MS (ESI) m/z [M + H]⁺ = 387 |
| D26 | 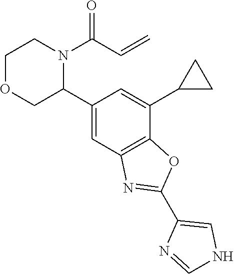 [SMILES: C=CC(=O)N1CCOCC1c1cc(C2CC2)c2oc(-c3c[nH]cn3)nc2c1] | 1-(3-(7-cyclopropyl-2-(1H- imidazol-4- yl)benzo[d]oxazol-5- yl)morpholino)prop-2-en- 1-one | ¹H NMR (600 MHz, DMSO-d₆) δ 12.85 (s, 1H), 8.05 (s, 1H), 7.98 (s, 1H), 7.43 (s, 1H), 6.98 (s, 1H), 6.90-6.80 (m, 1H), 6.21 (d, J = 16.6 Hz, 1H), 5.74 (d, J = 10.2 Hz, 1H), 5.65-5.25 (m, 1H), 4.48- 4.40 (m, 1H), 4.27-3.72 (m, 3H), 3.60-2.88 (m, 2H), 2.28-2.19 (m, 1H), 1.12-1.04 (m, 2H), 1.00- 0.88 (m, 2H). MS (ESI) m/z [M + H]⁺ = 365 |
| D27 | 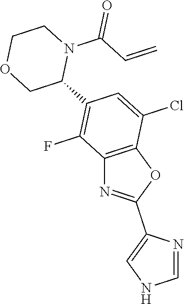 [SMILES: C=CC(=O)N1CCOC[C@H]1c1cc(Cl)c2oc(-c3c[nH]cn3)nc2c1F] | (R)-1-(3-(7-chloro-4- fluoro-2-(1H-imidazol-4- yl)benzo[d]oxazol-5- yl)morpholino)prop-2-en- 1-one (assumed) | ¹H NMR (600 MHz, DMSO-d₆) δ 12.93 (s, 1H), 8.23 (s, 1H), 7.96 (s, 1H), 7.59 (s, 1H), 6.95-6.80 (m, 1H), 6.22-6.15 (m, 1H), 5.78- 5.66 (m, 2H), 4.32-4.24 (m, 1H), 4.02-3.94 (m, 2H), 3.89-3.83 (m, 1H), 3.58-2.88 (m, 2H). MS (ESI) m/z [M + H]⁺ = 377 |

### Example 2-24: Synthesis of (S)-1-(7-(4-chloro-2-(1H-imidazol-4-yl)benzo[d]oxazol-6-yl)-5-oxa-8-azaspiro[3.5]nonan-8-yl)prop-2-en-1-one (L4A, Assumed) and (R)-1-(7-(4-chloro-2-(1H-imidazol-4-yl)benzo[d]oxazol-6-yl)-5-oxa-8-azaspiro[3.5]nonan-8-yl)prop-2-en-1-one (L4B, Assumed)

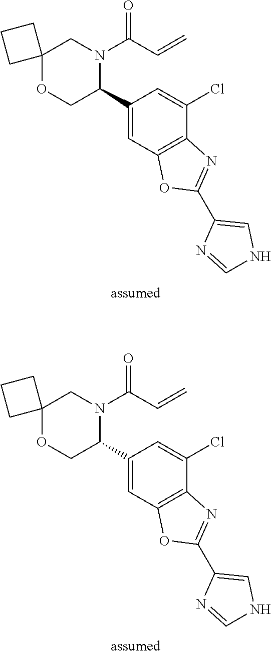 [SMILES: C=CC(=O)N1CC2(CCC2)OC[C@@H]1c1cc(Cl)c2nc(-c3c[nH]cn3)oc2c1 ; C=CC(=O)N1CC2(CCC2)OC[C@H]1c1cc(Cl)c2nc(-c3c[nH]cn3)oc2c1]

### Step 1: tert-butyl 7-[4-chloro-2-[1-(2-trimethylsilylethoxymethyl)imidazol-4-yl]-1,3-benzoxazol-6-yl]-5-oxa-8-azaspiro[3.5]non-6-ene-8-carboxylate (Assumed SEM Position)

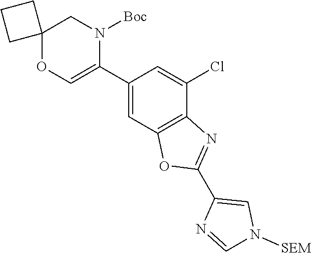 [SMILES: CC(C)(C)OC(=O)N1CC2(CCC2)OC=C1c1cc(Cl)c2nc(-c3cn(COCC[Si](C)(C)C)cn3)oc2c1]

A mixture of 2-[[4-[4-chloro-6-(4,4,5,5-tetramethyl-1,3,2-dioxaborolan-2-yl)-1,3-benzoxazol-2-yl]imidazol-1-yl]methoxy]ethyl-trimethyl-silane (500 mg, 1.05 mmol), tert-butyl 7-diphenoxyphosphoryloxy-5-oxa-8-azaspiro[3.5]non-6-ene-8-carboxylate (497 mg, 1.05 mmol), Pd(dtbpf)Cl₂ (89 mg, 0.10 mmol) and Na₂CO₃ (334 mg, 3.15 mmol) in 1,4-dioxane (15 mL) and H₂O (5 mL) was stirred at 90° C. for 2 h under nitrogen atmosphere. After cooled to rt, the resulting mixture was concentrated under vacuum. The residue was purified by flash column chromatography on silica gel eluted with 10%-80% EA in PE to give the product (300 mg, 50% yield). MS (ESI) m/z [M+H]⁺=573.

### Step 2: 4-chloro-2-(1H-imidazol-4-yl)-6-(5-oxa-8-azaspiro[3.5]nonan-7-yl)-1,3-benzoxazole

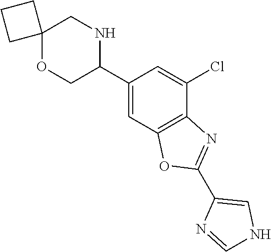 [SMILES: Clc1cc(C2COC3(CCC3)CN2)cc2oc(-c3c[nH]cn3)nc12]

To a solution of tert-butyl 7-[4-chloro-2-[1-(2-trimethylsilylethoxymethyl)imidazol-4-yl]-1,3-benzoxazol-6-yl]-5-oxa-8-azaspiro[3.5]non-6-ene-8-carboxylate (264 mg, 0.46 mmol) in HFIP (10 mL) was added MsOH (442 mg, 4.61 mmol) dropwise at rt. The resulting mixture was stirred for 1 h at rt. Then to the above was added MeOH (10 mL) and NaBH₃CN (54 mg, 0.88 mmol) at rt. The resulting mixture was stirred for another 1 h at rt and then upon completion of the reaction, the mixture was concentrated under reduced pressure to give the product (300 mg, crude). MS (ESI) m/z [M+H]⁺=345.

### Step 3: racemic 1-(7-(4-chloro-2-(1H-imidazol-4-yl)benzo[d]oxazol-6-yl)-5-oxa-8-azaspiro[3.5]nonan-8-yl)prop-2-en-1-one

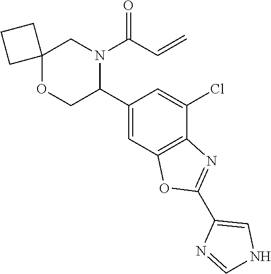 [SMILES: C=CC(=O)N1CC2(CCC2)OCC1c1cc(Cl)c2nc(-c3c[nH]cn3)oc2c1]

To a stirred mixture of 4-chloro-2-(1H-imidazol-4-yl)-6-(5-oxa-8-azaspiro[3.5]nonan-7-yl)-1,3-benzoxazole (300 mg, 0.44 mmol, crude) in ACN (10 mL) and NaHCO₃ (10 mL, 8 mmol, 0.8 mol/L) was added prop-2-enoyl chloride (39 mg, 0.44 mmol) dropwise at rt. The resulting mixture was stirred for 1 h at rt. Upon completion of the reaction, water was added to quench the reaction. The resulting mixture was extracted with DCM. The combined organic layers were dried over anhydrous Na₂SO₄. After filtration, the filtrate was concentrated under reduced pressure. The residue was purified by Prep-HPLC (column: Sunfire C18 (19×150 mm, 5 m); mobile phase: [H₂O (0.1% FA)-ACN]; gradient: 25%-40% B over 15.0 min) to give the product (50 mg).

### Step 4: Separation of (S)-1-(7-(4-chloro-2-(1H-imidazol-4-yl)benzo[d]oxazol-6-yl)-5-oxa-8-azaspiro[3.5]nonan-8-yl)prop-2-en-1-one (L4A, Assumed) and (R)-1-(7-(4-chloro-2-(1H-imidazol-4-yl)benzo[d]oxazol-6-yl)-5-oxa-8-azaspiro[3.5]nonan-8-yl)prop-2-en-1-one (L4B, Assumed)

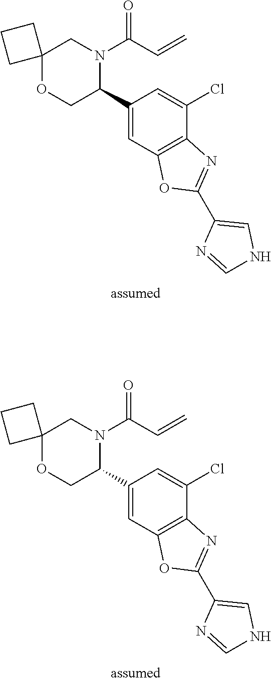 [SMILES: C=CC(=O)N1CC2(CCC2)OC[C@@H]1c1cc(Cl)c2nc(-c3c[nH]cn3)oc2c1 ; C=CC(=O)N1CC2(CCC2)OC[C@H]1c1cc(Cl)c2nc(-c3c[nH]cn3)oc2c1]

The racemic 1-(7-(4-chloro-2-(1H-imidazol-4-yl)benzo[d]oxazol-6-yl)-5-oxa-8-azaspiro[3.5]nonan-8-yl)prop-2-en-1-one was separated by SFC: column: Lux Cellulose-4, eluent: 40% MeOH (0.3% 2M NH₃ MeOH)/CO₂, flow rate: 90.0 mL/min.

Isomer 1: retention time=7.55 min. (13.67 mg, 8% yield) H NMR (600 MHz, DMSO-d6) δ 12.87 (brs, 1H), 8.16 (s, 1H), 7.95 (s, 1H), 7.70 (s, 1H), 7.40 (s, 1H), 6.96 (s, 1H), 6.29-6.26 (m, 1H), 5.81-5.79 (m, 1H), 5.61-5.32 (m, 1H), 4.32-4.30 (m, 1H), 4.05-3.81 (m, 2H), 3.04-2.75 (m, 1H), 2.01-1.92 (m, 3H), 1.90-1.87 (m, 1H), 1.76-1.74 (m, 2H). MS (ESI) m/z [M+H]⁺=399.

isomer 2: retention time=9.90 min; (15.91 mg, 9% Yield). ¹H NMR (600 MHz, DMSO-d6) δ 12.86 (brs, 1H), 8.17 (s, 1H), 7.95 (s, 1H), 7.70 (s, 1H), 7.40 (s, 1H), 6.95 (s, 1H), 6.29-6.26 (m, 1H), 5.81-5.79 (m, 1H), 5.61-5.32 (m, 1H), 4.32-4.30 (m, 1H), 4.06-3.81 (m, 2H), 3.12-3.04 (m, 1H), 1.96-1.92 (m, 3H), 1.90-1.87 (m, 1H), 1.76-1.74 (m, 2H). MS (ESI) m/z [M+H]⁺=399.

Compounds L3 and L10 in Table 2-9 below was synthesized by using corresponding starting materials according to procedures similar to those described for the synthesis of compounds L4.

TABLE 2-9

| Compound | Structure | Chemical Name | ¹H NMR data LC/MS m/z (M + H) |
|---|---|---|---|
| L3 | 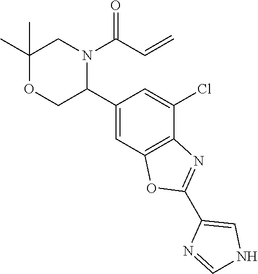 [SMILES: C=CC(=O)N1CC(C)(C)OCC1c1cc(Cl)c2nc(-c3c[nH]cn3)oc2c1] | 1-(5-(4-chloro-2-(1H- imidazol-4- yl)benzo[d]oxazol-6-yl)- 2,2- dimethylmorpholino) prop-2-en-1-one | ¹H NMR (600 MHz, DMSO-d₆) δ 12.86 (s, 1H), 8.17 (s, 1H), 7.95 (s, 1H), 7.71 (s, 1H), 7.41 (s, 1H), 6.94-6.70 (m, 1H), 6.23 (d, J = 16.5 Hz, 1H), 5.75 (s, 1H), 5.64-5.28 (m, 1H), 4.34-3.78 (m, 3H), 3.21-2.79 (m, 1H), 1.17 (s, 3H), 1.15 (s, 3H). MS (ESI) m/z [M + H]⁺ = 387. |
| L10 | 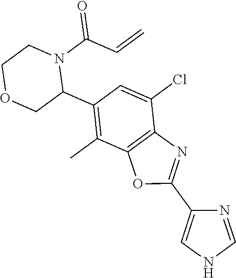 [SMILES: C=CC(=O)N1CCOCC1c1cc(Cl)c2nc(-c3c[nH]cn3)oc2c1C] | 1-(3-(4-chloro-2-(1H- imidazol-4-yl)-7- methylbenzo[d]oxazol-6- yl)morpholino)prop-2-en- 1-one | ¹H NMR (600 MHz, DMSO-d₆) δ 12.86 (s, 1H), 8.18 (s, 1H), 7.93 (s, 1H), 7.71 (s, 1H), 6.75 (s, 1H), 6.17 (d, J = 16.7 Hz, 1H), 5.72 (d, J = 10.2 Hz, 1H), 5.64 (s, 1H), 4.11-4.23 (m, 1H), 3.88- 4.08 (m, 2H), 3.78-3.87 (m, 1H), 3.54 (t, J = 10.6 Hz, 1H), 3.41 (t, J = 12.0 Hz, 1H), 2.46 (s, 3H). MS (ESI) m/z [M + H]⁺ = 373. |

### Example 2-25: Synthesis of (R)-1-(7-(4-chloro-2-(1H-imidazol-4-yl)-7-methoxybenzo[d]oxazol-6-yl)-5-oxa-8-azaspiro[3.5]nonan-8-yl)prop-2-en-1-one (L5A, Assumed) and (S)-1-(7-(4-chloro-2-(1H-imidazol-4-yl)-7-methoxybenzo[d]oxazol-6-yl)-5-oxa-8-azaspiro[3.5]nonan-8-yl)prop-2-en-1-one (L5B, Assumed)

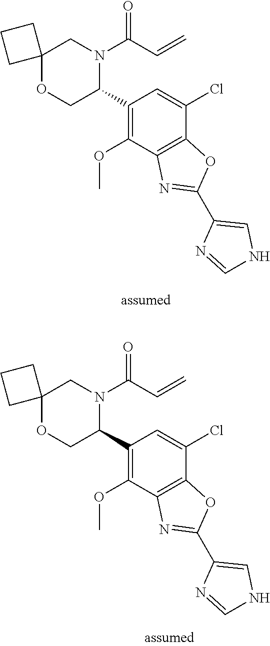 [SMILES: C=CC(=O)N1CC2(CCC2)OC[C@H]1c1cc(Cl)c2oc(-c3c[nH]cn3)nc2c1OC ; C=CC(=O)N1CC2(CCC2)OC[C@@H]1c1cc(Cl)c2oc(-c3c[nH]cn3)nc2c1OC]

### Step 1: 3-bromo-5-chloro-2-methoxy-benzaldehyde

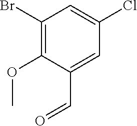

To a stirred mixture of 3-bromo-5-chloro-2-hydroxy-benzaldehyde (40 g, 169.87 mmol) and K₂CO₃ (70 g, 507.25 mmol) in DMF (400 mL) was added Mel (29 g, 204.22 mmol) dropwise at 0° C. The resulting mixture was stirred at rt for 16 h. Upon completion of the reaction, water was added to quench the reaction. The resulting mixture was extracted with DCM and the combined organic layers were dried over anhydrous Na₂SO₄. After filtration, the filtrate was concentrated under reduced pressure to give the product (40 g, 94% yield).

### Step 2: 3-bromo-5-chloro-2-methoxy-phenol

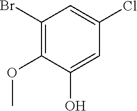

To a stirred mixture of H₂O₂ (55 g, 485.29 mmol, 30 mass %) in DCM (500 mL) was added TFAA (337 g, 1604.76 mmol) dropwise at 0° C. The resulting mixture was stirred at 0° C. for 1 h. The above solution was added to 3-bromo-5-chloro-2-methoxy-benzaldehyde (40 g, 160.32 mmol) in DCM (500 mL) dropwise at 0° C. The resulting mixture was stirred at 0° C. for 1 h. Upon completion of the reaction, NaHSO₃ (500 mL, sat) was added to quench the reaction, and the resulting mixture was extracted with DCM. The combined organic layers were dried over anhydrous Na₂SO₄. After filtration, the filtrate was concentrated under reduced pressure. The residue was re-dissolved in MeOH (300 mL), and 50 drops HCl (Con.) was added. The resulting mixture was stirred for 1 h at RT. The resulting mixture was concentrated under vacuum, and purified by silica gel column chromatography (10%-80% EA in PE) to give the product (35 g, 9w % yield).

### Step 3: 3-bromo-5-chloro-2-methoxy-6-nitro-phenol

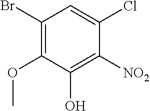

To a stirred mixture of 3-bromo-5-chloro-2-methoxy-phenol (15 g, 63.16 mmol) in Ac₂O (100 mL) was added HNO₃ (6.1 g, 63 mmol, 65 mass %) dropwise at 0° C. The resulting mixture was stirred for at 0° C. 1 h. Upon completion of the reaction, NaHCO₃ (100 mL, sat) was added to quench the reaction. The resulting mixture was extracted with DCM and the combined organic layers were dried over anhydrous Na₂SO₄. After filtration, the filtrate was concentrated under reduced pressure to give the product (20 g, crude).

### Step 4: 2-amino-5-bromo-3-chloro-6-methoxy-phenol

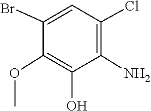

A mixture of 3-bromo-5-chloro-2-methoxy-6-nitro-phenol (20 g, 21.24 mmol), Iron powder (6 g, 107.14 mmol) and NH₄Cl (11 g, 207.54 mmol) in EtOH (40 mL) and H₂O (20 mL) was stirred at 80° C. for 2 h. After cooled to rt, the solid was filtered out. The filtrate was concentrated under reduced pressure and the residue was purified by silica gel column chromatography (10%-70% EA in PE) to give the product (5 g, 93% yield). MS (ESI) m/z [M+H]⁺=252, 254.

### Step 5: 6-bromo-4-chloro-2-(1H-imidazol-4-yl)-7-methoxy-1,3-benzoxazole

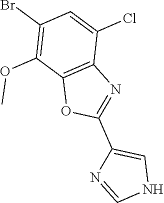 [SMILES: COc1c(Br)cc(Cl)c2nc(-c3c[nH]cn3)oc12]

A mixture of 2-amino-5-bromo-3-chloro-6-methoxy-phenol (4.5 g, 18 mmol) and 4-(trifluoromethyl)-1H-imidazole (2.4 g, 18 mmol) in aq. NaOH (100 mL, 50 mmol, 0.5 mol/L) was stirred at 80° C. for 16 h. After cooled to rt, the solid was collected by filtration to give the product (4.5 g, 77% yield). MS (ESI) m/z [M+H]⁺=328, 330.

### Step 6: 2-[[4-(6-bromo-4-chloro-7-methoxy-1,3-benzoxazol-2-yl)imidazol-1-yl]methoxy]ethyl-trimethyl-silane (Assumed SEM Position)

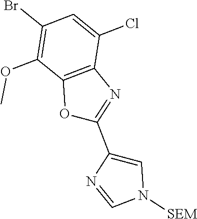 [SMILES: COc1c(Br)cc(Cl)c2nc(-c3cn(COCC[Si](C)(C)C)cn3)oc12]

To a stirred solution of 6-bromo-4-chloro-2-(1H-imidazol-4-yl)-7-methoxy-1,3-benzoxazole (1 g, 3.04 mmol) in DMF (10 mL) was added NaH (183 mg, 4.58 mmol, 60% in mineral oil) portion wise at 0° C. The resulting mixture was stirred at 0° C. for 1 h. Then to the above mixture was added SEM-Cl (758 mg, 4.57 mmol) dropwise at 0° C. The resulting mixture was stirred at rt for 1 h. Upon completion of the reaction, water (50 mL) was added to quench the reaction. The resulting mixture was extracted with DCM and the combined organic layers were dried over anhydrous Na₂SO₄. After filtration, the filtrate was concentrated under reduced pressure. The residue was purified by silica gel column chromatography (10%-50% EA in PE) to give the product (1 g, 72% yield). MS (ESI) m/z [M+H]⁺=458, 460.

### Step 7: 2-[[4-[4-chloro-7-methoxy-6-(4,4,5,5-tetramethyl-1,3,2-dioxaborolan-2-yl)-1,3-benzoxazol-2-yl]imidazol-1-yl]methoxy]ethyl-trimethyl-silane (Assumed SEM Position)

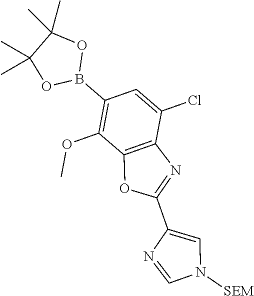 [SMILES: COc1c(B2OC(C)(C)C(C)(C)O2)cc(Cl)c2nc(-c3cn(COCC[Si](C)(C)C)cn3)oc12]

A mixture of 2-[[4-(6-bromo-4-chloro-7-methoxy-1,3-benzoxazol-2-yl)imidazol-1-yl]methoxy]ethyl-trimethyl-silane (1.4 g, 3.1 mmol), 4,4,5,5-tetramethyl-2-(4,4,5,5-tetramethyl-1,3,2-dioxaborolan-2-yl)-1,3,2-dioxaborolane (1.2 g, 4.7 mmol), Pd(dppf)Cl₂ (220 mg, 0.30 mmol) and KOAc (900 mg, 9.18 mmol) in 1,4-dioxane (15 mL) was stirred at 90° C. for 18 h under nitrogen atmosphere. After cooled to rt, the resulting mixture was concentrated under vacuum. The residue was purified by silica gel column chromatography (10%-80% EA in PE) to give the product (1 g, 65% yield).

### Step 8: tert-butyl 7-[4-chloro-7-methoxy-2-[1-(2-trimethylsilylethoxymethyl)imidazol-4-yl]-1,3-benzoxazol-6-yl]-5-oxa-8-azaspiro[3.5]non-6-ene-8-carboxylate (Assumed SEM Position)

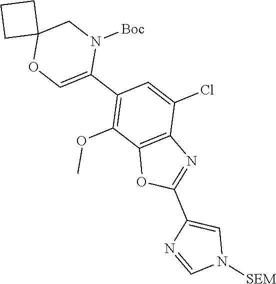 [SMILES: COc1c(C2=COC3(CCC3)CN2C(=O)OC(C)(C)C)cc(Cl)c2nc(-c3cn(COCC[Si](C)(C)C)cn3)oc12]

A mixture of 2-[[4-[4-chloro-7-methoxy-6-(4,4,5,5-tetramethyl-1,3,2-dioxaborolan-2-yl)-1,3-benzoxazol-2-yl]imidazol-1-yl]methoxy]ethyl-trimethyl-silane (400 mg, 0.79 mmol), tert-butyl 7-diphenoxyphosphoryloxy-5-oxa-8-azaspiro[3.5]non-6-ene-8-carboxylate (374 mg, 0.79 mmol), Pd(dppf)Cl₂ (58 mg, 0.079 mmol) and K₂CO₃ (327 mg, 2.37 mmol) in 1,4-dioxane (15 mL) and H₂O (5 mL) was stirred at 90° C. for 2 h under nitrogen atmosphere. After cooled to rt, the resulting mixture was concentrated under vacuum. The residue was purified by silica gel column chromatography (10%-80% EA in PE) to give the product (350 mg, 73% yield). MS (ESI) m/z [M+H]⁺=603.

### Step 9: 4-chloro-2-(1H-imidazol-4-yl)-7-methoxy-6-(5-oxa-8-azaspiro[3.5]nonan-7-yl)-1,3-benzoxazole

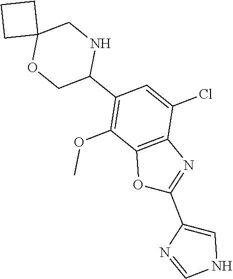 [SMILES: COc1c(C2COC3(CCC3)CN2)cc(Cl)c2nc(-c3c[nH]cn3)oc12]

To a stirred solution of tert-butyl 7-[4-chloro-7-methoxy-2-[1-(2-trimethylsilylethoxymethyl)imidazol-4-yl]-1,3-benzoxazol-6-yl]-5-oxa-8-azaspiro[3.5]non-6-ene-8-carboxylate (350 mg, 0.58 mmol) in HFIP (10 mL) was added MsOH (557 mg, 5.80 mmol) dropwise at rt. The resulting mixture was stirred at rt for 1 h. Upon completion of the reaction, to the above mixture was added MeOH (10 mL) and NaBH₃CN (58 mg, 0.94 mmol) at rt. The mixture was stirred for 1 h at rt. Upon completion of the reaction, the resulting mixture was concentrated under reduced pressure to give the product (300 mg, 0.40 mmol, crude). MS (ESI) m/z [M+H]⁺=375.

### Step 10: 1-[7-[4-chloro-2-(1H-imidazol-4-yl)-7-methoxy-1,3-benzoxazol-6-yl]-5-oxa-8-azaspiro[3.5]nonan-8-yl]prop-2-en-1-one

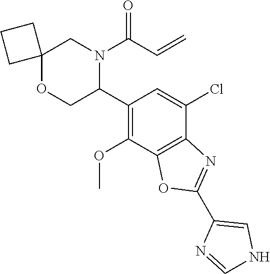 [SMILES: C=CC(=O)N1CC2(CCC2)OCC1c1cc(Cl)c2nc(-c3c[nH]cn3)oc2c1OC]

To a stirred solution of 4-chloro-2-(1H-imidazol-4-yl)-7-methoxy-6-(5-oxa-8-azaspiro[3.5]nonan-7-yl)-1,3-benzoxazole (300 mg, 0.40 mmol, crude) in ACN (5 mL) and NaHCO₃ (5 mL, 4 mmol, 0.8 mol/L) was added prop-2-enoyl chloride (36 mg, 0.40 mmol) dropwise at rt. The resulting mixture was stirred for 1 h at rt. Upon completion of the reaction, water was added and the resulting mixture was extracted with DCM. The combined organic layers were dried over anhydrous Na₂SO₄. After filtration, the filtrate was concentrated under reduced pressure. The residue was purified by Prep-HPLC (column: Sunfire C18 (19×150 mm, 5 μm); mobile phase: [H₂O (0.1% FA)-ACN]; gradient: 28%-48% B over 15.0 min) to give the product (100 mg, 58% yield).

### Step 11: Separation of (R)-1-(7-(4-chloro-2-(1H-imidazol-4-yl)-7-methoxybenzo[d]oxazol-6-yl)-5-oxa-8-azaspiro[3.5]nonan-8-yl)prop-2-en-1-one (Assumed) and (S)-1-(7-(4-chloro-2-(1H-imidazol-4-yl)-7-methoxybenzo[d]oxazol-6-yl)-5-oxa-8-azaspiro[3.5]nonan-8-yl)prop-2-en-1-one (Assumed)

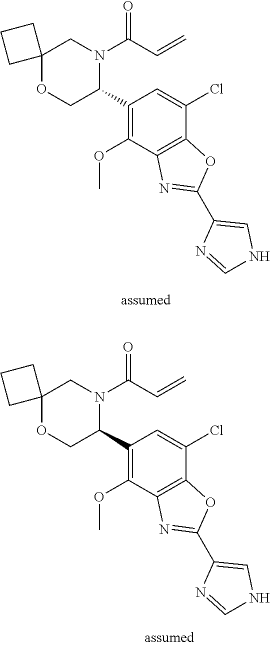 [SMILES: C=CC(=O)N1CC2(CCC2)OC[C@H]1c1cc(Cl)c2oc(-c3c[nH]cn3)nc2c1OC ; C=CC(=O)N1CC2(CCC2)OC[C@@H]1c1cc(Cl)c2oc(-c3c[nH]cn3)nc2c1OC]

The racemate 1-(7-(4-chloro-2-(1H-imidazol-4-yl)-7-methoxybenzo[d]oxazol-6-yl)-5-oxa-8-azaspiro[3.5]nonan-8-yl)prop-2-en-1-one was separated by SFC: Column: Lux Cellulose-4; Eluent: 45% MeOH (0.3% 2M NH₃ MeOH)/CO2, flow rate: 90.0 mL/min.

Isomer 1: retention time=4.70 min;

¹H NMR (600 MHz, DMSO-d₆) δ 12.88 (brs, 1H), 8.22 (s, 1H), 7.94 (s, 1H), 7.49 (s, 1H), 7.04-6.79 (m, 1H), 6.21-6.18 (m, 1H), 5.76-5.74 (m, 1H), 5.45-5.40 (m, 1H), 4.49-4.47 (m, 1H), 4.15 (s, 3H), 4.06-4.04 (m, 1H), 3.85-3.83 (m, 1H), 2.85-2.83 (m, 1H), 2.06-1.91 (m, 4H), 1.74-1.63 (m, 2H).

MS (ESI) m/z [M+H]⁺=429.

Isomer 2: retention time=6.42 min.

¹H NMR (600 MHz, DMSO-d₆) δ 12.89 (brs, 1H), 8.21 (s, 1H), 7.95 (s, 1H), 7.49 (s, 1H), 6.99-6.79 (m, 1H), 6.21-6.18 (m, 1H), 5.75 (d, J=9.9 Hz, 1H), 5.45-5.40 (m, 1H), 4.50-4.47 (m, 1H), 4.16 (s, 3H), 4.06-4.04 (m, 1H), 3.84-3.82 (m, 1H), 2.87-2.82 (m, 1H), 2.08-1.89 (s, 4H), 1.74-1.64 (m, 2H).

MS (ESI) m/z [M+H]⁺=429.

Compounds L6-L9 in Table 2-10 below were synthesized by using corresponding starting materials according to procedures similar to those described for the synthesis of compounds L5.

TABLE 2-10

| Compound | Structure | Chemical Name | ¹H NMR data LC/MS m/z (M + H) |
|---|---|---|---|
| L6 | 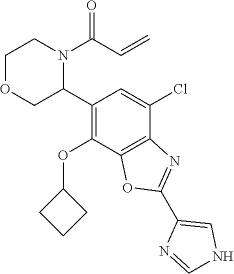 [SMILES: C=CC(=O)N1CCOCC1c1cc(Cl)c2nc(-c3c[nH]cn3)oc2c1OC1CCC1] | 1-(3-(4-chloro-7- cyclobutoxy-2-(1H-imidazol- 4-yl)benzo[d]oxazol-6- yl)morpholino)prop-2-en-1- one | ¹H NMR (600 MHz, DMSO-d6) δ 12.89 (s, 1H), 8.21 (s, 1H), 7.95 (s, 1H), 7.44 (s, 1H), 6.88-6.83 (m, 1H), 6.19-6.17 (m, 1H), 5.73 (d, J = 10.3 Hz, 1H), 5.67-5.38 (m, 1H), 5.26-5.24 (m, 1H), 4.22 (d, J = 12.0 Hz, 2H), 3.98-3.96 (m, 1H), 3.85- 3.83 (m, 1H), 3.53-3.50 (m, 1H), 3.29-2.71 (m, 1H), 2.40-2.37 (m, 2H), 2.28-2.12 (m, 2H), 1.78-1.76 (m, 1H), 1.64-1.59 (m, 1H). MS (ESI) m/z [M + H]⁺ = 429. |
| L7 | 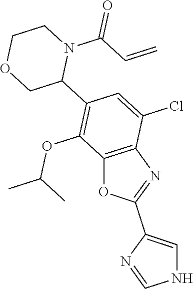 [SMILES: C=CC(=O)N1CCOCC1c1cc(Cl)c2nc(-c3c[nH]cn3)oc2c1OC(C)C] | 1-(3-(4-chloro-2-(1H- imidazol-4-yl)-7- isopropoxybenzo[d]oxazol-6- yl)morpholino)prop-2-en-1- one | ¹H NMR (600 MHz, DMSO-d6) δ 12.88 (brs, 1H), 8.20 (s, 1H), 7.95 (s, 1H), 7.44 (s, 1H), 6.86-6.81 (m, 1H), 6.19-6.16 (m, 1H), 5.73 (d, J = 10.1 Hz, 1H), 5.66-5.33 (m, 1H), 5.24-5.22 (m, 1H), 4.21 (d, J = 11.9 Hz, 1H), 3.98-3.96 (m, 1H), 3.83- 3.82 (m, 1H), 3.53-3.49 (m, 2H), 3.27-3.03 (m, 1H), 1.40-1.20 (m, 6H). MS (ESI) m/z [M + H]⁺ = 417. |
| L8 | 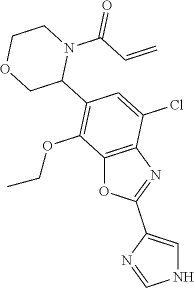 [SMILES: C=CC(=O)N1CCOCC1c1cc(Cl)c2nc(-c3c[nH]cn3)oc2c1OCC] | 1-(3-(4-chloro-7-ethoxy-2- (1H-imidazol-4- yl)benzo[d]oxazol-6- yl)morpholino)prop-2-en-1- one | ¹H NMR (600 MHz, DMSO-d6) δ 12.82 (brs, 1H), 8.21 (s, 1H), 7.95 (s, 1H), 7.44 (s, 1H), 6.87-6.83 (m, 1H), 6.18-6.15 (m, 1H), 5.72 (d, J = 9.5 Hz, 1H), 5.65-5.22 (m, 1H), 4.56-4.55 (m, 2H), 4.21 (d, J = 11.9 Hz, 2H), 3.99-3.82 (m, 3H), 3.53- 3.50 (m, 1H), 1.39-1.37 (m, 3H). MS (ESI) m/z [M + H]⁺ = 403. |
| L9 | 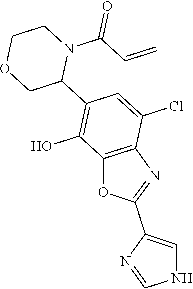 [SMILES: C=CC(=O)N1CCOCC1c1cc(Cl)c2nc(-c3c[nH]cn3)oc2c1O] | 1-(3-(4-chloro-7-hydroxy-2- (1H-imidazol-4- yl)benzo[d]oxazol-6- yl)morpholino)prop-2-en-1- one | ¹H NMR (600 MHz, DMSO-d6) δ 12.84 (brs, 1H), 10.82 (brs, 1H), 8.13 (s, 1H), 7.94 (s, 1H), 7.65-7.61 (m, 1H), 6.85 (s, 1H), 6.22 (s, 1H), 5.80- 5.78 (m, 1H), 5.52-5.50 (m, 1H), 4.27-4.25 (m, 1H), 4.03-4.01 (m, 1H), 3.90-3.88 (m, 1H), 3.53-3.50 (m, 2H), 2.87-2.85 (m, 1H). MS (ESI) m/z [M + H]⁺ = 375. |

### Example 2-26: Synthesis of (R)-1-(6-(4-chloro-7-fluoro-2-(1H-imidazol-4-yl)benzo[d]oxazol-6-yl)-4-oxa-7-azaspiro[2.5]octan-7-yl)prop-2-en-1-one (Assumed) (L11A) and (5)-1-(6-(4-chloro-7 -fluoro-2-(1H-imidazol-4-yl)benzo[d]oxazol-6-yl)-4-oxa-7-azaspiro[2.5]octan-7-yl)prop-2-en-1-one (Assumed) (L11B)

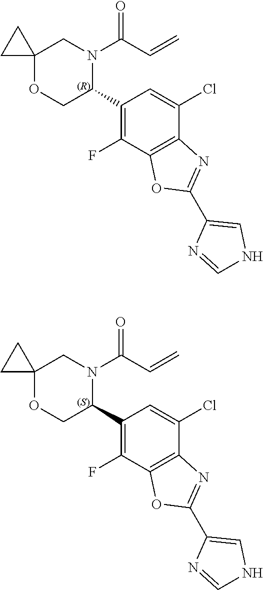

### Step 1: 3-bromo-5-chloro-2-fluorophenol

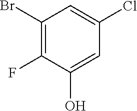

To a mixture of 2-bromo-4-chloro-1-fluorobenzene (22 g, 105.0 mmol), bis(pinacolato)diboron (26.7 g, 105.0 mmol) and 4-tert-butyl-2-(4-tert-butyl-2-pyridyl)pyridine (1.1 g, 4.2 mmol) in n-hexane (500 mL) was added [Ir(μ-OMe)(cod)₂](1.4 g, 2.1 mmol) at rt under N₂ atmosphere. The resulting mixture was stirred at rt for 12 h. Upon completion of the reaction, the solvent was removed in vacuo, and the residue was purified by silica gel column chromatography (EA/PE 1/100 to 3/17) to give 2-(3-bromo-5-chloro-2-fluorophenyl)-4,4,5,5-tetramethyl-1,3,2-dioxaborolane (32.8 g, crude).

To a solution of 2-(3-bromo-5-chloro-2-fluorophenyl)-4,4,5,5-tetramethyl-1,3,2-dioxaborolane (32.8 g, ca. 97.8 mmol) in THF (200 mL) was added a solution of NaOH (11.8 g, 295 mmol) in water (200 mL) and 35% H₂O₂ aq. (33.5 g, 345 mmol) at 0° C., and the resulting mixture was stirred at 0° C. for 2 h. Upon completion of the reaction, sat. aq. Na₂S₂O₃ was added slowly. After the pH of the solution was adjusted to 3 with 1N HCl, the resulting solution was extracted with EA. The combined organic layers were washed with brine, dried over anhydrous Na₂SO₄ and filtered. The solvent was removed in vacuo, and the residue was purified by silica gel column chromatography (EA/PE 1/100 to 3/17) to give the title compound (22 g, 99% over 2 steps). MS (ESI) m/z [M−H]⁻ 223, 225.

### Step 2: 2-amino-5-bromo-3-chloro-6-fluorophenol

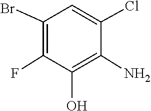 [SMILES: Nc1c(Cl)cc(Br)c(F)c1O]

To a solution of 3-bromo-5-chloro-2-fluorophenol (22 g, 97.6 mmol) in Ac₂O (320 mL) was added dropwise HNO₃ (7.2 g, 98 mmol, 85%) at 0° C., and the resulting mixture was stirred at 0° C. for 2 h. Upon completion of the reaction, the mixture was extracted with EA. The combined organic layers were washed with brine, dried over anhydrous Na₂SO₄ and filtered. The solvent was removed in vacuo to give crude 3-bromo-5-chloro-2-fluoro-6-nitrophenol. MS (ESI) m/z [M−H]⁻ 268, 270.

To a suspension of 3-bromo-5-chloro-2-fluoro-6-nitrophenol (ca. 97.6 mmol) in EtOH (180 mL) and sat. aq. NH₄Cl (180 mL) was added iron powder (27.3 g, 487.5 mmol) slowly at rt, and the resulting mixture was stirred at rt for 2 h. Upon completion of the reaction, the solids were filtered out and the filtrate was concentrated in vacuo. The residue was extracted with EA. The organic layers were washed with brine, dried over anhydrous Na₂SO₄ and filtered. The solvent was removed in vacuo, and the residue was dissolved in aq. NaOH (0.5 N, 400 mL). The solids were filtered out and the filtrate was used directly for next step. MS (ESI) m/z [M+H]⁺ 240, 242.

### Step 3: 6-bromo-4-chloro-7-fluoro-2-(1H-imidazol-4-yl)benzo[d]oxazole

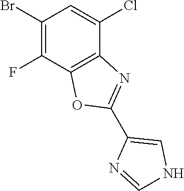 [SMILES: Fc1c(Br)cc(Cl)c2nc(-c3c[nH]cn3)oc12]

To a suspension of 2-amino-5-bromo-3-chloro-6-fluorophenol (ca. 97.7 mmol) in aq. NaOH (0.5 N, 400 mL) was added 4-(trifluoromethyl)-1H-imidazole (13.3 g, 97.7 mmol) at rt, and the resulting mixture was stirred at 80° C. for 2 h. Upon completion of the reaction, and the solid was collected by filtration to give the product (18.6 g, crude). MS (ESI) m/z [M+H]⁺ 316, 318.

### Step 4: 6-bromo-4-chloro-7-fluoro-2-(1-((2-(trimethylsilyl)ethoxy)methyl)-1H-imidazol-4-yl)benzo[d]oxazole (Assumed) and 6-bromo-4-chloro-7-fluoro-2-(1-((2-(trimethylsilyl)ethoxy)methyl)-1H-imidazol-5-yl)benzo[d]oxazole (Assumed)

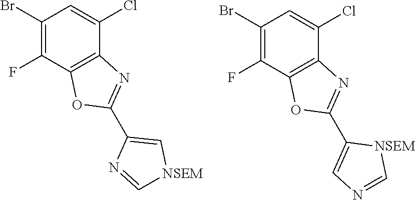 [SMILES: C[Si](C)(C)CCOCn1cnc(-c2nc3c(Cl)cc(Br)c(F)c3o2)c1 ; C[Si](C)(C)CCOCn1cncc1-c1nc2c(Cl)cc(Br)c(F)c2o1]

To a solution of 6-bromo-4-chloro-7-fluoro-2-(1H-imidazol-4-yl)benzo[d]oxazole (10 g, ca. 31.6 mmol) in dry DMF (150 mL) was added NaH (60% in mineral oil, 2.5 g, 63.3 mmol) batchwise at 0° C. The mixture was stirred at 0° C. for 0.5 h before 2-(chloromethoxy)ethyl)trimethylsilane (15.8 g, 94.8 mmol) was added, and then the resulting mixture was stirred at rt for another 3 h. Upon completion of the reaction, the mixture was slowly added to sat. aq. NH₄Cl at 0° C., and the mixture was extracted with EA. The combined organic layers were washed with brine, dried over Na₂SO₄, filtered and concentrated. The residue was purified by silica gel column chromatography (EA/PE 1/20 to 4/1) to give the title compound (5.5 g, 39% over 2 steps). MS (ESI) m/z [M+H]⁺ 446, 448.

### Step 5: 4-chloro-7-fluoro-6-(4,4,5,5-tetramethyl-1,3,2-dioxaborolan-2-yl)-2-(1-((2-(trimethylsilyl)ethoxy)methyl)-1H-imidazol-4-yl)benzo[d]oxazole (Assumed) and 4-chloro-7-fluoro-6-(4,4,5,5-tetramethyl-1,3,2-dioxaborolan-2-yl)-2-(1-((2-(trimethylsilyl)ethoxy)methyl)-1H-imidazol-5-yl)benzo[d]oxazole (Assumed)

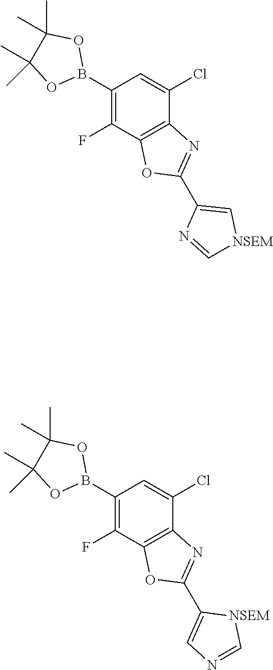 [SMILES: CC1(C)OB(c2cc(Cl)c3nc(-c4cn(COCC[Si](C)(C)C)cn4)oc3c2F)OC1(C)C ; CC1(C)OB(c2cc(Cl)c3nc(-c4cncn4COCC[Si](C)(C)C)oc3c2F)OC1(C)C]

A mixture of 6-bromo-4-chloro-7-fluoro-2-(1-((2-(trimethylsilyl)ethoxy)methyl)-1H-imidazol-4-yl)benzo[d]oxazole (assumed) and 6-bromo-4-chloro-7-fluoro-2-(1-((2-(trimethylsilyl)ethoxy)methyl)-1H-imidazol-5-yl)benzo[d]oxazole (assumed) (4.0 g, 9.0 mmol), bis(pinacolato)diboron (5.7 g, 22.0 mmol), Pd(dppf)Cl₂ (655 mg, 0.9 mmol) and KOAc (1.7 g, 17.9 mmol) in 1,4-dioxane (150 mL) was stirred at 90° C. for 12 h. After cooled to rt, the mixture was concentrated under reduced pressure. The crude product was purified by silica gel column chromatography (EA/PE 1/5 to 1/2) to give the title compound (3.8 g, 40% purity, 34% yield). MS (ESI) m/z [M+H]⁺ 494.

### Step 6: tert-butyl 6-(4-chloro-7-fluoro-2-(1-((2-(trimethylsilyl)ethoxy)methyl)-1H-imidazol-4-yl)benzo[d]oxazol-6-yl)-4-oxa-7-azaspiro[2.5]oct-5-ene-7-carboxylate (Assumed) and tert-butyl 6-(4-chloro-7-fluoro-2-(1-((2-(trimethylsilyl)ethoxy)methyl)-1H-imidazol-5-yl)benzo[d]oxazol-6-yl)-4-oxa-7-azaspiro[2.5]oct-5-ene-7-carboxylate (Assumed)

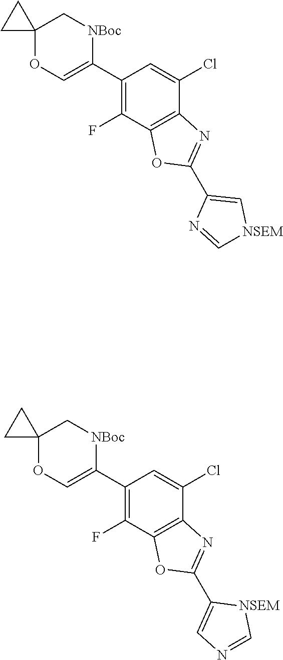 [SMILES: CC(C)(C)OC(=O)N1CC2(CC2)OC=C1c1cc(Cl)c2nc(-c3cn(COCC[Si](C)(C)C)cn3)oc2c1F ; CC(C)(C)OC(=O)N1CC2(CC2)OC=C1c1cc(Cl)c2nc(-c3cncn3COCC[Si](C)(C)C)oc2c1F]

A mixture of 4-chloro-7-fluoro-6-(4,4,5,5-tetramethyl-1,3,2-dioxaborolan-2-yl)-2-(1-((2-(trimethylsilyl)ethoxy)methyl)-1H-imidazol-4-yl)benzo[d]oxazole (Assumed) and 4-chloro-7-fluoro-6-(4,4,5,5-tetramethyl-1,3,2-dioxaborolan-2-yl)-2-(1-((2-(trimethylsilyl)ethoxy)methyl)-1H-imidazol-5-yl)benzo[d]oxazole (Assumed) (2.3 g, ca. 4.7 mmol), tert-butyl 6-((diphenoxyphosphoryl)oxy)-4-oxa-7-azaspiro[2.5]oct-5-ene-7-carboxylate (2.4 g, 5.2 mmol), K₂CO₃ (1.3 g, 9.4 mmol), Pd(dppf)Cl₂ (0.34 g, 0.5 mmol) in 1,4-dioxane (80 mL) and water (16 mL) was stirred at 90° C. for 2 h under N₂ atmosphere. After cooled to rt, the mixture was extracted with EA. The organic layers were washed with brine, dried over anhydrous Na₂SO₄ and filtered. The filtrate was concentrated in vacuo, and the residue was purified by silica gel column chromatography (EA/PE 1/100 to 1/1) to give the title compound (0.96 g, 45% purity, 16% yield). MS (ESI) m/z [M+H]⁺ 577.

### Step 7: 4-chloro-7-fluoro-2-(1H-imidazol-4-yl)-6-(4-oxa-7-azaspiro[2.5]octan-6-yl)benzo[d]oxazole

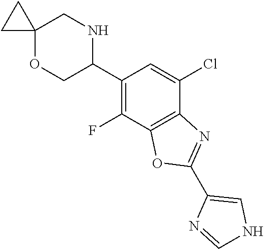 [SMILES: Fc1c(C2COC3(CC3)CN2)cc(Cl)c2nc(-c3c[nH]cn3)oc12]

To a solution of tert-butyl 6-(4-chloro-7-fluoro-2-(1-((2-(trimethylsilyl)ethoxy)methyl)-1H-imidazol-4-yl)benzo[d]oxazol-6-yl)-4-oxa-7-azaspiro[2.5]oct-5-ene-7-carboxylate (assumed) and tert-butyl 6-(4-chloro-7-fluoro-2-(1-((2-(trimethylsilyl)ethoxy)methyl)-1H-imidazol-5-yl)benzo[d]oxazol-6-yl)-4-oxa-7-azaspiro[2.5]oct-5-ene-7-carboxylate (assumed) (0.96 g, ca. 1.6 mmol) in HFIP (40 mL) was added MsOH (0.8 mL) at 0° C., and the resulting mixture was stirred at 0° C. for 0.5 h. Upon completion of the reaction, MeOH (40 mL) was added, followed by NaBH₃CN (157 mg, 2.5 mmol) at 0° C. The resulting mixture was stirred at 0° C. for another 0.5 h. Upon completion of the reaction, sat. aq. NaHCO₃ was added, and the mixture was extracted with MeOH/DCM (1/10) to give the title compound. The organic layers were used directly without further operation. MS (ESI) m/z [M+H]⁺ 349.

### Step 8: 1-(6-(4-chloro-7-fluoro-2-(1H-imidazol-4-yl)benzo[d]oxazol-6-yl)-4-oxa-7-azaspiro[2.5]octan-7-yl)prop-2-en-1-one

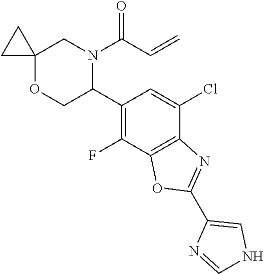 [SMILES: C=CC(=O)N1CC2(CC2)OCC1c1cc(Cl)c2nc(-c3c[nH]cn3)oc2c1F]

To a solution of 4-chloro-7-fluoro-2-(1H-imidazol-4-yl)-6-(4-oxa-7-azaspiro[2.5]octan-6-yl)benzo[d]oxazole (ca. 0.76 mmol) in MeOH/DCM (1/10, 250 mL) was added a solution of acryloyl chloride (35 mg, 0.4 mmol) dropwise in CH₂Cl₂ (3.5 mL) at rt. The resulting mixture was stirred at rt for 20 min. Upon completion of the reaction, the solvent was removed in vacuo, and the residue was purified by Prep-TLC (EA) first and then re-purified by C18 column (mobile phase, ACN in water (0.1% FA), 25% to 45%) to give the title compound (32 mg, 8.5% over 3 steps). ¹H NMR (600 MHz, DMSO-d₆) δ 12.94 (s, 1H), 8.26 (s, 1H), 7.97 (s, 1H), 7.65 (s, 1H), 6.79 (brs, 1H), 6.21 (d, J=16.6 Hz, 1H), 5.77 (brs, 1H), 5.75 (d, J=10.5 Hz, 1H), 4.30 (d, J=11.5 Hz, 1H), 3.99 (s, 1H), 3.87-3.66 (m, 1H), 3.63-3.40 (m, 1H), 0.94-0.86 (m, 1H), 0.81-0.73 (m, 1H), 0.72-0.66 (s, 1H), 0.56-0.46 (s, 1H). MS (ESI) m/z [M+H]⁺=403.

### Step 9: (S)-1-(6-(4-chloro-7-fluoro-2-(1H-imidazol-4-yl)benzo[d]oxazol-6-yl)-4-oxa-7-azaspiro[2.5]octan-7-yl)prop-2-en-1-one (Assumed) (L11B) and (R)-1-(6-(4-chloro-7-fluoro-2-(1H-imidazol-4-yl)benzo[d]oxazol-6-yl)-4-oxa-7-azaspiro[2.5]octan-7-yl)prop-2-en-1-one (Assumed) (L11A)

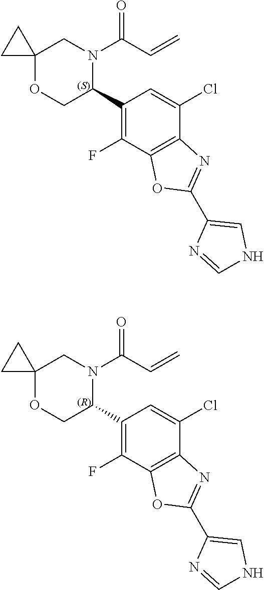

Racemic 1-(6-(4-chloro-7-fluoro-2-(1H-imidazol-4-yl)benzo[d]oxazol-6-yl)-4-oxa-7-azaspiro[2.5]octan-7-yl)prop-2-en-1-one was separated using the following conditions:

SFC: column: Lux i-Amylose-3; fluent: 32% MeOH (0.3% 2M NH₃ MeOH)/CO₂, isomer 1, retention time=5.72 min, isomer 2, retention time=7.98 min.

Isomer 1: ¹H NMR (600 MHz, DMSO-d₆) 12.91 (s, 1H), 8.24 (s, 1H), 7.96 (s, 1H), 7.65 (s, 1H), 6.83 (brs, 1H), 6.21 (d, J=16.5 Hz, 1H), 5.77 (brs, 1H), 5.75 (d, J=10.8 Hz, 1H), 4.30 (d, J=12.2 Hz, 1H), 4.00 (s, 1H), 3.87-3.68 (m, 1H), 3.68-3.50 (m, 1H), 0.95-0.87 (m, 1H), 0.81-0.73 (m, 1H), 0.73-0.66 (s, 1H), 0.56-0.47 (s, 1H). MS (ESI) m/z [M+H]⁺=403.

Isomer 2: ¹H NMR (600 MHz, DMSO-d₆) 12.90 (s, 1H), 8.24 (s, 1H), 7.96 (s, 1H), 7.65 (s, 1H), 6.82 (brs, 1H), 6.21 (d, J=16.6 Hz, 1H), 5.86 (brs, 1H), 5.75 (d, J=9.8 Hz, 1H), 4.30 (d, J=11.7 Hz, 1H), 4.00 (s, 1H), 3.85-3.63 (m, 1H), 3.57-3.45 (m, 1H), 0.93-0.87 (m, 1H), 0.81-0.74 (m, 1H), 0.73-0.66 (s, 1H), 0.56-0.48 (s, 1H). MS (ESI) m/z [M+H]⁺=403.

### Example 2-27: Synthesis of (S)-1-(3-(4-chloro-2-(1H-imidazol-4-yl)benzo[d]thiazol-6-yl)morpholino)prop-2-en-1-one (N1A, Assumed) and (R)-1-(3-(4-chloro-2-(1H-imidazol-4-yl)benzo[d]thiazol-6-yl)morpholino)prop-2-en-1-one (N1B, Assumed)

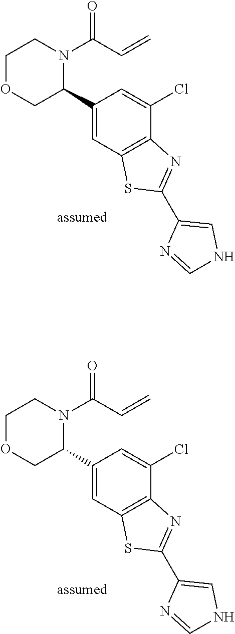 [SMILES: C=CC(=O)N1CCOC[C@@H]1c1cc(Cl)c2nc(-c3c[nH]cn3)sc2c1 ; C=CC(=O)N1CCOC[C@H]1c1cc(Cl)c2nc(-c3c[nH]cn3)sc2c1]

The racemic compound was synthesized using similar procedure as described in Example 2-26 steps 5-8. Racemic 1-(3-(4-chloro-2-(1H-imidazol-4-yl)benzo[d]thiazol-6-yl)morpholino)prop-2-en-1-one was separated using the following conditions:

Column: CHIRALPAK AD-H; 38% MeOH(0.3% 2M NH₃ MeOH)/CO₂, isomer 1, retention time=6.52 min, isomer 2, retention time=10.50 min.

Isomer 1: (S)-1-(3-(4-chloro-2-(1H-imidazol-4-yl)benzo[d]thiazol-6-yl)morpholino)prop-2-en-1-one (N1A, assumed)

¹H NMR (600 MHz, DMSO-d₆) δ 12.80 (s, 1H), 8.10-7.95 (m, 2H), 7.88 (s, 1H), 7.52 (s, 1H), 6.93-6.80 (m, 1H), 6.24 (d, J=16.2 Hz, 1H), 5.77 (d, J=9.8 Hz, 1H), 5.69-5.30 (m, 1H), 4.47 (d, J=12.2 Hz, 1H), 4.30-3.75 (m, 3H), 3.56-3.47 (m, 1H), 3.30-2.91 (m, 1H). MS (ESI) m/z [M+H]⁺=375.

Isomer 2: (R)-1-(3-(4-chloro-2-(1H-imidazol-4-yl)benzo[d]thiazol-6-yl)morpholino)prop-2-en-1-one (N1B, assumed)

¹H NMR (600 MHz, DMSO-d₆) δ 12.80 (s, 1H), 8.10-7.96 (m, 2H), 7.88 (s, 1H), 7.52 (s, 1H), 6.92-6.80 (m, 1H), 6.24 (d, J=16.6 Hz, 1H), 5.77 (d, J=10.0 Hz, 1H), 5.69-5.31 (m, 1H), 4.47 (d, J=12.2 Hz, 1H), 4.33-3.74 (m, 3H), 3.57-3.47 (m, 1H), 3.29-2.91 (m, 1H). m/z [M+H]⁺=375.

### Example 2-28: Synthesis of 1-(2-(7-chloro-2-(1H-1,2,4-triazol-3-yl)benzofuran-5-yl)tetrahydropyridazin-1(2H)-yl)prop-2-en-1-one (B43)

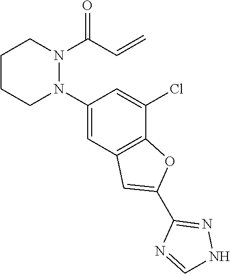 [SMILES: C=CC(=O)N1CCCCN1c1cc(Cl)c2oc(-c3nc[nH]n3)cc2c1]

### Step 1: tert-butyl 2-(7-chloro-2-(1-((2-(trimethylsilyl)ethoxy)methyl)-1H-1,2,4-triazol-3-yl)benzofuran-5-yl)tetrahydropyridazine-1(2H)-carboxylate

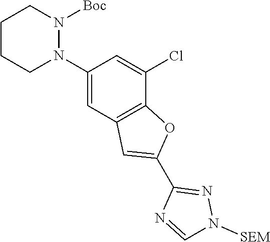 [SMILES: CC(C)(C)OC(=O)N1CCCCN1c1cc(Cl)c2oc(-c3ncn(COCC[Si](C)(C)C)n3)cc2c1]

A mixture of 2-[[3-(5-bromo-7-chloro-benzofuran-2-yl)-1,2,4-triazol-1-yl]methoxy]ethyl-trimethyl-silane (500 mg, 1.17 mmol), tert-butyl hexahydropyridazine-1-carboxylate (195 mg, 1.05 mmol), Pd(OAc)₂ (26 mg, 0.12 mmol), Xantphos (135 mg, 0.23 mmol) and t-BuONa (336 mg, 3.50 mmol) in toluene (20 mL) was degassed with N₂ for 3 times. The mixture was then heated at 90° C. for 2 h. Upon completion of the reaction, the mixture was cooled to rt and extracted with EA. The organic layer was dried over anhydrous Na₂SO₄ and concentrated in vacuo. The residue was purified by silica gel column chromatography (PE/EA 100:1 to 3:2) to give the title compound (240 mg, 39% yield). MS (ESI) m/z [M+H]⁺=534.

### Step 2: 1-(7-chloro-2-(1H-1,2,4-triazol-3-yl)benzofuran-5-yl)hexahydropyridazine

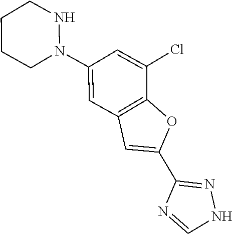 [SMILES: Clc1cc(N2CCCCN2)cc2cc(-c3nc[nH]n3)oc12]

To a solution of tert-butyl 2-[7-chloro-2-[1-(2-trimethylsilylethoxymethyl)-1,2,4-triazol-3-yl]benzofuran-5-yl]hexahydropyridazine-1-carboxylate (120 mg, 0.22 mmol) in HFIP (4 mL) was added methanesulfonic acid (65 mg, 0.6 mmol) at rt. The mixture was stirred at rt for 20 min. Upon completion of the reaction, the solvent was removed in vacuo, and the residue (68 mg) was used in next step without further purification. MS (ESI) m/z [M+H]⁺=304.

### Step 3:1-(2-(7-chloro-2-(1H-1,2,4-triazol-3-yl)benzofuran-5-yl)tetrahydropyridazin-1(2H)-yl)prop-2-en-1-one

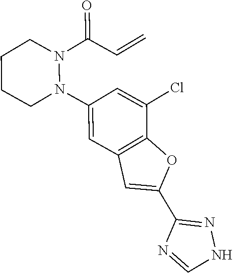 [SMILES: C=CC(=O)N1CCCCN1c1cc(Cl)c2oc(-c3nc[nH]n3)cc2c1]

To a solution of crude 1-(7-chloro-2-(1H-1,2,4-triazol-3-yl)benzofuran-5-yl)hexahydropyridazine (68 mg, ca. 0.22 mmol) in sat. aq. NaHCO₃ (3 mL), DCM (3 mL) and MeOH (3 mL) was added dropwise a solution of acryloyl chloride in DCM (10 mg/mL, 2 mL) at 0° C. The mixture was stirred at 0° C. for 1 h. Upon completion of the reaction, the mixture was quenched with H₂O and extracted with DCM. The combined organic layers were dried over anhydrous Na₂SO₄ and concentrated under reduced pressure. The residue was purified by C18 column (mobile phase, ACN in water (0.1% FA), 30% to 50%) to give the title compound (2.73 mg, 3.4% yield over 2 steps).

¹H NMR (600 MHz, DMSO-d₆) δ 14.48 (s, 1H), 8.66 (s, 1H), 7.39 (s, 1H), 7.11 (s, 1H), 7.03 (s, 1H), 6.73 (dd, J=17.1, 10.4 Hz, 1H), 6.29 (d, J=17.1 Hz, 1H), 5.75 (d, J=10.3 Hz, 1H), 4.34 (d, J=12.6 Hz, 1H), 4.27 (d, J=14.4 Hz, 1H), 3.22-3.14 (m, 1H), 2.88-2.77 (m, 1H), 1.75-1.62 (m, 3H), 1.58-1.49 (m, 1H). MS (ESI) m/z [M+H]⁺=358.

Compounds B38-B42 in table 2-11 were prepared from tert-butyl 3-(7-chloro-4-methoxy-2-(1-((2-(trimethylsilyl)ethoxy)methyl)-1H-1,2,4-triazol-5-yl)benzofuran-5-yl)morpholine-4-carboxylate, then it undergone de-methylation, Mitsunobu reaction, de-Boc, and reacted with acryloyl chloride to give the final compounds.

TABLE 2-11

| Compound | Structure | Chemical Name | ¹H NMR data LC/MS m/z (M + H) |
|---|---|---|---|
| B38 | 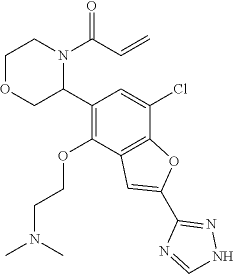 [SMILES: C=CC(=O)N1CCOCC1c1cc(Cl)c2oc(-c3nc[nH]n3)cc2c1OCCN(C)C] | 1-(3-(7-chloro-4-(2- (dimethylamino)ethoxy)- 2-(1H-1,2,4-triazol-3- yl)benzofuran-5- yl)morpholino)prop-2-en- 1-one | ¹H NMR (600 MHz, DMSO-d₆) δ 14.51 (s, 1H), 8.72 (s, 1H), 8.18 (s, 1H), 7.69 (s, 1H), 7.46 (s, 1H), 6.87 (dd, J = 16.3, 10.8 Hz, 1H), 6.18 (d, J = 16.6 Hz, 1H), 5.79-5.51 (m, 2H), 4.51-4.37 (m, 2H), 4.22 (d, J = 11.6 Hz, 2H), 4.03- 3.93 (m, 1H), 3.88-3.77 (m, 1H), 3.62- 3.57 (m, 1H), 2.73-2.58 (m, 2H), 2.25 (s, 6H). MS (ESI) m/z [M + H]⁺ = 446. |
| B39 | 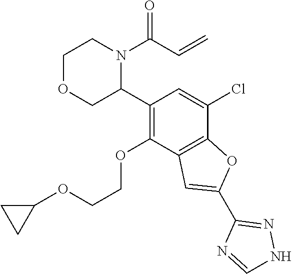 [SMILES: C=CC(=O)N1CCOCC1c1cc(Cl)c2oc(-c3nc[nH]n3)cc2c1OCCOC1CC1] | 1-(3-(7-chloro-4-(2- cyclopropoxyethoxy)-2- (1H-1,2,4-triazol-3- yl)benzofuran-5- yl)morpholino)prop-2-en- 1-one | ¹H NMR (600 MHz, DMSO-d₆) δ 14.55 (s, 1H), 8.73 (s, 1H), 7.68 (s, 1H), 7.43 (s, 1H), 6.91-6.74 (m, 1H), 6.17 (d, J = 16.2 Hz, 1H), 5.71 (d, J = 10.1 Hz, 1H), 5.65-5.36 (m, 1H), 4.55-4.41 (m, 2H), 4.21 (d, J = 11.9 Hz, 2H), 4.03- 3.93 (m, 1H), 3.87-3.67 (m, 4H), 3.57-3.49 (dd, J = 11.5, 8.7 Hz, 1H), 3.43-3.37 (m, 1H), 0.55-0.45 (m, 4H). MS (ESI) m/z [M + H]⁺ = 459. |
| B40 | 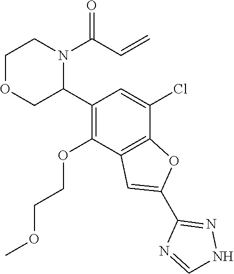 [SMILES: C=CC(=O)N1CCOCC1c1cc(Cl)c2oc(-c3nc[nH]n3)cc2c1OCCOC] | 1-(3-(7-chloro-4-(2- methoxyethoxy)-2-(1H- 1,2,4-triazol-3- yl)benzofuran-5- yl)morpholino)prop-2-en- 1-one | ¹H NMR (600 MHz, DMSO-d₆) δ 14.51 (s, 1H), 8.78 (s, 1H), 7.66 (s, 1H), 7.58- 7.30 (m, 1H), 6.91-6.79 (m, 1H), 6.17 (d, J = 16.5 Hz, 1H), 5.71 (d, J = 10.2 Hz, 1H), 5.65-5.36 (m, 1H), 4.54- 4.41 (m, 2H), 4.32-4.06 (m, 2H), 4.03-3.93 (m, 1H), 3.73-3.61 (m, 3H), 3.53 (t, J = 10.3 Hz, 1H), 3.46- 3.41 (m, 1H), 3.33 (s, 3H). MS (ESI) m/z [M + H]⁺ = 433. |
| B41 | 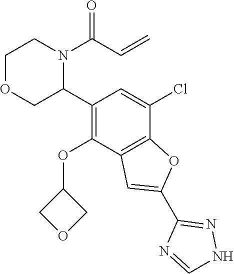 [SMILES: C=CC(=O)N1CCOCC1c1cc(Cl)c2oc(-c3nc[nH]n3)cc2c1OC1COC1] | 1-(3-(7-chloro-4-(oxetan- 3-yloxy)-2-(1H-1,2,4- triazol-3-yl)benzofuran-5- yl)morpholino)prop-2-en- 1-one | ¹H NMR (600 MHz, DMSO-d₆) δ 14.54 (s, 1H), 8.78 (s, 1H), 7.52 (s, 1H), 7.40 (s, 1H), 6.86-6.73 (m, 1H), 6.17 (d, J = 17.0 Hz, 1H), 5.88-5.54 (m, 2H), 5.32 (s, 1H), 4.96-4.81 (m, 2H), 4.76- 4.61 (m, 2H), 4.22 (d, J = 11.7 Hz, 1H), 4.04-3.81 (m, 2H), 3.70-3.49 (m, 3H). MS (ESI) m/z [M + H]⁺ = 431. |
| B42 | 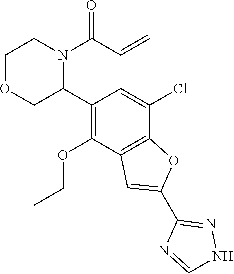 [SMILES: C=CC(=O)N1CCOCC1c1cc(Cl)c2oc(-c3nc[nH]n3)cc2c1OCC] | 1-(3-(7-chloro-4-ethoxy- 2-(1H-1,2,4-triazol-3- yl)benzofuran-5- yl)morpholino)prop-2-en- 1-one | ¹H NMR (600 MHz, DMSO-d₆) δ 14.47 (s, 1H), 8.71 (s, 1H), 7.65 (s, 1H), 7.49 (s, 1H), 6.84 (dd, J = 16.6, 10.3 Hz, 1H), 6.17 (d, J = 16.6 Hz, 1H), 5.72 (d, J = 9.8 Hz, 1H), 5.67-5.36 (m, 1H), 4.48-4.35 (m, 2H), 4.20 (d, J = 11.8 Hz, 2H), 4.03-3.91 (m, 1H), 3.87- 3.77 (m, 1H), 3.57-3.45 (m, 2H), 1.38 (t, J = 6.8 Hz, 3H). MS (ESI) m/z [M + H]⁺ = 403. |

### Example 2-29: Synthesis of (R)-1-(6-(7 chloro-4-fluoro-2-(1H-imidazol-4-yl)benzo[d]oxazol-5-yl)-4-oxa-7-azaspiro[2.5]octan-7-yl)prop-2-en-1-one (D28A, Assumed) and (S)-1-(6-(7-chloro-4-fluoro- 2-(1H-imidazol-4-yl)benzo[d]oxazol-5-yl)-4-oxa-7-azaspiro[2.5]octan-7-yl)prop-2-en-1-one (D28B, Assumed)

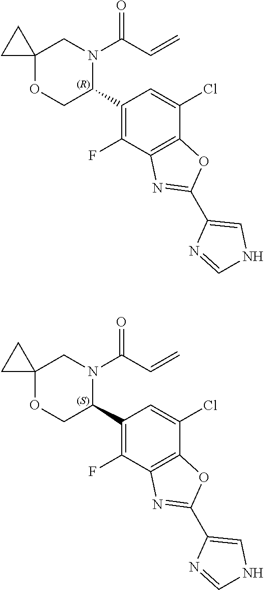

### Step 1: 4-bromo-6-chloro-3-fluoro-2-nitro-phenol

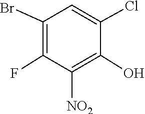 [SMILES: O=[N+]([O-])c1c(O)c(Cl)cc(Br)c1F]

To a stirred mixture of 4-bromo-2-chloro-5-fluoro-phenol (25.0 g, 110.89 mmol) in HOAc (250 mL) was added dropwise HNO₃ (10.0 g, 103.17 mmol, 65% purity) at rt. The resulting mixture was stirred at rt for 1 h. Upon completion of the reaction, ice water (80 mL) was added. The solid was collected by filtration and the filter cake was dried in vacuo to give the title compound (22.0 g, 73.3% yield). MS (ESI) m/z [M+1]⁺=270, 272.

### Step 2: 2-amino-4-bromo-6-chloro-3-fluoro-phenol

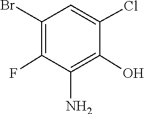 [SMILES: Nc1c(O)c(Cl)cc(Br)c1F]

A mixture of 4-bromo-6-chloro-3-fluoro-2-nitro-phenol (22.0 g, 81.35 mmol), Fe powder (23.0 g, 410.7 mmol) and NH₄Cl (43.0 g, 811.3 mmol) in EtOH (150 mL) and H₂O (150 mL) was stirred at 80° C. for 1 h. After cooling to rt, the reaction mixture was filtered, and the filter cake was washed with MeOH. The filtrate was concentrated under reduced pressure. The residue was purified by flash chromatography on silica gel (EA/PE 1/9 to 7/3) to give the title compound (8.0 g, 40.9% yield). MS (ESI) m/z [M+1]⁺=240, 242.

### Step 3: 5-bromo-7-chloro-4-fluoro-2-(1H-imidazol-4-yl)-1,3-benzoxazole

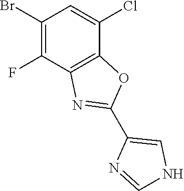 [SMILES: Fc1c(Br)cc(Cl)c2oc(-c3c[nH]cn3)nc12]

A mixture of 2-amino-4-bromo-6-chloro-3-fluoro-phenol (1.2 g, 5.0 mmol) and 4-(trifluoromethyl)-1H-imidazole (1.4 g, 10.3 mmol) in aq. NaOH (0.5 M/L, 12 mL) was stirred at 80° C. for 2 h. After cooling to rt, the solid was collected by filtration to give the title compound (1.3 g, 82.0% yield). MS (ESI) m/z [M+1]⁺=316, 318.

### Step 4: 2-[[4-(5-bromo-7-chloro-4-fluoro-1,3-benzoxazol-2-yl)imidazol-1-yl]methoxy]ethyl-trimethyl-silane (Assumed) and 5-bromo-7-chloro-4-fluoro-2-(1-((2-(trimethylsilyl)ethoxy)methyl)-1H-imidazol-5-yl)benzo[d]oxazole (Assumed)

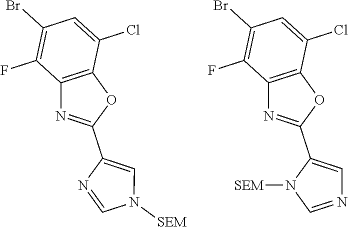 [SMILES: C[Si](C)(C)CCOCn1cnc(-c2nc3c(F)c(Br)cc(Cl)c3o2)c1 ; C[Si](C)(C)CCOCn1cncc1-c1nc2c(F)c(Br)cc(Cl)c2o1]

To a solution of 5-bromo-7-chloro-4-fluoro-2-(1H-imidazol-4-yl)-1,3-benzoxazole (1.3 g, 4.10 mmol) and TEA (1.2 g, 11.9 mmol) in DMF (20 mL) was added dropwise SEM-Cl (1.0 g, 6.0 mmol) at rt. The resulting mixture was stirred at rt for 16 h. Upon completion of the reaction, ice water (50 mL) was added. The mixture was extracted with EA. The combined organic layers were dried over anhydrous Na₂SO₄ and concentrated. The residue was purified by flash chromatography on silica gel (EA/PE 1/9 to 3/2) to give the title compound (1.4 g, 76.0% yield, ratio=2/3). MS (ESI) m/z [M+1]⁺=446, 448.

### Step 5: 2-[[4-[7-chloro-4-fluoro-5-(4,4,5,5-tetramethyl-1,3,2-dioxaborolan-2-yl)-1,3-benzoxazol-2-yl]imidazol-1-yl]methoxy]ethyl-trimethyl-silane (Assumed) and 7-chloro-4-fluoro-5-(4,4,5,5-tetramethyl-1,3,2-dioxaborolan-2-yl)-2-(1-((2-(trimethylsilyl)ethoxy)methyl)-1H-imidazol-5-yl)benzo[d]oxazole (Assumed)

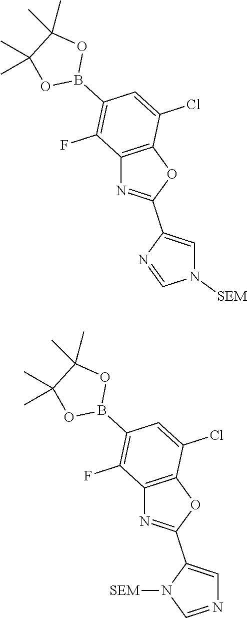 [SMILES: CC1(C)OB(c2cc(Cl)c3oc(-c4cn(COCC[Si](C)(C)C)cn4)nc3c2F)OC1(C)C ; CC1(C)OB(c2cc(Cl)c3oc(-c4cncn4COCC[Si](C)(C)C)nc3c2F)OC1(C)C]

A mixture of 2-[[4-(5-bromo-7-chloro-4-fluoro-1,3-benzoxazol-2-yl)imidazol-1-yl]methoxy]ethyl-trimethyl-silane and 5-bromo-7-chloro-4-fluoro-2-(1-((2-(trimethylsilyl)ethoxy)methyl)-1H-imidazol-5-yl)benzo[d]oxazole (1.0 g, 2.24 mmol), 4,4,5,5-tetramethyl-2-(4,4,5,5-tetramethyl-1,3,2-dioxaborolan-2-yl)-1,3,2-dioxaborolane (853 mg, 3.36 mmol), Pd(dppf)Cl₂ (164 mg, 0.22 mmol) and KOAc (658 mg, 6.71 mmol) in 1,4-dioxane (15 mL) was stirred at 90° C. for 5 h under N₂ atmosphere. After cooling to rt, the resulting mixture was concentrated under vacuum. The residue was purified by flash chromatography on silica gel (EA/PE 1/9 to 4/1) to give the title compounds (1.0 g, 90.5% yield, ratio=2/3). MS (ESI) m/z [M+1]⁺=494.

### Step 6: tert-butyl 6-[7-chloro-4-fluoro-2-[1-(2-trimethylsilylethoxymethyl)imidazol-4-yl]-1,3-benzoxazol-5-yl]-4-oxa-7-azaspiro[2.5]oct-5-ene-7-carboxylate (Assumed) and tert-butyl 6-(7-chloro-4-fluoro-2-(1-((2-(trimethylsilyl)ethoxy)methyl)-1H-imidazol-5-yl)benzo[d]oxazol-5-yl)-4-oxa-7-azaspiro[2.5]oct-5-ene-7-carboxylate (Assumed)

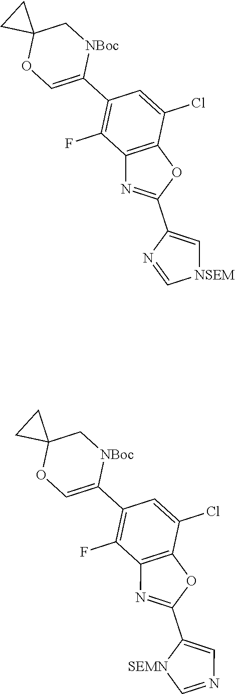 [SMILES: CC(C)(C)OC(=O)N1CC2(CC2)OC=C1c1cc(Cl)c2oc(-c3cn(COCC[Si](C)(C)C)cn3)nc2c1F ; CC(C)(C)OC(=O)N1CC2(CC2)OC=C1c1cc(Cl)c2oc(-c3cncn3COCC[Si](C)(C)C)nc2c1F]

A mixture of 2-[[4-[7-chloro-4-fluoro-5-(4,4,5,5-tetramethyl-1,3,2-dioxaborolan-2-yl)-1,3-benzoxazol-2-yl]imidazol-1-yl]methoxy]ethyl-trimethyl-silane and 7-chloro-4-fluoro-5-(4,4,5,5-tetramethyl-1,3,2-dioxaborolan-2-yl)-2-(1-((2-(trimethylsilyl)ethoxy)methyl)-1H-imidazol-5-yl)benzo[d]oxazole (1.0 g, 2.0 mmol), tert-butyl 6-diphenoxyphosphoryloxy-4-oxa-7-azaspiro[2.5]oct-5-ene-7-carboxylate (1.4 g, 3.0 mmol), Pd(dppf)Cl₂ (148 mg, 0.20 mmol) and K₂CO₃ (838 mg, 6.07 mmol) in 1,4-dioxane (20 mL) and H₂O (4 mL) was stirred at 90° C. for 2 h under nitrogen atmosphere. After cooling to rt, the resulting mixture was concentrated under vacuum. The residue was purified by flash chromatography on silica gel (EA/PE 1/9 to 4/1) to give the title compound (1.0 g, 86% yield). MS (ESI) m/z [M+1]⁺=577.

### Step 7: 7-chloro-4-fluoro-2-(1H-imidazol-4-yl)-5-(4-oxa-7-azaspiro[2.5]octan-6-yl)-1,3-benzoxazole

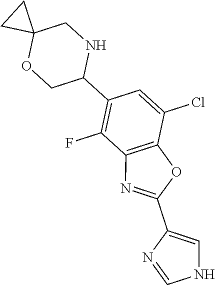 [SMILES: Fc1c(C2COC3(CC3)CN2)cc(Cl)c2oc(-c3c[nH]cn3)nc12]

To a solution of tert-butyl 6-[7-chloro-4-fluoro-2-[1-(2-trimethylsilylethoxymethyl)imidazol-4-yl]-1,3-benzoxazol-5-yl]-4-oxa-7-azaspiro[2.5]oct-5-ene-7-carboxylate and tert-butyl 6-(7-chloro-4-fluoro-2-(1-((2-(trimethylsilyl)ethoxy)methyl)-1H-imidazol-5-yl)benzo[d]oxazol-5-yl)-4-oxa-7-azaspiro[2.5]oct-5-ene-7-carboxylate (1.0 g, 1.7 mmol) in HFIP (10 mL) was added dropwise MsOH (1.7 g, 18.0 mmol) at rt. The resulting mixture was stirred at rt for 1 h followed by the addition of MeOH (15 mL) and NaBH₃CN (181 mg, 2.87 mmol) at rt. The resulting mixture was stirred at rt for another 1 h. Upon completion of the reaction, the solvent was removed in vacuo to give the crude title compound (900 mg). MS (ESI) m/z [M+1]⁺=349.

### Step 8: 1-(6-(7-chloro-4-fluoro-2-(1H-imidazol-4-yl)benzo[d]oxazol-5-yl)-4-oxa-7-azaspiro[2.5]octan-7-yl)prop-2-en-1-one

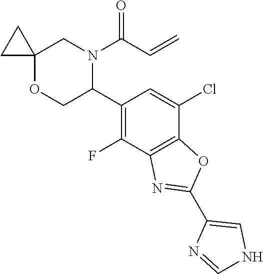 [SMILES: C=CC(=O)N1CC2(CC2)OCC1c1cc(Cl)c2oc(-c3c[nH]cn3)nc2c1F]

To a stirred mixture of 7-chloro-4-fluoro-2-(1H-imidazol-4-yl)-5-(4-oxa-7-azaspiro[2.5]octan-6-yl)-1,3-benzoxazole (crude, 300 mg, ca. 0.43 mmol) in ACN (5 mL) and aq. NaHCO₃ (0.8 M/L, 5 mL) was added dropwise acryloyl chloride (117 mg, 1.29 mmol) at rt. The reaction mixture was stirred at rt for 1 h. Upon completion of the reaction, ice water (50 mL) was added. The resulting mixture was extracted with DCM. The combined organic layers were dried over anhydrous Na₂SO₄, filtered and concentrated. The residue was purified by C18 column (mobile phases, ACN in water (0.1% FA), 25% to 45%) to give the title compound (100 mg, 42% yield over 2 steps). MS (ESI) m/z [M+H]⁺=405.

### Step 9: (R)-1-(6-(7-chloro-4-fluoro-2-(1H-imidazol-4-yl)benzo[d]oxazol-5-yl)-4-oxa-7-azaspiro[2.5]octan-7-yl)prop-2-en-1-one (D28A, Assumed) and (S)-1-(6-(7-chloro-4-fluoro-2-(1H-imidazol-4-yl)benzo[d]oxazol-5-yl)-4-oxa-7-azaspiro[2.5]octan-7-yl)prop-2-en-1-one (D28B, Assumed)

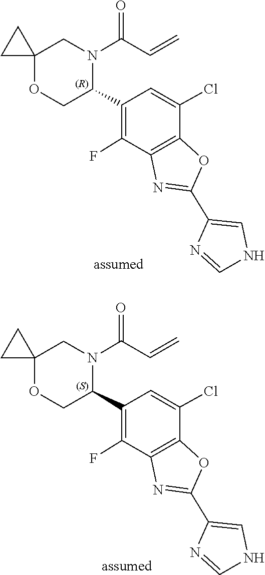

The racemic 1-(6-(7-chloro-4-fluoro-2-(1H-imidazol-4-yl)benzo[d]oxazol-5-yl)-4-oxa-7-azaspiro[2.5]octan-7-yl)prop-2-en-1-one was separated using the following conditions:

SFC: column: YMC Cellulose-SB, 20 mm×250 mm, 5 um; fluent: 32% EtOH/CO₂, flow rate: 40.0 mL/min; isomer 1, retention time=4.17 min; isomer 2, retention time=5.05 min;

Isomer 1:

¹H NMR (600 MHz, DMSO-d₆) δ 12.93 (brs, 1H), 8.24 (s, 1H), 7.97 (s, 1H), 7.65 (d, J=5.3 Hz, 1H), 6.90-6.81 (m, 1H), 6.21 (dd, J=16.6, 2.1 Hz, 1H), 5.95-5.90 (m, 1H), 5.75 (d, J=9.9 Hz, 1H), 4.29 (d, J=12.0 Hz, 1H), 3.98 (d, J=10.2 Hz, 1H), 3.81 (brs, 1H), 3.53 (brs, 1H), 0.93-0.86 (m, 1H), 0.79-0.76 (m, 1H), 0.71-0.68 (m, 1H), 0.53-0.50 (m, 1H). MS (ESI) m/z [M+1]⁺=403.

Isomer 2:

¹H NMR (600 MHz, DMSO-d₆) δ 12.94 (brs, 1H), 8.24 (s, 1H), 7.97 (s, 1H), 7.65 (d, J=5.2 Hz, 1H), 6.91-6.80 (m, 1H), 6.21 (d, J=16.6 Hz, 1H), 5.94-5.88 (m, 1H), 5.75 (d, J=10.1 Hz, 1H), 4.29 (d, J=12.0 Hz, 1H), 3.98 (d, J=9.6 Hz, 1H), 3.82 (brs, 1H), 3.51 (brs, 1H), 0.93-0.86 (m, 1H), 0.79-0.75 (m, 1H), 0.71-0.67 (m, 1H), 0.53-0.49 (m, 1H). MS (ESI) m/z [M+1]⁺=403.

### Example 2-30: Synthesis of (R)-1-(5-(7-chloro-4-fluoro-2-(1H-imidazol-4-yl)benzo[d]oxazol-5-yl)-2,2-dimethylmorpholino)prop-2-en-1-one (D29A, Assumed) and (S)-1-(5-(7-chloro-4-fluoro-2-(1H-imidazol-4-yl)benzo[d]oxazol-5-yl)-2,2-dimethylmorpholino)prop-2-en-1-one (D29B, Assumed)

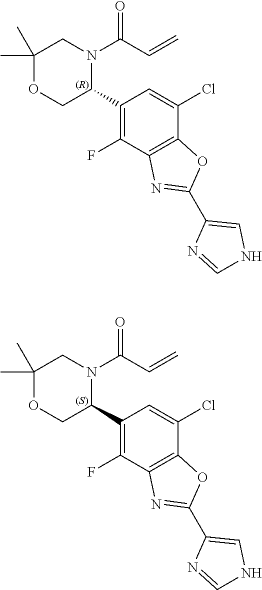

### Step 1: tert-butyl 5-[7-chloro-4-fluoro-2-[1-(2-trimethylsilylethoxymethyl)imidazol-4-yl]-1,3-benzoxazol-5-yl]-2,2-dimethyl-3H-1,4-oxazine-4-carboxylate (Assumed) and tert-butyl 5-(7-chloro-4-fluoro-2-(1-((2-(trimethylsilyl)ethoxy)methyl)-1H-imidazol-5-yl)benzo[d]oxazol-5-yl)-2,2-dimethyl-2,3-dihydro-4H-1,4-oxazine-4-carboxylate (Assumed)

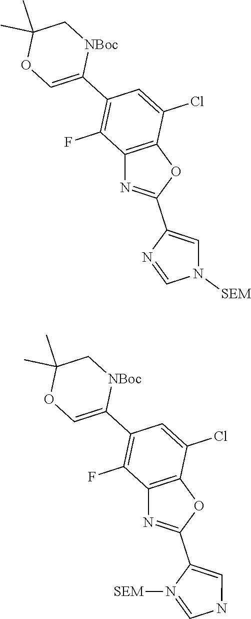

A mixture of 2-[[4-[7-chloro-4-fluoro-5-(4,4,5,5-tetramethyl-1,3,2-dioxaborolan-2-yl)-1,3-benzoxazol-2-yl]imidazol-1-yl]methoxy]ethyl-trimethyl-silane (assumed) and 7-chloro-4-fluoro-5-(4,4,5,5-tetramethyl-1,3,2-dioxaborolan-2-yl)-2-(1-((2-(trimethylsilyl)ethoxy)methyl)-1H-imidazol-5-yl)benzo[d]oxazole (assumed) (300 mg, 0.61 mmol), tert-butyl 5-diphenoxyphosphoryloxy-2,2-dimethyl-3H-1,4-oxazine-4-carboxylate (420 mg, 0.91 mmol), Pd(PPh₃)₄ (70 mg, 0.06 mmol) and Na₂CO₃ (193 mg, 1.82 mmol) in 1,4-dioxane (15 mL) and H₂O (5 mL) was stirred at 90° C. for 2 h under nitrogen atmosphere. After cooling to rt, the reaction mixture was concentrated under vacuum. The residue was purified by flash chromatography on silica (EA/PE 1/9 to 4/1) to give the title compounds (300 mg, 85% yield). MS (ESI) m/z [M+1]⁺=579.

### Step 2: 7-chloro-5-(6,6-dimethylmorpholin-3-yl)-4-fluoro-2-(1H-imidazol-4-yl)-1,3-benzoxazole

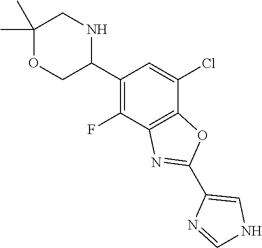 [SMILES: CC1(C)CNC(c2cc(Cl)c3oc(-c4c[nH]cn4)nc3c2F)CO1]

To a solution of tert-butyl 5-[7-chloro-4-fluoro-2-[1-(2-trimethylsilylethoxymethyl)imidazol-4-yl]-1,3-benzoxazol-5-yl]-2,2-dimethyl-3H-1,4-oxazine-4-carboxylate and tert-butyl 5-(7-chloro-4-fluoro-2-(1-((2-(trimethylsilyl)ethoxy)methyl)-1H-imidazol-5-yl)benzo[d]oxazol-5-yl)-2,2-dimethyl-2,3-dihydro-4H-1,4-oxazine-4-carboxylate (200 mg, 0.34 mmol) in HFIP (10 mL) was added dropwise MsOH (331 mg, 3.45 mmol) at rt. The resulting mixture was stirred at rt for 1 h followed by MeOH (10 mL) and NaBH₃CN (36 mg, 0.57 mmol) at rt. The resulting mixture was stirred at rt for another 1 h. Upon completion of the reaction, the solvent was removed in vacuo to give the crude title compound (180 mg). MS (ESI) m/z [M+1]⁺=351.

### Step 3:1-(5-(7-chloro-4-fluoro-2-(1H-imidazol-4-yl)benzo[d]oxazol-5-yl)-2,2-dimethylmorpholino)prop-2-en-1-one

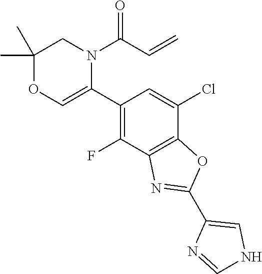 [SMILES: C=CC(=O)N1CC(C)(C)OC=C1c1cc(Cl)c2oc(-c3c[nH]cn3)nc2c1F]

To a solution of 7-chloro-5-(6,6-dimethylmorpholin-3-yl)-4-fluoro-2-(1H-imidazol-4-yl)-1,3-benzoxazole (crude, 180 mg, ca. 0.25 mmol) in ACN (5 mL) and NaHCO₃ (0.8 mol/L, 5 mL) was added dropwise acryloyl chloride (23 mg, 0.25 mmol) at rt. The resulting mixture was stirred at rt for 1 h. Upon completion of the reaction, ice water (50 mL) was added and extracted with DCM. The combined organic layers were dried over anhydrous Na₂SO₄, filtered and concentrated. The residue was purified by C18 column (mobile phases, ACN in water (0.1% FA), 27% to 43%) to give the title compound (50 mg, 36% yield over 2 steps). MS (ESI) m/z [M+H]⁺=405.

### Step 4: 1-[(5R)-5-[7-chloro-4-fluoro-2-(1H-imidazol-4-yl)-1,3-benzoxazol-5-yl]-2,2-dimethyl-morpholin-4-yl]prop-2-en-1-one (D29A, Assumed) and 1-[(5S)-5-[7-chloro-4-fluoro-2-(1H-imidazol-4-yl)-1,3-benzoxazol-5-yl]-2,2-dimethyl-morpholin-4-yl]prop-2-en-1-one (D29B, Assumed)

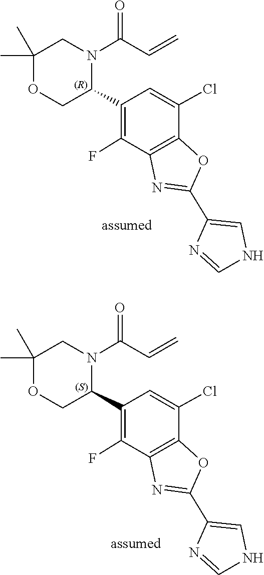

The racemic 1-(5-(7-chloro-4-fluoro-2-(1H-imidazol-4-yl)benzo[d]oxazol-5-yl)-2,2-dimethylmorpholino)prop-2-en-1-one was separated using the following conditions:

SFC: column: YMC Cellulose-SB, 20 mm×250 mm, 5 um; fluent: 30% EtOH/CO₂, flow rate: 40.0 mL/min; isomer 1, retention time=4.28 min; isomer 2, retention time=5.47 min.

Isomer 1:

¹H NMR (600 MHz, DMSO-d₆) δ 12.95 (brs, 1H), 8.22 (s, 1H), 7.98 (s, 1H), 7.58 (s, 1H), 6.92-6.75 (m, 1H), 6.19 (d, J=16.6 Hz, 1H), 5.73 (d, J=10.4 Hz, 1H), 5.63-5.59 (m, 1H), 3.98 (brs, 2H), 3.41-3.38 (m, 2H), 1.20 (s, 3H), 1.14 (s, 3H). MS (ESI) m/z [M+1]⁺=405.

Isomer 2:

¹H NMR (600 MHz, DMSO-d₆) δ 12.95 (brs, 1H), 8.22 (s, 1H), 7.98 (s, 1H), 7.59 (s, 1H), 6.91-6.73 (m, 1H), 6.19 (d, J=16.6 Hz, 1H), 5.73 (d, J=10.4 Hz, 1H), 5.65-5.58 (m, 1H), 3.98 (brs, 2H), 3.54-3.34 (m, 2H), 1.20 (s, 3H), 1.14 (s, 3H). MS (ESI) m/z [M+1]⁺=405.

### Example 2-31: Synthesis of 1-(6-(7-chloro-4-ethyl-2-(1H-imidazol-4-yl)benzo[d]oxazol-5-yl)-4-oxa-7-azaspiro[2.5]octan-7-yl)prop-2-en-1-one (D30)

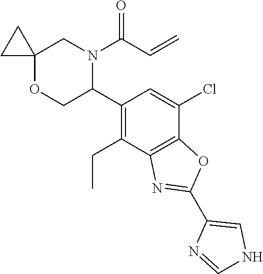 [SMILES: C=CC(=O)N1CC2(CC2)OCC1c1cc(Cl)c2oc(-c3c[nH]cn3)nc2c1CC]

### Step 1: 2-[[4-(5-bromo-7-chloro-4-vinyl-1,3-benzoxazol-2-yl)imidazol-1-yl]methoxy]ethyl-trimethyl-silane

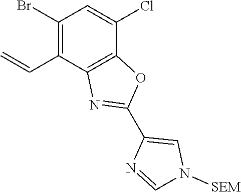 [SMILES: C=Cc1c(Br)cc(Cl)c2oc(-c3cn(COCC[Si](C)(C)C)cn3)nc12]

A mixture of 2-[[4-(5-bromo-7-chloro-4-iodo-1,3-benzoxazol-2-yl)imidazol-1-yl]methoxy]ethyl-trimethyl-silane (1.0 g, 1.80 mmol), 4,4,5,5-tetramethyl-2-vinyl-1,3,2-dioxaborolane (830 mg, 5.39 mmol), Pd(dppf)Cl₂ (130 mg, 0.18 mmol) and K₂CO₃ (750 mg, 5.43 mmol) in 1,4-dioxane (10 mL) and H₂O (3 mL) was stirred at 70° C. for 2 h under nitrogen atmosphere. After cooling to room temperature, the resulting mixture was concentrated under vacuum. The residue was purified by flash chromatography on silica gel (EA/PE 1/9 to 4/1) to give the title compound (700 mg, 85% yield). MS (ESI) m/z [M+1]⁺=454, 456.

### Step 2: 2-[[4-(5-bromo-7-chloro-4-ethyl-1,3-benzoxazol-2-yl)imidazol-1-yl]methoxy]ethyl-trimethyl-silane

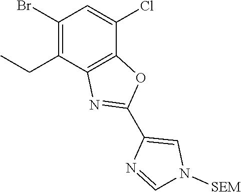 [SMILES: CCc1c(Br)cc(Cl)c2oc(-c3cn(COCC[Si](C)(C)C)cn3)nc12]

To a solution of 2-[[4-(5-bromo-7-chloro-4-vinyl-1,3-benzoxazol-2-yl)imidazol-1-yl]methoxy]ethyl-trimethyl-silane (700 mg, 1.54 mmol) in EA (10 mL) was added PtO₂ (70 mg) under nitrogen atmosphere. The mixture was stirred at rt for 1 h under hydrogen atmosphere using a hydrogen balloon (2 atm). Upon completion of the reaction, the catalyst was filtered out and the filtration was concentrated in vacuo. The residue was purified by flash chromatography on silica gel (EA/PE 1/9 to 4/1) to give the title compound (500 mg, 71.1% yield). MS (ESI) m/z [M+1]⁺=456, 458.

### Step 3: 2-[[4-[7-chloro-4-ethyl-5-(4,4,5,5-tetramethyl-1,3,2-dioxaborolan-2-yl)-1,3-benzoxazol-2-yl]imidazol-1-yl]methoxy]ethyl-trimethyl-silane

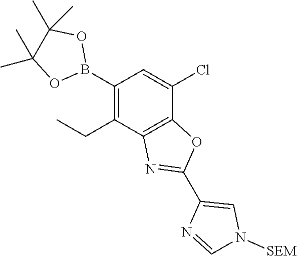 [SMILES: CCc1c(B2OC(C)(C)C(C)(C)O2)cc(Cl)c2oc(-c3cn(COCC[Si](C)(C)C)cn3)nc12]

A mixture of 2-[[4-(5-bromo-7-chloro-4-ethyl-1,3-benzoxazol-2-yl)imidazol-1-yl]methoxy]ethyl-trimethyl-silane (300 mg, 0.66 mmol), 4,4,5,5-tetramethyl-2-(4,4,5,5-tetramethyl-1,3,2-dioxaborolan-2-yl)-1,3,2-dioxaborolane (250 mg, 0.98 mmol), Pd(dppf)Cl₂ (48 mg, 0.066 mmol) and KOAc (193 mg, 1.97 mmol) in 1,4-dioxane (10 mL) was stirred at 90° C. for 12 h under nitrogen atmosphere. After cooling to room temperature, the resulting mixture was concentrated under vacuum. The residue was purified by flash chromatography on silica gel (EA/PE 1/9 to 4/1) to give the title compound (200 mg, 60.4% yield). MS (ESI) m/z [M+1]⁺=504.

### Step 4: tert-butyl 6-[7-chloro-4-ethyl-2-[1-(2-trimethylsilylethoxymethyl)imidazol-4-yl]-1,3-benzoxazol-5-yl]-4-oxa-7-azaspiro[2.5]oct-5-ene-7-carboxylate

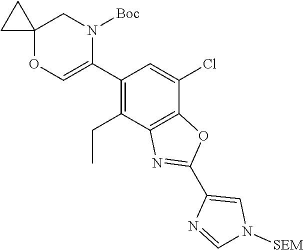 [SMILES: CCc1c(C2=COC3(CC3)CN2C(=O)OC(C)(C)C)cc(Cl)c2oc(-c3cn(COCC[Si](C)(C)C)cn3)nc12]

A mixture of 2-[[4-[7-chloro-4-ethyl-5-(4,4,5,5-tetramethyl-1,3,2-dioxaborolan-2-yl)-1,3-benzoxazol-2-yl]imidazol-1-yl]methoxy]ethyl-trimethyl-silane (80 mg, 0.16 mmol), tert-butyl 6-diphenoxyphosphoryloxy-4-oxa-7-azaspiro[2.5]oct-5-ene-7-carboxylate (109 mg, 0.24 mmol), Pd(PPh₃)₄ (18 mg, 0.016 mmol) and Na₂CO₃ (50 mg, 0.48 mmol) in 1,4-dioxane (5 mL) and H₂O (2 mL) was stirred at 90° C. for 2 h under nitrogen atmosphere. After cooling to rt, the resulting mixture was concentrated under vacuum. The residue was purified by flash chromatography on silica gel (EA/PE 1/9 to 4/1) to give the title compound (50 mg, 53.64% yield). MS (ESI) m/z [M+1]⁺=587.

### Step 5: 7-chloro-4-ethyl-2-(1H-imidazol-4-yl)-5-(4-oxa-7-azaspiro[2.5]octan-6-yl)-1,3-benzoxazole

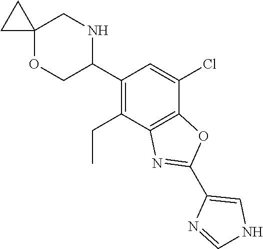 [SMILES: CCc1c(C2COC3(CC3)CN2)cc(Cl)c2oc(-c3c[nH]cn3)nc12]

To a solution of tert-butyl 6-[7-chloro-4-ethyl-2-[1-(2-trimethylsilylethoxymethyl) imidazol-4-yl]-1,3-benzoxazol-5-yl]-4-oxa-7-azaspiro[2.5]oct-5-ene-7-carboxylate (50 mg, 0.085 mmol) in HFIP (3 mL) was added dropwise MsOH (82 mg, 0.85 mmol) at rt. The resulting mixture was stirred at rt for 1 h followed by MeOH (5 mL) and NaBH₃CN (9 mg, 0.14 mmol) at rt. The resulting mixture was stirred at rt for 1 h. Upon completion of the reaction, the solvent was removed in vacuo to give the crude title compound (40 mg). MS (ESI) m/z [M+1]⁺=359.

### Step 6: 1-[6-[7-chloro-4-ethyl-2-(1H-imidazol-4-yl)-1,3-benzoxazol-5-yl]-4-oxa-7-azaspiro[2.5]octan-7-yl]prop-2-en-1-one

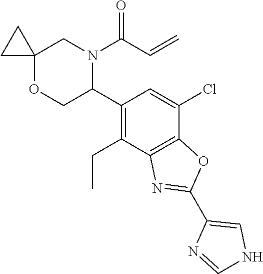 [SMILES: C=CC(=O)N1CC2(CC2)OCC1c1cc(Cl)c2oc(-c3c[nH]cn3)nc2c1CC]

To a stirred solution of 7-chloro-4-ethyl-2-(1H-imidazol-4-yl)-5-(4-oxa-7-azaspiro[2.5]octan-6-yl)-1,3-benzoxazole (crude, 40 mg, ca. 0.056 mmol) in ACN (2 mL) and NaHCO₃ (0.8 mol/L, 2 mL) was added dropwise acryloyl chloride (5 mg, 0.056 mmol) at rt. The resulting mixture was stirred at rt for 1 h. Upon completion of the reaction, ice water (50 mL) was added and extracted with DCM (3×50 mL). The combined organic layers were dried over anhydrous Na₂SO₄, filtered and concentrated. The residue was purified by C18 column (mobile phases, ACN in water (0.1% FA), 27% to 43%) to give the title compound (5 mg, 14% yield over 2 steps).

¹H NMR (600 MHz, DMSO-d₆) δ 12.78 (brs, 1H), 8.09 (s, 1H), 7.87 (s, 1H), 7.77 (brs, 1H), 6.68 (dd, J=16.6, 10.4 Hz, 1H), 6.17 (d, J=16.4 Hz, 1H), 5.76 (brs, 1H), 5.66 (dd, J=10.4, 2.2 Hz, 1H), 4.18 (d, J=11.4 Hz, 1H), 3.90 (d, J=9.9 Hz, 1H), 3.66 (d, J=14.1 Hz, 1H), 3.41-3.36 (m, 1H), 2.93-2.83 (m, 2H), 1.08-1.05 (m, 3H), 0.88-0.82 (m, 1H), 0.73-0.69 (m, 1H), 0.62-0.59 (m, 1H), 0.41-0.37 (m, 1H). MS (ESI) m/z [M+1]⁺=413.

### Example 2-32: Synthesis of 1-(2-(7-chloro-2-(1H-imidazol-5-yl)benzo[d]oxazol-5-yl)tetrahydropyridazin-1(2H)-yl)prop-2-en-1-one (D33)

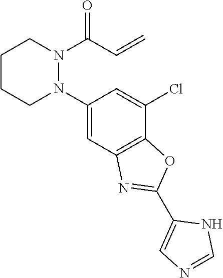 [SMILES: C=CC(=O)N1CCCCN1c1cc(Cl)c2oc(-c3cnc[nH]3)nc2c1]

### Step 1: tert-butyl 2-[7-chloro-2-[3-(2-trimethylsilylethoxymethyl)imidazol-4-yl]-1,3-benzoxazol-5-yl]hexahydropyridazine-1-carboxylate

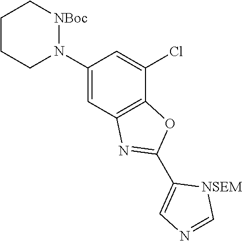 [SMILES: CC(C)(C)OC(=O)N1CCCCN1c1cc(Cl)c2oc(-c3cncn3COCC[Si](C)(C)C)nc2c1]

A mixture of 2-[[5-(5-bromo-7-chloro-1,3-benzoxazol-2-yl)imidazol-1-yl]methoxy]ethyl-trimethyl-silane (300 mg, 0.69 mmol), tert-butyl hexahydropyridazine-1-carboxylate (130 mg, 0.69 mmol), PEPPSI-Pd (68 mg, 0.069 mmol) and Cs₂CO₃ (684 mg, 2.09 mmol) in 1,4-dioxane (15 mL) was stirred at 100° C. for 16 h under nitrogen atmosphere. After cooling to room temperature, the resulting mixture was concentrated under vacuum. The residue was purified by flash chromatography on silica gel (EA/PE 1/9 to 4/1) to give the title compound (50 mg, 13.4% yield). MS (ESI) m/z [M+1]⁺=534.

### Step 2: 7-chloro-2-(1H-imidazol-5-yl)-5-(tetrahydropyridazin-1(2H)-yl)benzo[d]oxazole

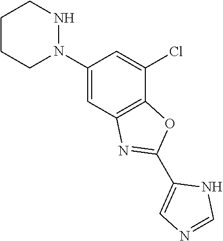 [SMILES: Clc1cc(N2CCCCN2)cc2nc(-c3cnc[nH]3)oc12]

To a solution of tert-butyl 2-[7-chloro-2-[3-(2-trimethylsilylethoxymethyl)imidazol-4-yl]-1,3-benzoxazol-5-yl]hexahydropyridazine-1-carboxylate (50 mg, 0.093 mmol) in HFIP (3 mL) was added dropwise MsOH (89 mg, 0.93 mmol) at rt. The resulting mixture was stirred at rt for 1 h. Upon completion of the reaction, the solvent was removed in vacuo to give the crude title compound (50 mg). MS (ESI) m/z [M+1]⁺=304.

### Step 3:1-[2-[7-chloro-2-(1H-imidazol-5-yl)-1,3-benzoxazol-5-yl]hexahydropyridazin-1-yl]prop-2-en-1-one

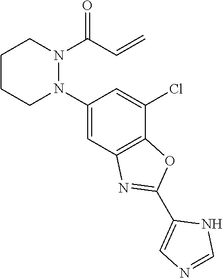 [SMILES: C=CC(=O)N1CCCCN1c1cc(Cl)c2oc(-c3cnc[nH]3)nc2c1]

To a solution of 7-chloro-5-hexahydropyridazin-1-yl-2-(1H-imidazol-5-yl)-1,3-benzoxazole (crude, 20 mg, ca. 0.032 mmol) in ACN (2 mL) and NaHCO₃ (0.8 M/L, 2 mL) was added dropwise acryloyl chloride (4 mg, 0.049 mmol) at rt. The resulting mixture was stirred at rt for 1 h. Upon completion of the reaction, ice water (50 mL) was added and extracted with DCM. The combined organic layers were dried over anhydrous Na₂SO₄, filtered and concentrated. The residue was purified by C18 column (mobile phases, ACN in water (0.1% FA), 25% to 40%) to give the title compound (2.49 mg, 19% yield over 2 steps).

¹H NMR (600 MHz, DMSO-d₆) δ 12.84 (brs, 1H), 8.10 (s, 1H), 7.93 (s, 1H), 7.12 (s, 1H), 6.91 (s, 1H), 6.72 (dd, J=16.7, 10.3 Hz, 1H), 6.29 (d, J=17.8 Hz, 1H), 5.75 (d, J=9.9 Hz, 1H), 4.32 (t, J=14.6 Hz, 2H), 3.12-3.00 (m, 1H), 2.82-2.80 (m, 1H), 1.72-1.63 (m, 3H), 1.58-1.48 (m, 1H). MS (ESI) m/z [M+1]⁺=358.

Compounds B32, B35 in table 2-12 were synthesized from corresponding starting materials using procedure in examples 2-31 and 2-32, respectively.

TABLE 2-12

| Compound | Structure | Chemical Name | ¹H NMR data LC/MS m/z (M + H) |
|---|---|---|---|
| B32A | 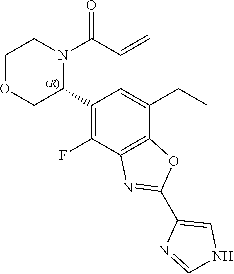 | (R)-1-(3-(7-ethyl-4- fluoro-2-(1H-imidazol- 4-yl)benzo[d]oxazol-5- yl)morpholino)prop-2- en-1-one (assumed) | ¹H NMR (600 MHz, DMSO-d6) δ 12.65 (brs, 1H), 8.12 (s, 1H), 7.93 (s, 1H), 7.32 (d, J = 6.3 Hz, 1H),, 6.85 (dd, J = 16.6, 10.5 Hz, 1H), 6.17 (dd, J = 16.6, 2.0 Hz, 1H), 5.73 (d, J = 10.4 Hz, 1H), 5.67-5.58 (m, 1H), 4.26 (d, J = 12.0 Hz, 1H), 4.15-4.11 (m, 1H), 3.98 (d, J = 8.6 Hz, 1H), 3.87 (d, J = 9.2 Hz, 1H), 3.54 (t, J = 10.2 Hz, 1H), 3.24- 3.14 (m, 1H), 2.90-2.87 (m, 2H), 1.31- 1.29 (m, 3H). MS (ESI) m/z [M + 1]⁺ = 371. |
| B32B | 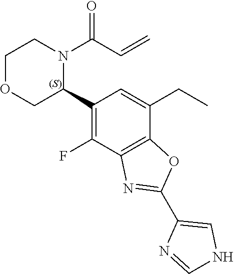 | (S)-1-(3-(7-ethyl-4- fluoro-2-(1H-imidazol- 4-yl)benzo[d]oxazol-5- yl)morpholino)prop-2- en-1-one (assumed) | ¹H NMR (600 MHz, DMSO-d₆) δ 12.84 (brs, 1H), 8.15 (s, 1H), 7.93 (s, 1H), 7.32 (d, J = 6.1 Hz, 1H), 6.85 (dd, J = 16.6, 10.5 Hz, 1H), 6.17 (dd, J = 16.6, 2.0 Hz, 1H), 5.73 (d, J = 10.4 Hz, 1H), 5.67-5.59 (m, 1H), 4.26 (d, J = 12.0 Hz, 1H), 4.14-4.10 (m, 1H), 3.98 (d, J = 8.8 Hz, 1H), 3.87 (d, J = 9.6 Hz, 1H), 3.54 (t, J = 10.3 Hz, 1H), 3.28-3.17 (m, 1H), 2.91-2.87 (m, 2H), 1.31- 1.29 (m, 3H). MS (ESI) m/z [M + 1]⁺ 371. |
| D35 | 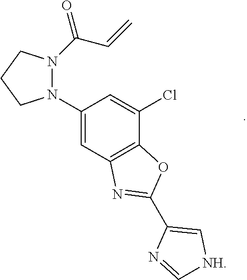 [SMILES: C=CC(=O)N1CCCN1c1cc(Cl)c2oc(-c3c[nH]cn3)nc2c1] | 1-(2-(7-chloro-2-(1H- imidazol-4- yl)benzo[d]oxazol-5- yl)pyrazolidin-1- yl)prop-2-en-1-one | 1H NMR (600 MHz, DMSO-d6) δ 12.86 (s, 1H), 8.11 (s, 1H), 7.93 (s, 1H), 7.23 (s, 1H), 7.11 (s, 1H), 6.62 (dd, J = 17.0, 10.7 Hz, 1H), 6.24 (d, J = 17.0 Hz, 1H), 5.68 (d, J = 9.6 Hz, 1H), 4.02-3.73 (m, 2H), 3.53-3.36 (m, 2H), 2.09-1.84 (m, 2H). MS (ESI) m/z [M + H]⁺ = 344. |

### Example 2-33: Synthesis of (R)-1-(3-(4-chloro-7-ethyl-2-(H-imidazol-4-yl)benzo[d]oxazol-6-yl)morpholino)prop-2-en-1-one (L12A, Assumed) and (S)-1-(3-(4-chloro-7-ethyl-2-(1H-imidazol-4-yl)benzo[d]oxazol-6-yl)morpholino)prop-2-en-1-one (L12B3, Assumed)

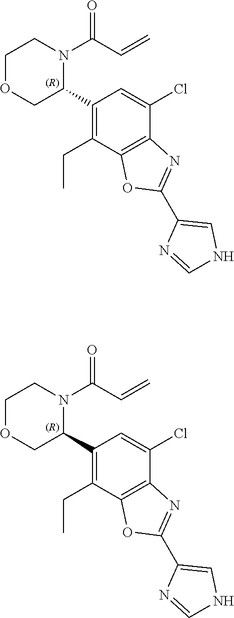

### Step 1: tert-butyl 5-(4-chloro-7-methoxy-2-(1-((2-(trimethylsilyl)ethoxy)methyl)-1H-imidazol-4-yl)benzo[d]oxazol-6-yl)-2,3-dihydro-4H-1,4-oxazine-4-carboxylate

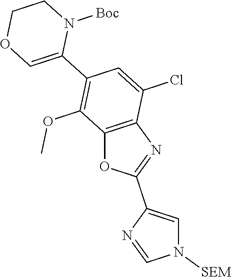 [SMILES: COc1c(C2=COCCN2C(=O)OC(C)(C)C)cc(Cl)c2nc(-c3cn(COCC[Si](C)(C)C)cn3)oc12]

A mixture of 2-[[4-[4-chloro-7-methoxy-6-(4,4,5,5-tetramethyl-1,3,2-dioxaborolan-2-yl)-1,3-benzoxazol-2-yl]imidazol-1-yl]methoxy]ethyl-trimethyl-silane (1.20 g, 2.40 mmol), tert-butyl 5-diphenoxyphosphoryloxy-2,3-dihydro-1,4-oxazine-4-carboxylate (1.00 g, 2.31 mmol), Pd(dppf)Cl₂ (170 mg, 0.23 mmol) and K₂CO₃ (980 mg, 7.10 mmol) in 1,4-dioxane (15 mL) and H₂O (3 mL) was stirred at 85° C. for 2 h under nitrogen atmosphere. After cooled to room temperature, the resulting mixture was concentrated under vacuum. The residue was purified by flash chromatography on silica gel eluted with 10%-90% EA in PE to give title compound (800 mg, 60% yield). MS (ESI) m/z [M+H]⁺=563.

### Step 2: 4-chloro-2-(1H-imidazol-4-yl)-7-methoxy-6-(morpholin-3-yl)benzo[d]oxazole

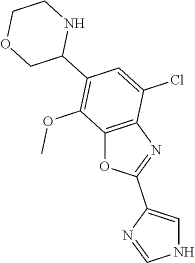 [SMILES: COc1c(C2COCCN2)cc(Cl)c2nc(-c3c[nH]cn3)oc12]

To a stirred mixture of tert-butyl 5-(4-chloro-7-methoxy-2-(1-((2-(trimethylsilyl)ethoxy)methyl)-1H-imidazol-4-yl)benzo[d]oxazol-6-yl)-2,3-dihydro-4H-1,4-oxazine-4-carboxylate (800 mg, 1.42 mmol) in HFIP (10 mL) was added MsOH (511 mg, 5.33 mmol) dropwise at rt. The resulting mixture was stirred at rt for 1 h. Then to the above was added MeOH (10 mL) and NaBH4CN (149 mg, 2.40 mmol) at rt. The resulting mixture was stirred at rt for 1 h. Upon completion of the reaction, the resulting mixture was concentrated under reduced pressure to give the title compound (800 mg). MS (ESI) m/z [M+H]⁺=335.

### Step 3: tert-butyl 3-(4-chloro-2-(1H-imidazol-4-yl)-7-methoxybenzo[d]oxazol-6-yl) morpholine-4-carboxylate

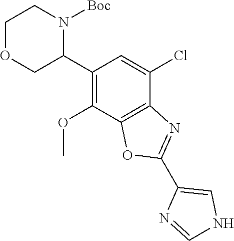 [SMILES: COc1c(C2COCCN2C(=O)OC(C)(C)C)cc(Cl)c2nc(-c3c[nH]cn3)oc12]

To a stirred mixture of 4-chloro-2-(1H-imidazol-4-yl)-7-methoxy-6-(morpholin-3-yl)benzo[d]oxazole (800 mg, 1.19 mmol, crude) in ACN (8 mL) and NaHCO₃ (8 mL, 6 mmol, 0.8 mol/L) was added tert-butoxycarbonyl tert-butyl carbonate (391 mg, 1.79 mmol) dropwise at rt. The resulting mixture was stirred at rt for 1 h. Upon completion of the reaction, the reaction was quenched with water (50 mL). The resulting mixture was extracted with DCM (3×50 mL). The combined organic layers were dried over anhydrous Na₂SO₄. After filtration, the filtrate was concentrated under reduced pressure. The residue was purified by flash chromatography on silica gel eluted with 10%-70% EA in PE to give the title compound (500 mg, 96% yield). MS (ESI) m/z [M+H]⁺=435.

### Step 4: tert-butyl 3-(4-chloro-7-methoxy-2-(1-((2-(trimethylsilyl)ethoxy)methyl)-1H-imidazol-4-yl)benzo[d]oxazol-6-yl)morpholine-4-carboxylate

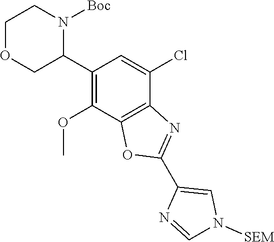 [SMILES: COc1c(C2COCCN2C(=O)OC(C)(C)C)cc(Cl)c2nc(-c3cn(COCC[Si](C)(C)C)cn3)oc12]

To a stirred mixture of tert-butyl 3-(4-chloro-2-(1H-imidazol-4-yl)-7-methoxybenzo[d]oxazol-6-yl) morpholine-4-carboxylate (460 mg, 1.06 mmol) in DMF (10 mL) was added NaH (63 mg, 1.58 mmol, 60% in mineral oil) in portion-wise at 0° C. The resulting mixture was stirred for 1 h at 0° C. To the above was added SEM-Cl (263 mg, 1.58 mmol) at 0° C. The resulting mixture was stirred for 1 h at rt. The reaction was quenched with water (50 mL). The resulting mixture was extracted with DCM (3×50 mL). The combined organic layers were dried over anhydrous Na₂SO₄. After filtration, the filtrate was concentrated under reduced pressure. The residue was purified by flash chromatography on silica gel eluted with 10%-80% EA in PE to give the product (500 mg, 83% yield). MS (ESI) m/z [M+H]⁺=565.

### Step 5: tert-butyl 3-(4-chloro-7-hydroxy-2-(1-((2-(trimethylsilyl)ethoxy)methyl)-1H-imidazol-4-yl)benzo[d]oxazol-6-yl)morpholine-4-carboxylate

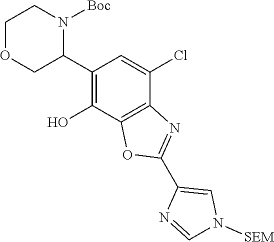 [SMILES: CC(C)(C)OC(=O)N1CCOCC1c1cc(Cl)c2nc(-c3cn(COCC[Si](C)(C)C)cn3)oc2c1O]

A mixture of tert-butyl 3-(4-chloro-7-methoxy-2-(1-((2-(trimethylsilyl)ethoxy)methyl)-1H-imidazol-4-yl)benzo[d]oxazol-6-yl)morpholine-4-carboxylate (500 mg, 0.88 mmol) and NaSEt (380 mg, 4.42 mmol) in DMF (10 mL) was stirred at 60° C. under microware for 2 h. After cooled to rt, the resulting mixture was concentrated under vacuum. The residue was purified by flash chromatography on silica gel eluted with 1%-10% MeOH in DCM to give the title compound (413 mg, 85% yield). MS (ESI) m/z [M+H]⁺=551.

### Step 6: tert-butyl 3-(4-chloro-7-(((trifluoromethyl)sulfonyl)oxy)-2-(1-((2-(trimethylsilyl)ethoxy)methyl)-1H-imidazol-4-yl)benzo[d]oxazol-6-yl)morpholine-4-carboxylate

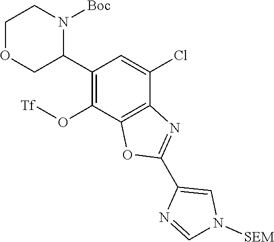

To a stirred mixture of tert-butyl 3-(4-chloro-7-hydroxy-2-(1-((2-(trimethylsilyl)ethoxy)methyl)-1H-imidazol-4-yl)benzo[d]oxazol-6-yl)morpholine-4-carboxylate (300 mg, 0.54 mmol), TEA (165 mg, 1.63 mmol) and DMAP (66 mg, 0.54 mmol) in THF (8 mL) was added N-phenyl-bis(trifluoromethanesulfonimide) (291 mg, 0.81 mmol) at 0° C. The resulting mixture was stirred for 1 h at rt. Upon completion of the reaction, the resulting mixture was concentrated under vacuum. The residue was purified by flash chromatography on silica gel eluted with 10%-100% EA in PE to give title compound (300 mg, 80.67% Yield). MS (ESI) m/z [M+H]⁺=683.

### Step 7: tert-butyl 3-(4-chloro-2-(1-((2-(trimethylsilyl)ethoxy)methyl)-1H-imidazol-4-yl)-7-vinylbenzo[d]oxazol-6-yl)morpholine-4-carboxylate

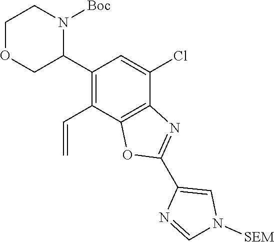 [SMILES: C=Cc1c(C2COCCN2C(=O)OC(C)(C)C)cc(Cl)c2nc(-c3cn(COCC[Si](C)(C)C)cn3)oc12]

A mixture of tert-butyl 3-[4-chloro-7-(trifluoromethylsulfonyloxy)-2-[1-(2-trimethylsilylethoxymethyl)imidazol-4-yl]-1,3-benzoxazol-6-yl]morpholine-4-carboxylate (200 mg, 0.29 mmol), potassium vinyltrifluoroborate (194.3 mg, 1.45 mmol), Pd(dppf)Cl₂ (42.4 mg, 0.058 mmol), K₃PO₄ (165.4 mg, 0.78 mmol) in 1,4-dioxane (8 mL) and H₂O (2 mL) was stirred at 90° C. for 3 h under N₂ atmosphere. Upon completion of the reaction, the mixture was cooled to rt and concentrated in vacuo, and the residue was purified by silica gel column chromatography (PE/EA 5:1 to 1:1) to give the product (120 mg, 73% yield). MS (ESI) m/z [M+H]⁺=561.

### Step 8: tert-butyl 3-(4-chloro-7-ethyl-2-(1-((2-(trimethylsilyl)ethoxy)methyl)-1H-imidazol-4-yl)benzo[d]oxazol-6-yl)morpholine-4-carboxylate

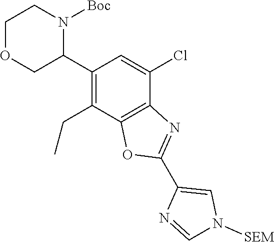 [SMILES: CCc1c(C2COCCN2C(=O)OC(C)(C)C)cc(Cl)c2nc(-c3cn(COCC[Si](C)(C)C)cn3)oc12]

A mixture of tert-butyl 3-[4-chloro-2-[3-(2-trimethylsilylethoxymethyl)imidazol-4-yl]-7-vinyl-1,3-benzoxazol-6-yl]morpholine-4-carboxylate (120 mg, 0.21 mmol) and PtO₂ (20 mg, 0.09 mmol) in THF (10 mL) was stirred at rt for 2 h under H₂ atmosphere. Upon completion of the reaction, the catalyst was filtered out and the filtration was concentrated in vacuo to give the crude product (110 mg, 0.91% yield). MS (ESI) m/z [M+H]⁺=563.

### Step 9: 4-chloro-7-ethyl-2-(1H-imidazol-4-yl)-6-(morpholin-3-yl)benzo[d]oxazole

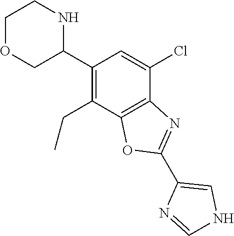 [SMILES: CCc1c(C2COCCN2)cc(Cl)c2nc(-c3c[nH]cn3)oc12]

To a solution of tert-butyl 3-[4-chloro-7-ethyl-2-[3-(2-trimethylsilylethoxymethyl)imidazol-4-yl]-1,3-benzoxazol-6-yl]morpholine-4-carboxylate (110 mg, 0.195 mmol) in DCM (5 mL) was added TFA (2 mL) at rt. After stirring at rt for 2 h, the solvent was removed in vacuo, and the residue was used in next step without further purification. MS (ESI) m/z [M+H]⁺=333.

### Step 10: 1-(3-(4-chloro-7-ethyl-2-(1H-imidazol-4-yl)benzo[d]oxazol-6-yl)morpholino)prop-2-en-1-one

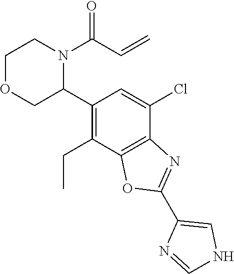 [SMILES: C=CC(=O)N1CCOCC1c1cc(Cl)c2nc(-c3c[nH]cn3)oc2c1CC]

To a solution of 4-chloro-7-ethyl-2-(1H-imidazol-4-yl)-6-(morpholin-3-yl)benzo[d]oxazole (50 mg, 0.15 mmol) and NaHCO₃ (70 mg, 0.83 mmol) in MeOH (5 mL) and H₂O (5 mL) was added dropwise acryloyl chloride (20 mg, 0.22 mmol) at 0° C. After stirring at 0° C. for 2 h, the mixture was extracted with DCM. The organic layer was dried over anhydrous Na₂SO₄ and concentrated in vacuo. The residue was purified by C18 column (mobile phases, ACN in water (0.1% FA), 27% to 43%) to give the title compound (15 mg, 25.8% yield). MS (ESI) m/z [M+H]⁺=387.

### Step 11: (R)-1-(3-(4-chloro-7-ethyl-2-(1H-imidazol-4-yl)benzo[d]oxazol-6-yl)morpholino)prop-2-en-1-one (L12A, Assumed) and (S)-1-(3-(4-chloro-7-ethyl-2-(1H-imidazol-4-yl)benzo[d]oxazol-6-yl)morpholino)prop-2-en-1-one (L12B, Assumed)

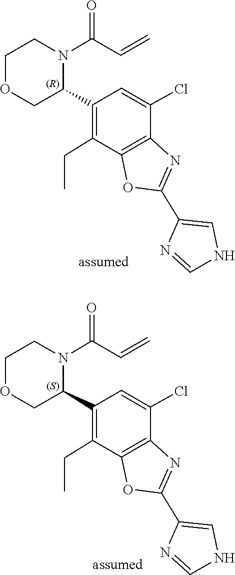

Racemic 1-(3-(4-chloro-7-ethyl-2-(1H-imidazol-4-yl)benzo[d]oxazol-6-yl)morpholino)prop-2-en-1-one was separated using the following conditions:

SFC: column: Lux Cellulose-4, 30 mm×250 mm, 5 um; fluent: 38% MeOH (0.3% 2M NH₃/MeOH)/CO2 isomer 1, retention time=9.82 min; isomer 2, retention time=11.93 min.

Isomer 1: (2 mg, 0.018 mmol, 3.4% yield).

¹H NMR (600 MHz, DMSO-d₆) δ 12.84 (s, 1H), 8.16 (s, 1H), 7.91 (s, 1H), 7.74 (s, 1H), 6.76 (dd, J=16.4, 10.4 Hz, 1H), 6.17 (d, J=14.9 Hz, 1H), 5.75-5.64 (m, 2H), 4.23-4.12 (m, 1H), 4.04-3.96 (m, 1H), 3.90-3.79 (m, 2H), 3.57-3.45 (m, 2H), 3.01-2.79 (m, 2H), 1.15 (t, J=7.3 Hz, 3H). MS (ESI) m/z [M+H]⁺=387.

Isomer 2: (1.8 mg, 0.017 mmol, 3.0% yield).

¹H NMR (600 MHz, DMSO-d₆) δ 12.84 (s, 1H), 8.16 (s, 1H), 7.91 (s, 1H), 7.74 (s, 1H), 6.76 (dd, J=16.4, 10.4 Hz, 1H), 6.17 (d, J=14.9 Hz, 1H), 5.77-5.62 (m, 2H), 4.23-4.12 (m, 1H), 4.04-3.97 (m, 1H), 3.90-3.79 (m, 2H), 3.57-3.45 (m, 2H), 3.01-2.78 (m, 2H), 1.15 (t, J=7.3 Hz, 3H). MS (ESI) m/z [M+H]⁺=387.

### Example 2-34: Synthesis of (R)-1-(5-(4-chloro-7-fluoro-2-(1H-imidazol-4-yl)benzo[d]oxazol-6-yl)-2,2-dimethylmorpholino)prop-2-en-1-one (L13A, Assumed) and (S)-1-(5-(4-chloro-7-fluoro-2-(1H-imidazol-4-yl)benzo[d]oxazol-6-yl)-2,2-dimethylmorpholino)prop-2-en-1-one (L13B, Assumed)

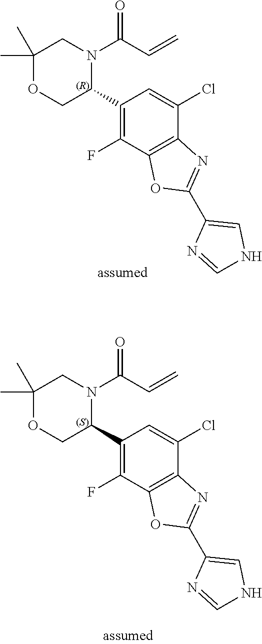

### Step 1: 2-Amino-5-bromo-3-chloro-6-fluorophenol

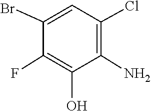 [SMILES: Nc1c(Cl)cc(Br)c(F)c1O]

A solution of 6-bromo-4-chloro-7-fluoro-3H-1,3-benzoxazol-2-one (2.5 g, 9.4 mmol) in aq. NaOH (3% wt., 100 mL) was stirred at rt for 4 h. Upon completion of the reaction, the solid was collected by filtration and dried in vacuo to give the title compound (2.2 g, 98% yield). MS (ESI) m/z [M+H]⁺=240, 242.

### Step 2: 6-bromo-4-chloro-7-fluoro-2-(1H-imidazol-4-yl)benzo[d]oxazole

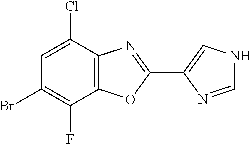 [SMILES: Fc1c(Br)cc(Cl)c2nc(-c3c[nH]cn3)oc12]

A solution of 2-amino-5-bromo-3-chloro-6-fluoro-phenol (2.2 g, 9.1 mmol) and 5-(trifluoromethyl)-1H-imidazole (3 g, 22.04 mmol) in aq. NaOH (0.5 M/L, 100 mL) was stirred at rt for overnight. The solid was collected by filtration and the filter cake was washed with H₂O. The crude product was used in next step without purification. MS (ESI) m/z [M+H]⁺=316, 318.

### Step 3: 6-bromo-4-chloro-7-fluoro-2-(1-((2-(trimethylsilyl)ethoxy)methyl)-1H-imidazol-4-yl)benzo[d]oxazole (Assumed SEM Position)

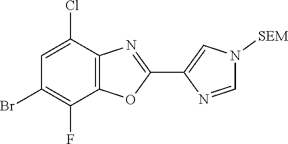 [SMILES: C[Si](C)(C)CCOCn1cnc(-c2nc3c(Cl)cc(Br)c(F)c3o2)c1]

A solution of 6-bromo-4-chloro-7-fluoro-2-(1H-imidazol-4-yl)benzo[d]oxazole (6.4 g, 20 mmol), SEMCl (6.74 g, 40.4 mmol) and TEA (6.13 g, 60.7 mmol) in DMF (100 mL) was stirred at rt for overnight. The mixture was extracted with EA. The organic layers were washed with brine, dried over anhydrous Na₂SO₄ and filtered. The solvent was concentrated in vacuo. The residue was purified by silica gel column chromatography (PE/EA 2/1 to 1/1) to give the title product (3.2 g, 35% yield over 2 steps). MS (ESI) m/z [M+H]⁺=446, 448.

### Step 4: tert-butyl 5-(4-chloro-7-fluoro-2-(1-((2-(trimethylsilyl)ethoxy)methyl)-1H-imidazol-4-yl)benzo[d]oxazol-6-yl)-2,2-dimethyl-2,3-dihydro-4H-1,4-oxazine-4-carboxylate

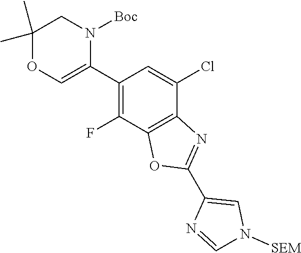 [SMILES: CC(C)(C)OC(=O)N1CC(C)(C)OC=C1c1cc(Cl)c2nc(-c3cn(COCC[Si](C)(C)C)cn3)oc2c1F]

A mixture of 2-[[5-(6-bromo-4-chloro-7-fluoro-1,3-benzoxazol-2-yl)imidazol-1-yl]methoxy]ethyl-trimethyl-silane (assumed SEM position) (3.2 g, 7.2 mmol), 4,4,5,5-tetramethyl-2-(4,4,5,5-tetramethyl-1,3,2-dioxaborolan-2-yl)-1,3,2-dioxaborolane (2.18 g, 8.58 mmol), Pd(dppf)Cl₂ (523.9 mg, 0.72 mmol) and KOAc (1.4 g, 14 mmol) in 1,4-dioxane (100 mL) was stirred at 100° C. for 5 h under N₂ atmosphere. Upon completion of the reaction, the mixture was cooled to rt. Then tert-butyl 5-diphenoxyphosphoryloxy-2,2-dimethyl-3H-1,4-oxazine-4-carboxylate (3.97 g, 8.60 mmol), Pd(dppf)Cl₂ (523.9 mg, 0.72 mmol) and K₃PO₄ (4.55 g, 21.5 mmol) were added following by H₂O (20 mL). The mixture was stirred at 80° C. for 3 h under N₂ atmosphere. Upon completion of the reaction, the mixture was cooled to rt and diluted with EA. The organic layer was collected and washed with H₂O, dried over anhydrous Na₂SO₄ and concentrated. The residue was purified by silica gel column chromatography (PE/EA 5/1 to 1/1) to give the title compound (830 mg, 20% yield over 2 steps). MS (ESI) m/z [M+H]⁺=579.

### Step 5: 4-chloro-6-(6,6-dimethylmorpholin-3-yl)-7-fluoro-2-(1H-imidazol-4-yl)benzo[d]oxazole

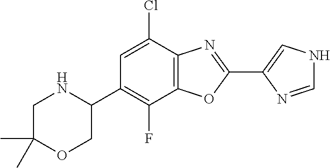 [SMILES: CC1(C)CNC(c2cc(Cl)c3nc(-c4c[nH]cn4)oc3c2F)CO1]

To a solution of tert-butyl 5-(4-chloro-7-fluoro-2-(1-((2-(trimethylsilyl)ethoxy)methyl)-1H-imidazol-4-yl)benzo[d]oxazol-6-yl)-2,2-dimethyl-2,3-dihydro-4H-1,4-oxazine-4-carboxylate (830 mg, 1.43 mmol) in DCM (20 mL) was added a solution HCl in 1,4-dioxane (4 M/L, 10 mL). The mixture was stirred at rt for overnight. The solvent was removed in vacuo directly, and the crude product was redissolved in MeOH (50 mL). The mixture was cooled to 0° C. before NaBH4 (100 mg, 2.63 mmol) was added portion wise. The mixture was stirred at 0° C. for 1 h. Upon completion of the reaction, the mixture was used in next step directly. MS (ESI) m/z [M+H]⁺=351.

### Step 6: 1-(5-(4-chloro-7-fluoro-2-(1H-imidazol-4-yl)benzo[d]oxazol-6-yl)-2,2-dimethylmorpholino)prop-2-en-1-one

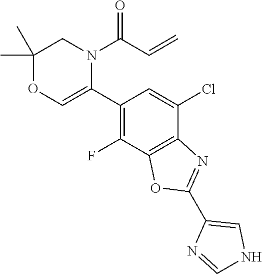 [SMILES: C=CC(=O)N1CC(C)(C)OC=C1c1cc(Cl)c2nc(-c3c[nH]cn3)oc2c1F]

To a solution of 4-chloro-6-(6,6-dimethylmorphinan-3-yl)-7-fluoro-2-(1H-imidazol-4-yl)benzo[d]oxazole above (ca.1.43 mmol) in MeOH (50 mL) and H₂O (30 mL) was added NaHCO₃ (400 mg, 4.76 mmol). The mixture was cooled to 0° C. before acryloyl chloride (150 mg, 1.66 mmol) was added dropwise. The mixture was stirred at 0° C. for 1 h and the resulting mixture was extracted with DCM. The organic layer was dried over anhydrous Na₂SO₄ and concentrated in vacuo. The residue was purified by silica gel column chromatography (DCM/MeOH 20/1 to 10/1) to give crude product. The crude product was further purified by C18 column (mobile phases, ACN in water (0.1% FA), 20% to 40%). MS (ESI) m/z [M+H]⁺=405.

### Step 7: (R)-1-(5-(4-chloro-7-fluoro-2-(1H-imidazol-4-yl)benzo[d]oxazol-6-yl)-2,2-dimethylmorpholino)prop-2-en-1-one (L13A, Assumed) and (S)-1-(5-(4-chloro-7-fluoro-2-(1H-imidazol-4-yl)benzo[d]oxazol-6-yl)-2,2-dimethylmorpholino)prop-2-en-1-one (L13B, Assumed)

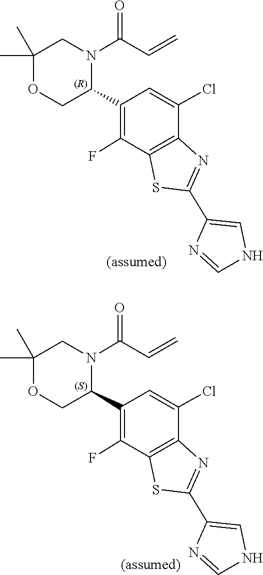

The racemic 1-(5-(4-chloro-7-fluoro-2-(1H-imidazol-4-yl)benzo[d]oxazol-6-yl)-2,2-dimethylmorpholino)prop-2-en-1-one was separated using the following conditions:

Column: CHIRAL ART Amylose-SB, 30 mm×250 mm, 5 um; fluent: mobile phase A is hexane (0.2% DEA), mobile phase B is EtOH (70% to 30%); isomer 1, retention time=6.5 min; isomer 2, retention time=8.0 min.

Isomer 1: (4.9 mg, 0.82% yield over 3 steps).

¹H NMR (600 MHz, DMSO-d₆) δ 12.94 (s, 1H), 8.25 (s, 1H), 7.97 (s, 1H), 7.58 (s, 1H), 6.90-6.76 (m, 1H), 6.19 (d, J=16.4 Hz, 1H), 5.74 (d, J=10.3 Hz, 1H), 5.68-5.51 (m, 1H), 4.08-3.84 (m, 3H), 3.48-3.16 (m, 1H), 1.20 (s, 3H), 1.14 (s, 3H). MS (ESI) m/z [M+H]⁺=405.

Isomer 2: (4.95 mg, 0.85% yield over 3 steps).

¹H NMR (600 MHz, DMSO-d₆) δ 12.94 (s, 1H), 8.26 (s, 1H), 7.98 (s, 1H), 7.59 (s, 1H), 6.90-6.76 (m, 1H), 6.20 (d, J=16.4 Hz, 1H), 5.74 (d, J=10.4 Hz, 1H), 5.68-5.51 (m, 1H), 4.08-3.84 (m, 3H), 3.48-3.16 (m, 1H), 1.20 (s, 3H), 1.14 (s, 3H). MS (ESI) m/z [M+H]⁺=405.

Compound L14 in table 2-13 was synthesized from corresponding starting materials using procedure in Example 2-32.

TABLE 2-13

| Compound | Structure | Chemical Name | ¹H NMR data LC/MS m/z (M + H) |
|---|---|---|---|
| L14 | 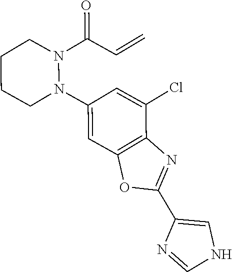 [SMILES: C=CC(=O)N1CCCCN1c1cc(Cl)c2nc(-c3c[nH]cn3)oc2c1] | 1-(2-(4-chloro-2-(1H- imidazol-4- yl)benzo[d]oxazol-6- yl)tetrahydropyridazin- 1(2H)-yl)prop-2-en-1- one | ¹H NMR (600 MHz, DMSO-d₆) δ 12.80 (s, 1H), 8.06 (s, 1H), 7.93 (s, 1H), 7.25 (s, 1H), 6.91 (s, 1H), 6.69 (dd, J = 17.1, 10.4 Hz, 1H), 6.29 (d, J = 17.0 Hz, 1H), 5.75 (d, J = 10.3 Hz, 1H), 4.33 (t, J = 14.8 Hz, 2H), 3.24-3.12 (m, 1H), 2.91- 2.75 (m, 1H), 1.74-1.46 (m, 4H). MS (ESI) m/z [M + H]⁺ = 358. |

### Example 2-35: Synthesis of (S)-1-(5-(4-chloro-2-(1H-imidazol-4-yl)benzo[d]thiazol-6-yl)-2,2-dimethylmorpholino)prop-2-en-1-one (N2B, Assumed) and (R)-1-(5-(4-chloro-2-(1H-imidazol-4-yl)benzo[d]thiazol-6-yl)-2,2-dimethylmorpholino)prop-2-en-1-one (N2A, Assumed)

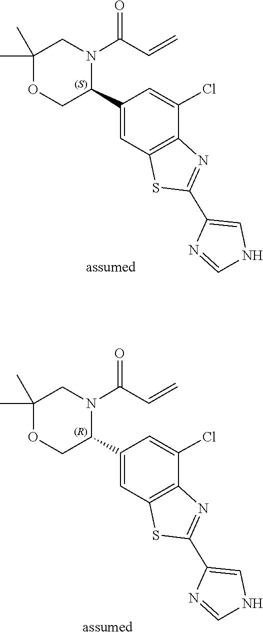

The racemic 1-(5-(4-chloro-2-(1H-imidazol-4-yl)benzo[d]thiazol-6-yl)-2,2-dimethylmorpholino)prop-2-en-1-one was synthesized following the procedure of Example 2-27. The racemic title compound was separated using the following conditions: Column: Lux Cellulose-4; 38% MeOH (0.3% 2M NH₃/MeOH)/CO₂; isomer 1, retention time=4.051 min; isomer 2, retention time=4.458 min.

Isomer 1:

¹H NMR (600 MHz, DMSO-d₆) δ 12.79 (s, 1H), 8.04 (s, 1H), 8.02 (s, 1H), 7.88 (s, 1H), 7.52 (s, 1H), 6.96-6.68 (m, 1H), 6.24 (d, J=16.7 Hz, 1H), 5.76 (s, 1H), 5.63-5.30 (m, 1H), 4.32-4.08 (m, 1H), 4.04-3.98 (m, 1H), 3.96-3.42 (m, 1H), 3.20-2.84 (m, 1H), 1.17 (s, 3H), 1.14 (s, 3H). MS (ESI) m/z [M+H]⁺=403.

Isomer 2:

¹H NMR (600 MHz, DMSO-d₆) δ 12.79 (s, 1H), 8.04 (s, 1H), 8.02 (s, 1H), 7.88 (s, 1H), 7.52 (s, 1H), 6.98-6.64 (m, 1H), 6.24 (d, J=16.4 Hz, 1H), 5.76 (s, 1H), 5.60-5.31 (m, 1H), 4.31-4.10 (m, 1H), 4.05-3.95 (m, 1H), 3.92-3.74 (m, 1H), 3.21-2.85 (m, 1H), 1.17 (s, 3H), 1.14 (s, 3H). MS (ESI) m/z [M+H]⁺=403.

### Example 2-36: Synthesis of (S)-1-(6-(4-chloro-2-(1H-imidazol-4-yl)benzo[d]thiazol-6-yl)-4-oxa-7-azaspiro[2.5]octan-7-yl)prop-2-en-1-one (N3B, Assumed) and (R)-1-(6-(4-chloro-2-(1H-imidazol-4-yl)benzo[d]thiazol-6-yl)-4-oxa-7-azaspiro[2.5]octan-7-yl)prop-2-en-1-one (N3A, Assumed)

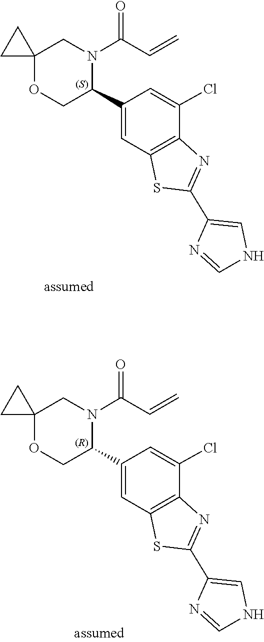

The racemic 1-(6-(4-chloro-2-(1H-imidazol-4-yl)benzo[d]thiazol-6-yl)-4-oxa-7-azaspiro[2.5]octan-7-yl)prop-2-en-1-one was synthesized following the procedure of Example 2-27. The racemic title compound was separated using the following conditions: SFC: column: Lux Cellulose-4; fluent: 45% MeOH (0.3% 2M NH₃/MeOH)/CO₂; isomer 1, retention time=2.340 min; isomer 2, retention time=3.372 min.

Isomer 1:

¹H NMR (600 MHz, DMSO-d₆) δ 12.80 (s, 1H), 8.08 (s, 1H), 8.03 (s, 1H), 7.88 (s, 1H), 7.55 (s, 1H), 6.93-6.78 (m, 1H), 6.26 (d, J=16.3 Hz, 1H), 5.77 (d, J=10.1 Hz, 1H), 5.75-5.43 (m, 1H), 4.46 (d, J=11.6 Hz, 1H), 4.03-3.88 (m, 1H), 3.83-3.68 (m, 1H), 3.55-3.49 (m, 1H), 0.89-0.81 (m, 1H), 0.75-0.65 (m, 2H), 0.61-0.44 (m, 1H). MS (ESI) m/z [M+H]⁺=401.

Isomer 2:

¹H NMR (600 MHz, DMSO-d₆) δ 12.80 (s, 1H), 8.08 (s, 1H), 8.03 (s, 1H), 7.88 (s, 1H), 7.55 (s, 1H), 6.86 (brs, 1H), 6.26 (d, J=16.6 Hz, 1H), 5.77 (d, J=10.4 Hz, 1H), 5.74-5.40 (m, 1H), 4.46 (d, J=12.1 Hz, 1H), 4.00-3.88 (m, 1H), 3.82-3.64 (m, 1H), 3.54-3.44 (m, 1H), 0.88-0.81 (m, 1H), 0.75-0.65 (m, 2H), 0.59-0.44 (m, 1H). MS (ESI) m/z [M+H]⁺=401.

### Example 2-37: Synthesis of (R)-1-(3-(4-chloro-7-fluoro-2-(1H-imidazol-4-yl)benzo[d]thiazol-6-yl)morpholino)prop-2-en-1-one (N5A, Assumed) and (S)-1-(3-(4-chloro-7-fluoro-2-(1H-imidazol-4-yl)benzo[d]thiazol-6-yl)morpholino)prop-2-en-1-one (N5B, Assumed)

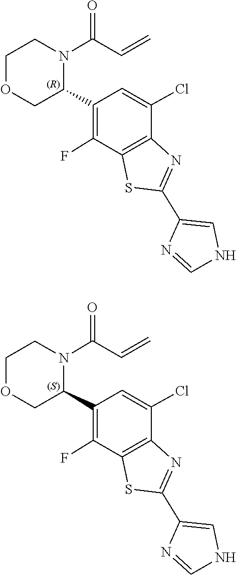

### Step 1: 2-chloro-5-fluoro-4-(4,4,5,5-tetramethyl-1,3,2-dioxaborolan-2-yl)aniline

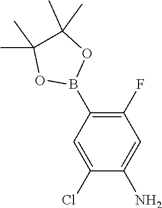 [SMILES: CC1(C)OB(c2cc(Cl)c(N)cc2F)OC1(C)C]

A mixture of 4-bromo-2-chloro-5-fluoro-aniline (5.0 g, 22.28 mmol), 4,4,5,5-tetramethyl-2-(4,4,5,5-tetramethyl-1,3,2-dioxaborolan-2-yl)-1,3,2-dioxaborolane (6.8 g, 26.73 mmol), Pd(dppf)Cl₂ (1.6 g, 2.22 mmol) and KOAc (6.5 g, 66.83 mmol) in 1,4-dioxane (100 mL) was stirred at 110° C. for 3 h under N₂ atmosphere. Upon completion of the reaction, the mixture was concentrated in vacuo, and the residue was purified by silica gel column chromatography (PE/EA=4/1) to give the title compound (4.3 g, 71% yield). MS (ESI) m/z [M+H]⁺=272.

### Step 2: tert-butyl 5-(4-amino-5-chloro-2-fluorophenyl)-2,3-dihydro-4H-1,4-oxazine-4-carboxylate

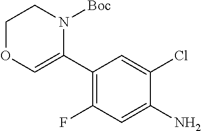 [SMILES: CC(C)(C)OC(=O)N1CCOC=C1c1cc(Cl)c(N)cc1F]

A mixture of 2-chloro-5-fluoro-4-(4,4,5,5-tetramethyl-1,3,2-dioxaborolan-2-yl)aniline (4.3 g, 16 mmol), tert-butyl 5-diphenoxyphosphoryloxy-2,3-dihydro-1,4-oxazine-4-carboxylate (6.9 g, 16 mmol), Pd(dppf)Cl₂ (1.2 g, 1.6 mmol) and Na₂CO₃ (3.4 g, 32 mmol) in 1,4-dioxane (100 mL) and H₂O (25 mL) was degassed with N₂ for 3 times. The mixture was then stirred at 90° C. for 2 h under N₂ atmosphere. Upon completion of the reaction, the mixture was cooled to rt and extracted with EA. The combined organic layer was dried over anhydrous Na₂SO₄. The solvent was removed in vacuo and the residue was purified by silica gel column chromatography (PE/EA from 100/1 to 3/2) to give the title compound (4.3 g, 83% yield). MS (ESI) m/z [M+H−56]⁺=273.

### Step 3: 2-chloro-4-(5,6-dihydro-2H-1,4-oxazin-3-yl)-5-fluoroaniline

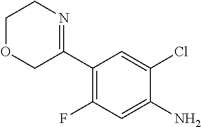 [SMILES: Nc1cc(F)c(C2=NCCOC2)cc1Cl]

To a solution of tert-butyl 5-(4-amino-5-chloro-2-fluoro-phenyl)-2,3-dihydro-1,4-oxazine-4-carboxylate (4.3 g, 13.1 mmol) in DCM (40 mL) was added TFA (6 mL) at rt. After stirring at rt for 12 h, the solvent was concentrated in vacuo. The residue was used in next step without purification (ca. 13.12 mmol). MS (ESI) m/z [M+H]⁺=229.

### Step 4: 2-chloro-5-fluoro-4-(morpholin-3-yl)aniline

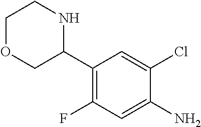 [SMILES: Nc1cc(F)c(C2COCCN2)cc1Cl]

To a solution of crude 2-chloro-4-(3,6-dihydro-2H-1,4-oxazin-5-yl)-5-fluoro-aniline (ca.13.12 mmol) in MeOH (40 mL) was added NaBH₃CN (0.99 g, 15.74 mmol) in portion at rt. The reaction mixture was stirred at rt for 1 h. Upon completion of the reaction, the mixture was concentrated in vacuo, and the residue was used for next step without further purification. MS (ESI) m/z [M+H]⁺=231.

### Step 5: tert-butyl 3-(4-amino-5-chloro-2-fluorophenyl)morpholine-4-carboxylate

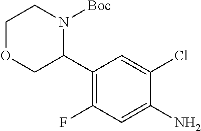 [SMILES: CC(C)(C)OC(=O)N1CCOCC1c1cc(Cl)c(N)cc1F]

To a solution of crude 2-chloro-5-fluoro-4-(morpholin-3-yl)aniline (ca. 13.12 mmol) in 1,4-dioxane (40 mL) and sat. aq NaHCO₃ (40 mL) was added (Boc)₂O (3.15 g, 14.43 mmol) dropwise at rt. The mixture was stirred at rt for 2 h. Upon completion of the reaction, the mixture was extracted with EA. The organic layer was dried over anhydrous Na₂SO₄. The solvent was removed in vacuo, and the residue was purified by silica gel column chromatography (PE/EA=4/1) to give the title compound (3.64 g, 69% yield over 4 steps). MS (ESI) m/z [M+Na]⁺=353.

### Step 6: tert-butyl 3-(2-amino-4-chloro-7-fluorobenzo[d]thiazol-6-yl)morpholine-4-carboxylate

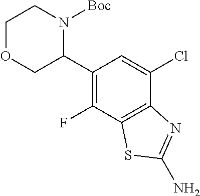 [SMILES: CC(C)(C)OC(=O)N1CCOCC1c1cc(Cl)c2nc(N)sc2c1F]

To a solution of tert-butyl 3-(4-amino-5-chloro-2-fluoro-phenyl)morpholine-4-carboxylate (3.64 g, 11.0 mmol) in AcOH (50 mL) was added KSCN (4.28 g, 44.0 mmol) at rt. To this mixture was added a solution of Br₂ (3.52 g, 22.0 mmol) in acetic acid (5 mL) (ca. 5 min) dropwise at rt. The reaction mixture was stirred at rt for 12 h. Upon completion of the reaction, the mixture was quenched with aq. Na₂S₂O₃ and extracted with EA. The organic layer was combined, dried over anhydrous Na₂SO₄. The solvent was removed in vacuo, and the residue was purified by silica gel column chromatography (PE/EA=1/5) to give the title compound (1.96 g, 45.9% yield). MS (ESI) m/z [M+H]⁺=388.

### Step 7: tert-butyl 3-(2-bromo-4-chloro-7-fluorobenzo[d]thiazol-6-yl)morpholine-4-carboxylate

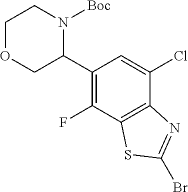 [SMILES: CC(C)(C)OC(=O)N1CCOCC1c1cc(Cl)c2nc(Br)sc2c1F]

A solution of tert-butyl 3-(2-amino-4-chloro-7-fluoro-1,3-benzothiazol-6-yl)morpholine-4-carboxylate (1.36 g, 3.51 mmol), NaNO₂ (0.73 g, 10.52 mmol) and NBS (1.24 g, 7.01 mmol) in DMF (20 mL) was stirred at rt for 3 h. Upon completion of the reaction, the mixture was quenched with aq. Na₂S₂O₃ and extracted with EA. The organic layer was separated and dried over anhydrous Na₂SO₄. The solvent was removed, and the residue was purified by silica gel column chromatography (PE/EA=4/1) to give the title compound (0.52 g, 32.8% yield). MS (ESI) m/z [M+H]⁺=451, 453.

### Step 8: tert-butyl 3-(4-chloro-7-fluoro-2-(1-(tetrahydro-2H-pyran-2-yl)-1H-imidazol-4-yl)benzo[d]thiazol-6-yl)morpholine-4-carboxylate

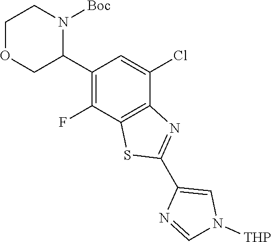

A mixture of tert-butyl 3-(2-bromo-4-chloro-7-fluoro-1,3-benzothiazol-6-yl)morpholine-4-carboxylate (588 mg, 1.30 mmol), 1-tetrahydropyran-2-yl-5-(4,4,5,5-tetramethyl-1,3,2-dioxaborolan-2-yl)imidazole (724 mg, 2.60 mmol), Pd(dppf)Cl₂ (95 mg, 0.13 mmol) and K₂CO₃ (359 mg, 2.60 mmol) in 1,4-dioxane (20 mL) and H₂O (5 mL) was stirred at 90° C. for 2 h under N₂ atmosphere. Upon completion of the reaction, the mixture was cooled to rt and extracted with EA. The combined organic layer was dried over anhydrous Na₂SO₄. The solvent was removed in vacuo, and the residue was purified by silica gel column chromatography (PE/EA from 100/1 to 3/2) to give the title compound (522 mg, 76.7% yield). MS (ESI) m/z [M+Na]⁺=545.

### Step 9: 3-(4-chloro-7-fluoro-2-(1H-imidazol-4-yl)benzo[d]thiazol-6-yl)morpholine

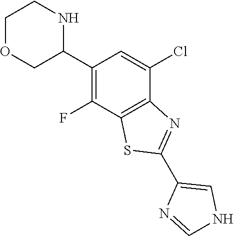 [SMILES: Fc1c(C2COCCN2)cc(Cl)c2nc(-c3c[nH]cn3)sc12]

To a solution of tert-butyl 3-[4-chloro-7-fluoro-2-(1-tetrahydropyran-2-ylimidazol-4-yl)-1,3-benzothiazol-6-yl]morpholine-4-carboxylate (610 mg, 1.17 mmol) in DCM (6 mL) was added TFA (2 mL) at rt. After stirring at rt for 2 h, the reaction mixture was concentrated, and the residue was used for next step without purification. MS (ESI) m/z [M+H]⁺=339.

### Step 10: 1-(3-(4-chloro-7-fluoro-2-(1H-imidazol-4-yl)benzo[d]thiazol-6-yl)morpholino)prop-2-en-1-one

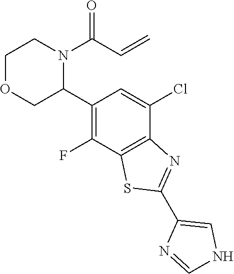 [SMILES: C=CC(=O)N1CCOCC1c1cc(Cl)c2nc(-c3c[nH]cn3)sc2c1F]

To a solution of 3-[4-chloro-7-fluoro-2-(1H-imidazol-4-yl)-1,3-benzothiazol-6-yl]morpholine (395 mg, 1.17 mmol) in MeOH (5 mL) and sat. aq. NaHCO₃ (5 mL) was added dropwise a solution of acryloyl chloride in DCM (10 mg/mL, 10.6 mL) at 0° C. and the resulting mixture was stirred at this temperature for 1 h. Upon completion of the reaction, the mixture was quenched by H₂O and extracted with DCM. The combined organic layer was dried over anhydrous Na₂SO₄ and the solvent was removed in vacuo. The residue was purified by C18 column (mobile phase, ACN in water (0.1% FA), 60%) to give the title compound (194 mg, 42% over 2 steps). MS (ESI) m/z [M+H]⁺=393.

### Step 11: (R)-1-(3-(4-chloro-7-fluoro-2-(1H-imidazol-4-yl)benzo[d]thiazol-6-yl)morpholino)prop-2-en-1-one (N5A, Assumed) and (S)-1-(3-(4-chloro-7-fluoro-2-(1H-imidazol-4-yl)benzo[d]thiazol-6-yl)morpholino)prop-2-en-1-one (N5B, Assumed)

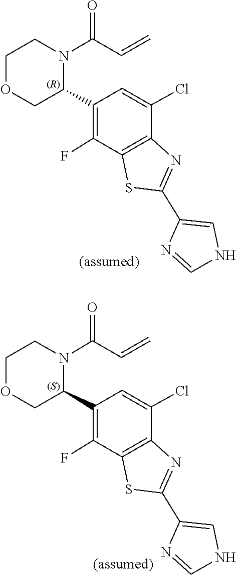

Racemic 1-(3-(4-chloro-7-fluoro-2-(1H-imidazol-4-yl)benzo[d]thiazol-6-yl)morpholino)prop-2-en-1-one was separated using the following conditions:

Chiral column: CHIRAL ART Cellulose-C; Mobile phase A is Hexane, mobile phase B is EtOH, fluent: A/B 70/30 (v/v); isomer 1, retention time=6.5 min; isomer 2, retention time=8.0 min.

Isomer 1: (74.39 mg, 38.3% yield).

¹H NMR (600 MHz, DMSO-d₆) δ 12.89 (s, 1H), 8.11 (s, 1H), 7.92 (s, 1H), 7.74 (d, J=6.1 Hz, 1H), 6.88 (s, 1H), 6.20 (d, J=16.5 Hz, 1H), 5.76 (d, J=10.4 Hz, 1H), 5.74-5.57 (m, 1H), 4.37-4.28 (m, 1H), 4.26-4.00 (m, 1H), 4.00-3.95 (m, 1H), 3.91-3.81 (m, 1H), 3.55 (t, J=10.5 Hz, 1H), 3.30-2.95 (m, 1H). MS (ESI) m/z [M+H]⁺=393.

Isomer 2: (78.85 mg, 40.6% yield).

¹H NMR (600 MHz, DMSO-d₆) δ 12.89 (s, 1H), 8.11 (s, 1H), 7.92 (s, 1H), 7.74 (d, J=6.1 Hz, 1H), 6.88 (s, 1H), 6.20 (d, J=16.5 Hz, 1H), 5.76 (d, J=10.4 Hz, 1H), 5.74-5.52 (m, 1H), 4.40-4.29 (m, 1H), 4.26-4.00 (m, 1H), 4.00-3.94 (m, 1H), 3.90-3.81 (m, 1H), 3.55 (t, J=10.5 Hz, 1H), 3.30-2.94 (m, 1H). MS (ESI) m/z [M+H]⁺=393.

Compound N9 in table 2-14 was synthesized from corresponding starting materials using procedure in example 2-32.

TABLE 2-14

| Compound | Structure | Chemical Name | ¹H NMR data LC/MS m/z (M + H) |
|---|---|---|---|
| N9 | 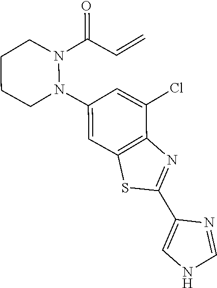 [SMILES: C=CC(=O)N1CCCCN1c1cc(Cl)c2nc(-c3c[nH]cn3)sc2c1] | 1-(2-(4-chloro-2-(1H- imidazol-4- yl)benzo[d]thiazol-6- yl)tetrahydropyridazin- 1(2H)-yl)prop-2-en- 1-one | ¹H NMR (600 MHz, DMSO-d₆) δ 12.73 (s, 1H), 7.95 (s, 1H), 7.85 (s, 1H), 7.56 (d, J = 1.7 Hz, 1H), 7.07 (d, J = 1.7 Hz, 1H), 6.70 (dd, J = 17.1, 10.4 Hz, 1H), 6.29 (dd, J = 17.1, 2.2 Hz, 1H), 5.75 (dd, J = 10.4, 2.2 Hz, 1H), 4.35 (d, J = 13.2 Hz, 1H), 4.29 (d, J = 14.4 Hz, 1H), 3.23-3.16 (m, 1H), 2.89-2.78 (m, 1H), 1.74-1.62 (m, 3H), 1.60-1.50 (m, 1H). MS (ESI) m/z [M + H]⁺ = 374. |
| N10 | 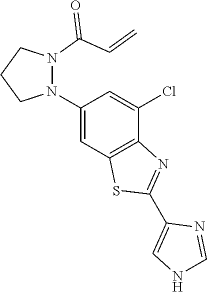 [SMILES: C=CC(=O)N1CCCN1c1cc(Cl)c2nc(-c3c[nH]cn3)sc2c1] | 1-(2-(4-chloro-2-(1H- imidazol-4- yl)benzo[d]thiazol-6- yl)pyrazolidin-1- yl)prop-2-en-1-one | 1H NMR (600 MHz, DMSO-d6) 8 12.74 (s, 1H), 7.96 (s, 1H), 7.85 (s, 1H), 7.66 (s, 1H), 7.25 (s, 1H), 6.62 (dd, J = 16.4, 10.6 Hz, 1H), 6.24 (dd, J = 17.1, 1.9 Hz, 1H), 5.68 (d, J = 9.7 Hz, 1H), 4.01-3.81 (m, 2H), 3.50-3.40 (m, 2H), 2.11-2.03 (m, 1H), 1.96- 1.83 (m, 1H). MS (ESI) m/z [M + H]⁺ = 360. |

### Example 3

### Biological Assay

### Example 3-1: KEAP1 and CUL3 Biochemical Assay

Protein-protein interactions (PPI) between recombinant KEAP1 and CUL3 were performed using Homogeneous Time Resolved Fluorescence (HTRF) assay, which measures the binding activity of the Keap1 and CUL3 proteins. Recombinant human KEAP1 protein (1-624, Full-length) was expressed in sf9 cell using a baculovirus expression system. Recombinant human CUL3 protein (1-388) was expressed in sf9 cell using a baculovirus expression system.

Testing for the enhancement of binding of KEAP1 to CUL3 by various compounds disclosed herein was carried out at rt in assay buffer containing 10 mM HEPES Ph7.4, 150 mM NaCl, 0.5 mM EDTA, 1 mM DTT and 0.005% Tween-20. Compounds in DMSO were dispensed into wells of a black, 384-well plate (Corning 4514) using ECH0555 (Beckman). The final concentration ranges of the test compounds were 0.25-5,000 nM or 2.5-50,000 nM. Five μL of KEAP1 protein solution was added to wells, and the plate was incubated for 1 hour at rt (RT). After incubation for 1 hour at RT, 5 μL CUL3 solution was added to the wells to initiate reaction, and the plate was incubated. After 1 hour reaction, 5 μL detection reagent were added to the wells, and the plate was incubated for 1 hour. Detection reagent containing 0.025 test/μL Streptavidin-XL665 (PerkinElmer #610SAXLG), 0.025 test/μL Mab Anti 6HIS-Tb (PerkinElmer #61HI2TLB). HTRF was measured on a microplate reader (PHERAstar FSX, BMG labtech). The EC50s were calculated based on activation of proteins interactions in the presence of increasing concentrations of test compounds, and the results were shown in Table 3.

Table 3 below describes the binding activity in the form of EC₅₀ for compounds tested. “A” refers to an EC₅₀<10 nM, “B” means 10 nM≤EC₅₀≤20 nM, “C” means 20 nM≤EC₅₀≤50 nM, and “D” means 50 nM≤EC₅₀≤1 μM. “AA1” refers to 500<Emax %≤1000, “BB1” means 400≤Emax %≤500, “CC1” means 300<Emax %<400, “DD1” means 200≤Emax %≤300

TABLE 3
EC₅₀ values of compounds in biochemical
KEAP1-CUL3 binding assays

| | KEAP1- | | | KEAP1- | |
|---|---|---|---|---|---|
| | CUL3 | | | CUL3 | |
| | binding | Emax | | binding | Emax |
| Compound | EC50, nM | % | Compound | EC50, nM | % |
| B1 | A | BB1 | B2 | A | AA1 |
| B3 | A | BB1 | B4 | A | BB1 |
| B5 | A | BB1 | B6 | A | BB1 |
| B7 | A | AA1 | B7A | A | AA1 |
| B10 | A | CC1 | B15A | A | BB1 |
| B15B | B | BB1 | B16A | A | AA1 |
| B16B | A | AA1 | B17 | A | BB1 |
| B18 | A | BB1 | B26A | A | AA1 |
| B26B | A | BB1 | B27A | A | CC1 |
| B27B | B | CC1 | B29A | A | AA1 |
| B29B | D | BB1 | B32A | A | AA1 |
| B32B | D | CC1 | B33 | A | BB1 |
| B34 | A | BB1 | B35 | A | BB1 |
| B36 | A | BB1 | C1 | A | BB1 |
| B38 | B | BB1 | B39 | A | AA1 |
| B40 | A | BB1 | B41 | B | BB1 |
| B42 | A | BB1 | B43 | A | AA1 |
| C2 | A | BB1 | D1 | A | CC1 |
| D2 | A | CC1 | D11 | A | BB1 |
| D7A | A | BB1 | D7B | A | BB1 |
| D15A | A | BB1 | D15B | D | CC1 |
| D16 | A | AA1 | D17 | A | BB1 |
| D18A | A | BB1 | D18B | A | CC1 |
| D22A | A | AA1 | D22B | A | BB1 |
| D12 | A | AA1 | D13 | A | BB1 |
| D14 | A | BB1 | K1 | D | CC1 |
| D24A | A | AA1 | D24B | D | DD1 |
| D25 | A | AA1 | D26 | A | AA1 |
| D27 | A | AA1 | H1 | C | BB1 |
| D28A | A | AA1 | D28B | D | CC1 |
| D29A | A | AA1 | D29B | D | BB1 |
| D30 | A | AA1 | D31 | A | AA1 |
| D32A | A | BB1 | D32B | D | BB1 |
| D33 | A | AA1 | D34 | A | AA1 |
| H2 | B | CC1 | H3 | A | AA1 |
| H4 | A | AA1 | L1A | A | AA1 |
| L1B | A | BB1 | L2 | A | AA1 |
| L2B | A | CC1 | L4A | A | BB1 |
| L4B | A | AA1 | L5A | A | AA1 |
| L5B | D | CC1 | L6 | A | AA1 |
| L7 | A | AA1 | L8 | A | AA1 |
| L9 | A | AA1 | L10 | A | AA1 |
| L11A | D | DD1 | L11B | A | AA1 |
| L12A | A | AA1 | L12B | D | BB1 |
| L13A | A | AA1 | L13B | A | AA1 |
| L14 | A | AA1 | N2A | A | AA1 |
| M1A | A | BB1 | M1B | A | DD1 |
| N1A | A | BB1 | N1B | A | AA1 |
| N2B | A | AA1 | N3A | A | AA1 |
| N3B | A | AA1 | N5A | A | AA1 |
| N5B | D | CC1 | | | |

We claim:

1. A compound selected from 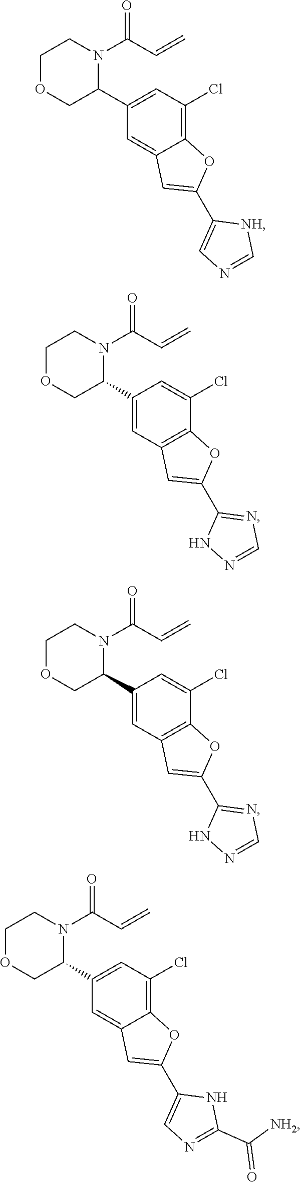 [SMILES: C=CC(=O)N1CCOCC1c1cc(Cl)c2oc(-c3cnc[nH]3)cc2c1 ; C=CC(=O)N1CCOC[C@H]1c1cc(Cl)c2oc(-c3ncn[nH]3)cc2c1 ; C=CC(=O)N1CCOC[C@@H]1c1cc(Cl)c2oc(-c3ncn[nH]3)cc2c1 ; C=CC(=O)N1CCOC[C@H]1c1cc(Cl)c2oc(-c3cnc(C(N)=O)[nH]3)cc2c1] 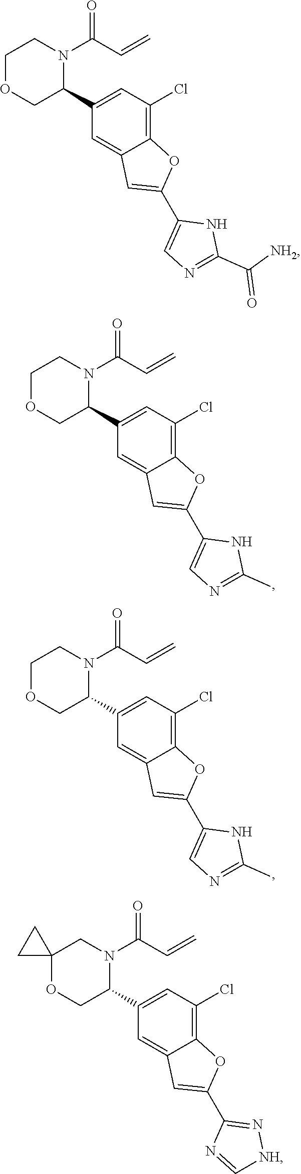 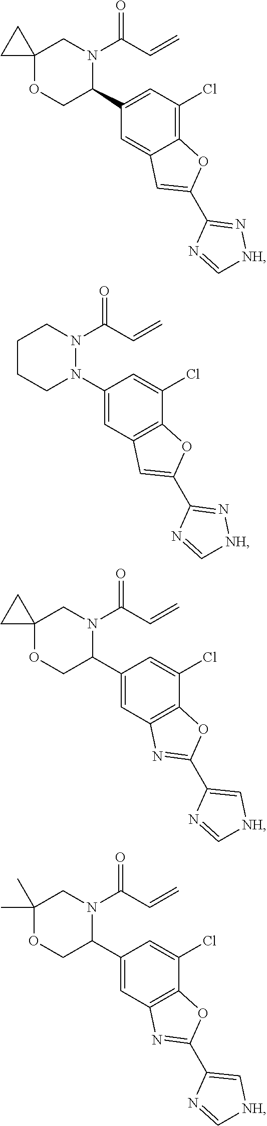 [SMILES: C=CC(=O)N1CC2(CC2)OC[C@@H]1c1cc(Cl)c2oc(-c3nc[nH]n3)cc2c1 ; C=CC(=O)N1CCCCN1c1cc(Cl)c2oc(-c3nc[nH]n3)cc2c1 ; C=CC(=O)N1CC2(CC2)OCC1c1cc(Cl)c2oc(-c3c[nH]cn3)nc2c1 ; C=CC(=O)N1CC(C)(C)OCC1c1cc(Cl)c2oc(-c3c[nH]cn3)nc2c1] 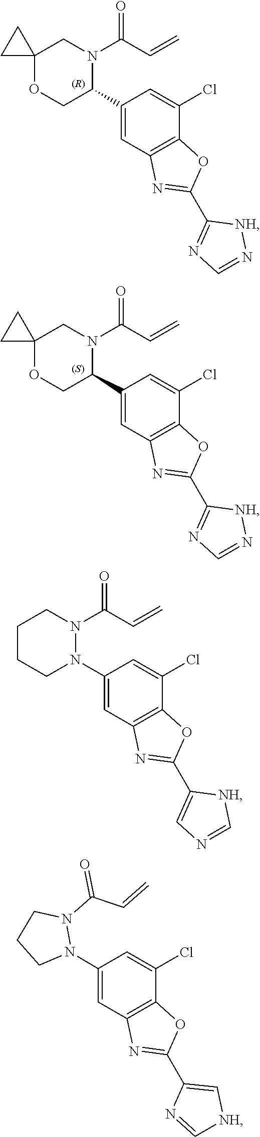 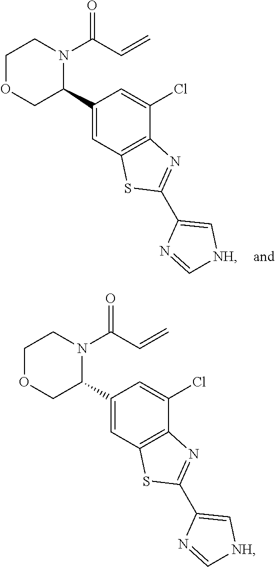 [SMILES: C=CC(=O)N1CCOC[C@@H]1c1cc(Cl)c2nc(-c3c[nH]cn3)sc2c1 ; C=CC(=O)N1CCOC[C@H]1c1cc(Cl)c2nc(-c3c[nH]cn3)sc2c1]
or a pharmaceutically acceptable salt thereof, or a hydrate thereof, or a solvate thereof.

2. The compound of claim 1, or a pharmaceutically acceptable salt thereof, or a hydrate thereof, or a solvate thereof, wherein the compound is

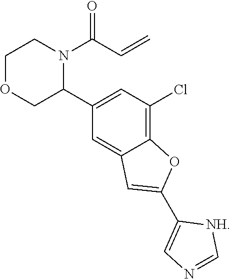 [SMILES: C=CC(=O)N1CCOCC1c1cc(Cl)c2oc(-c3cnc[nH]3)cc2c1]

3. The compound of claim 1, or a pharmaceutically acceptable salt thereof, or a hydrate thereof, or a solvate thereof, wherein the compound is

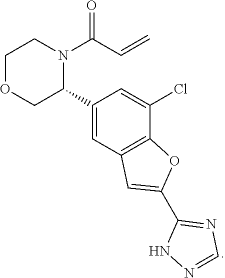 [SMILES: C=CC(=O)N1CCOC[C@H]1c1cc(Cl)c2oc(-c3ncn[nH]3)cc2c1]

4. The compound of claim 1, or a pharmaceutically acceptable salt thereof, or a hydrate thereof, or a solvate thereof, wherein the compound is

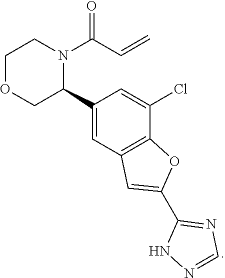 [SMILES: C=CC(=O)N1CCOC[C@@H]1c1cc(Cl)c2oc(-c3ncn[nH]3)cc2c1]

5. The compound of claim 1, or a pharmaceutically acceptable salt thereof, or a hydrate thereof, or a solvate thereof, wherein the compound is

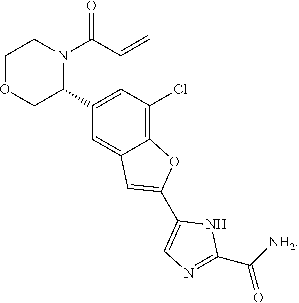 [SMILES: C=CC(=O)N1CCOC[C@H]1c1cc(Cl)c2oc(-c3cnc(C(N)=O)[nH]3)cc2c1]

6. The compound of claim 1, or a pharmaceutically acceptable salt thereof, or a hydrate thereof, or a solvate thereof, wherein the compound is

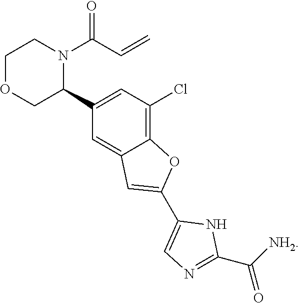 [SMILES: C=CC(=O)N1CCOC[C@@H]1c1cc(Cl)c2oc(-c3cnc(C(N)=O)[nH]3)cc2c1]

7. The compound of claim 1, or a pharmaceutically acceptable salt thereof, or a hydrate thereof, or a solvate thereof, wherein the compound is

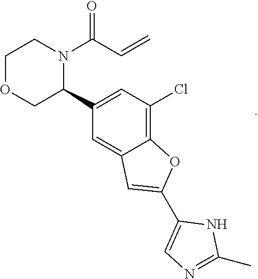 [SMILES: C=CC(=O)N1CCOC[C@@H]1c1cc(Cl)c2oc(-c3cnc(C)[nH]3)cc2c1]

8. The compound of claim 1, or a pharmaceutically acceptable salt thereof, or a hydrate thereof, or a solvate thereof, wherein the compound is

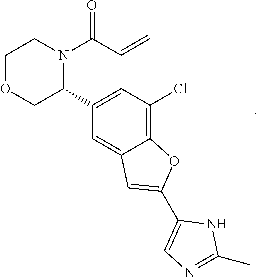 [SMILES: C=CC(=O)N1CCOC[C@H]1c1cc(Cl)c2oc(-c3cnc(C)[nH]3)cc2c1]

9. The compound of claim 1, or a pharmaceutically acceptable salt thereof, or a hydrate thereof, or a solvate thereof, wherein the compound is

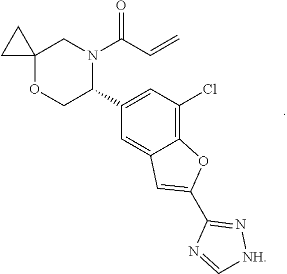 [SMILES: C=CC(=O)N1CC2(CC2)OC[C@H]1c1cc(Cl)c2oc(-c3nc[nH]n3)cc2c1]

10. The compound of claim 1, or a pharmaceutically acceptable salt thereof, or a hydrate thereof, or a solvate thereof, wherein the compound is

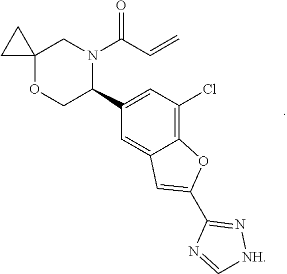 [SMILES: C=CC(=O)N1CC2(CC2)OC[C@@H]1c1cc(Cl)c2oc(-c3nc[nH]n3)cc2c1]

11. The compound of claim 1, or a pharmaceutically acceptable salt thereof, or a hydrate thereof, or a solvate thereof, wherein the compound is

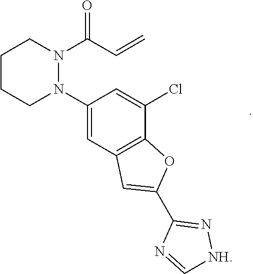 [SMILES: C=CC(=O)N1CCCCN1c1cc(Cl)c2oc(-c3nc[nH]n3)cc2c1]

12. The compound of claim 1, or a pharmaceutically acceptable salt thereof, or a hydrate thereof, or a solvate thereof, wherein the compound is

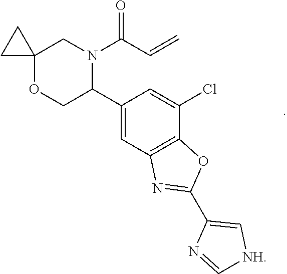 [SMILES: C=CC(=O)N1CC2(CC2)OCC1c1cc(Cl)c2oc(-c3c[nH]cn3)nc2c1]

13. The compound of claim 1, or a pharmaceutically acceptable salt thereof, or a hydrate thereof, or a solvate thereof, wherein the compound is

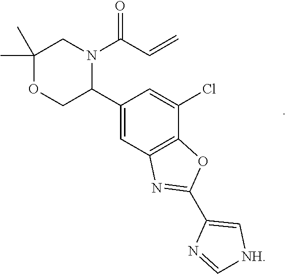 [SMILES: C=CC(=O)N1CC(C)(C)OCC1c1cc(Cl)c2oc(-c3c[nH]cn3)nc2c1]

14. The compound of claim 1, or a pharmaceutically acceptable salt thereof, or a hydrate thereof, or a solvate thereof, wherein the compound is

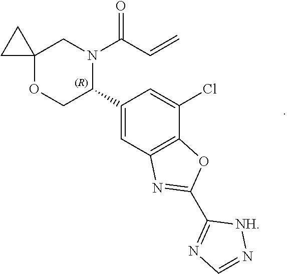

15. The compound of claim 1, or a pharmaceutically acceptable salt thereof, or a hydrate thereof, or a solvate thereof, wherein the compound is

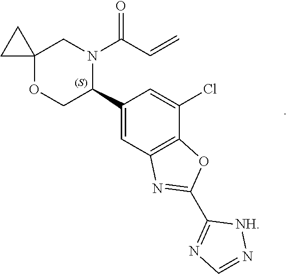

16. The compound of claim 1, or a pharmaceutically acceptable salt thereof, or a hydrate thereof, or a solvate thereof, wherein the compound is

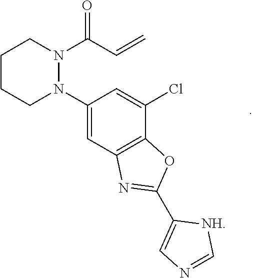 [SMILES: C=CC(=O)N1CCCCN1c1cc(Cl)c2oc(-c3cnc[nH]3)nc2c1]

17. The compound of claim 1, or a pharmaceutically acceptable salt thereof, or a hydrate thereof, or a solvate thereof, wherein the compound is

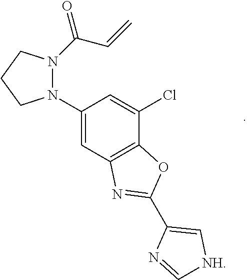 [SMILES: C=CC(=O)N1CCCN1c1cc(Cl)c2oc(-c3c[nH]cn3)nc2c1]

18. The compound of claim 1, or a pharmaceutically acceptable salt thereof, or a hydrate thereof, or a solvate thereof, wherein the compound is

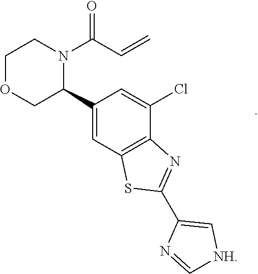 [SMILES: C=CC(=O)N1CCOC[C@@H]1c1cc(Cl)c2nc(-c3c[nH]cn3)sc2c1]

19. The compound of claim 1, or a pharmaceutically acceptable salt thereof, or a hydrate thereof, or a solvate thereof, wherein the compound is

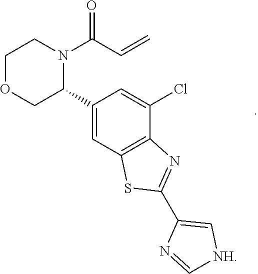 [SMILES: C=CC(=O)N1CCOC[C@H]1c1cc(Cl)c2nc(-c3c[nH]cn3)sc2c1]

20. A pharmaceutical composition, comprising a compound of claim 1, or a pharmaceutically acceptable salt thereof, or a hydrate thereof, or a solvate thereof, and a pharmaceutically acceptable excipient.
